(19) United States
(12) Patent Application Publication
(10) Pub. No.: US 2021/0005825 A1
(43) Pub. Date: Jan. 7, 2021

(54) ORGANIC ELECTROLUMINESCENT ELEMENT AND ELECTRONIC DEVICE

(71) Applicant: IDEMITSU KOSAN CO., LTD., Chiyoda-ku (JP)

(72) Inventors: Satomi TASAKI, Chiba-shi (JP); Ryota TAKAHASHI, Chiba-shi (JP); Yuki NAKANO, Kisarazu-shi (JP); Yuichiro KAWAMURA, Chiba-shi (JP)

(73) Assignee: IDEMITSU KOSAN CO., LTD., Chiyoda-ku (JP)

(21) Appl. No.: 16/499,939

(22) PCT Filed: Apr. 3, 2018
(86) PCT No.: PCT/JP2018/014202

§ 371 Date: Oct. 1, 2019

(30) Foreign Application Priority Data
Apr. 3, 2017 (JP) 2017-074065

(51) Int. Cl. H01L 51/00; C07D 487/06; C09K 11/06; C07D 487/22

(52) U.S. Cl. CPC H01L 51/0072; C07D 487/06; H01L 51/5012; C07D 487/22; H01L 51/0061; C09K 11/06

(57) ABSTRACT

An organic electroluminescence device comprising a cathode, an anode and an organic layer disposed between the cathode and the anode, wherein the organic layer comprises a fluorescent emitting layer and the fluorescent emitting layer comprises at least one first compound selected from the compounds represented by formulae (19), (21), (22), and (23), a second compound selected from the compounds represented by formula (3a), and a dopant material selected from the compounds represented by formulae (D1) and (D2) is excellent in its performance.

### TECHNICAL FIELD

The present invention relates to organic electroluminescence devices and electronic devices.

### BACKGROUND ART

An organic electroluminescence device (hereinafter may be simply referred to as “organic EL device”) generally comprises an anode, a cathode, and one or more organic thin film layers sandwiched between the anode and the cathode. When a voltage is applied between the electrodes, electrons from the cathode and holes from the anode are injected into a light emitting region. The injected electrons recombine with the injected holes in the light emitting region to form excited state. When the excited state returns to the ground state, the energy is released as light.

Many researches have been made on the applications of organic EL device to display, etc. because of its possibility of wide selection of emission colors by using various emitting materials in a light emitting layer. Particularly, the research on the emitting materials which emit three primary red, green, and blue colors and other materials for organic EL device have been made actively.

The materials for organic EL devices and organic EL devices have been proposed, for example, in Patent Literatures 1 to 7.

### CITATION LIST

### Patent Literature

Patent Literature 1: JP 2014-73965A
Patent Literature 2: WO 2016/006925
Patent Literature 3: CN 104119347B
Patent Literature 4: WO 2011/128017
Patent Literature 5: KR 10-2015-0135125B
Patent Literature 6: WO 2013/077344
Patent Literature 7: WO 2016/195441

### SUMMARY OF INVENTION

### Technical Problem

An object of the invention is to provide an organic EL device having a lifetime further improved.

### Solution to Problem

As a result of extensive research, the inventors have found that the above problem is solved by a light emitting layer comprising a specific dopant material, a specific material (first compound), and another specific material (second compound) structurally different from the first compound.
(1) In an aspect of the invention, provided is an organic electroluminescence device comprising a cathode, an anode and an organic layer disposed between the cathode and the anode, wherein the organic layer comprises a fluorescent emitting layer and the fluorescent emitting layer comprises:

at least one first compound selected from the compounds represented by formulae (19), (21), (22), and (23);

a second compound selected from the compound represented by formula (3a); and

a dopant material selected from the compounds represented by formulae (D1) and (D2):

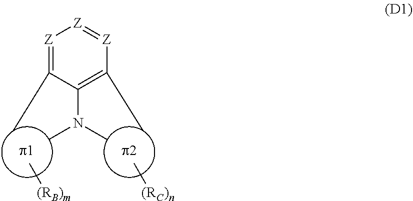

wherein:

Z is CR_{A} or N;

a ring π1 is a substituted or unsubstituted aromatic hydrocarbon ring having 6 to 50 ring carbon atoms or a substituted or unsubstituted aromatic heterocyclic ring having 5 to 50 ring atoms;

a ring π2 is a substituted or unsubstituted aromatic hydrocarbon ring having 6 to 50 ring carbon atoms or a substituted or unsubstituted aromatic heterocyclic ring having 5 to 50 ring atoms;

R_{A}, R_{B}, and R_{C} are each independently a hydrogen atom or a substituent, wherein the substituent is a halogen atom, a cyano group, a substituted or unsubstituted alkyl group having 1 to 20 carbon atoms, a substituted or unsubstituted alkenyl group having 1 to 20 carbon atoms, a substituted or unsubstituted alkynyl group having 1 to 20 carbon atoms, a substituted or unsubstituted cycloalkyl group having 3 to 20 ring carbon atoms, a substituted or unsubstituted alkoxy group having 1 to 20 carbon atoms, a substituted or unsubstituted aryloxy group having 6 to 50 ring carbon atoms, a substituted or unsubstituted alkylthio group having 1 to 20 carbon atoms, a substituted or unsubstituted arylthio group having 6 to 50 ring carbon atoms, a group represented by —Si(R₁₀₁)(R₁₀₂)(R₁₀₃), a group represented by —N(R₁₀₄)(R₁₀₅), a substituted or unsubstituted aryl group having 6 to 50 ring carbon atoms, or a substituted or unsubstituted heteroaryl group having 5 to 50 ring atoms;

R₁₀₁ to R₁₀₅ are each independently a hydrogen atom, a substituted or unsubstituted alkyl group having 1 to 20 carbon atoms, a substituted or unsubstituted cycloalkyl group having 3 to 20 ring carbon atoms, a substituted or unsubstituted aryl group having 6 to 50 ring carbon atoms, or a substituted or unsubstituted heteroaryl group having 5 to 50 ring atoms;

n and m are each independently an integer of 1 to 4;

adjacent two R_{A}'s are bonded to each other to form a substituted or unsubstituted ring structure or not bonded to each other, thereby failing to form a ring structure;

adjacent two R_{B}'s are bonded to each other to form a substituted or unsubstituted ring structure or not bonded to each other, thereby failing to form a ring structure;

adjacent two R_{C}'s are bonded to each other to form a substituted or unsubstituted ring structure or not bonded to each other, thereby failing to form a ring structure;

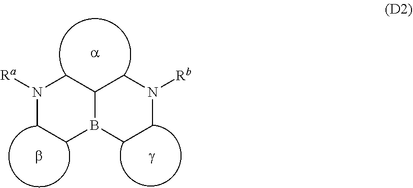

wherein:

a ring α, a ring β, and a ring γ are each independently a substituted or unsubstituted aromatic hydrocarbon ring having 6 to 50 ring carbon atoms [symbol] a substituted or unsubstituted aromatic heterocyclic ring having 5 to 50 ring atoms;

R^{a} and R^{b} are each independently a substituted or unsubstituted aryl group having 6 to 50 ring carbon atoms, a substituted or unsubstituted heteroaryl group having 5 to 50 ring atoms, or a substituted or unsubstituted alkyl group having 1 to 20 carbon atoms;

R^{a} may be bonded to one or both of the ring α and the ring β directly or via a linker;

R^{b} may be bonded to one or both of the ring α and the ring β directly or via a linker;

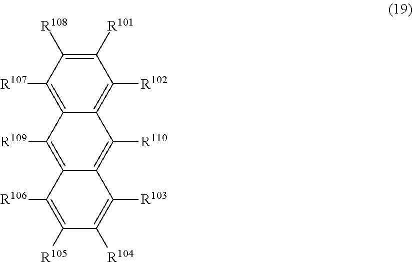

wherein:

R¹⁰¹ to R¹¹⁰ are each independently a hydrogen atom or a substituent, wherein the substituent is as described above with respect to the substituent of R_{A}, R_{B}, and R_{C};

provided that at least one of R¹⁰¹ to R¹¹⁰ is -L-Ar;

each L is independently a single bond or a linker, wherein the linker is a substituted or unsubstituted arylene group having 6 to 30 ring carbon atoms or a substituted or unsubstituted heteroarylene group having 5 to 30 ring atoms;

each Ar is independently a substituted or unsubstituted single ring group having 5 to 50 ring atoms, a substituted or unsubstituted fused ring group having 8 to 50, or a monovalent group wherein two or more selected from the single ring and the fused ring are bonded to each other via a single bond;

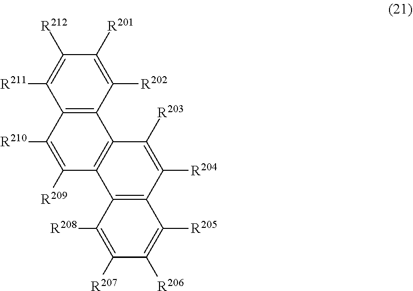

wherein:

R²⁰¹ to R²¹² are each independently a hydrogen atom or a substituent, wherein the substituent is as described above with respect to the substituent of R_{A}, R_{B}, and R_{C};

provided that at least one of R²⁰¹ to R²¹² is -L²-Ar²¹;

each L² is independently a single bond or a linker, wherein the linker is a substituted or unsubstituted arylene group having 6 to 30 ring carbon atoms or a substituted or unsubstituted heteroarylene group having 5 to 30 ring atoms; and

each Ar²¹ is independently a substituted or unsubstituted single ring group having 5 to 50 ring atoms, a substituted or unsubstituted fused ring group having 8 to 50 ring atoms, or a monovalent group wherein two or more selected from the single ring and the fused ring are bonded to each other via a single bond;

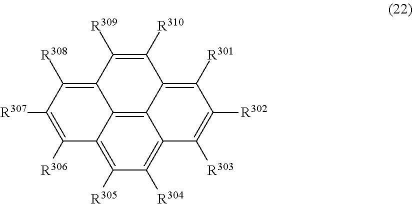

wherein:

R³⁰¹ to R³¹⁰ are each independently a hydrogen atom or a substituent, wherein the substituent is as described above with respect to the substituent of R_{A}, R_{B}, and R_{C};

provided that at least one of R³⁰¹ to R³¹⁰ is -L³-Ar³¹;

each L³ is independently a single bond or a linker, wherein the linker is a substituted or unsubstituted arylene group having 6 to 30 ring carbon atoms or a substituted or unsubstituted heteroarylene group having 5 to 30 ring atoms; and

each Ar³¹ is independently a substituted or unsubstituted single ring group having 5 to 50 ring atoms, a substituted or unsubstituted fused ring group having 8 to 50 ring atoms, or a monovalent group wherein two or more selected from the single ring and the fused ring are bonded to each other via a single bond;

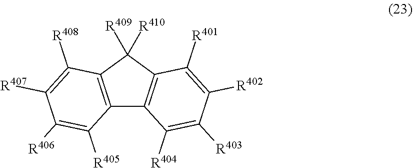

wherein:

R⁴⁰¹ to R⁴¹⁰ are each independently a hydrogen atom or a substituent, wherein the substituent is as described above with respect to the substituent of R_{A}, R_{B}, and R_{C};

provided that at least one of R⁴⁰¹ to R⁴¹⁰ is -L⁴-Ar⁴¹;

each L⁴ is independently a single bond or a linker, wherein the linker is a substituted or unsubstituted arylene group having 6 to 30 ring carbon atoms or a substituted or unsubstituted heteroarylene group having 5 to 30 ring atoms;

each Ar⁴¹ is independently a substituted or unsubstituted single ring group having 5 to 50 ring atoms, a substituted or unsubstituted fused ring group having 8 to 50 ring atoms, or a monovalent group wherein two or more selected from the single ring and the fused ring are bonded to each other via a single bond; and

adjacent two selected from R⁴⁰¹ and R⁴⁰², R⁴⁰² and R⁴⁰³, R⁴⁰³ and R⁴⁰⁴, R⁴⁰⁵ and R⁴⁰⁶, R⁴⁰⁶ and R⁴⁰⁷, and R⁴⁰⁷ and R⁴⁰⁸ may be bonded to each other to form a substituted or unsubstituted ring structure;

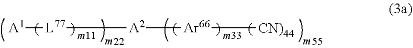

wherein:

L⁷⁷ is a substituted or unsubstituted arylene group having 6 to 50 ring carbon atoms or a substituted or unsubstituted heteroarylene group having 5 to 50 ring atoms;

Ar⁶⁶ is a di- to tetra-valent residue of an aromatic hydrocarbon ring having 6 to 50 ring carbon atoms or an aromatic heterocyclic ring having 5 to 50 ring atoms, each optionally having a substituent;

m11 is 0, 1, or 2, when m11 is 0, L⁷⁷ is a single bond, and when m11 is 2, two L⁷⁷'s may be the same or different;

m22 is 0 or 1, when m22 is 0, A¹-(L⁷⁷)ₘ₁₁- is not present and a hydrogen atom is bonded to A²;

m33 is 0, 1, 2, or 3, when m33 is 0, Ar⁶⁶ is a single bond, and when m33 is 2 or 3, two or three Ar⁶⁶'s may be the same or different;

m44 is 0, 1, 2, or 3, when m44 is 0, CN is not present and a hydrogen atom is bonded to A⁶⁶;

m55 is 1, 2, or 3, when m55 is 2 or 3, two or three —(Ar⁶⁶)ₘ₃₃—(CN)ₘ₅₅ may be the same or different;

A¹ is a monovalent group selected from formulae (A-1) to (A-12); and

A² is a di- to tetra-valent group selected from formulae (A-1) to (A-12);

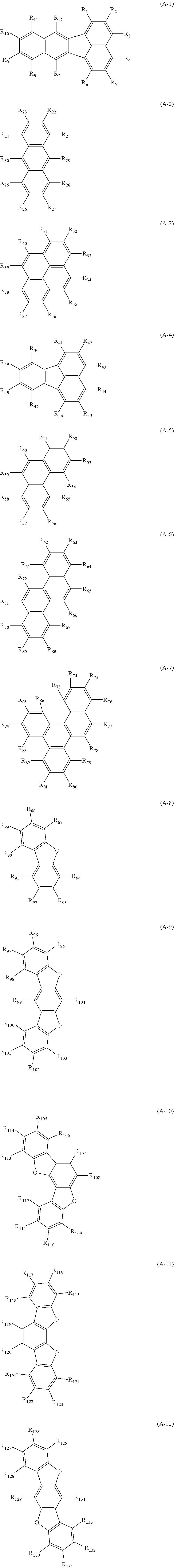

one selected from R₁ to R₁₂, one selected from R₂₁ to R₃₀, one selected from R₃₁ to R₄₀, one selected from R₄₁ to R₅₀, one selected from R₅₁ to R₆₀, one selected from R₆₁ to R₇₂, one selected from R₇₃ to R₈₆, one selected from R₈₇ to R₉₄, one selected from R₉₅ to R₁₀₄, one selected from R₁₀₅ to R₁₁₄, one selected from R₁₁₅ to R₁₂₄, and one selected from R₁₂₅ to R₁₃₃ are single bonds each bonded to L⁷⁷;

or, two to four selected from R₁ to R₁₂, two to four selected from R₂₁ to R₃₀, two to four selected from R₃₁ to R₄₀, two to four selected from R₄₁ to R₅₀, two to four selected from R₅₁ to R₆₀, two to four selected from R₆₁ to R₇₂, two to four selected from R₇₃ to R₈₆, two to four selected from R₈₇ to R₉₄, two to four selected from R₉₅ to R₁₀₄, two to four selected from R₁₀₅ to R₁₁₄, two to four selected from R₁₁₅ to R₁₂₄, and two to four selected from R₁₂₅ to R₁₃₃ are single bonds, wherein one of the single bonds is bonded to L⁷⁷ and the other single bonds are bonded to Ar⁶⁶;

R₁ to R₁₂, R₂₁ to R₃₀, R₃₁ to R₄₀, R₄₁ to R₅₀, R₅₁ to R₆₀, R₆₁ to R₇₂, R₇₃ to R₈₆, R₈₇ to R₉₄, R₉₅ to R₁₀₄, R₁₀₅ to R₁₁₄, R₁₁₅ to R₁₂₄, and R₁₂₅ to R₁₃₃ each not the single bond are each independently a hydrogen atom, a halogen atom, a cyano group, a substituted or unsubstituted alkyl group having 1 to 20 carbon atoms, a substituted or unsubstituted cycloalkyl group having 3 to 20 ring carbon atoms, a group represented by —Si(R₁₀₁)(R₁₀₂)(R₁₀₃), or a substituted or unsubstituted aryl group having 6 to 50 ring carbon atoms; and

adjacent two selected from R₁ to R₁₂, R₂₁ to R₃₀, R₃₁ to R₄₀, R₄₁ to R₅₀, R₅₁ to R₆₀, R₆₁ to R₇₂, R₇₃ to R₈₃, R₈₇ to R₉₄, R₉₅ to R₁₀₄, R₁₀₅ to R₁₁₄, R₁₁₅ to R₁₂₄, and R₁₂₅ to R₁₃₃ each not the single bond may be bonded to each other to form a substituted or unsubstituted ring structure.
(2) In another aspect of the invention, an electronic device comprising the organic EL device mentioned above in (1) is provided.

### Advantageous Effects of Invention

The organic EL device of the invention has an excellent lifetime.

### BRIEF DESCRIPTION OF DRAWINGS

FIG. 1 is a schematic view showing the structure of an organic electroluminescence device in an embodiment of the invention.

### DESCRIPTION OF EMBODIMENTS

The term of “XX to YY carbon atoms” referred to by “a substituted or unsubstituted group ZZ having XX to YY carbon atoms” used herein is the number of carbon atoms of the unsubstituted group ZZ and does not include any carbon atom in the substituent of the substituted group ZZ.

The term of “XX to YY atoms” referred to by “a substituted or unsubstituted group ZZ having XX to YY atoms” used herein is the number of atoms of the unsubstituted group ZZ and does not include any atom in the substituent of the substituted group ZZ.

The number of “ring carbon atoms” referred to herein means the number of the carbon atoms included in the atoms which are members forming the ring itself of a compound in which a series of atoms is bonded to form the ring (for example, a monocyclic compound, a fused ring compound, a cross-linked compound, a carbocyclic compound, and a heterocyclic compound). If the ring has a substituent, the carbon atom in the substituent is not included in the ring carbon atom. The same applies to the number of “ring carbon atom” described below, unless otherwise noted. For example, a benzene ring has 6 ring carbon atoms, a naphthalene ring has 10 ring carbon atoms, a pyridinyl group has 5 ring carbon atoms, and a furanyl group has 4 ring carbon atoms. If a benzene ring or a naphthalene ring has, for example, an alkyl substituent, the carbon atom in the alkyl substituent is not counted as the ring carbon atom of the benzene or naphthalene ring. In case of a fluorene ring to which a fluorene substituent is bonded (inclusive of a spirofluorene ring), the carbon atom in the fluorene substituent is not counted as the ring carbon atom of the fluorene ring.

The number of “ring atom” referred to herein means the number of the atoms which are members forming the ring itself (for example, a monocyclic ring, a fused ring, and a ring assembly) of a compound in which a series of atoms is bonded to form the ring (for example, a monocyclic compound, a fused ring compound, a cross-linked compound, a carbocyclic compound, and a heterocyclic compound). The atom not forming the ring (for example, hydrogen atom(s) for saturating the valence of the atom which forms the ring) and the atom in a substituent, if the ring is substituted, are not counted as the ring atom. The same applies to the number of “ring atoms” described below, unless otherwise noted. For example, a pyridine ring has 6 ring atoms, a quinazoline ring has 10 ring atoms, and a furan ring has 5 ring atoms. The hydrogen atom on the ring carbon atom of a pyridine ring or a quinazoline ring and the atom in a substituent are not counted as the ring atom. In case of a fluorene ring to which a fluorene substituent is bonded (inclusive of a spirofluorene ring), the atom in the fluorene substituent is not counted as the ring atom of the fluorene ring.

The definition of “hydrogen atom” used herein includes isotopes different in the neutron numbers, i.e., light hydrogen (protium), heavy hydrogen (deuterium), and tritium.

### Organic EL Device

The organic EL device of the invention comprises a cathode, an anode, and an organic layer disposed between the cathode and the anode, wherein the organic layer comprises a fluorescent emitting layer.

The fluorescent emitting layer comprises at least one first compound selected from the compounds represented by formulae (19), (21), (22), and (23) each described below, a second compound selected from the compound represented by formula (3a), and a dopant material selected from the compounds represented by the following formulae (D1) and (D2).

The content of the dopant material in the fluorescent emitting layer is preferably 10% by mass or less, more preferably 1 to 10% by mass, and still more preferably 1 to 8% by mass, each based on the total amount of the first compound, the second compound and the dopant material.

The content of the second compound in the fluorescent emitting layer is preferably less than that of the first compound in the fluorescent emitting layer.

The content of the second compound in the fluorescent emitting layer is preferably 30% by mass or less, more preferably 2 to 30% by mass, and still more preferably 2 to 20% by mass, each based on the total amount of the first compound, the second compound, and the dopant material. Within the above ranges, the region of high excitation density comes close to the central portion of the fluorescent emitting layer to increase the lifetime.

### Dopant Material

The dopant material used in the organic EL device of the invention is at least one compound selected from the compound represented by formula (D1) (“dopant material 1”) and the compound represented by formula (D2) (“dopant material 2”) and preferably at least one compound selected from the compound represented by formula (D1) (“dopant material 1”).

The dopant material 1 is represented by formula (D1):

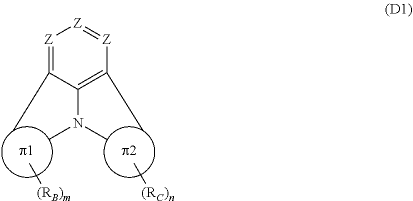

wherein:

each Z is independently CR_{A} or N;

a ring π1 is a substituted or unsubstituted aromatic hydrocarbon ring having 6 to 50 ring carbon atoms or a substituted or unsubstituted aromatic heterocyclic ring having 5 to 50 ring atoms;

a ring π2 is a substituted or unsubstituted aromatic hydrocarbon ring having 6 to 50 ring carbon atoms or a substituted or unsubstituted aromatic heterocyclic ring having 5 to 50 ring atoms;

R_{A}, R_{B}, and R_{C} are each independently a hydrogen atom or a substituent, wherein the substituent is a halogen atom, a cyano group, a substituted or unsubstituted alkyl group having 1 to 20 carbon atoms, a substituted or unsubstituted alkenyl group having 1 to 20 carbon atoms, a substituted or unsubstituted alkynyl group having 1 to 20 carbon atoms, a substituted or unsubstituted cycloalkyl group having 3 to 20 ring carbon atoms, a substituted or unsubstituted alkoxy group having 1 to 20 carbon atoms, a substituted or unsubstituted aryl group having 6 to 50 ring carbon atoms, a substituted or unsubstituted aryloxy group having 6 to 50 ring carbon atoms, a substituted or unsubstituted alkylthio group having 1 to 20 carbon atoms, a substituted or unsubstituted arylthio group having 6 to 50 ring carbon atoms, a substituted or unsubstituted heteroaryl group having 5 to 50 ring atoms, a group represented by —Si(R₁₀₁)(R₁₀₂)(R₁₀₃), or a group represented by —N(R₁₀₄)(R₁₀₅);

R₁₀₁ to R₁₀₅ are each independently a hydrogen atom, a substituted or unsubstituted alkyl group having 1 to 20 carbon atoms, a substituted or unsubstituted cycloalkyl group having 3 to 20 ring carbon atoms, a substituted or unsubstituted aryl group having 6 to 50 ring carbon atoms, or a substituted or unsubstituted heteroaryl group having 5 to 50 ring atoms;

n and m are each independently an integer of 1 to 4;

adjacent two R_{A}'s are bonded to each other to form a substituted or unsubstituted ring structure or not bonded to each other, thereby failing to form a ring structure;

adjacent two R_{B}'s are bonded to each other to form a substituted or unsubstituted ring structure or not bonded to each other, thereby failing to form a ring structure; and

adjacent two R_{C}'s are bonded to each other to form a substituted or unsubstituted ring structure or not bonded to each other, thereby failing to form a ring structure.

The ring π1 and the ring π2 are each independently an aromatic hydrocarbon ring having 6 to 50, preferably 6 to 24, and more preferably 6 to 18 ring carbon atoms or an aromatic heterocyclic ring having 5 to 50, preferably 5 to 24, and more preferably 5 to 13 ring atoms.

Examples of the aromatic hydrocarbon ring having 6 to 50 ring carbon atoms include a benzene ring, a naphthalene ring, an anthracene ring, a benzanthracene ring, a phenanthrene ring, a benzophenanthrene ring, a fluorene ring, a benzofluorene ring, a dibenzofluorene ring, a picene ring, a tetracene ring, a pentacene ring, a pyrene ring, a chrysene ring, a benzochrysene ring, a s-indacene ring, an as-indacene ring, a fluoranthene ring, a benzofluoranthene ring, a triphenylene ring, a benzotriphenylene ring, a perylene ring, a coronene ring, and a dibenzanthracene ring.

Examples of the aromatic heterocyclic ring having 5 to 50 ring atoms include a pyrrole ring, a pyrazole ring, an isoindole ring, a benzofuran ring, a benzothiophene ring, an isobenzofuran ring, a dibenzothiophene ring, an isoquinoline ring, a cinnoline ring, a quinoxaline ring, a phenanthridine ring, a phenanthroline ring, a pyridine ring, a pyrazine ring, a pyrimidine ring, a pyridazine ring, a triazine ring, an imidazopyridine ring, an indole ring, an indazole ring, a benzimidazole ring, a quinoline ring, an acridine ring, a pyrrolidine ring, a dioxane ring, a piperidine ring, a morpholine ring, a piperazine ring, a carbazole ring, a furan ring, a thiophene ring, an oxazole ring, an oxadiazole ring, a benzoxazole ring, a thiazole ring, a thiadiazole ring, a benzothiazole ring, a triazole ring, an imidazole ring, a benzimidazole ring, a pyran ring, a dibenzofuran ring, a benzo[c]dibenzofuran ring, a purine ring, and an acridine ring.

Each R_{B} is bonded to a ring atom of the aromatic hydrocarbon ring or the aromatic heterocyclic ring (ring π1). Each R_{C} is bonded to a ring atom of the aromatic hydrocarbon ring or the aromatic heterocyclic ring (ring π2).

The substituents represented by R_{A}, R_{B}, and R_{C} are described below.

The halogen atom is a fluorine atom, a chlorine atom, a bromine atom, or an iodine atom.

Examples of the alkyl group of the substituted or unsubstituted alkyl group having 1 to 20, preferably 1 to 10, and more preferably 1 to 6 carbon atoms include a methyl group, an ethyl group, a n-propyl group, an isopropyl group, a n-butyl group, an isobutyl group, a s-butyl group, a t-butyl group, a pentyl group (inclusive of isomeric groups), a hexyl group (inclusive of isomeric groups), a heptyl group (inclusive of isomeric groups), an octyl group (inclusive of isomeric groups), a nonyl group (inclusive of isomeric groups), a decyl group (inclusive of isomeric groups), an undecyl group (inclusive of isomeric groups), and a dodecyl group (inclusive of isomeric groups). Preferred are a methyl group, an ethyl group, a n-propyl group, an isopropyl group, a n-butyl group, an isobutyl group, a s-butyl group, a t-butyl group, and a pentyl group (inclusive of isomeric groups), more preferred are a methyl group, an ethyl group, a n-propyl group, an isopropyl group, a n-butyl group, an isobutyl group, a s-butyl group, and a t-butyl group, and still more preferred are a methyl group, an ethyl group, an isopropyl group, and a t-butyl group.

The substituted alkyl group is preferably a fluoroalkyl group having 1 to 20, preferably 1 to 10, and more preferably 1 to 6 carbon atoms. The fluoroalkyl group is a group derived from the above alkyl group having 1 to 20 carbon atoms by replacing at least one hydrogen atom, preferably 1 to 7 hydrogen atoms, or all hydrogen atoms with a fluorine atom. The fluoroalkyl group is preferably a heptafluoropropyl group (inclusive of isomeric groups), a pentafluoroethyl group, a 2,2,2-trifluoroethyl group, or a trifluoromethyl group, more preferably a pentafluoroethyl group, a 2,2,2-trifluoroethyl group, or a trifluoromethyl group, and still more preferably a trifluoromethyl group.

Examples of the alkenyl group of the substituted or unsubstituted alkenyl group having 1 to 20, preferably 1 to 10, and more preferably 1 to 6 carbon atoms include a vinyl group, a 2-propenyl group, a 2-butenyl group, a 3-butenyl group, a 4-pentenyl group, a 2-methyl-2-propenyl group, a 2-methyl-2-butenyl group, and a 3-methyl-2-butenyl group.

Examples of the alkynyl group of the substituted or unsubstituted alkynyl group having 1 to 20, preferably 1 to 10, and more preferably 1 to 6 carbon atoms include a 2-propynyl group, a 2-butynyl group, a 3-butynyl group, a 4-pentynyl group, a 5-hexynyl group, a 1-methyl-2-propynyl group, a 1-methyl-2-butynyl group, and a 1,1-dimethyl-2-propynyl group.

Examples of the cycloalkyl group of the substituted or unsubstituted cycloalkyl group having 3 to 20, preferably 3 to 6, and more preferably 5 or 6 ring carbon atoms include a cyclopropyl group, a cyclobutyl group, a cyclopentyl group, a cyclohexyl group, a cycloheptyl group, a cyclooctyl group, and an adamantyl group, with a cyclopentyl group and a cyclohexyl group being preferred.

The details of the alkyl portion of the substituted or unsubstituted alkoxy group having 1 to 20, preferably 1 to 10, and more preferably 1 to 6 carbon atoms are as described above with respect to the alkyl group having 1 to 20 carbon atoms.

The substituted alkoxy group having 1 to 20, preferably 1 to 10, and more preferably 1 to 6 carbon atoms is preferably a fluoroalkoxy group. The details of the fluoroalkyl portion of the fluoroalkoxy group are as described above with respect to the fluoroalkyl group having 1 to 20 carbon atoms.

The aryl group of the substituted or unsubstituted aryl group having 6 to 50, preferably 6 to 30, more preferably 6 to 24, and still more preferably 6 to 18 may be a fused aryl group or a non-fused aryl group. Examples thereof include a phenyl group, a biphenylyl group, a terphenylyl group, a naphthyl group, an acenaphthylenyl group, an anthryl group, a benzanthryl group, an aceanthryl group, a phenanthryl group, a benzo[c]phenanthryl group, a phenalenyl group, a fluorenyl group, a picenyl group, a pentaphenyl group, a pyrenyl group, a chrysenyl group, a benzo[g]chrysenyl group, a s-indacenyl group, an as-indacenyl group, a fluoranthenyl group, a benzo[k]fluoranthenyl group, a triphenylenyl group, a benzo[b]triphenylenyl group, and a perylenyl group. Preferred are a phenyl group, a biphenylyl group, a terphenylyl group, a naphthyl group, an anthryl group, a pyrenyl group, and a fluoranthenyl group, with a phenyl group, a biphenylyl group, and a terphenylyl group being more preferred and a phenyl group being still more preferred.

The substituted aryl group is preferably a 9,9-dimethylfluorenyl group, a 9,9-diphenyl fluorenyl group, a 9,9′-spirobifluorenyl group, a 9,9-di(4-methylphenyl)fluorenyl group, a 9,9-di(4-isopropylphenyl)fluorenyl group, a 9,9-di(4-t-butylphenyl)fluorenyl group, a para-methylphenyl group, a meta-methylphenyl group, an ortho-methylphenyl group, a para-isopropylphenyl group, a meta-isopropylphenyl group, an ortho-isopropylphenyl group, a para-t-butylphenyl group, a meta-t-butylphenyl group, or an ortho-t-butylphenyl group.

The details of the aryl portion of the aryloxy group in the substituted or unsubstituted aryloxy group having 6 to 50, preferably 6 to 30, more preferably 6 to 24, and still more preferably 6 to 18 are as described above with respect to the aryl group having 6 to 50 ring carbon atoms.

The details of the alkyl portion of the alkylthio group in the substituted or unsubstituted alkylthio group having 1 to 20, preferably 1 to 10, and more preferably 1 to 6 carbon atoms are as described above with respect to the alkyl group having 1 to 20 carbon atoms.

The details of the aryl portion of the arylthio group in the substituted or unsubstituted arylthio group having 6 to 50, preferably 6 to 30, more preferably 6 to 24, and still more preferably 6 to 18 are as described above with respect to the aryl group having 6 to 50 ring carbon atoms.

The heteroaryl group of the substituted or unsubstituted heteroaryl group having 5 to 50, preferably 5 to 30, more preferably 5 to 18, and still more preferably 5 to 13 ring atoms includes at least one, preferably 1 to 5, more preferably 1 to 4, and still more preferably 1 to 3 ring hetero atoms. Examples of the ring hetero atom include a nitrogen atom, a sulfur atom, and an oxygen atom, with a nitrogen atom and an oxygen atom being preferred. The free valance of the heteroaryl group is present on a ring carbon atom or may be present on a ring nitrogen atom, if structurally possible.

Examples the heteroaryl group include the a pyrrolyl group, a furyl group, a thienyl group, a pyridyl group, an imidazopyridyl group, a pyridazinyl group, a pyrimidinyl group, a pyrazinyl group, a triazinyl group, an imidazolyl group, an oxazolyl group, a thiazolyl group, a pyrazolyl group, an isoxazolyl group, an isothiazolyl group, an oxadiazolyl group, a thiadiazolyl group, a triazolyl group, a tetrazolyl group, an indolyl group, an isoindolyl group, a benzofuranyl group, an isobenzofuranyl group, a benzothiophenyl group (a benzothienyl group), an isobenzothiophenyl group (an isobenzothienyl group), an indolizinyl group, a quinolizinyl group, a quinolyl group, an isoquinolyl group, a cinnolyl group, a phthalazinyl group, a quinazolinyl group, a quinoxalinyl group, a benzimidazolyl group, a benzoxazolyl group, a benzothiazolyl group, an indazolyl group, a benzisoxazolyl group, a benzisothiazolyl group, a dibenzofuranyl group, a dibenzothiophenyl group (a dibenzothienyl group), a carbazolyl group, a phenanthridinyl group, an acridinyl group, a phenanthrolinyl group, a phenazinyl group, a phenothiazinyl group, a phenoxazinyl group, and a xanthenyl group.

Other examples of the heteroaryl group include the following groups:

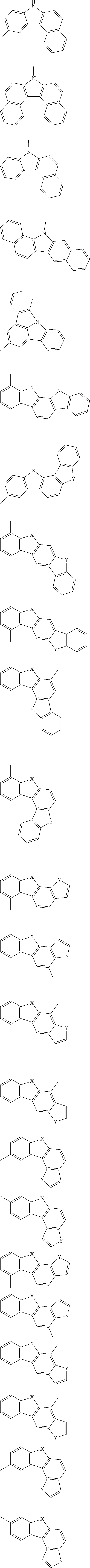

wherein X is an oxygen atom or a sulfur atom, Y is an oxygen atom, a sulfur atom, NR^{a}, or CR^{b}₂, and each of R^{a} and R^{b} is a hydrogen atom.

Preferred heteroaryl groups are a pyridyl group, an imidazopyridyl group, a pyridazinyl group, a pyrimidinyl group, a pyrazinyl group, a triazinyl group, a benzimidazolyl group, a dibenzofuranyl group, a dibenzothiophenyl group, a carbazolyl group, a phenanthrolinyl group, and a quinazolinyl group.

Examples of the substitute heteroaryl group include a (9-phenyl)carbazolyl group, a (9-biphenylyl)carbazolyl group, a (9-phenyl)phenylcarbazolyl group, a (9-naphthyl)carbazolyl group, a diphenylcarbazole-9-yl group, a phenyldibenzofuranyl group, a phenyldibenzothiophenyl group (phenyldibenzothienyl group), and the following groups:

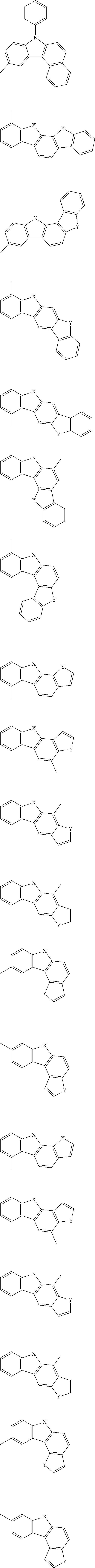

wherein X is an oxygen atom or a sulfur atom, Y is NR^{a} or CR^{b}₂, and R^{a} and R^{b} are each independently selected from the alkyl group having 1 to 20 carbon atoms mentioned above and the aryl group having 6 to 50 ring carbon atoms mentioned above.

In the group represented by —Si(R₁₀₁)(R₁₀₂)(R₁₀₃) and the group represented by —N(R₁₀₄)(R₁₀₅), R₁₀₁ to R₁₀₅ are each independently a hydrogen atom, a substituted or unsubstituted alkyl group having 1 to 20 carbon atoms, a substituted or unsubstituted cycloalkyl group having 3 to 20 ring carbon atoms, a substituted or unsubstituted aryl group having 6 to 50 ring carbon atoms, or a substituted or unsubstituted heteroaryl group having 5 to 50 ring atoms.

The details of the substituted or unsubstituted alkyl group having 1 to 20 carbon atoms, the substituted or unsubstituted cycloalkyl group having 3 to 20 ring carbon atoms, the substituted or unsubstituted aryl group having 6 to 50 ring carbon atoms, and the substituted or unsubstituted heteroaryl group having 5 to 50 ring atoms are as described above.

Examples of the group represented by —Si(R₁₀₁)(R₁₀₂)(R₁₀₃) include a monoalkylsilyl group, a dialkylsilyl group, a trialkylsilyl group, a monoarylsilyl group, a diarylsilyl group, a triarylsilyl group, a monoalkyldiarylsilyl group, and a dialkylmonoarylsilyl group.

Preferred are a trialkylsilyl group and a triarylsilyl group and more preferred are a trimethylsilyl group, a triethylsilyl group, a triisopropylsilyl group, a t-butyldimethylsilyl group, a triphenylsilyl group, and a tritolylsilyl group.

Examples of the group represented by —N(R₁₀₄)(R₁₀₅) include an amino group, a monoalkylamino group, a dialkylamino group, a monoarylamino group, a diarylamino group, a monoheteroarylamino group, a diheteroarylamino group, a monoalkylmonoarylamino group, a monoalkylmonoheteroarylamino group, and a monoarylmonoheteroarylamino group. Preferred are a dialkylamino group, a diarylamino group, a diheteroarylamino group, and a monoarylmonoheteroarylamino group and more preferred are a dimethylamino group, a diethylamino group, a diisopropylamino group, a diphenylamino group, a bis(alkyl-substituted phenyl)amino group, and a bis(aryl-substituted phenyl)amino group.

Two or more groups represented by —Si(R₁₀₁)(R₁₀₂)(R₁₀₃) in formula (D1) may be the same or different. Two or more groups represented by —N(R₁₀₄)(R₁₀₅) in formula (D1) may be the same or different.

The compound represented by formula (D1) preferably includes a compound represented by formula (D1a):

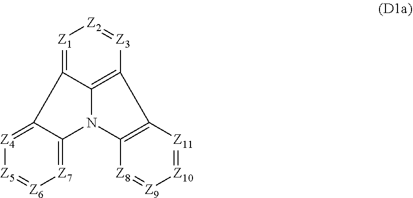

wherein:

Z₁ is CR₁ or N, Z₂ is CR₂ or N, Z₃ is CR₃ or N, Z₄ is CR₄ or N, Z₅ is CR₅ or N, Z₆ is CR₆ or N, Z₇ is CR₇ or N, Z₈ is CR₈ or N, Z₉ is CR₉ or N, Z₁₀ is CR₁₀ or N, and Z₁₁ is CR₁₁ or N;

R₁ to R₁₁ are each independently a hydrogen atom or a substituent, wherein the substituent is as described above with respect to the substituent of R_{A}, R_{B}, and R_{C} of formula (D1);

adjacent two selected from R₁ to R₃ may be bonded to each other to form a substituted or unsubstituted ring structure or not bonded to each other, thereby failing to form a ring structure;

adjacent two selected from R₄ to R₇ may be bonded to each other to form a substituted or unsubstituted ring structure or not bonded to each other, thereby failing to form a ring structure;

adjacent two selected from R₈ to R₁₁ may be bonded to each other to form a substituted or unsubstituted ring structure or not bonded to each other, thereby failing to form a ring structure.

The compound represented by formula (D1) preferably includes a compound represented by formula (1):

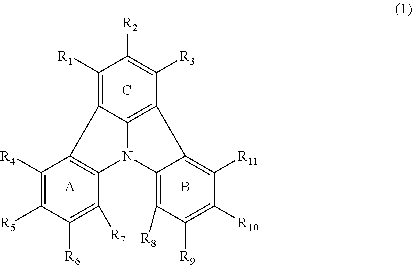

wherein:

Rₙ and Rₙ₊₁ (n is an integer selected from 1, 2, 4 to 6, and 8 to 10) may be bonded to each other to form, together with two ring carbon atoms to which Rₙ and Rₙ₊₁ are bonded, a substituted or unsubstituted ring structure having 3 or more ring atoms, or Rₙ and Rₙ₊₁ may be not bonded to each other, thereby failing to form a ring structure;

the ring atom is selected from a carbon atom, an oxygen atom, a sulfur atom, and a nitrogen atom;

an optional substituent of the ring structure having 3 or more ring atoms is as described above with respect to the substituent of R_{A}, R_{B}, and R_{C} of formula (D1) and adjacent two optional substituents may be bonded to each other to form a substituted or unsubstituted ring structure; and

R₁ to R₁₁ not forming the substituted or unsubstituted ring structure having 3 or more ring atoms is a hydrogen atom or a substituent, wherein the substituent is as described above with respect to the substituent of R_{A}, R_{B}, and R_{C} of formula (D1).

When Rₙ and Rₙ₊₁, i.e., R₁ and R₂, R₂ and R₃, R₄ and R₅, R₅ and R₆, R₆ and R₇, R₈ and R₉, R₉ and R₁₀, or R₁₀ and R₁₁, are bonded to each other to form, together with two ring carbon atoms to which Rₙ and Rₙ₊₁ are bonded, the substituted or unsubstituted ring structure having 3 or more ring atoms, Rₙ-Rₙ₊₁, i.e., R₁-R₂, R₂-R₃, R₄-R₅, R₅-R₆, R₆-R₇, R₈-R₉, R₉-R₁₀, or R₁₀-R₁₁ represents CH₂, NH, O, or S, or represents a group of atoms wherein two or more selected from CH₂, CH, NH, N, O, and S are successively bonded to each other via a single bond, a double bond, or an aromatic bond. The hydrogen atom of CH₂, CH, and NH may be substituted by the substituent mentioned above. The aromatic bond is a bond bonding two adjacent atoms in an aromatic ring and having a bond order between 1 and 2 (about 1.5).

In an embodiment of the invention, the compound of formula (1) preferably has two substituted or unsubstituted ring structures each having 3 or more ring atoms.

In another embodiment of the invention, the compound of formula (1) preferably has three ring structures and more preferably has one ring structure on each of the three different benzene rings, i.e., one ring structure on each of the ring A, the ring B, and the ring C.

In still another embodiment of the invention, the compound of formula (1) preferably has four or more ring structures.

In an embodiment of the invention, a pair of Rₚ and Rₚ₊₁ and a pair of Rₚ₊₁ and Rₚ₊₂ (wherein p is 1, 4, 5, 8, or 9) preferably do not form the substituted or unsubstituted ring structure having 3 or more ring atoms at the same time. Namely, a pair of R₁ and R₂ and a pair of R₂ and R₃; a pair of R₄ and R₅ and a pair of R₅ and R₆; a pair of R₅ and R₆ and a pair of R₆ and R₇; a pair of R₈ and R₉ and a pair of R₉ and R₁₀; and a pair of R₉ and R₁₀ and a pair of R₁₀ and R₁₁ preferably do not form the ring structure at the same time.

In an embodiment of the invention, when the compound of formula (1) has two or more substituted or unsubstituted ring structures each having 3 or more ring atoms, the two or more ring structures are preferably present on two or three rings selected from the ring A, the ring B, and the ring C. The two or more ring structures may be the same or different.

The details of the optional substituent of the substituted or unsubstituted ring structure having 3 or more ring atoms are as described above with respect to the substituent of R_{A}, R_{B}, and R_{C} of formula (D1).

The number of ring atoms of the substituted or unsubstituted ring structure having 3 or more ring atoms is preferably 3 to 7 and more preferably 5 or 6, although not limited thereto.

The substituted or unsubstituted ring structure having 3 or more ring atoms is preferably a ring structure represented by any of formulae (2) to (8):

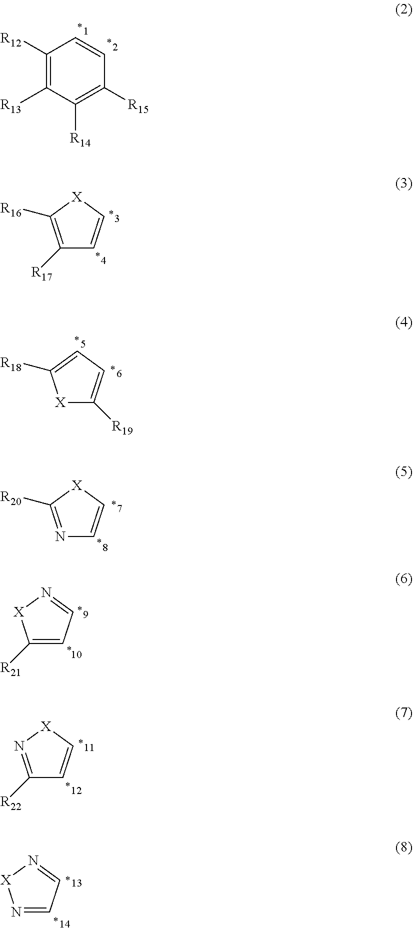

wherein:

*1 and *2, *3 and *4, *5 and *6, *7 and *8, *9 and *10, *11 and *12, and *13 and *14 are two ring carbon atoms to which Rₙ and Rₙ₊₁ are bonded, wherein Rₙ may be bonded to either of the two ring carbon atoms;

X is selected from C(R₂₃)(R₂₄), NR₂₅, O, and S;

R₁₂ to R₂₅ are each independently a hydrogen atom or a substituent, wherein the substituent is as described above with respect to the substituent of R_{A}, R_{B}, and R_{C}; and

adjacent two selected from R₁₂ to R₁₅, R₁₆ and R₁₇, and R₂₃ and R₂₄ may be bonded to each other to form a substituted or unsubstituted ring structure.

A ring structure selected from formulae (9) to (11) are also preferred as the substituted or unsubstituted ring structure having 3 or more ring atoms:

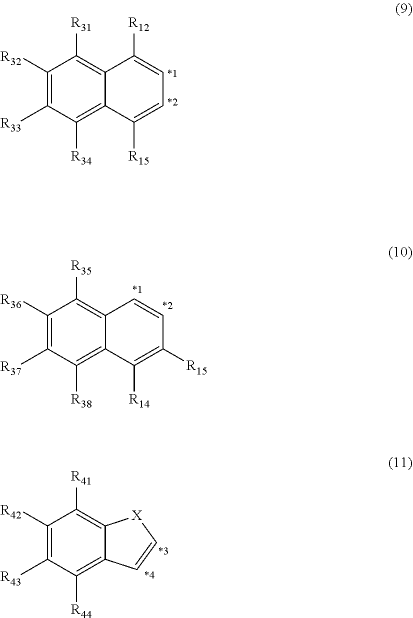

wherein:

*1 and *2, and *3 and *4 are as defined above;

R₁₂, R₁₄, R₁₅, and X are as defined above;

R₃₁ to R₃₈ and R₄₁ to R₄₄ are each independently a hydrogen atom or a substituent, wherein the substituent is as described above with respect to the substituent of R_{A}, Rs, and R_{C} of formula (D1); and

adjacent two selected from R₁₂, R₁₅, and R₃₁ to R₃₄, adjacent two selected from R₁₄, R₁₅, and R₃₅ to R₃₈, and adjacent two selected from R₄₁ to R₄₄ may be bonded to each other to form a substituted or unsubstituted ring structure.

Preferably, in formula (1), at least one of R₂, R₄, R₅, R₁₀, and R₁₁, preferably at least one of R₂, R₅, and R₁₀, and more preferably R₂ does not form the substituted or unsubstituted ring structure having 3 or more ring atoms.

Preferably, in formula (1), the optional substituent of the ring structure having 3 or more ring atoms is independently a substituted or unsubstituted alkyl group having 1 to 20 carbon atoms, a group represented by —N(R₁₀₄)(R₁₀₅), a substituted or unsubstituted aryl group having 6 to 50 ring carbon atoms, a substituted or unsubstituted heteroaryl group having 5 to 50 ring atoms, or any of the following groups:

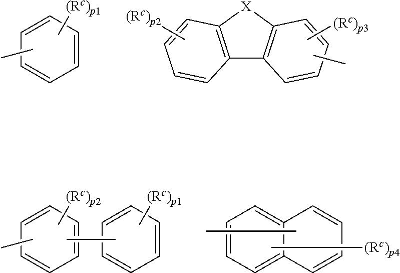

wherein:

each R^{c} is independently a hydrogen atom or a substituent, wherein the substituent is as described above with respect to the substituent of R_{A}, R_{B}, and R_{C} of formula (D1);

X is as defined above;

p1 is an integer of 0 to 5, p2 is an integer of 0 to 4, p3 is an integer of 0 to 3, and p4 is an integer of 0 to 7.

Preferably, R₁ to R₁₁ of formula (1) not forming the substituted or unsubstituted ring structure having 3 or more ring atoms and R₁₂ to R₂₂, R₃₁ to R₃₈, and R₄₁ to R₄₄ of formulae (2) to (11) are independently a hydrogen atom, a substituted or unsubstituted alkyl group having 1 to 20 carbon atoms, a group represented by —N(R₁₀₄)(R₁₀₅), a substituted or unsubstituted aryl group having 6 to 50 ring carbon atoms, a substituted or unsubstituted heteroaryl group having 5 to 50 ring atoms, or any of the following groups:

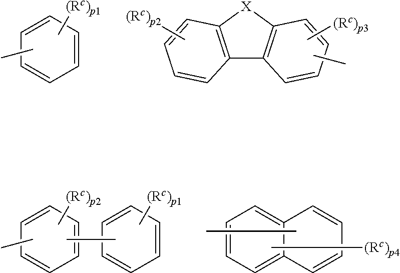

wherein R^{c}, X, p1, p2, p3, and p4 are as defined above.

The compound of formula (1) is preferably represented by any of formulae (1-1) to (1-6), more preferably represented by any of formulae (1-1) to (1-3) and (1-5), and still more preferably represented by formula (1-1) or (1-5):

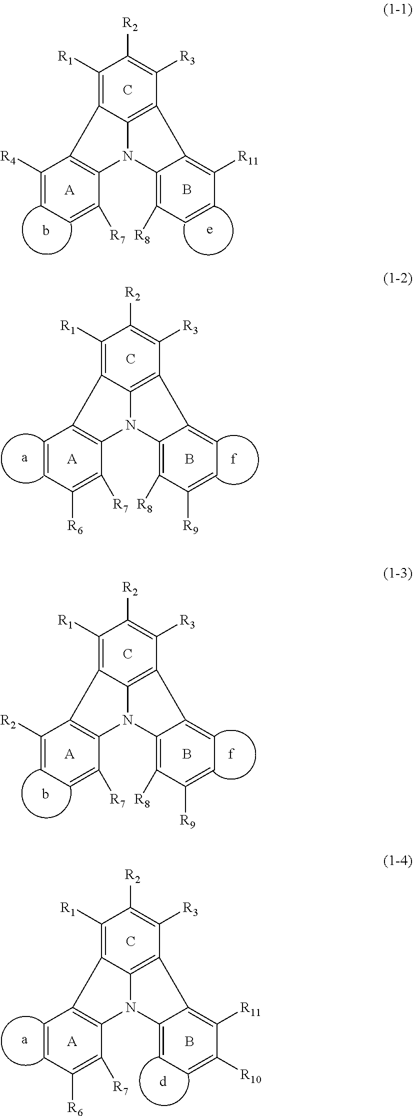

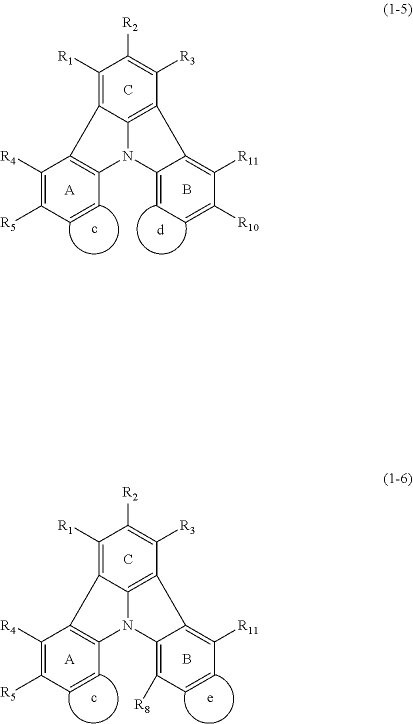

wherein:

R₁ to R₁₁ are as defined above; and

the rings a to f are each independently the substituted or unsubstituted ring structure having 3 or more ring atoms.

In formulae (1-1) to (1-6), adjacent two optional substituents on the ring structure having 3 or more ring atoms may be bonded to each other to form a substituted or unsubstituted ring structure.

The number of ring atoms of the rings a to f is preferably 3 to 7 and more preferably 5 or 6, although not limited thereto. Preferably, the rings a to f are each independently any of the rings selected from formulae (2) to (11).

The compound of formula (1) is preferably represented by any of formulae (2-1) to (2-6) and more preferably represented by formula (2-2) or (2-5):

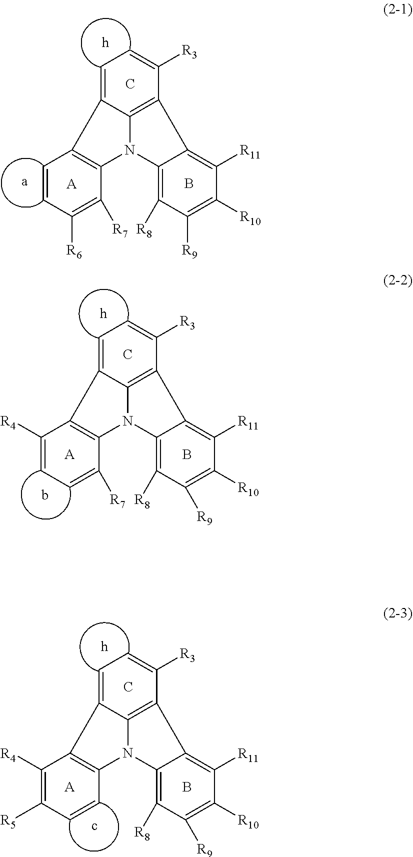

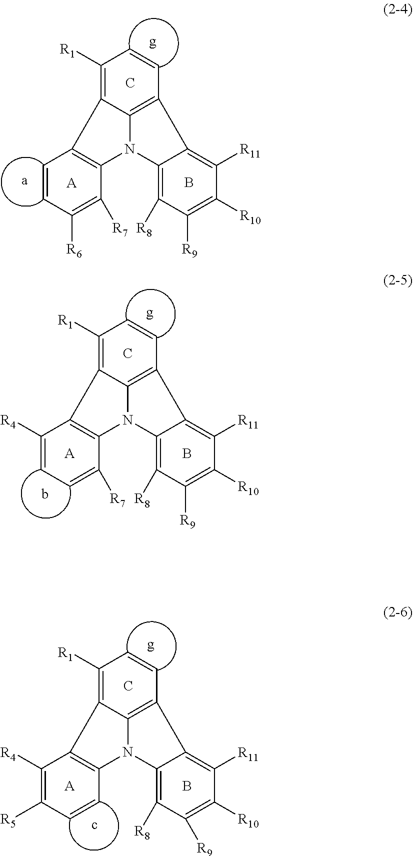

wherein:

R₁ and R₃ to R₁₁ are as defined above;

the rings a to c are as defined above; and

the rings g and h are each independently the substituted or unsubstituted ring structure having 3 or more ring atoms.

In formulae (2-1) to (2-6), adjacent two optional substituents on the ring structure having 3 or more ring atoms may be bonded to each other to form a substituted or unsubstituted ring structure.

The number of ring atoms of the rings a to c, g, and h is preferably 3 to 7 and more preferably 5 or 6, although not limited thereto. Preferably, the rings a to c, g, and h are each independently any of the rings selected from formulae (2) to (11).

The compound of formula (1) is more preferably represented by any of formulae (3-1) to (3-9) and still more preferably represented by formula (3-1):

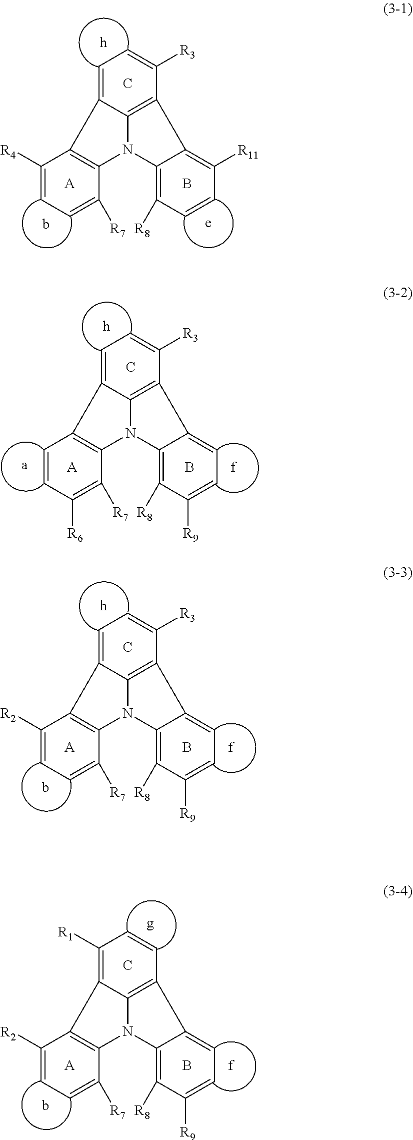

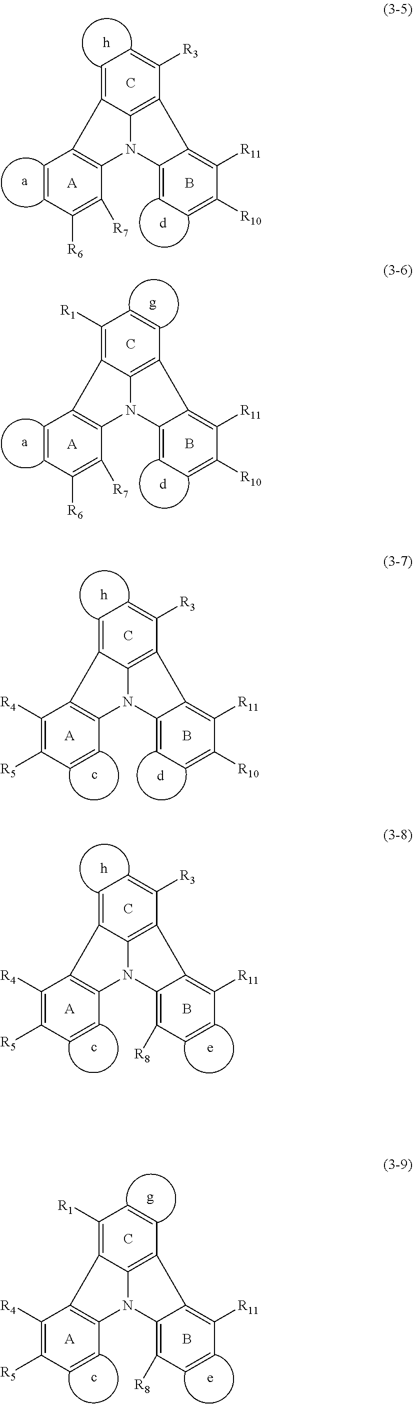

wherein R₁, R₃ to R₁₁, and the rings a to h are as defined above.

Preferably, in formulae (1-1) to (1-6), (2-1) to (2-6), and (3-1) to (3-9), the optional substituent of the rings a to h is independently a substituted or unsubstituted alkyl group having 1 to 20 carbon atoms, a group represented by —N(R₁₀₄)(R₁₀₅), a substituted or unsubstituted aryl group having 6 to 50 ring carbon atoms, a substituted or unsubstituted heteroaryl group having 5 to 50 ring atoms, or any of the following groups:

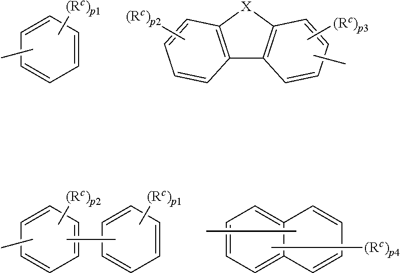

wherein R^{c}, X, p1, p2, p3, and p4 are as defined above.

Preferably, in formulae (1-1) to (1-6), (2-1) to (2-6), and (3-1) to (3-9), R₁ to R₁₁ not forming the rings a to h is independently a hydrogen atom, a substituted or unsubstituted alkyl group having 1 to 20 carbon atoms, a group represented by —N(R₁₀₄)(R₁₀₅), a substituted or unsubstituted aryl group having 6 to 50 ring carbon atoms, a substituted or unsubstituted heteroaryl group having 5 to 50 ring atoms, or any of the following groups:

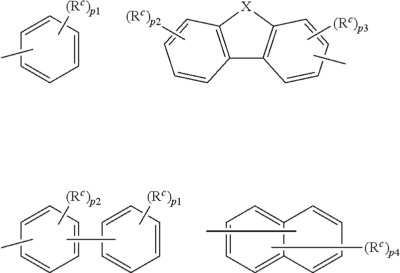

wherein R^{c}, X, p1, p2, p3, and p4 are as defined above.

The compound of formula (1) is preferably represented by any of formulae (4-1) to (4-4):

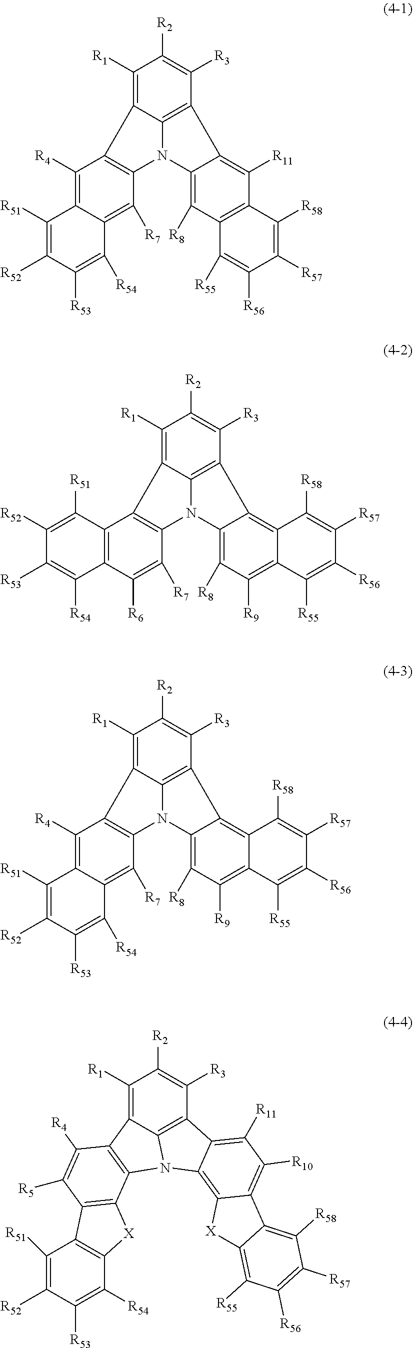

wherein:

R₁ to R₁₁ and X are as defined above; and

R₅₁ to R₅₈ are each independently a hydrogen atom or a substituent, wherein the substituent is as described above with respect to the substituent of R_{A}, R_{B}, and R_{C} of formula (D1).

The compound of formula (1) is preferably represented by formula (5-1):

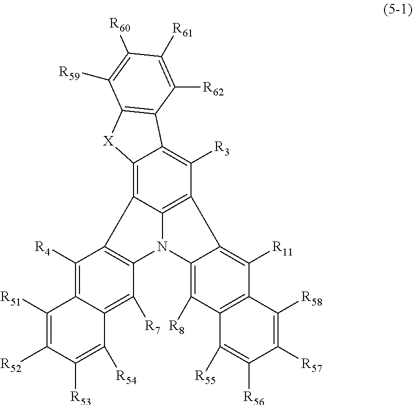

wherein:

R₃, R₄, R₇, R₈, R₁₁, and R₅₁ to R₅₈ are as defined above; and

R₅₉ to R₆₂ are each independently a hydrogen atom or a substituent, wherein the substituent is as described above with respect to the substituent of R_{A}, R_{B}, and R_{C} of formula (D1).

Examples of the dopant material represented by formula (D1) which is used in the present invention are shown below, although not limited thereto. In the following exemplary compounds, Ph is a phenyl group and D is a heavy hydrogen atom.

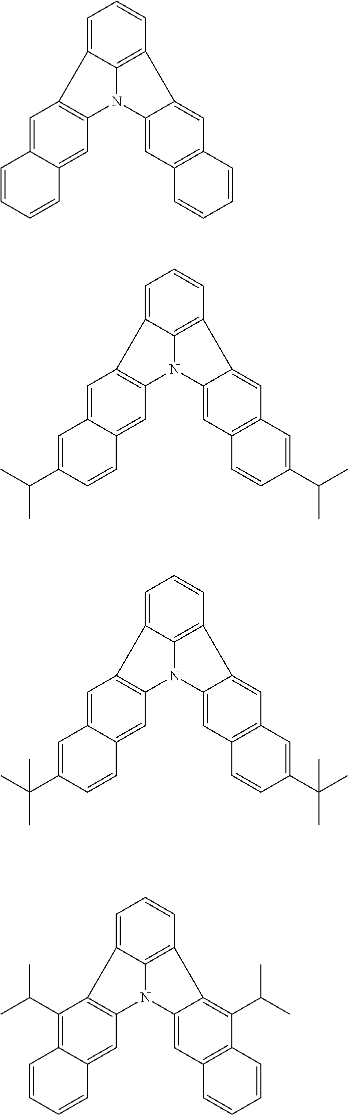

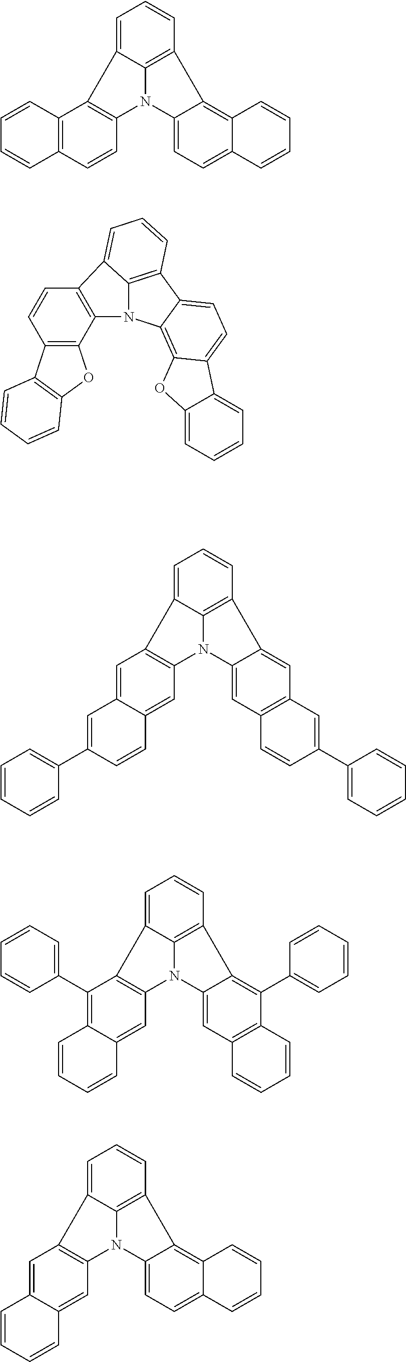 [SMILES: c1ccc2c(c1)ccc1c2c2cccc3c4c5ccccc5ccc4n1c23 ; c1ccc2c(c1)oc1c2ccc2c3cccc4c5ccc6c7ccccc7oc6c5n(c34)c21 ; c1ccc(-c2ccc3cc4c(cc3c2)c2cccc3c5cc6cc(-c7ccccc7)ccc6cc5n4c23)cc1 ; c1ccc(-c2c3ccccc3cc3c2c2cccc4c5c(-c6ccccc6)c6ccccc6cc5n3c24)cc1 ; c1ccc2cc3c(cc2c1)c1cccc2c4c5ccccc5ccc4n3c12]

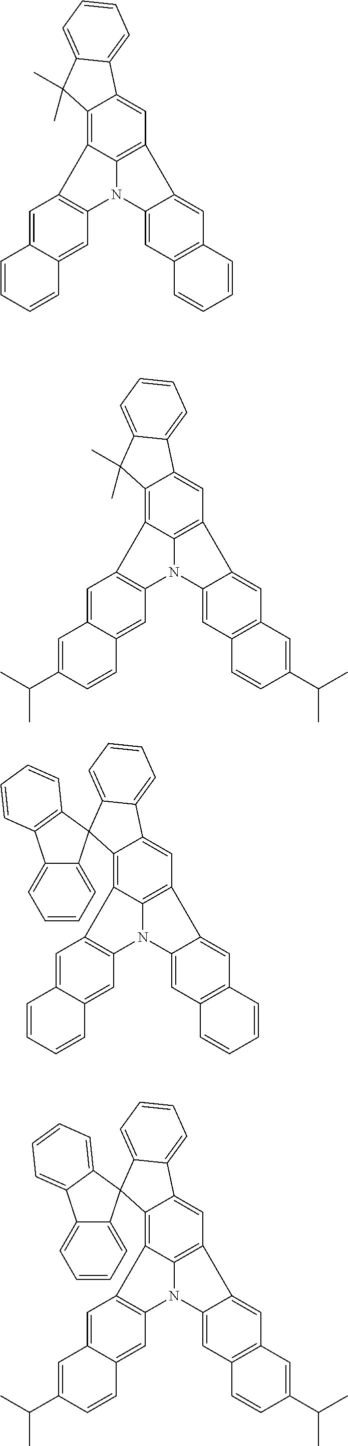

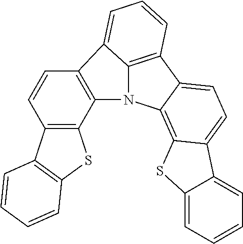 [SMILES: c1ccc2c(c1)sc1c2ccc2c3cccc4c5ccc6c7ccccc7sc6c5n(c34)c21]

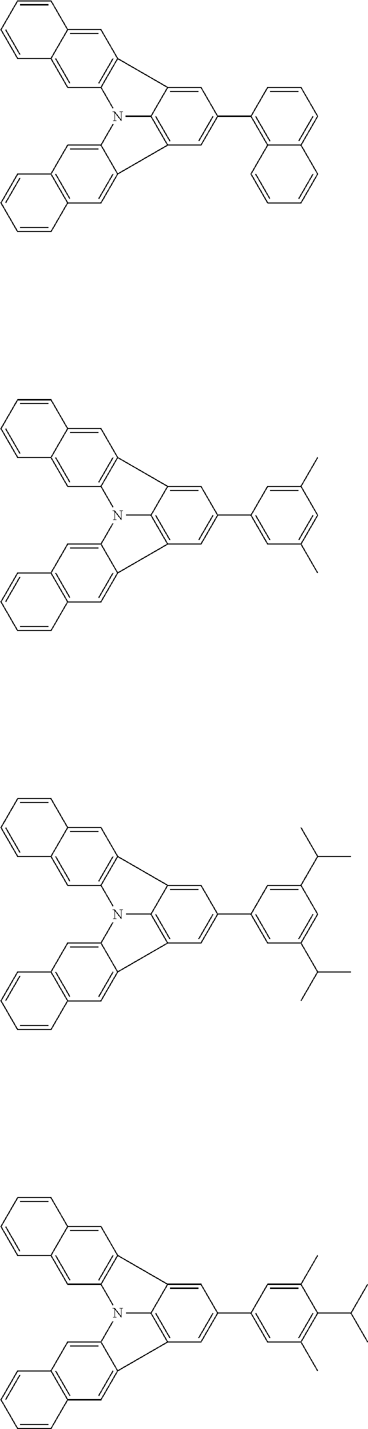 [SMILES: c1ccc2cc3c(cc2c1)c1cc(-c2cccc4ccccc24)cc2c4cc5ccccc5cc4n3c12 ; Cc1cc(C)cc(-c2cc3c4cc5ccccc5cc4n4c5cc6ccccc6cc5c(c2)c34)c1 ; CC(C)c1cc(-c2cc3c4cc5ccccc5cc4n4c5cc6ccccc6cc5c(c2)c34)cc(C(C)C)c1 ; Cc1cc(-c2cc3c4cc5ccccc5cc4n4c5cc6ccccc6cc5c(c2)c34)cc(C)c1C(C)C]

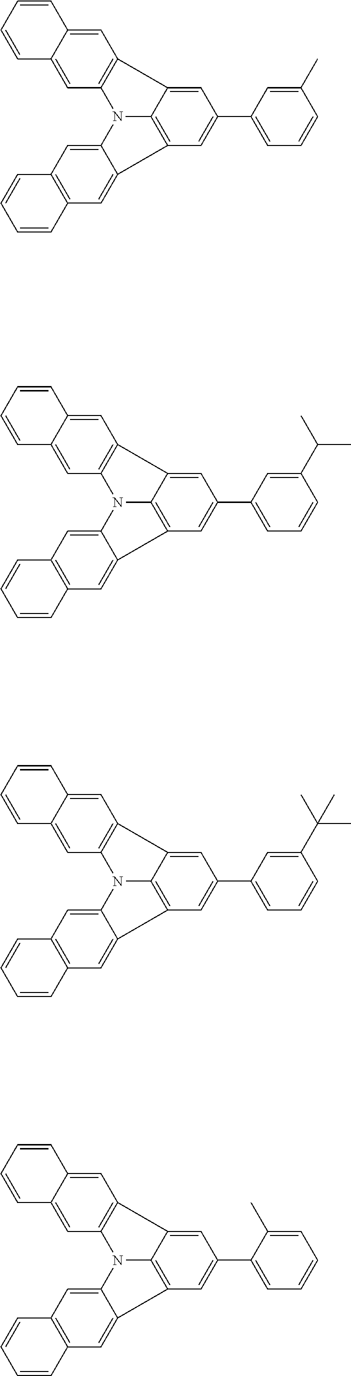 [SMILES: Cc1cccc(-c2cc3c4cc5ccccc5cc4n4c5cc6ccccc6cc5c(c2)c34)c1 ; CC(C)c1cccc(-c2cc3c4cc5ccccc5cc4n4c5cc6ccccc6cc5c(c2)c34)c1 ; CC(C)(C)c1cccc(-c2cc3c4cc5ccccc5cc4n4c5cc6ccccc6cc5c(c2)c34)c1 ; Cc1ccccc1-c1cc2c3cc4ccccc4cc3n3c4cc5ccccc5cc4c(c1)c23]

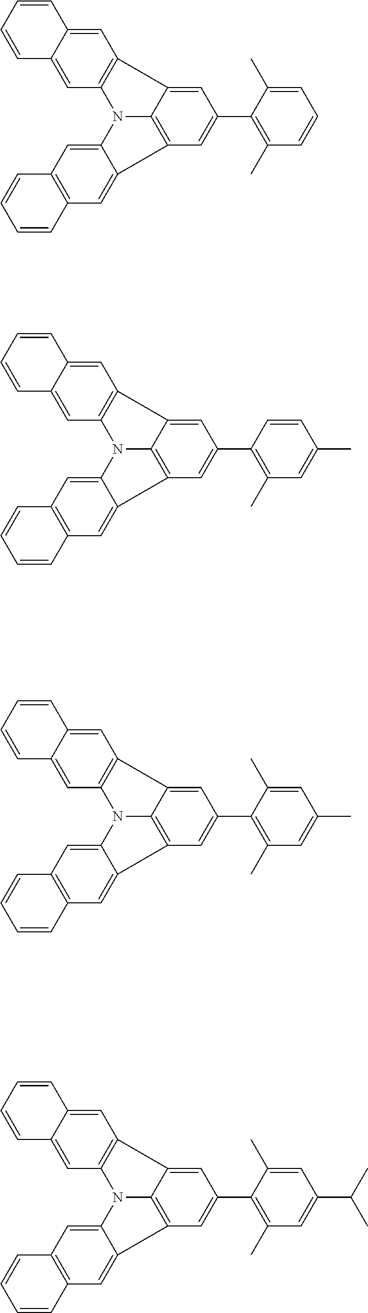 [SMILES: Cc1cccc(C)c1-c1cc2c3cc4ccccc4cc3n3c4cc5ccccc5cc4c(c1)c23 ; Cc1ccc(-c2cc3c4cc5ccccc5cc4n4c5cc6ccccc6cc5c(c2)c34)c(C)c1 ; Cc1cc(C)c(-c2cc3c4cc5ccccc5cc4n4c5cc6ccccc6cc5c(c2)c34)c(C)c1 ; Cc1cc(C(C)C)cc(C)c1-c1cc2c3cc4ccccc4cc3n3c4cc5ccccc5cc4c(c1)c23]

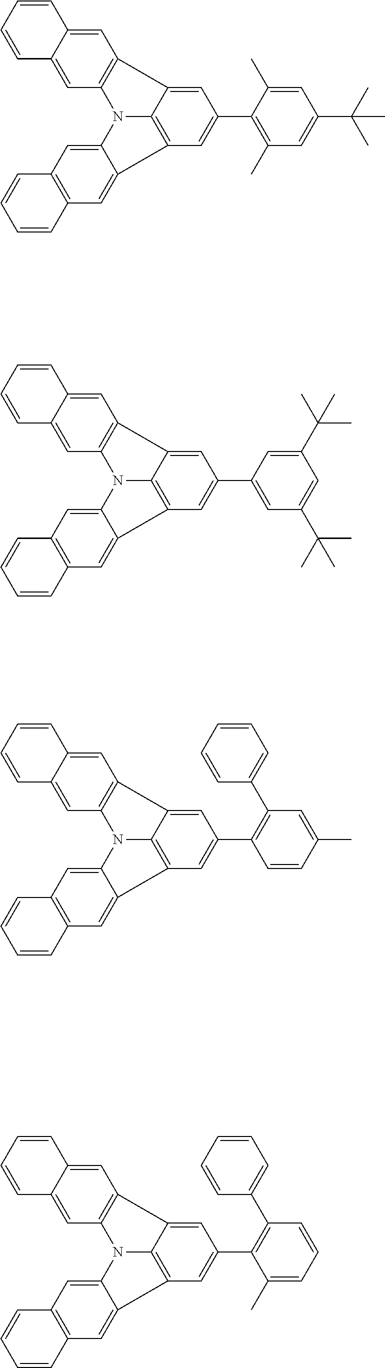 [SMILES: Cc1cc(C(C)(C)C)cc(C)c1-c1cc2c3cc4ccccc4cc3n3c4cc5ccccc5cc4c(c1)c23 ; CC(C)(C)c1cc(-c2cc3c4cc5ccccc5cc4n4c5cc6ccccc6cc5c(c2)c34)cc(C(C)(C)C)c1 ; Cc1ccc(-c2cc3c4cc5ccccc5cc4n4c5cc6ccccc6cc5c(c2)c34)c(-c2ccccc2)c1 ; Cc1cccc(-c2ccccc2)c1-c1cc2c3cc4ccccc4cc3n3c4cc5ccccc5cc4c(c1)c23]

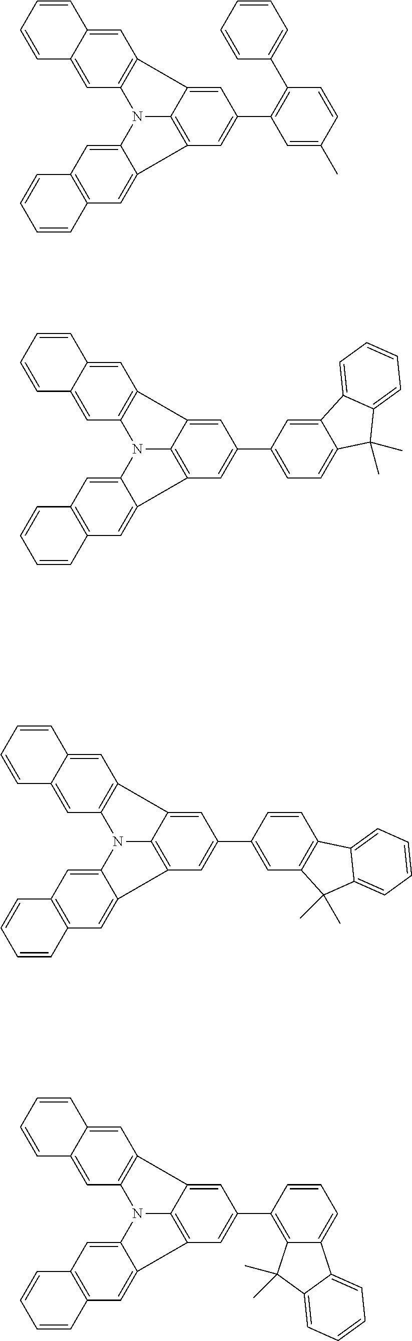 [SMILES: Cc1ccc(-c2ccccc2)c(-c2cc3c4cc5ccccc5cc4n4c5cc6ccccc6cc5c(c2)c34)c1 ; CC1(C)c2ccccc2-c2cc(-c3cc4c5cc6ccccc6cc5n5c6cc7ccccc7cc6c(c3)c45)ccc21 ; CC1(C)c2ccccc2-c2ccc(-c3cc4c5cc6ccccc6cc5n5c6cc7ccccc7cc6c(c3)c45)cc21 ; CC1(C)c2ccccc2-c2cccc(-c3cc4c5cc6ccccc6cc5n5c6cc7ccccc7cc6c(c3)c45)c21]

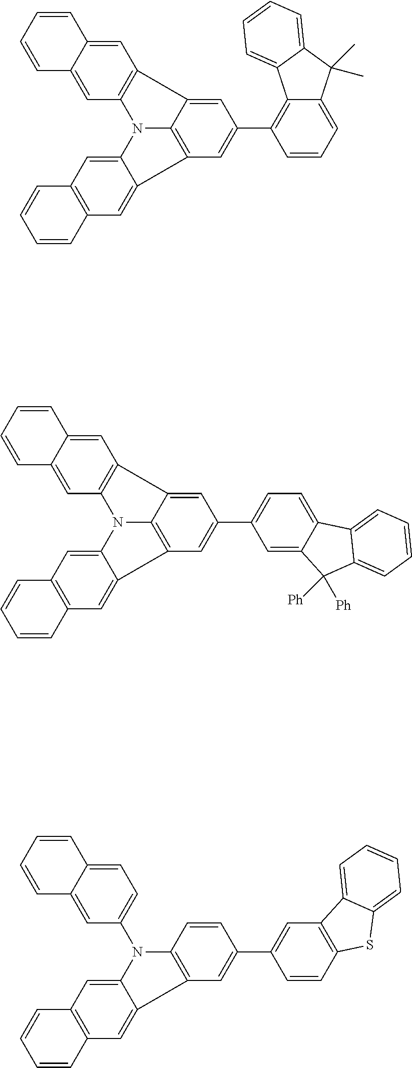 [SMILES: CC1(C)c2ccccc2-c2c(-c3cc4c5cc6ccccc6cc5n5c6cc7ccccc7cc6c(c3)c45)cccc21 ; c1ccc(C2(c3ccccc3)c3ccccc3-c3ccc(-c4cc5c6cc7ccccc7cc6n6c7cc8ccccc8cc7c(c4)c56)cc32)cc1 ; c1ccc2cc(-n3c4ccc(-c5ccc6sc7ccccc7c6c5)cc4c4cc5ccccc5cc43)ccc2c1]

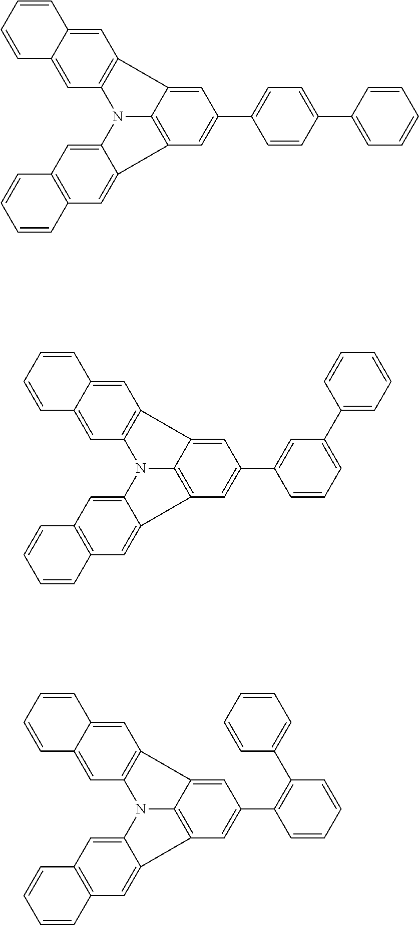 [SMILES: c1ccc(-c2ccc(-c3cc4c5cc6ccccc6cc5n5c6cc7ccccc7cc6c(c3)c45)cc2)cc1 ; c1ccc(-c2cccc(-c3cc4c5cc6ccccc6cc5n5c6cc7ccccc7cc6c(c3)c45)c2)cc1 ; c1ccc(-c2ccccc2-c2cc3c4cc5ccccc5cc4n4c5cc6ccccc6cc5c(c2)c34)cc1]

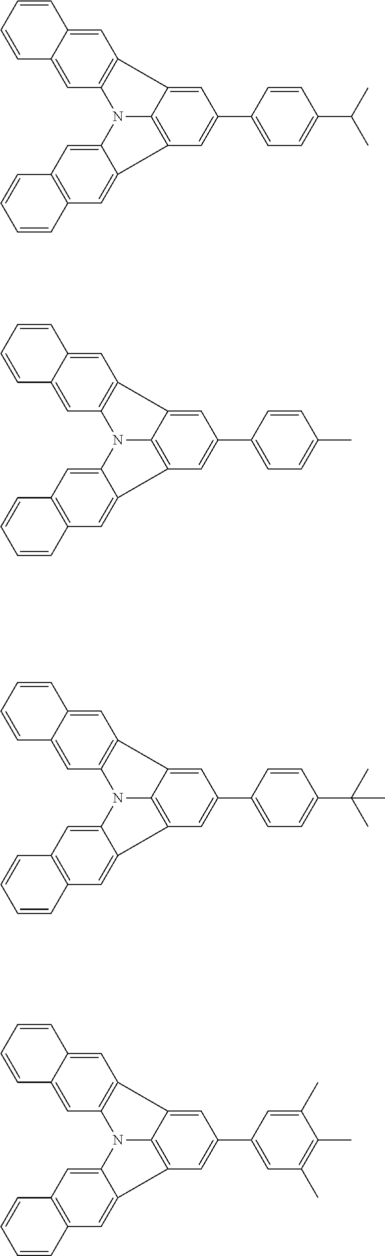 [SMILES: CC(C)c1ccc(-c2cc3c4cc5ccccc5cc4n4c5cc6ccccc6cc5c(c2)c34)cc1 ; Cc1ccc(-c2cc3c4cc5ccccc5cc4n4c5cc6ccccc6cc5c(c2)c34)cc1 ; CC(C)(C)c1ccc(-c2cc3c4cc5ccccc5cc4n4c5cc6ccccc6cc5c(c2)c34)cc1 ; Cc1cc(-c2cc3c4cc5ccccc5cc4n4c5cc6ccccc6cc5c(c2)c34)cc(C)c1C]

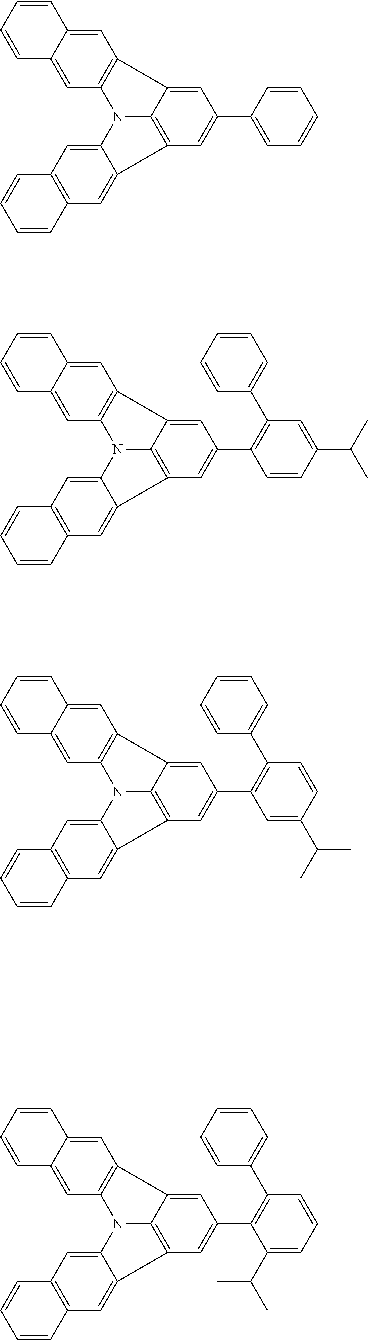 [SMILES: c1ccc(-c2cc3c4cc5ccccc5cc4n4c5cc6ccccc6cc5c(c2)c34)cc1 ; CC(C)c1ccc(-c2cc3c4cc5ccccc5cc4n4c5cc6ccccc6cc5c(c2)c34)c(-c2ccccc2)c1 ; CC(C)c1ccc(-c2ccccc2)c(-c2cc3c4cc5ccccc5cc4n4c5cc6ccccc6cc5c(c2)c34)c1 ; CC(C)c1cccc(-c2ccccc2)c1-c1cc2c3cc4ccccc4cc3n3c4cc5ccccc5cc4c(c1)c23]

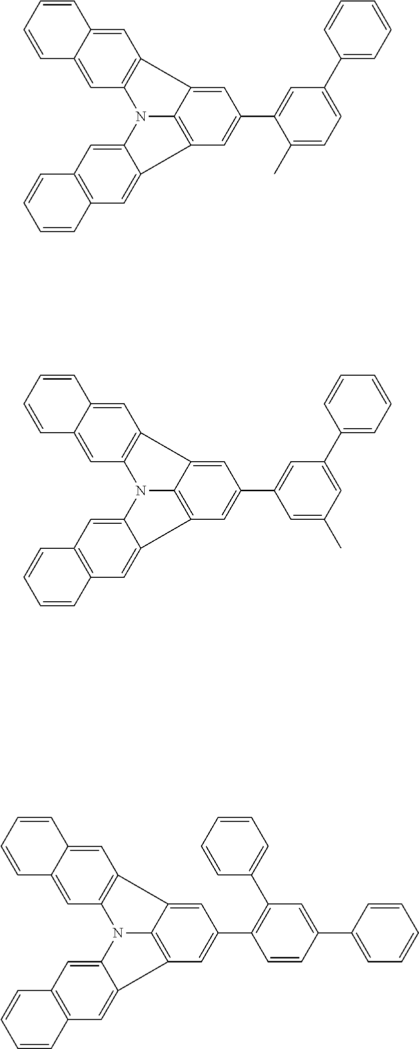 [SMILES: Cc1ccc(-c2ccccc2)cc1-c1cc2c3cc4ccccc4cc3n3c4cc5ccccc5cc4c(c1)c23 ; Cc1cc(-c2ccccc2)cc(-c2cc3c4cc5ccccc5cc4n4c5cc6ccccc6cc5c(c2)c34)c1 ; c1ccc(-c2ccc(-c3cc4c5cc6ccccc6cc5n5c6cc7ccccc7cc6c(c3)c45)c(-c3ccccc3)c2)cc1]

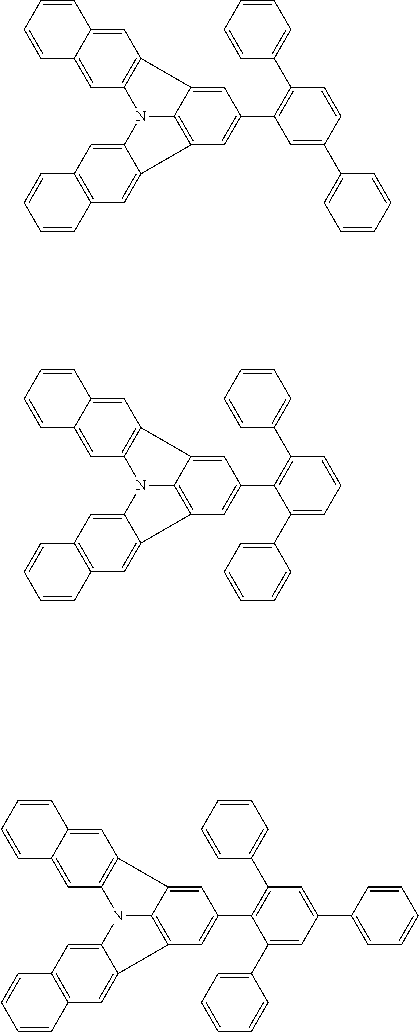 [SMILES: c1ccc(-c2ccc(-c3ccccc3)c(-c3cc4c5cc6ccccc6cc5n5c6cc7ccccc7cc6c(c3)c45)c2)cc1 ; c1ccc(-c2cccc(-c3ccccc3)c2-c2cc3c4cc5ccccc5cc4n4c5cc6ccccc6cc5c(c2)c34)cc1 ; c1ccc(-c2cc(-c3ccccc3)c(-c3cc4c5cc6ccccc6cc5n5c6cc7ccccc7cc6c(c3)c45)c(-c3ccccc3)c2)cc1]

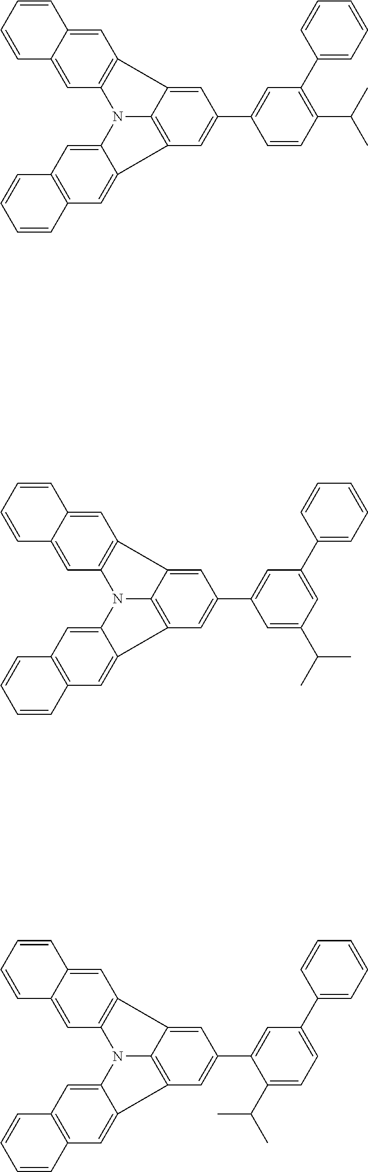 [SMILES: CC(C)c1ccc(-c2cc3c4cc5ccccc5cc4n4c5cc6ccccc6cc5c(c2)c34)cc1-c1ccccc1 ; CC(C)c1cc(-c2ccccc2)cc(-c2cc3c4cc5ccccc5cc4n4c5cc6ccccc6cc5c(c2)c34)c1 ; CC(C)c1ccc(-c2ccccc2)cc1-c1cc2c3cc4ccccc4cc3n3c4cc5ccccc5cc4c(c1)c23]

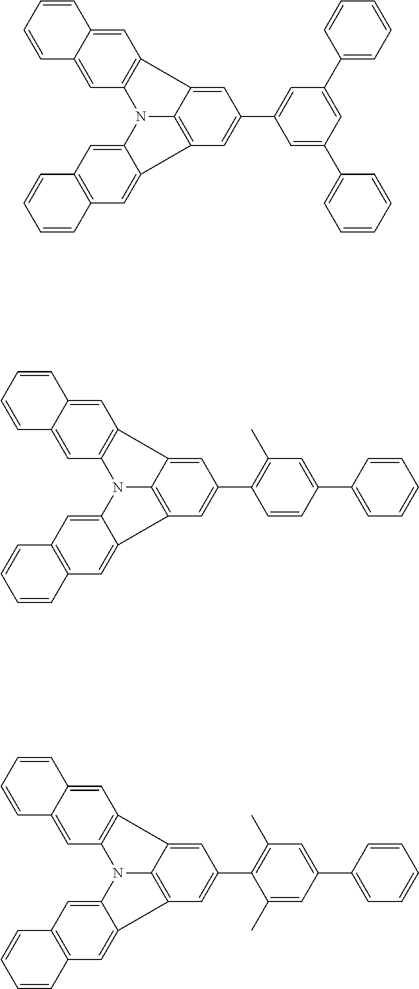 [SMILES: c1ccc(-c2cc(-c3ccccc3)cc(-c3cc4c5cc6ccccc6cc5n5c6cc7ccccc7cc6c(c3)c45)c2)cc1 ; Cc1cc(-c2ccccc2)ccc1-c1cc2c3cc4ccccc4cc3n3c4cc5ccccc5cc4c(c1)c23 ; Cc1cc(-c2ccccc2)cc(C)c1-c1cc2c3cc4ccccc4cc3n3c4cc5ccccc5cc4c(c1)c23]

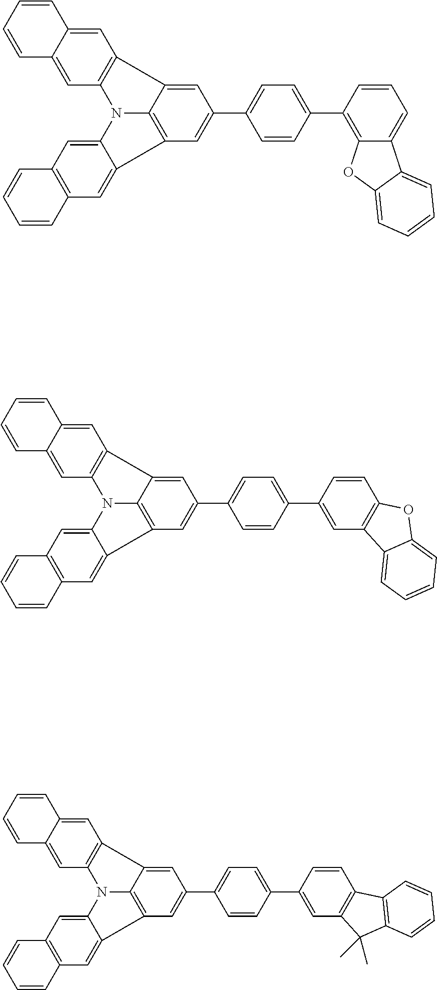 [SMILES: c1ccc2cc3c(cc2c1)c1cc(-c2ccc(-c4cccc5c4oc4ccccc45)cc2)cc2c4cc5ccccc5cc4n3c12 ; c1ccc2cc3c(cc2c1)c1cc(-c2ccc(-c4ccc5oc6ccccc6c5c4)cc2)cc2c4cc5ccccc5cc4n3c12 ; CC1(C)c2ccccc2-c2ccc(-c3ccc(-c4cc5c6cc7ccccc7cc6n6c7cc8ccccc8cc7c(c4)c56)cc3)cc21]

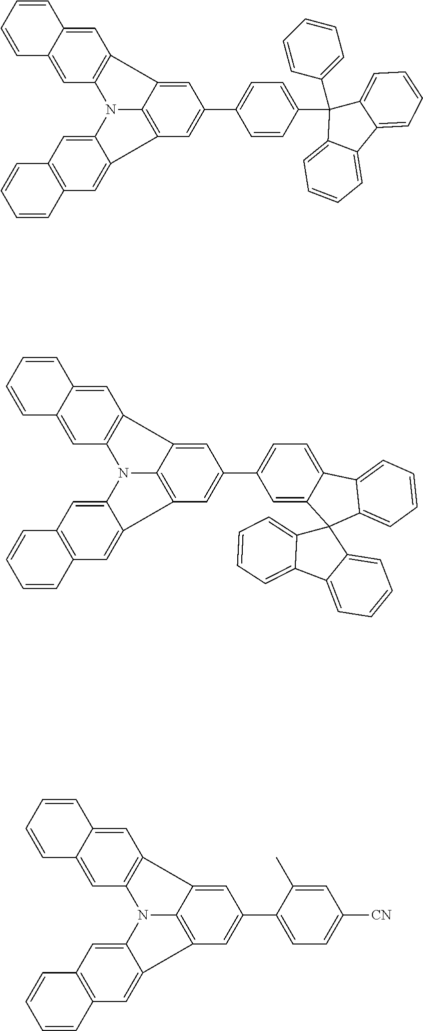 [SMILES: c1ccc(C2(c3ccc(-c4cc5c6cc7ccccc7cc6n6c7cc8ccccc8cc7c(c4)c56)cc3)c3ccccc3-c3ccccc32)cc1 ; c1ccc2c(c1)-c1ccccc1C21c2ccccc2-c2ccc(-c3cc4c5cc6ccccc6cc5n5c6cc7ccccc7cc6c(c3)c45)cc21 ; Cc1cc(C#N)ccc1-c1cc2c3cc4ccccc4cc3n3c4cc5ccccc5cc4c(c1)c23]

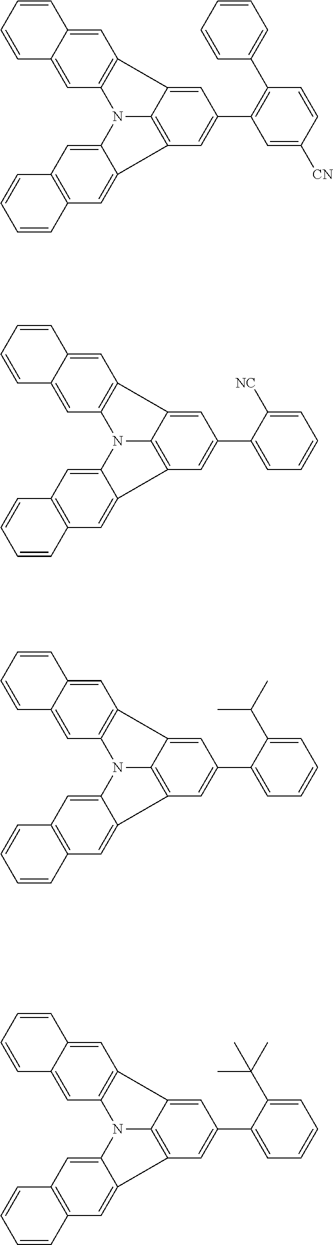 [SMILES: N#Cc1ccc(-c2ccccc2)c(-c2cc3c4cc5ccccc5cc4n4c5cc6ccccc6cc5c(c2)c34)c1 ; N#Cc1ccccc1-c1cc2c3cc4ccccc4cc3n3c4cc5ccccc5cc4c(c1)c23 ; CC(C)c1ccccc1-c1cc2c3cc4ccccc4cc3n3c4cc5ccccc5cc4c(c1)c23 ; CC(C)(C)c1ccccc1-c1cc2c3cc4ccccc4cc3n3c4cc5ccccc5cc4c(c1)c23]

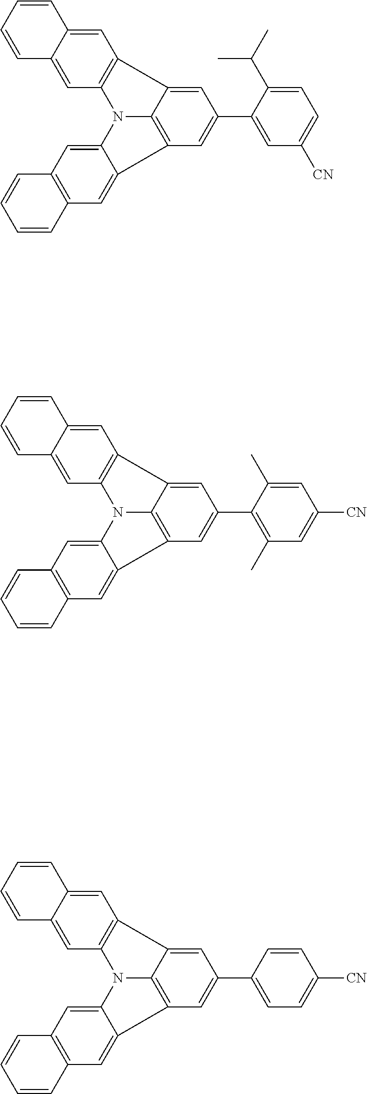 [SMILES: CC(C)c1ccc(C#N)cc1-c1cc2c3cc4ccccc4cc3n3c4cc5ccccc5cc4c(c1)c23 ; Cc1cc(C#N)cc(C)c1-c1cc2c3cc4ccccc4cc3n3c4cc5ccccc5cc4c(c1)c23 ; N#Cc1ccc(-c2cc3c4cc5ccccc5cc4n4c5cc6ccccc6cc5c(c2)c34)cc1]

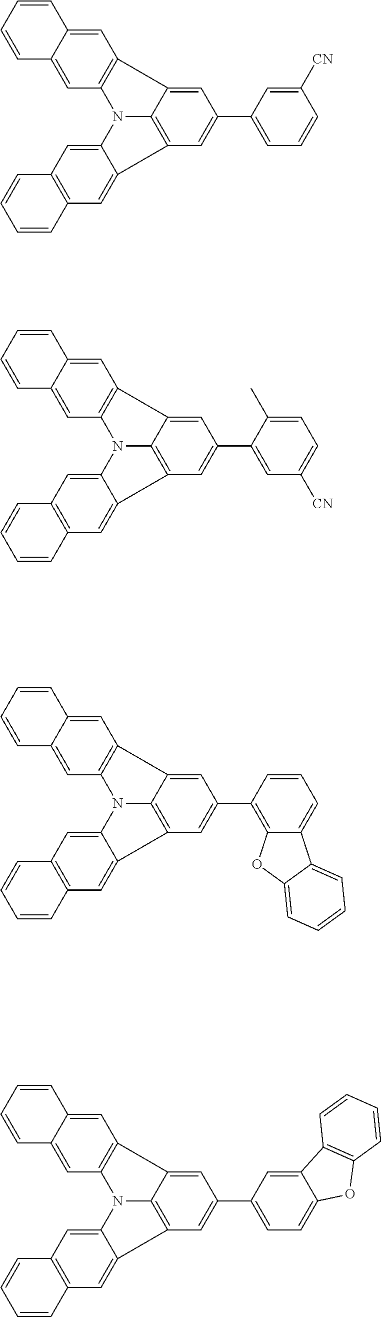 [SMILES: N#Cc1cccc(-c2cc3c4cc5ccccc5cc4n4c5cc6ccccc6cc5c(c2)c34)c1 ; Cc1ccc(C#N)cc1-c1cc2c3cc4ccccc4cc3n3c4cc5ccccc5cc4c(c1)c23 ; c1ccc2cc3c(cc2c1)c1cc(-c2cccc4c2oc2ccccc24)cc2c4cc5ccccc5cc4n3c12 ; c1ccc2cc3c(cc2c1)c1cc(-c2ccc4oc5ccccc5c4c2)cc2c4cc5ccccc5cc4n3c12]

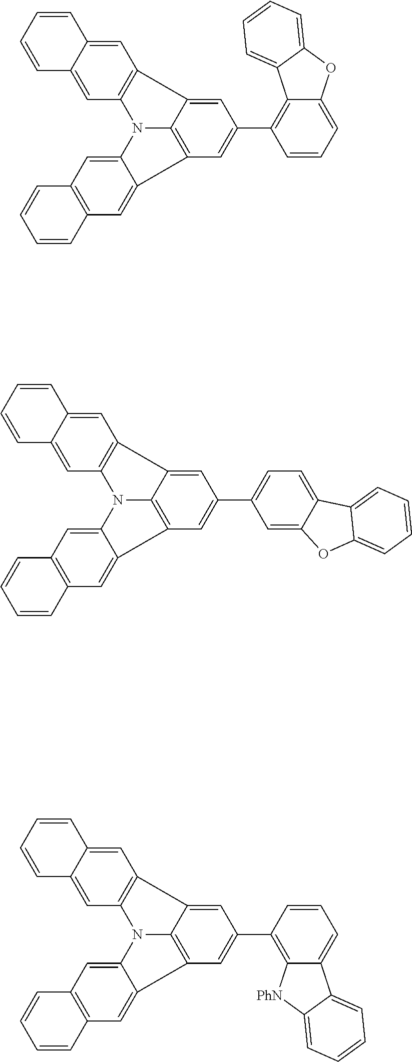

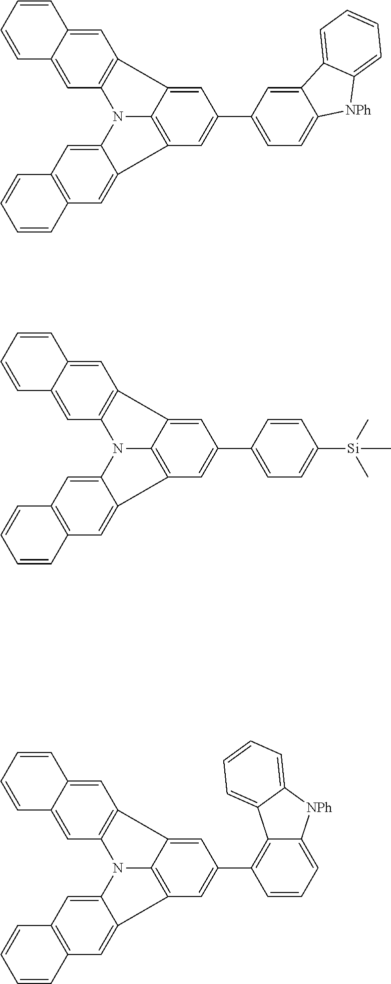

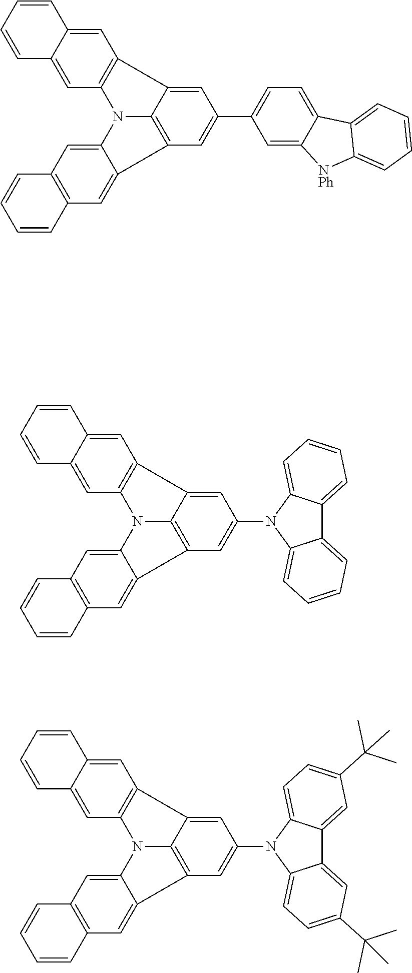

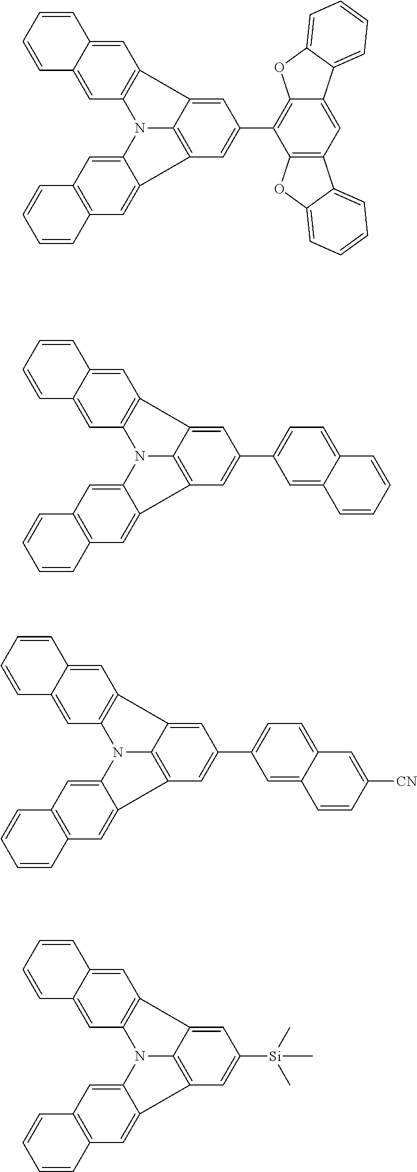 [SMILES: c1ccc2cc3c(cc2c1)c1cc(-c2c4oc5ccccc5c4cc4c2oc2ccccc24)cc2c4cc5ccccc5cc4n3c12 ; c1ccc2cc(-c3cc4c5cc6ccccc6cc5n5c6cc7ccccc7cc6c(c3)c45)ccc2c1 ; N#Cc1ccc2cc(-c3cc4c5cc6ccccc6cc5n5c6cc7ccccc7cc6c(c3)c45)ccc2c1 ; C[Si](C)(C)c1cc2c3cc4ccccc4cc3n3c4cc5ccccc5cc4c(c1)c23]

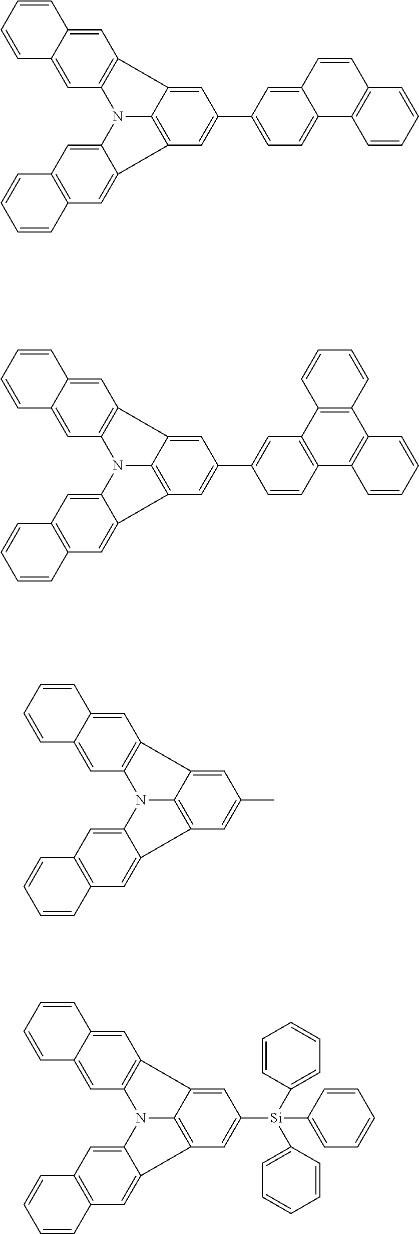 [SMILES: c1ccc2cc3c(cc2c1)c1cc(-c2ccc4c(ccc5ccccc54)c2)cc2c4cc5ccccc5cc4n3c12 ; c1ccc2cc3c(cc2c1)c1cc(-c2ccc4c5ccccc5c5ccccc5c4c2)cc2c4cc5ccccc5cc4n3c12 ; Cc1cc2c3cc4ccccc4cc3n3c4cc5ccccc5cc4c(c1)c23 ; c1ccc([Si](c2ccccc2)(c2ccccc2)c2cc3c4cc5ccccc5cc4n4c5cc6ccccc6cc5c(c2)c34)cc1]

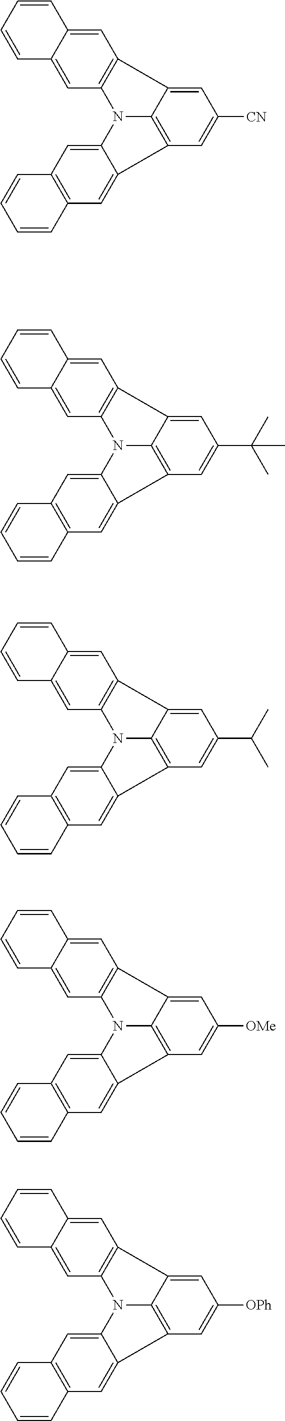 [SMILES: N#Cc1cc2c3cc4ccccc4cc3n3c4cc5ccccc5cc4c(c1)c23 ; CC(C)(C)c1cc2c3cc4ccccc4cc3n3c4cc5ccccc5cc4c(c1)c23 ; CC(C)c1cc2c3cc4ccccc4cc3n3c4cc5ccccc5cc4c(c1)c23 ; COc1cc2c3cc4ccccc4cc3n3c4cc5ccccc5cc4c(c1)c23 ; c1ccc(Oc2cc3c4cc5ccccc5cc4n4c5cc6ccccc6cc5c(c2)c34)cc1]

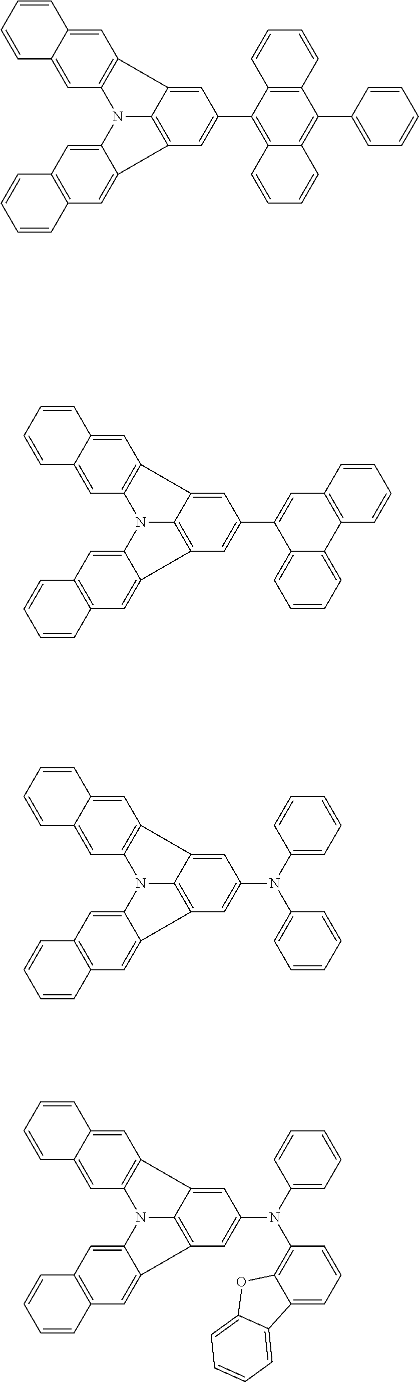 [SMILES: c1ccc(-c2c3ccccc3c(-c3cc4c5cc6ccccc6cc5n5c6cc7ccccc7cc6c(c3)c45)c3ccccc23)cc1 ; c1ccc2cc3c(cc2c1)c1cc(-c2cc4ccccc4c4ccccc24)cc2c4cc5ccccc5cc4n3c12 ; c1ccc(N(c2ccccc2)c2cc3c4cc5ccccc5cc4n4c5cc6ccccc6cc5c(c2)c34)cc1 ; c1ccc(N(c2cc3c4cc5ccccc5cc4n4c5cc6ccccc6cc5c(c2)c34)c2cccc3c2oc2ccccc23)cc1]

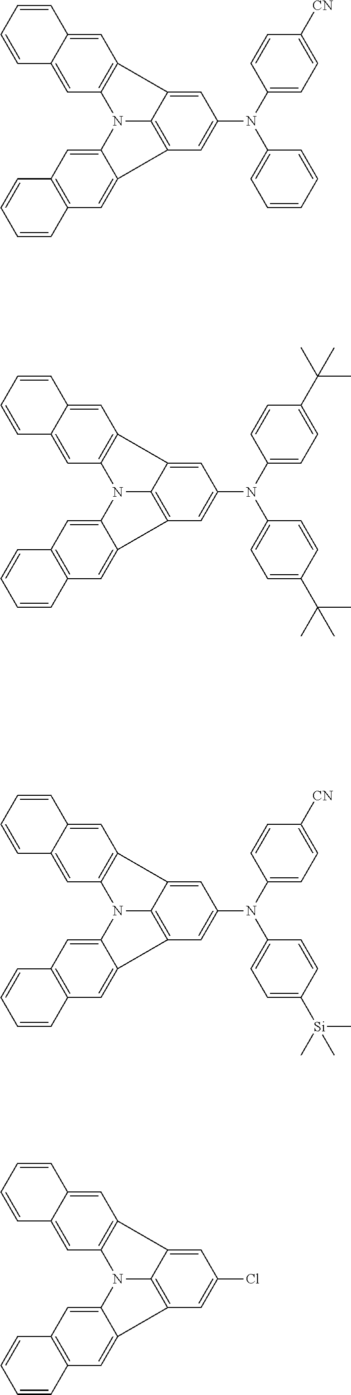 [SMILES: N#Cc1ccc(N(c2ccccc2)c2cc3c4cc5ccccc5cc4n4c5cc6ccccc6cc5c(c2)c34)cc1 ; CC(C)(C)c1ccc(N(c2ccc(C(C)(C)C)cc2)c2cc3c4cc5ccccc5cc4n4c5cc6ccccc6cc5c(c2)c34)cc1 ; C[Si](C)(C)c1ccc(N(c2ccc(C#N)cc2)c2cc3c4cc5ccccc5cc4n4c5cc6ccccc6cc5c(c2)c34)cc1 ; Clc1cc2c3cc4ccccc4cc3n3c4cc5ccccc5cc4c(c1)c23]

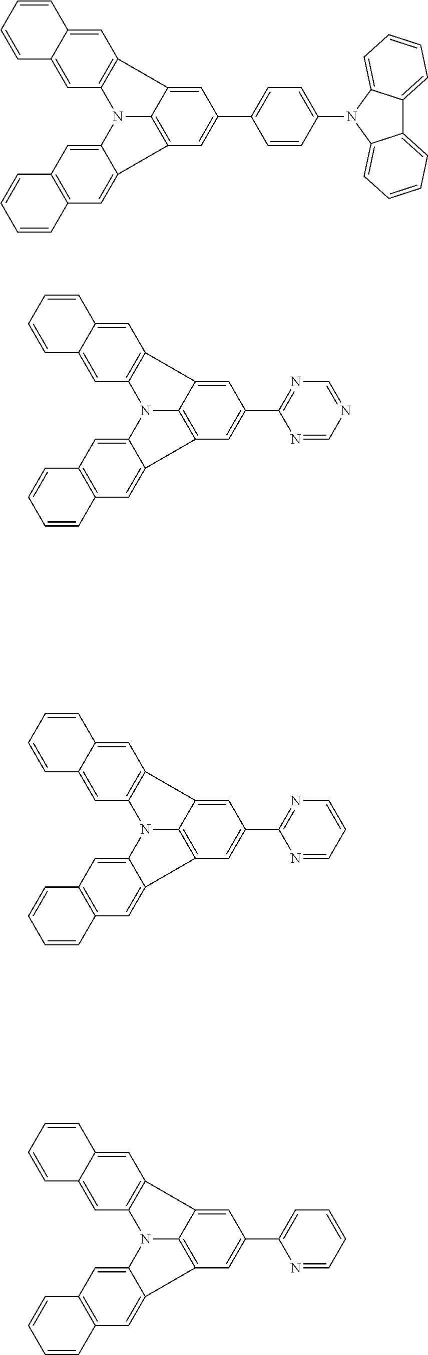 [SMILES: c1ccc2cc3c(cc2c1)c1cc(-c2ccc(-n4c5ccccc5c5ccccc54)cc2)cc2c4cc5ccccc5cc4n3c12 ; c1ccc2cc3c(cc2c1)c1cc(-c2ncncn2)cc2c4cc5ccccc5cc4n3c12 ; c1cnc(-c2cc3c4cc5ccccc5cc4n4c5cc6ccccc6cc5c(c2)c34)nc1 ; c1ccc(-c2cc3c4cc5ccccc5cc4n4c5cc6ccccc6cc5c(c2)c34)nc1]

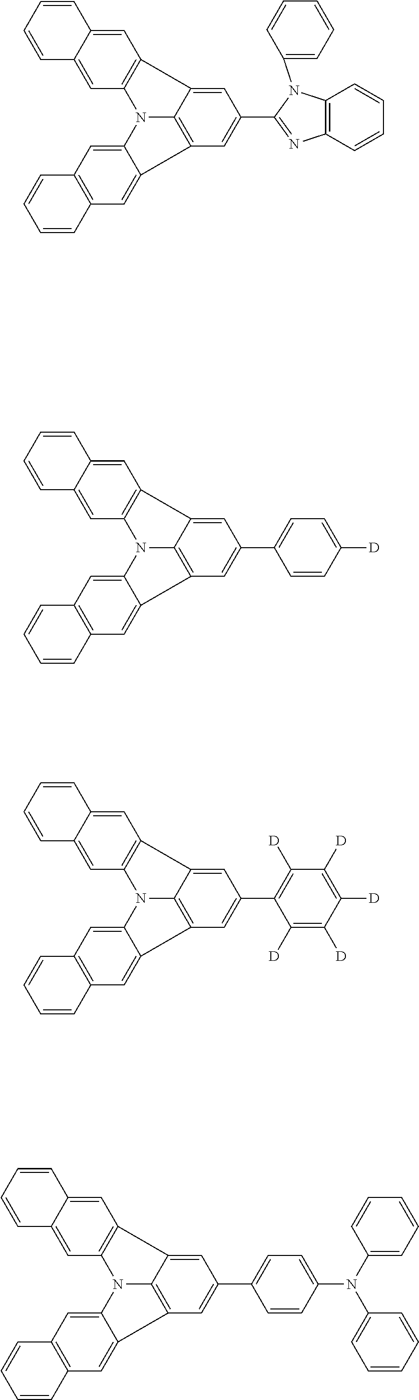 [SMILES: c1ccc(-n2c(-c3cc4c5cc6ccccc6cc5n5c6cc7ccccc7cc6c(c3)c45)nc3ccccc32)cc1 ; [2H]c1ccc(-c2cc3c4cc5ccccc5cc4n4c5cc6ccccc6cc5c(c2)c34)cc1 ; [2H]c1c([2H])c([2H])c(-c2cc3c4cc5ccccc5cc4n4c5cc6ccccc6cc5c(c2)c34)c([2H])c1[2H] ; c1ccc(N(c2ccccc2)c2ccc(-c3cc4c5cc6ccccc6cc5n5c6cc7ccccc7cc6c(c3)c45)cc2)cc1]

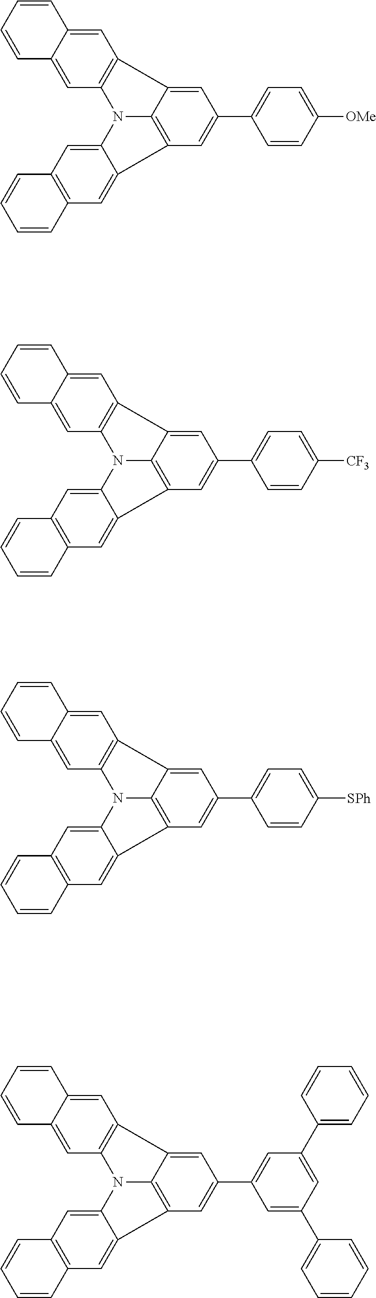

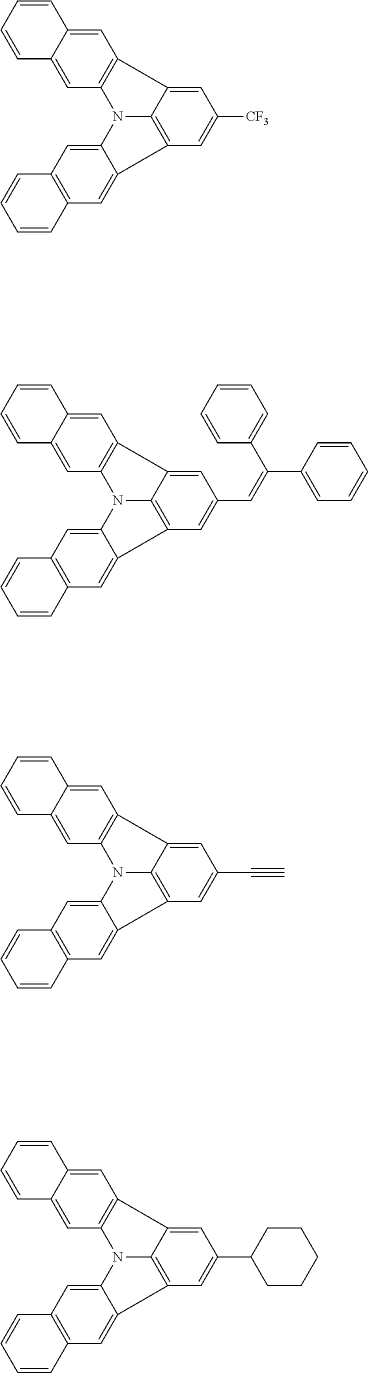 [SMILES: FC(F)(F)c1cc2c3cc4ccccc4cc3n3c4cc5ccccc5cc4c(c1)c23 ; C(=C(c1ccccc1)c1ccccc1)c1cc2c3cc4ccccc4cc3n3c4cc5ccccc5cc4c(c1)c23 ; C#Cc1cc2c3cc4ccccc4cc3n3c4cc5ccccc5cc4c(c1)c23 ; c1ccc2cc3c(cc2c1)c1cc(C2CCCCC2)cc2c4cc5ccccc5cc4n3c12]

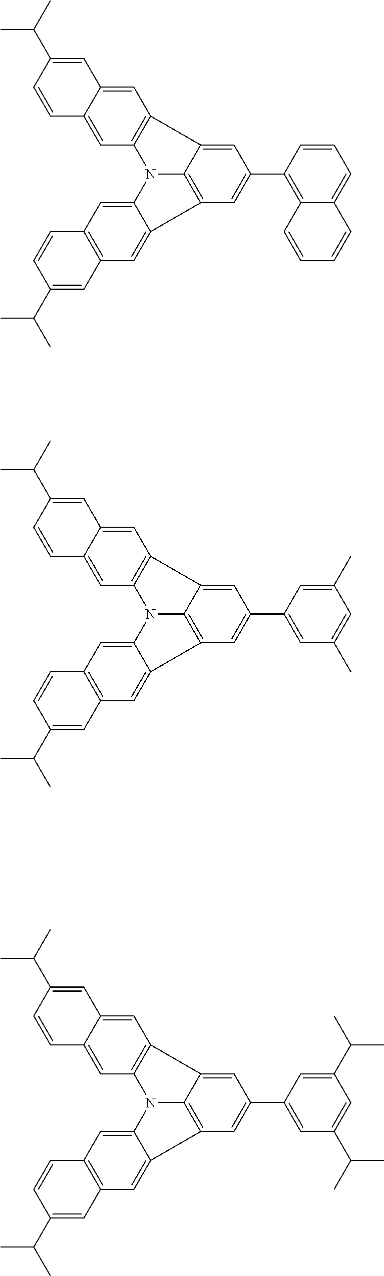

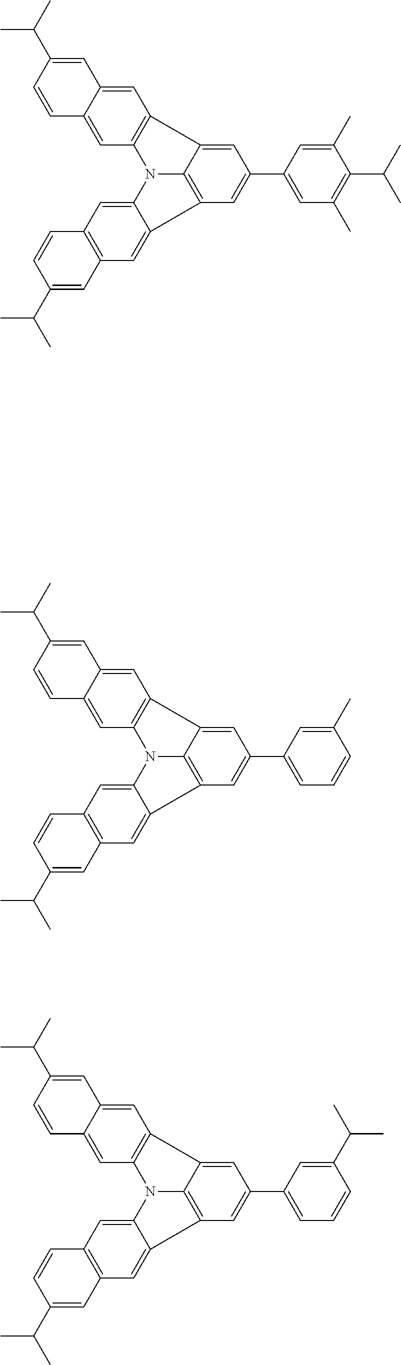

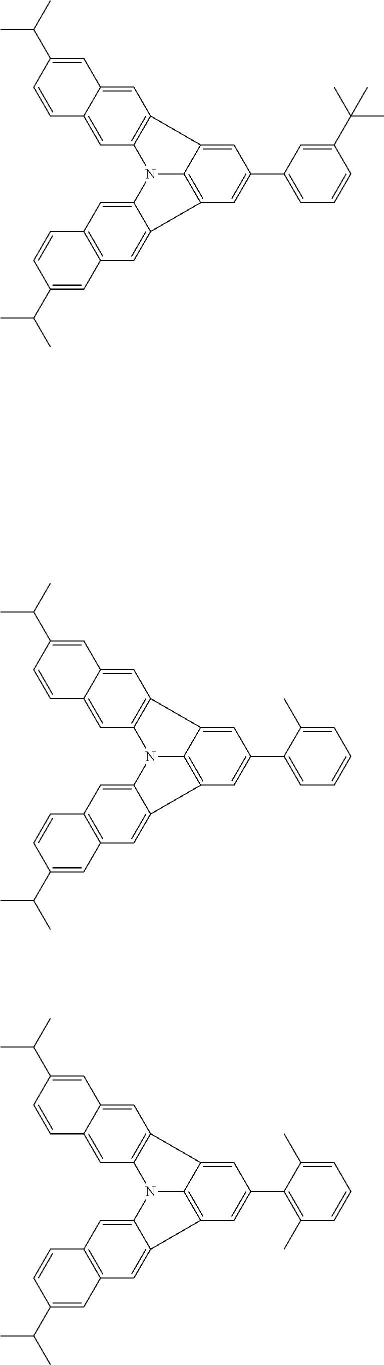

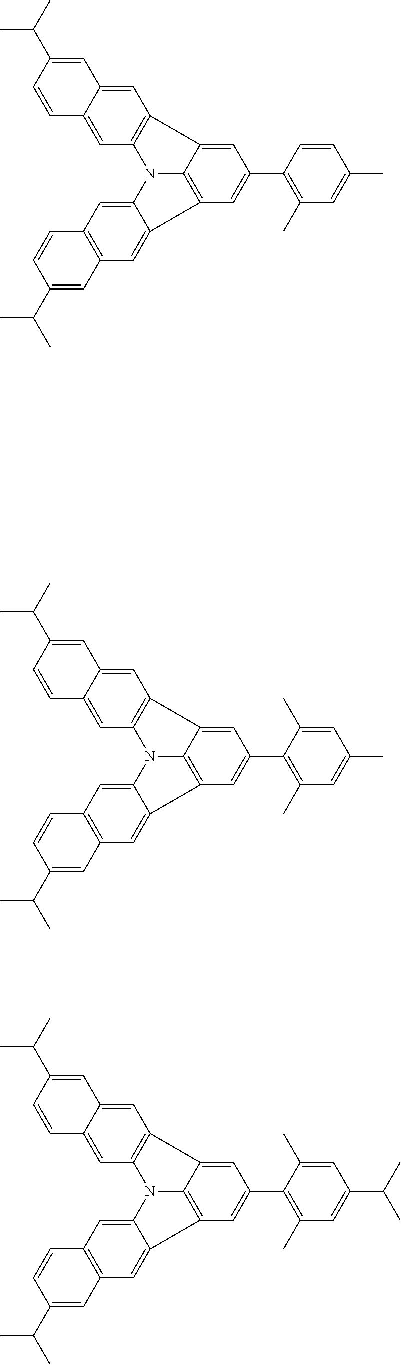

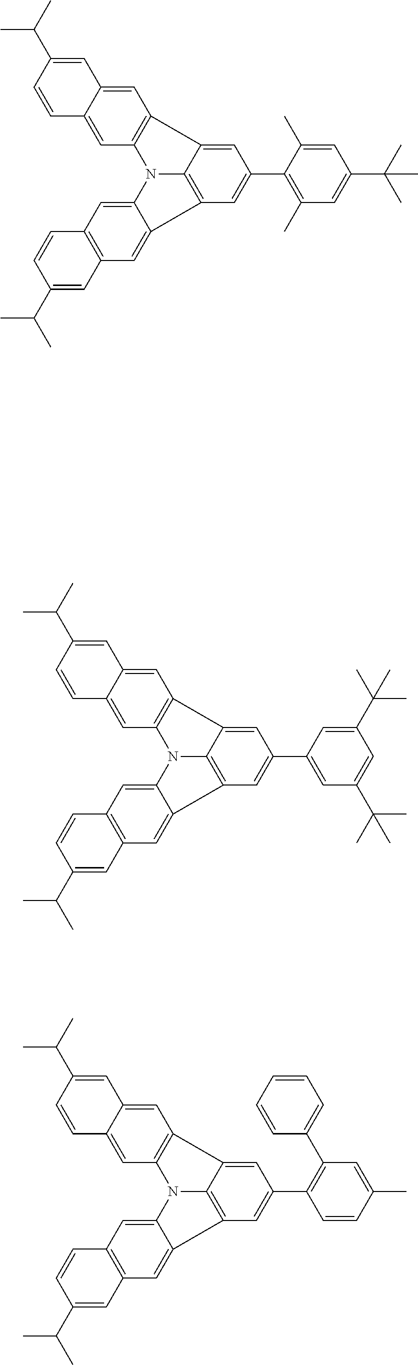

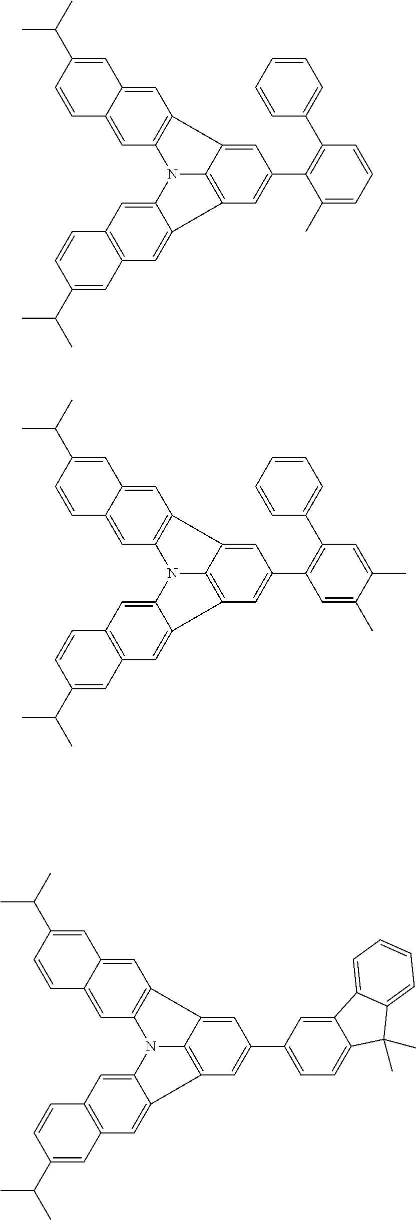

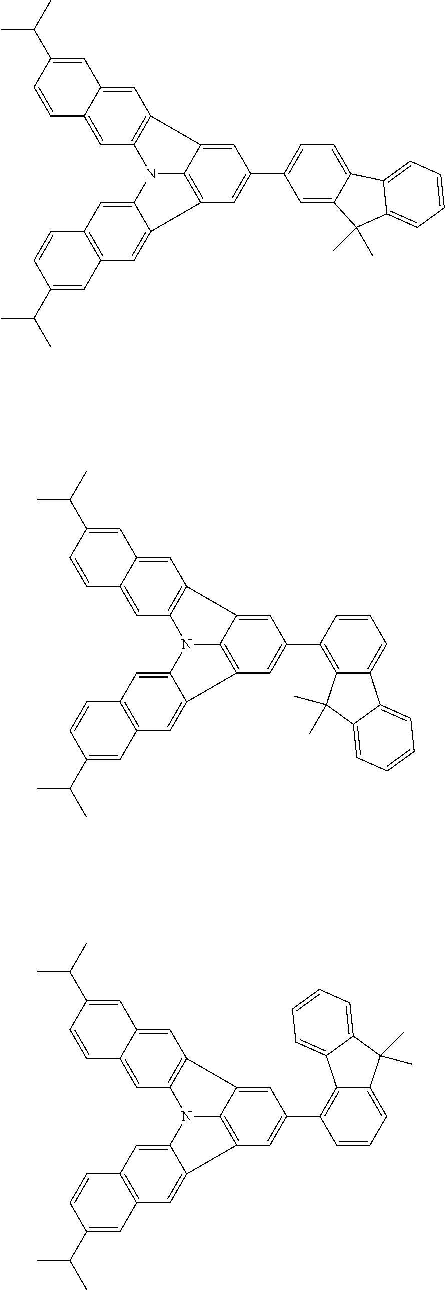 [SMILES: CC(C)c1ccc2cc3c(cc2c1)c1cc(-c2ccc4c(c2)C(C)(C)c2ccccc2-4)cc2c4cc5cc(C(C)C)ccc5cc4n3c12 ; CC(C)c1ccc2cc3c(cc2c1)c1cc(-c2cccc4c2C(C)(C)c2ccccc2-4)cc2c4cc5cc(C(C)C)ccc5cc4n3c12 ; CC(C)c1ccc2cc3c(cc2c1)c1cc(-c2cccc4c2-c2ccccc2C4(C)C)cc2c4cc5cc(C(C)C)ccc5cc4n3c12]

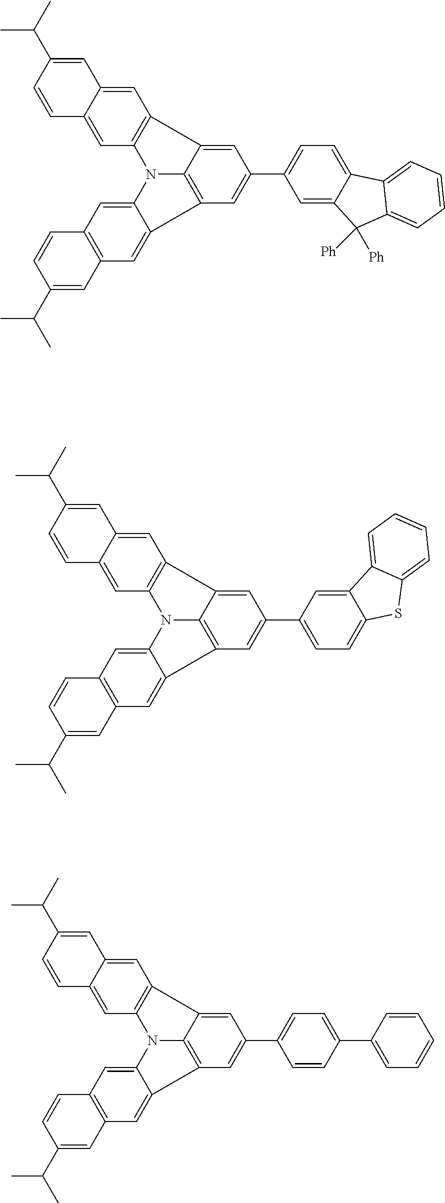 [SMILES: CC(C)c1ccc2cc3c(cc2c1)c1cc(-c2ccc4c(c2)C(c2ccccc2)(c2ccccc2)c2ccccc2-4)cc2c4cc5cc(C(C)C)ccc5cc4n3c12 ; CC(C)c1ccc2cc3c(cc2c1)c1cc(-c2ccc4sc5ccccc5c4c2)cc2c4cc5cc(C(C)C)ccc5cc4n3c12 ; CC(C)c1ccc2cc3c(cc2c1)c1cc(-c2ccc(-c4ccccc4)cc2)cc2c4cc5cc(C(C)C)ccc5cc4n3c12]

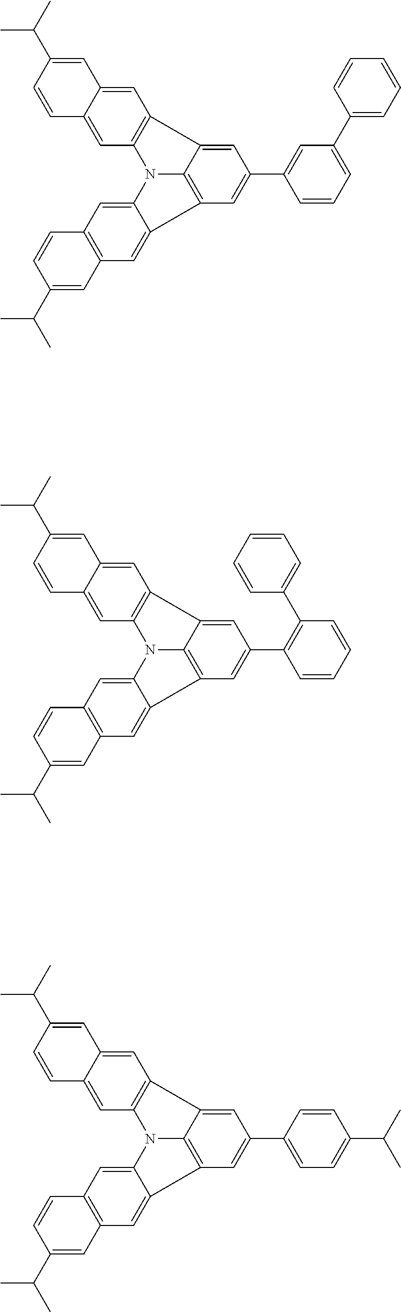 [SMILES: CC(C)c1ccc2cc3c(cc2c1)c1cc(-c2cccc(-c4ccccc4)c2)cc2c4cc5cc(C(C)C)ccc5cc4n3c12 ; CC(C)c1ccc2cc3c(cc2c1)c1cc(-c2ccccc2-c2ccccc2)cc2c4cc5cc(C(C)C)ccc5cc4n3c12 ; CC(C)c1ccc(-c2cc3c4cc5cc(C(C)C)ccc5cc4n4c5cc6ccc(C(C)C)cc6cc5c(c2)c34)cc1]

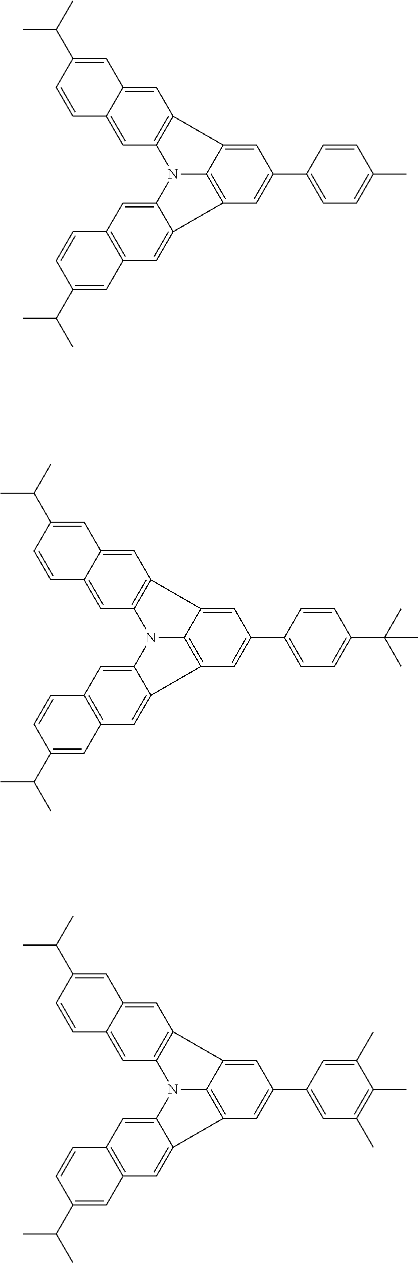

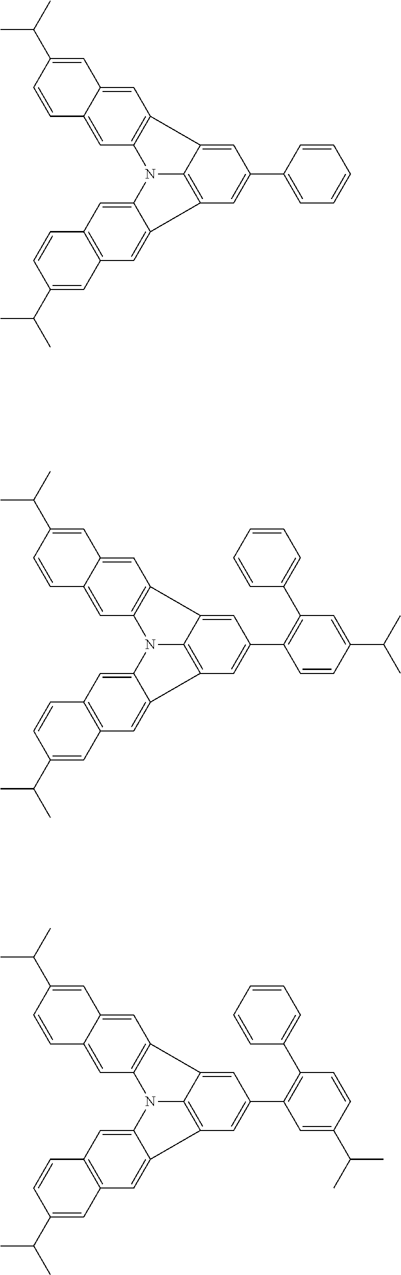

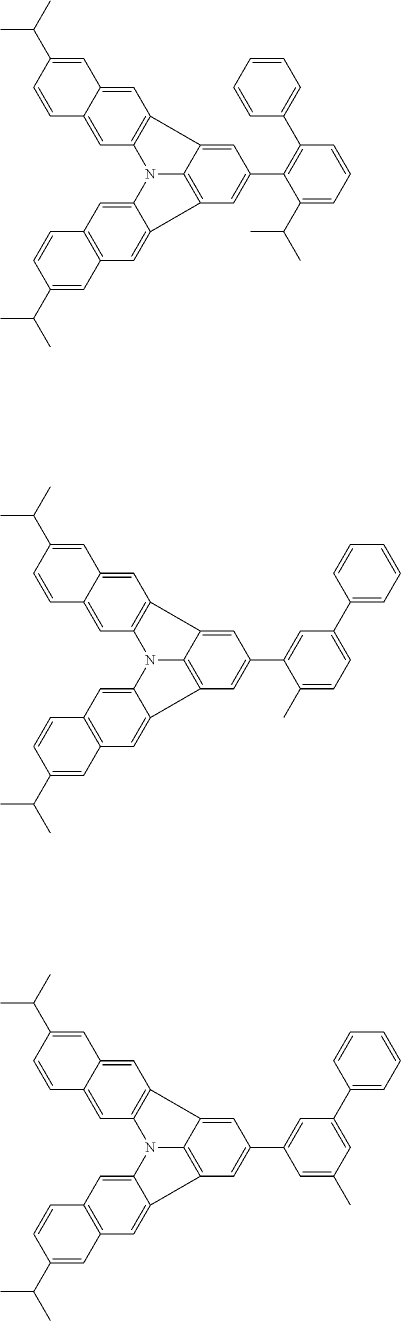 [SMILES: CC(C)c1ccc2cc3c(cc2c1)c1cc(-c2c(-c4ccccc4)cccc2C(C)C)cc2c4cc5cc(C(C)C)ccc5cc4n3c12 ; Cc1ccc(-c2ccccc2)cc1-c1cc2c3cc4cc(C(C)C)ccc4cc3n3c4cc5ccc(C(C)C)cc5cc4c(c1)c23 ; Cc1cc(-c2ccccc2)cc(-c2cc3c4cc5cc(C(C)C)ccc5cc4n4c5cc6ccc(C(C)C)cc6cc5c(c2)c34)c1]

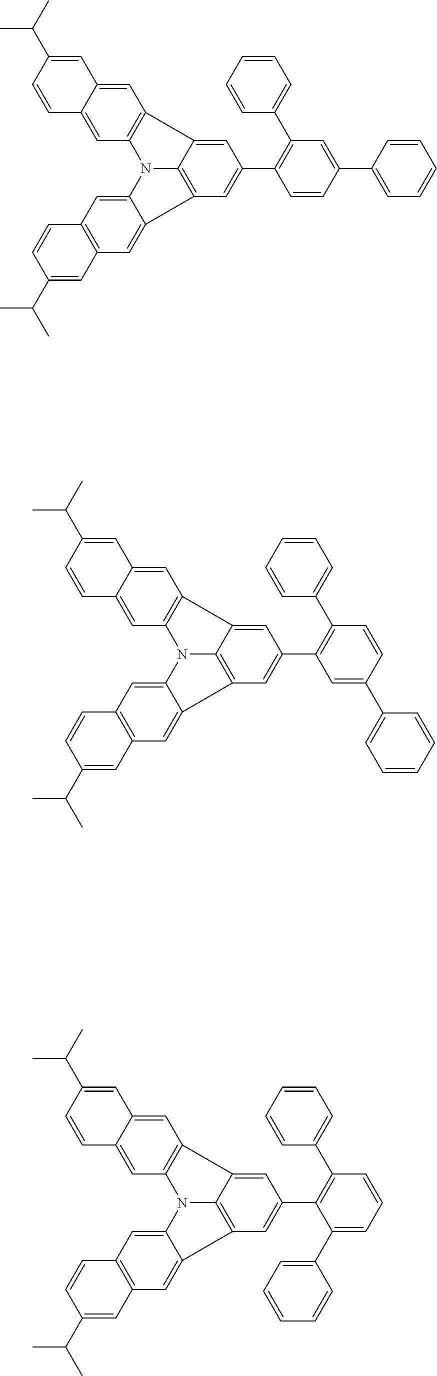 [SMILES: CC(C)c1ccc2cc3c(cc2c1)c1cc(-c2ccc(-c4ccccc4)cc2-c2ccccc2)cc2c4cc5cc(C(C)C)ccc5cc4n3c12 ; CC(C)c1ccc2cc3c(cc2c1)c1cc(-c2cc(-c4ccccc4)ccc2-c2ccccc2)cc2c4cc5cc(C(C)C)ccc5cc4n3c12 ; CC(C)c1ccc2cc3c(cc2c1)c1cc(-c2c(-c4ccccc4)cccc2-c2ccccc2)cc2c4cc5cc(C(C)C)ccc5cc4n3c12]

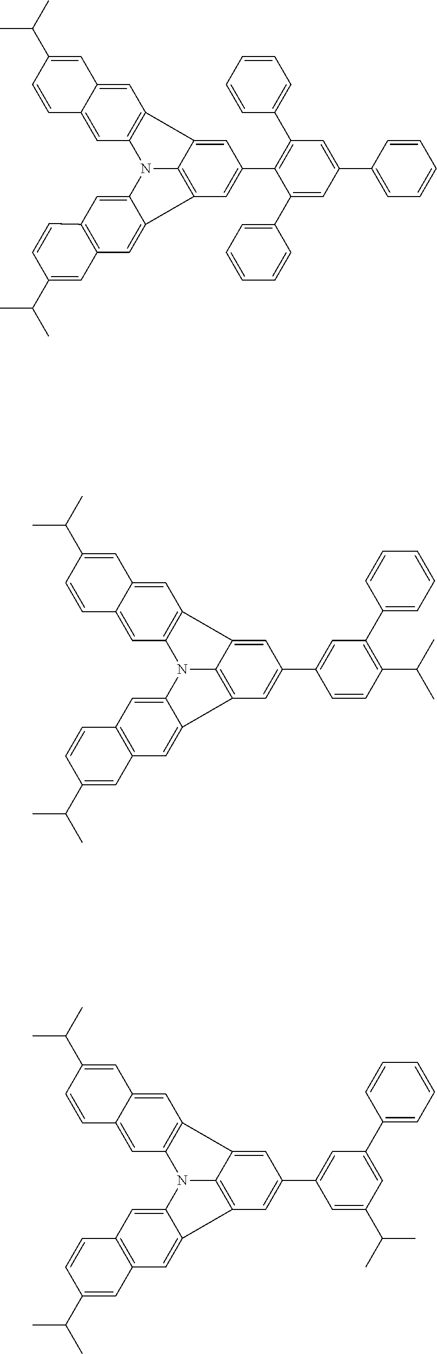 [SMILES: CC(C)c1ccc2cc3c(cc2c1)c1cc(-c2c(-c4ccccc4)cc(-c4ccccc4)cc2-c2ccccc2)cc2c4cc5cc(C(C)C)ccc5cc4n3c12 ; CC(C)c1ccc2cc3c(cc2c1)c1cc(-c2ccc(C(C)C)c(-c4ccccc4)c2)cc2c4cc5cc(C(C)C)ccc5cc4n3c12 ; CC(C)c1cc(-c2ccccc2)cc(-c2cc3c4cc5cc(C(C)C)ccc5cc4n4c5cc6ccc(C(C)C)cc6cc5c(c2)c34)c1]

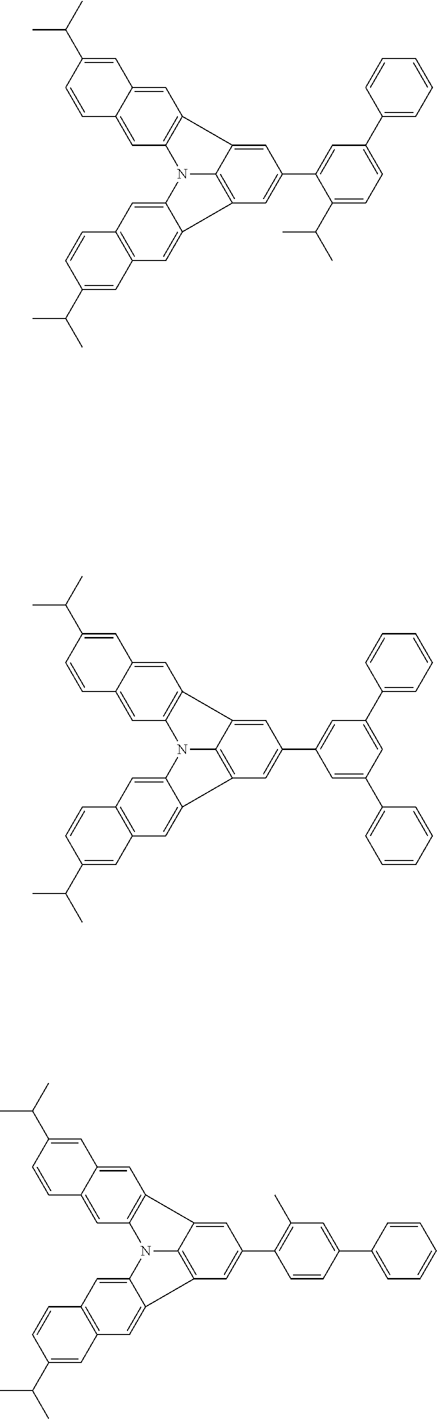 [SMILES: CC(C)c1ccc2cc3c(cc2c1)c1cc(-c2cc(-c4ccccc4)ccc2C(C)C)cc2c4cc5cc(C(C)C)ccc5cc4n3c12 ; CC(C)c1ccc2cc3c(cc2c1)c1cc(-c2cc(-c4ccccc4)cc(-c4ccccc4)c2)cc2c4cc5cc(C(C)C)ccc5cc4n3c12 ; Cc1cc(-c2ccccc2)ccc1-c1cc2c3cc4cc(C(C)C)ccc4cc3n3c4cc5ccc(C(C)C)cc5cc4c(c1)c23]

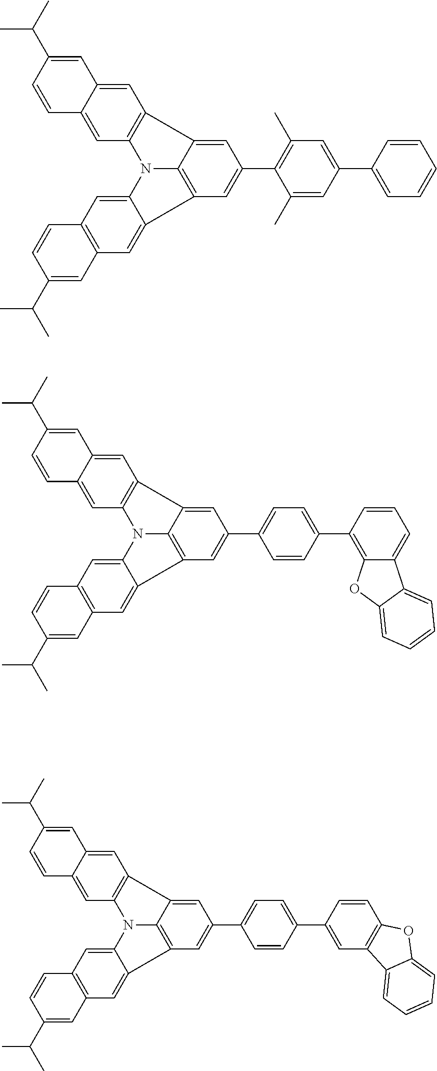

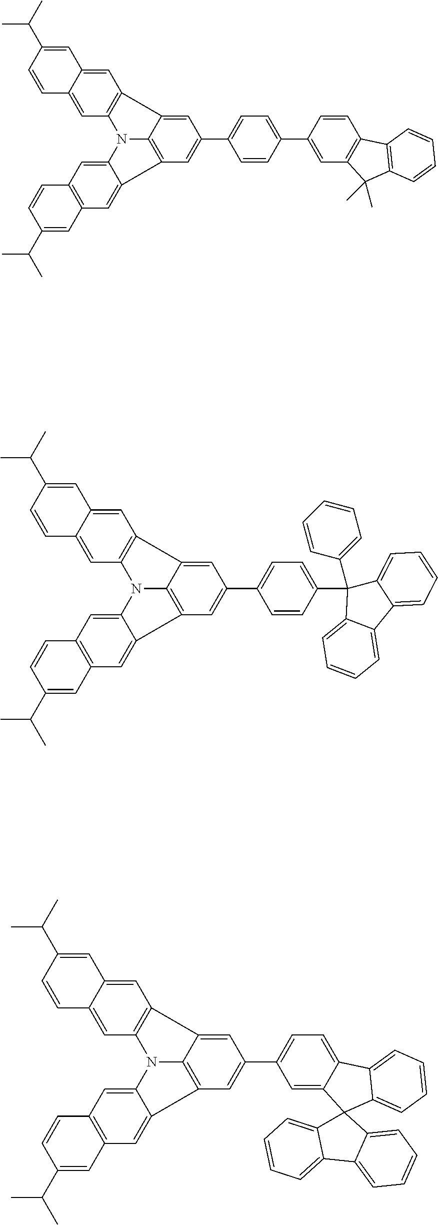 [SMILES: CC(C)c1ccc2cc3c(cc2c1)c1cc(-c2ccc(-c4ccc5c(c4)C(C)(C)c4ccccc4-5)cc2)cc2c4cc5cc(C(C)C)ccc5cc4n3c12 ; CC(C)c1ccc2cc3c(cc2c1)c1cc(-c2ccc(C4(c5ccccc5)c5ccccc5-c5ccccc54)cc2)cc2c4cc5cc(C(C)C)ccc5cc4n3c12 ; CC(C)c1ccc2cc3c(cc2c1)c1cc(-c2ccc4c(c2)C2(c5ccccc5-c5ccccc52)c2ccccc2-4)cc2c4cc5cc(C(C)C)ccc5cc4n3c12]

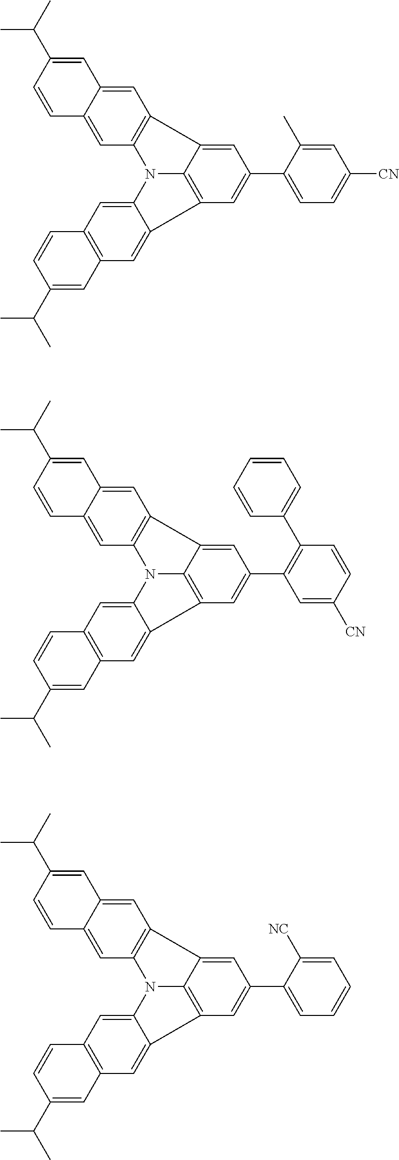 [SMILES: Cc1cc(C#N)ccc1-c1cc2c3cc4cc(C(C)C)ccc4cc3n3c4cc5ccc(C(C)C)cc5cc4c(c1)c23 ; CC(C)c1ccc2cc3c(cc2c1)c1cc(-c2cc(C#N)ccc2-c2ccccc2)cc2c4cc5cc(C(C)C)ccc5cc4n3c12 ; CC(C)c1ccc2cc3c(cc2c1)c1cc(-c2ccccc2C#N)cc2c4cc5cc(C(C)C)ccc5cc4n3c12]

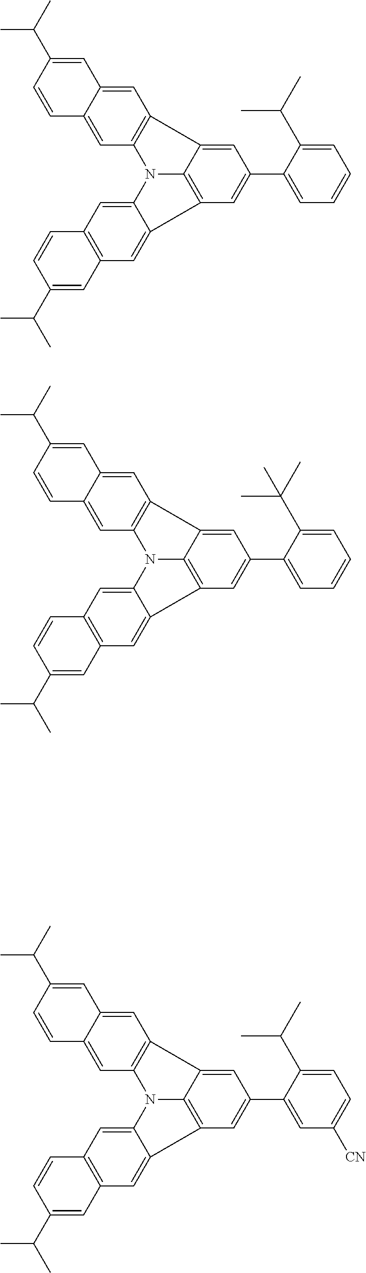

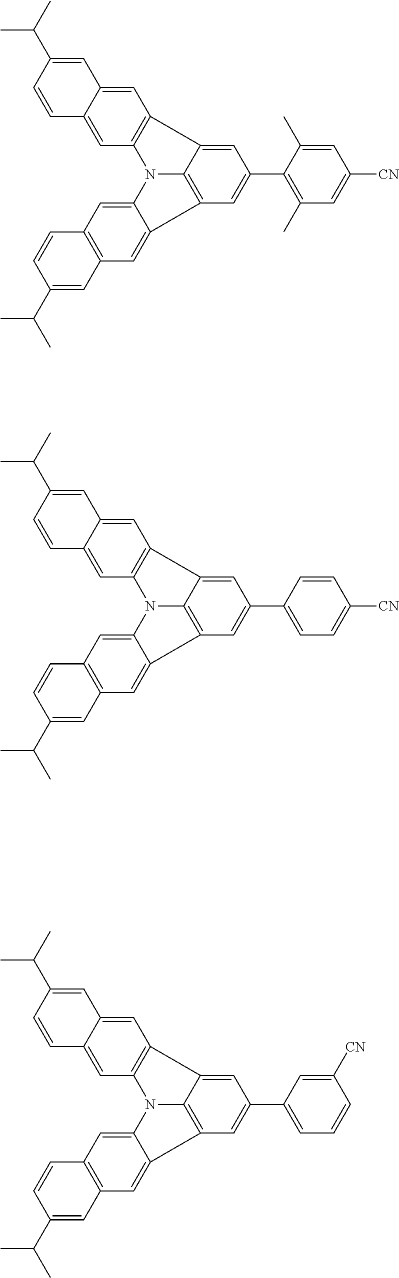 [SMILES: Cc1cc(C#N)cc(C)c1-c1cc2c3cc4cc(C(C)C)ccc4cc3n3c4cc5ccc(C(C)C)cc5cc4c(c1)c23 ; CC(C)c1ccc2cc3c(cc2c1)c1cc(-c2ccc(C#N)cc2)cc2c4cc5cc(C(C)C)ccc5cc4n3c12 ; CC(C)c1ccc2cc3c(cc2c1)c1cc(-c2cccc(C#N)c2)cc2c4cc5cc(C(C)C)ccc5cc4n3c12]

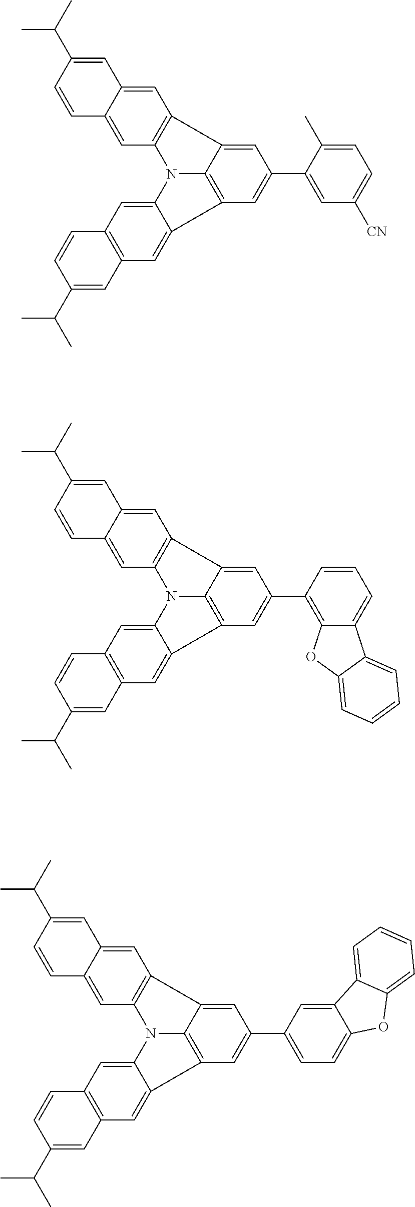 [SMILES: Cc1ccc(C#N)cc1-c1cc2c3cc4cc(C(C)C)ccc4cc3n3c4cc5ccc(C(C)C)cc5cc4c(c1)c23 ; CC(C)c1ccc2cc3c(cc2c1)c1cc(-c2cccc4c2oc2ccccc24)cc2c4cc5cc(C(C)C)ccc5cc4n3c12 ; CC(C)c1ccc2cc3c(cc2c1)c1cc(-c2ccc4oc5ccccc5c4c2)cc2c4cc5cc(C(C)C)ccc5cc4n3c12]

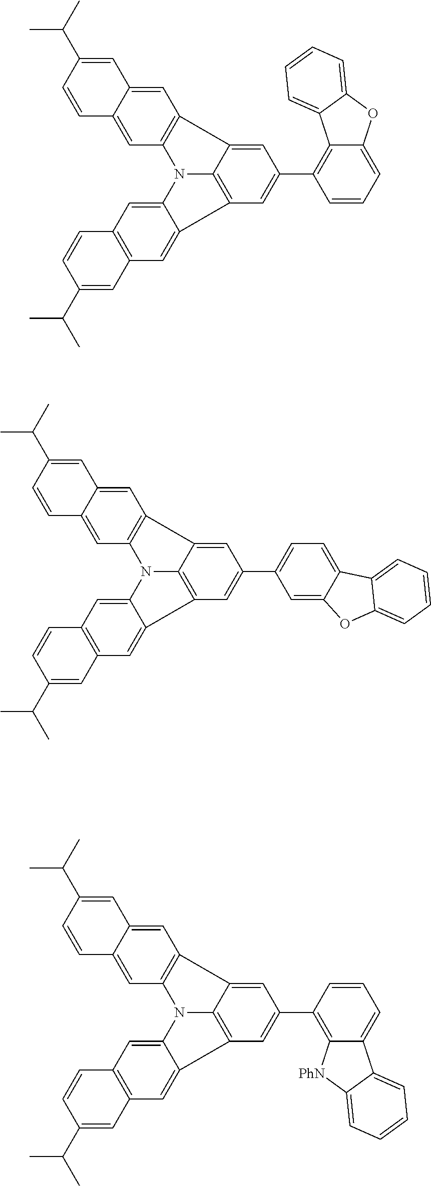

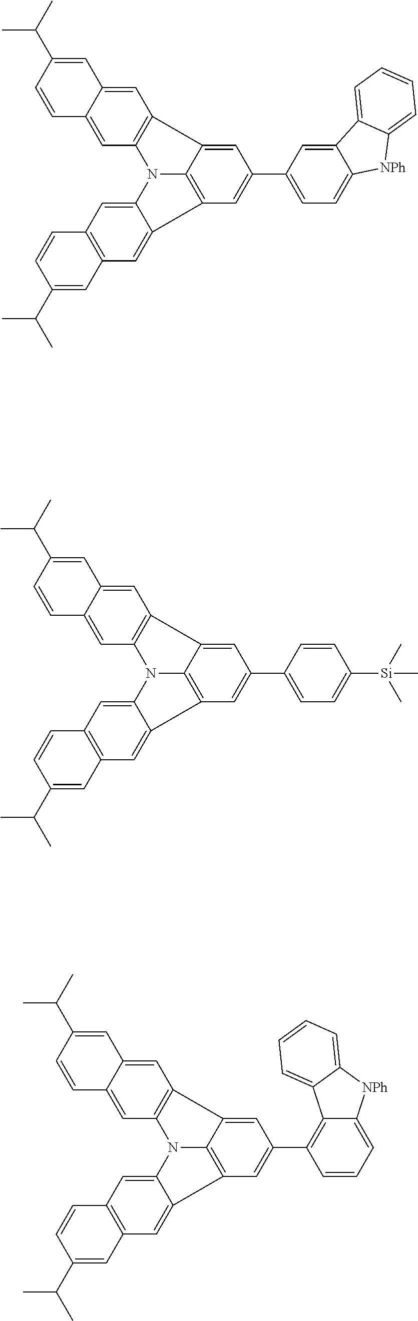

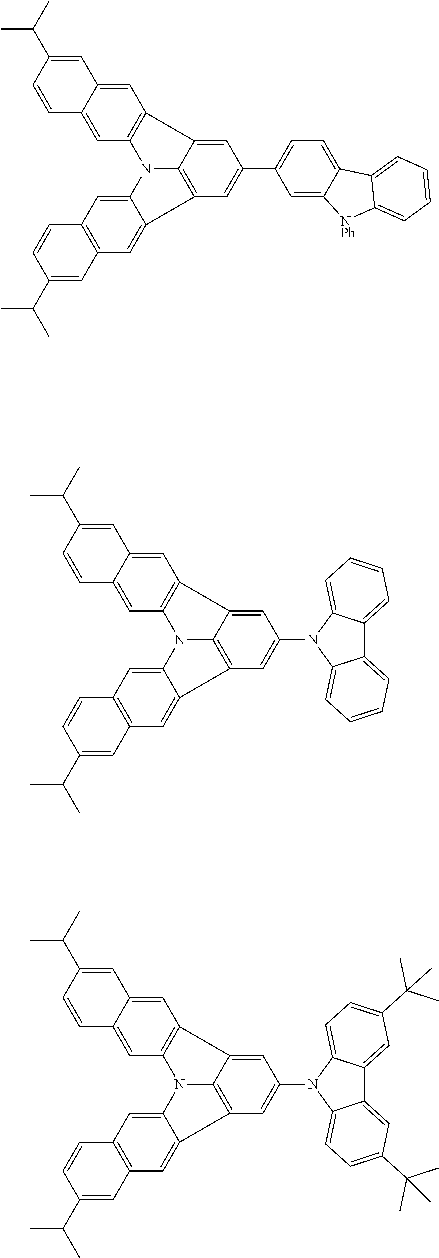

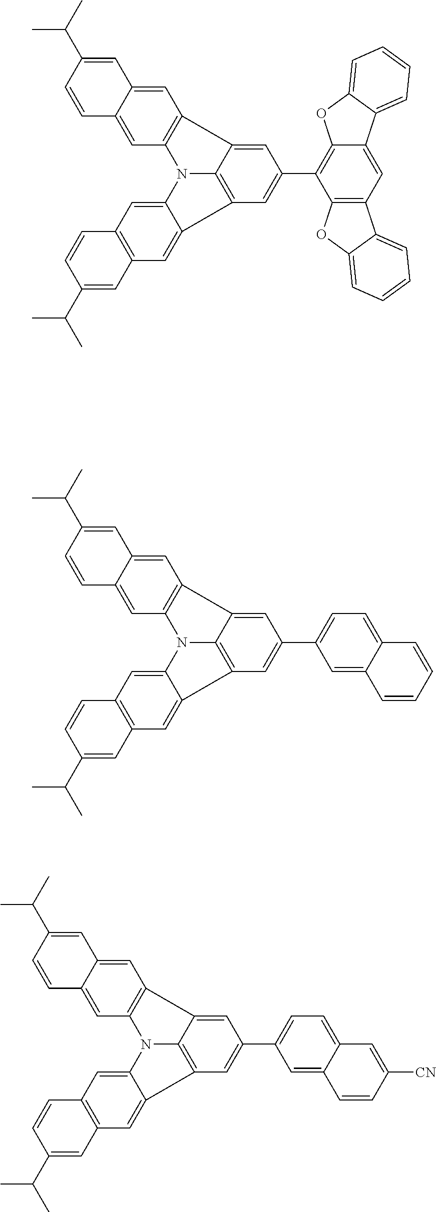 [SMILES: CC(C)c1ccc2cc3c(cc2c1)c1cc(-c2c4oc5ccccc5c4cc4c2oc2ccccc24)cc2c4cc5cc(C(C)C)ccc5cc4n3c12 ; CC(C)c1ccc2cc3c(cc2c1)c1cc(-c2ccc4ccccc4c2)cc2c4cc5cc(C(C)C)ccc5cc4n3c12 ; CC(C)c1ccc2cc3c(cc2c1)c1cc(-c2ccc4cc(C#N)ccc4c2)cc2c4cc5cc(C(C)C)ccc5cc4n3c12]

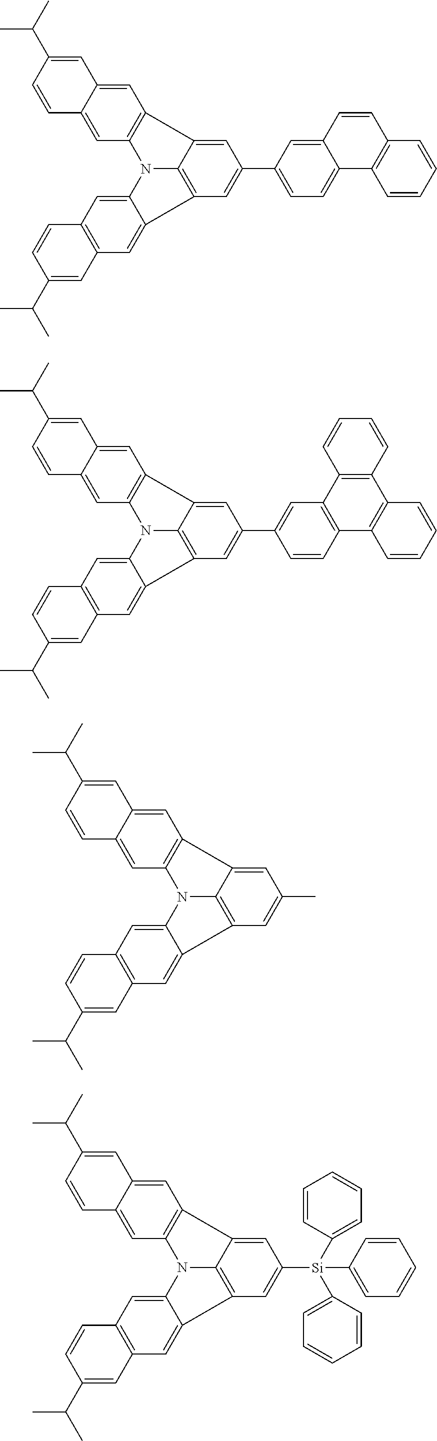

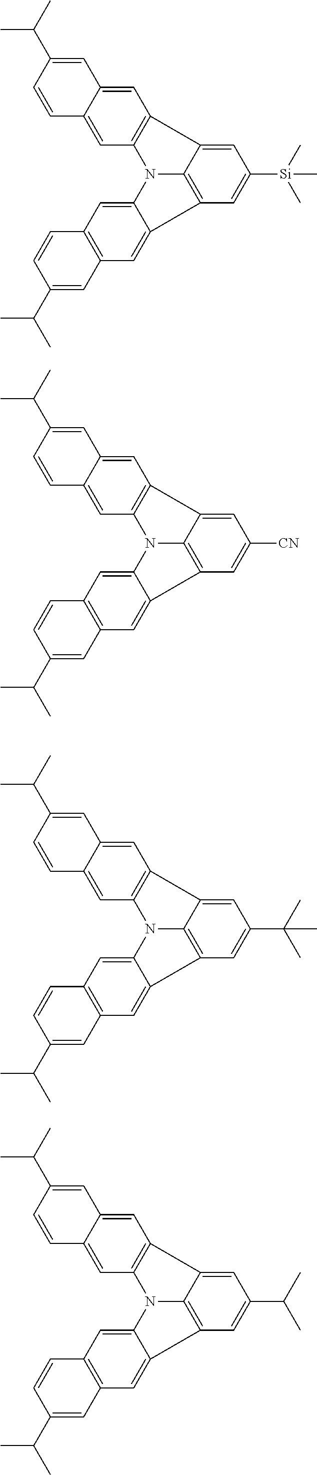

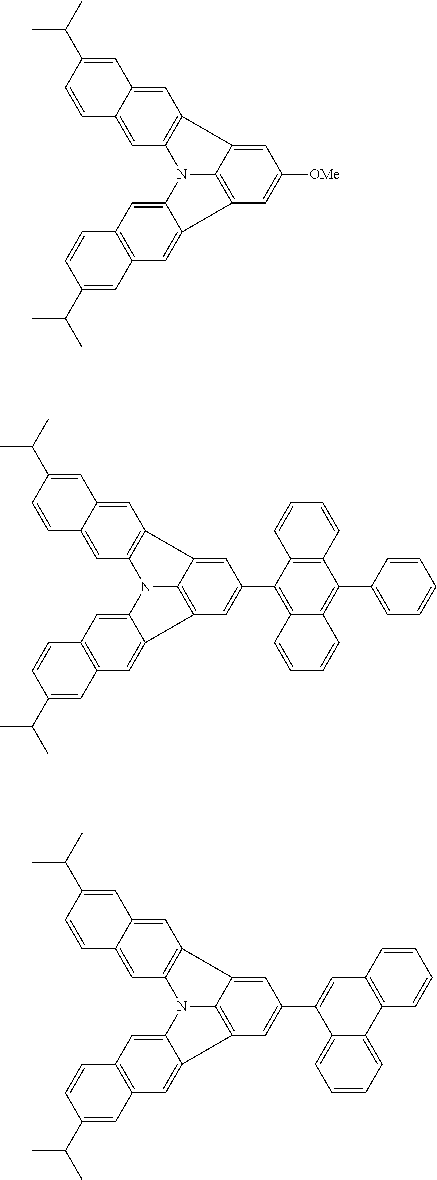 [SMILES: COc1cc2c3cc4cc(C(C)C)ccc4cc3n3c4cc5ccc(C(C)C)cc5cc4c(c1)c23 ; CC(C)c1ccc2cc3c(cc2c1)c1cc(-c2c4ccccc4c(-c4ccccc4)c4ccccc24)cc2c4cc5cc(C(C)C)ccc5cc4n3c12 ; CC(C)c1ccc2cc3c(cc2c1)c1cc(-c2cc4ccccc4c4ccccc24)cc2c4cc5cc(C(C)C)ccc5cc4n3c12]

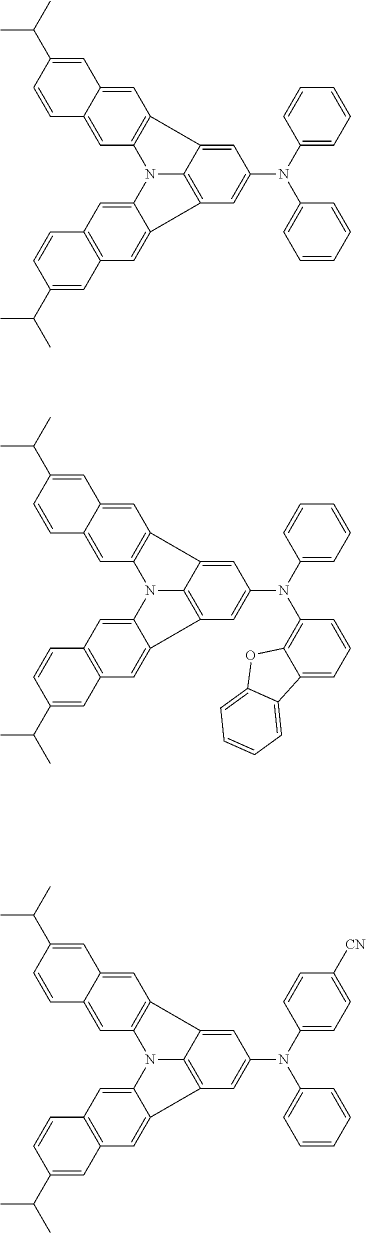 [SMILES: CC(C)c1ccc2cc3c(cc2c1)c1cc(N(c2ccccc2)c2ccccc2)cc2c4cc5cc(C(C)C)ccc5cc4n3c12 ; CC(C)c1ccc2cc3c(cc2c1)c1cc(N(c2ccccc2)c2cccc4c2oc2ccccc24)cc2c4cc5cc(C(C)C)ccc5cc4n3c12 ; CC(C)c1ccc2cc3c(cc2c1)c1cc(N(c2ccccc2)c2ccc(C#N)cc2)cc2c4cc5cc(C(C)C)ccc5cc4n3c12]

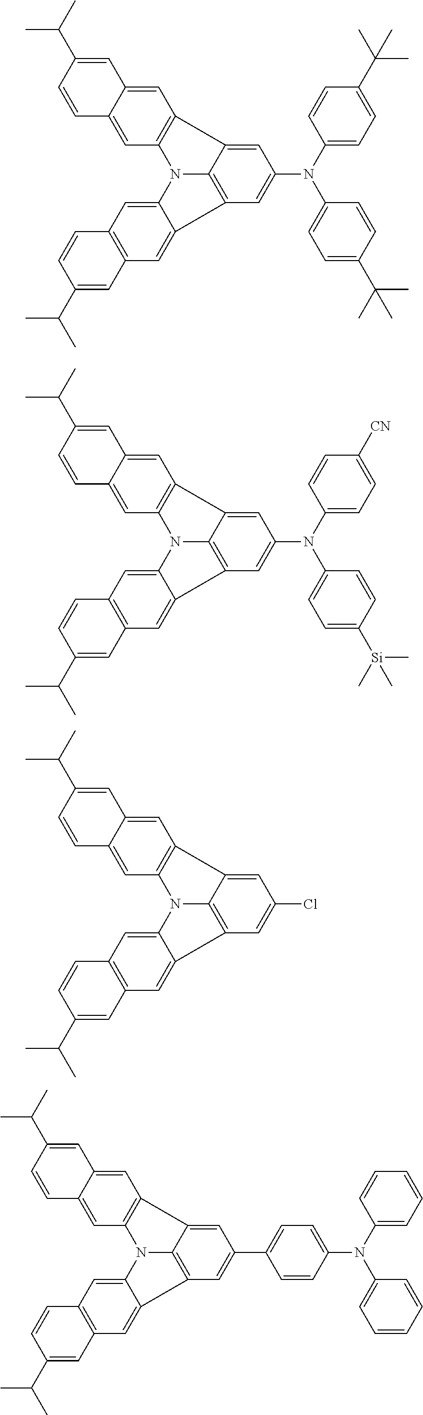

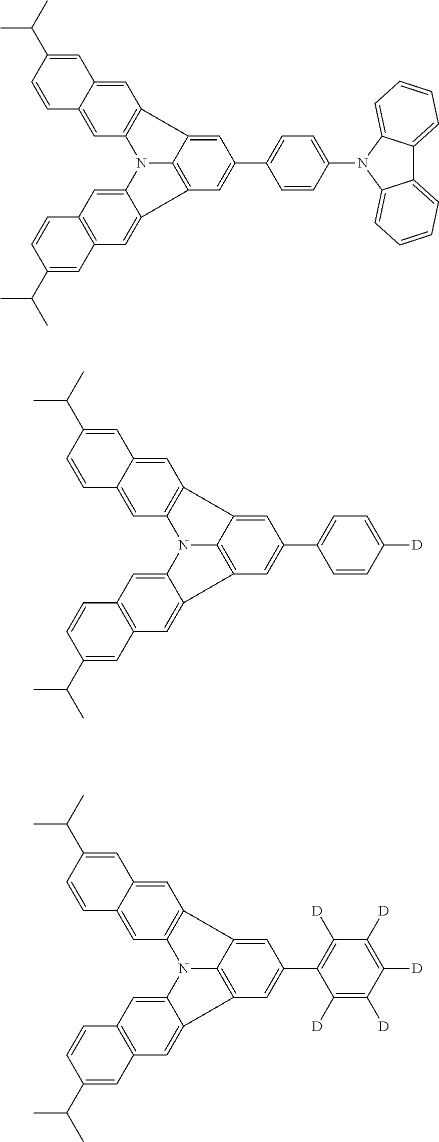 [SMILES: CC(C)c1ccc2cc3c(cc2c1)c1cc(-c2ccc(-n4c5ccccc5c5ccccc54)cc2)cc2c4cc5cc(C(C)C)ccc5cc4n3c12 ; [2H]c1ccc(-c2cc3c4cc5cc(C(C)C)ccc5cc4n4c5cc6ccc(C(C)C)cc6cc5c(c2)c34)cc1 ; [2H]c1c([2H])c([2H])c(-c2cc3c4cc5cc(C(C)C)ccc5cc4n4c5cc6ccc(C(C)C)cc6cc5c(c2)c34)c([2H])c1[2H]]

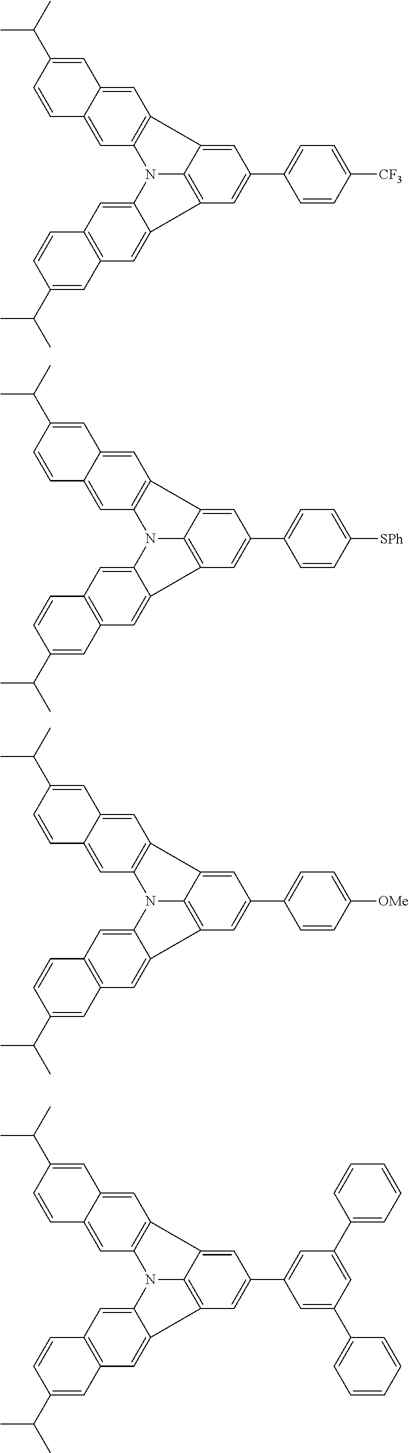

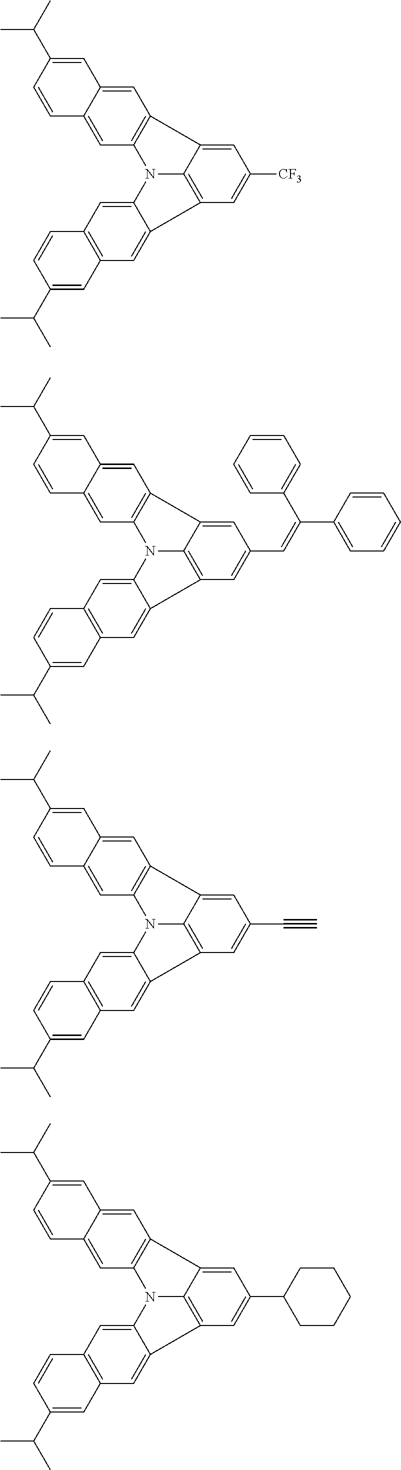

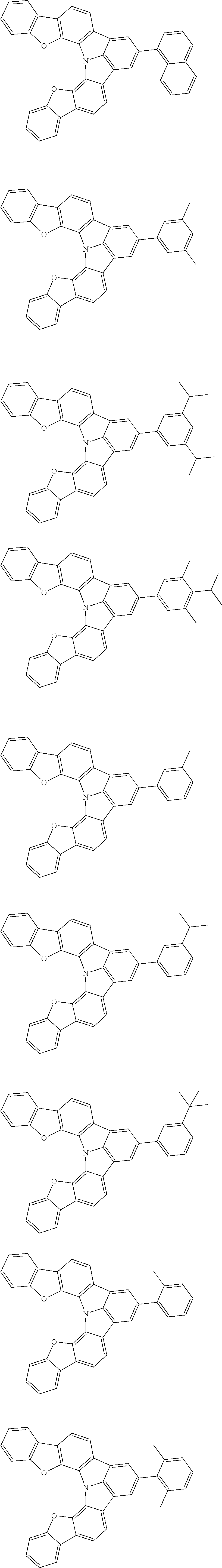

[chemical structure]

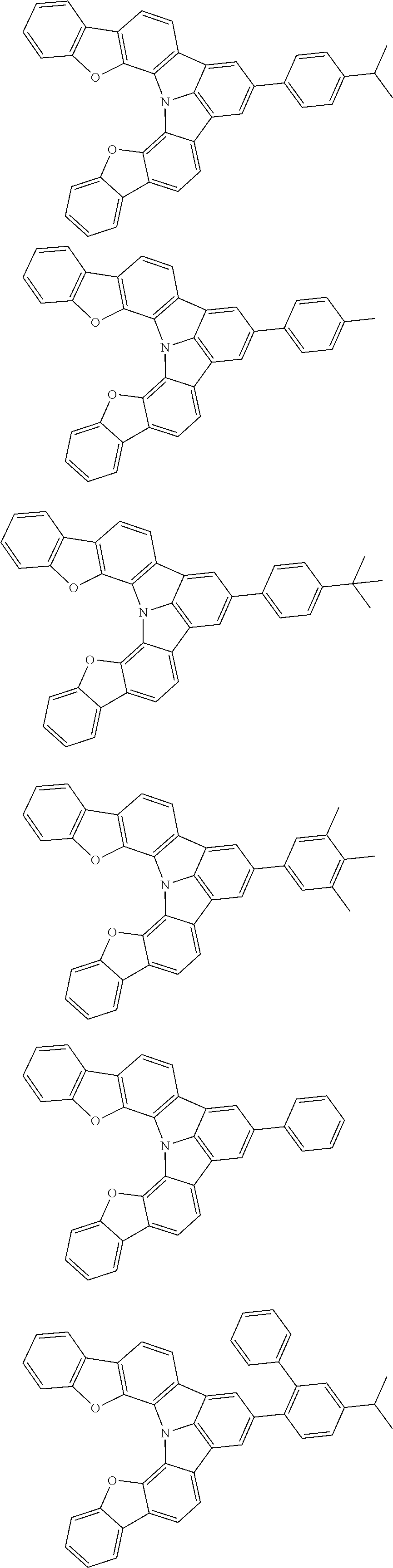

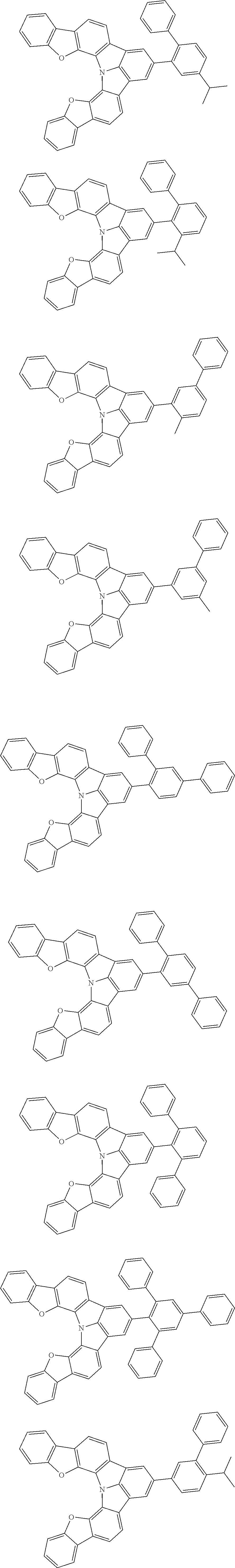

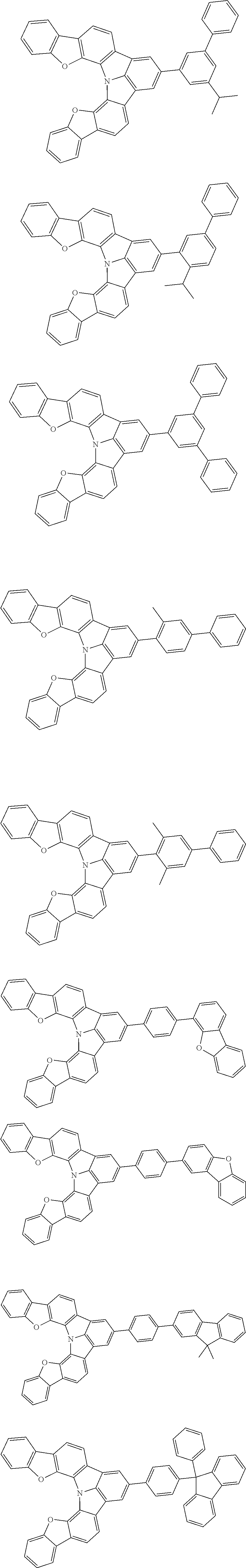

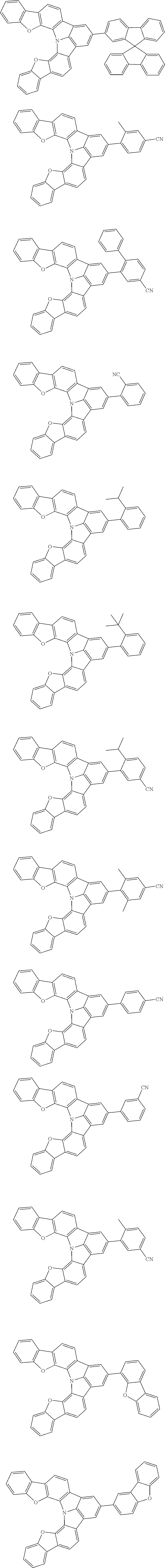

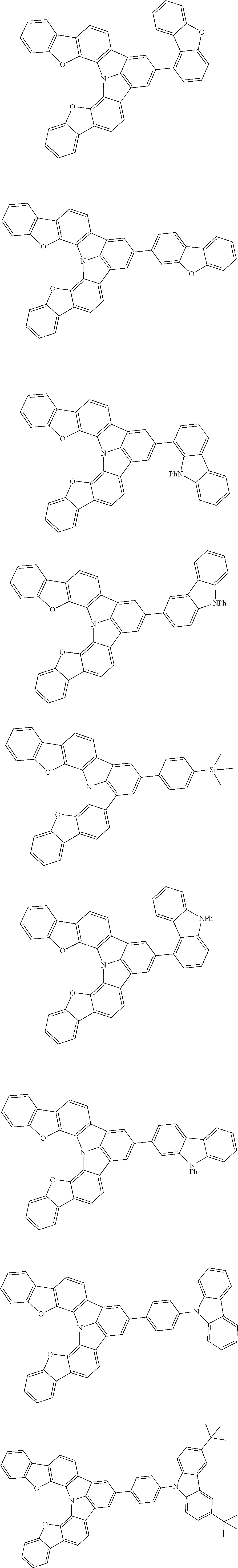

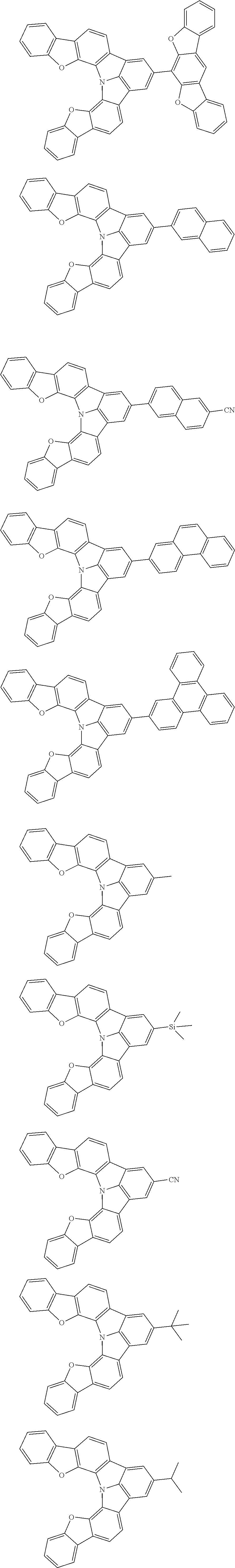

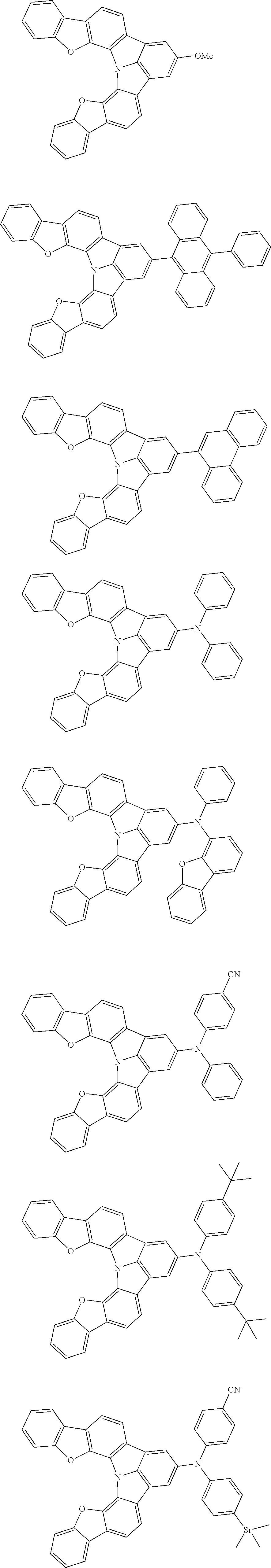

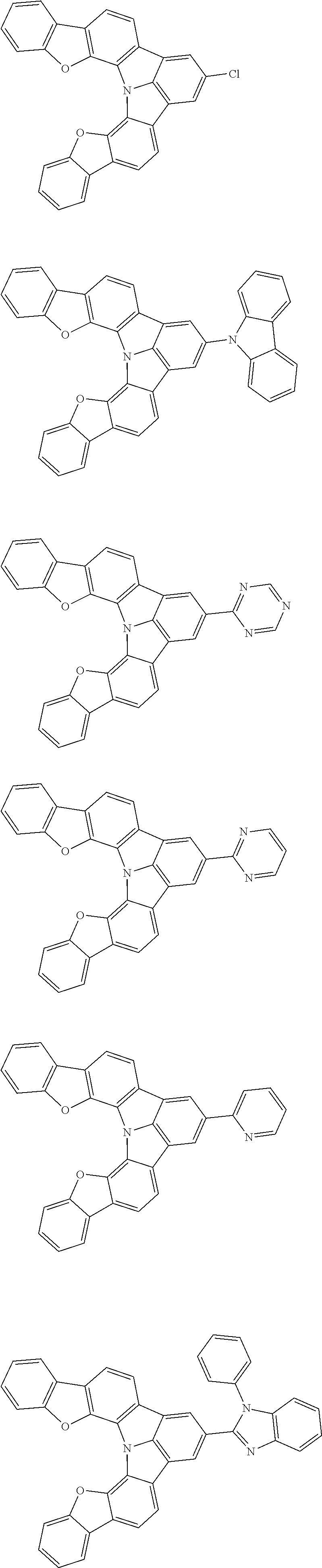 [SMILES: Clc1cc2c3ccc4c5ccccc5oc4c3n3c2c(c1)c1ccc2c4ccccc4oc2c13 ; c1ccc2c(c1)oc1c2ccc2c3cc(-n4c5ccccc5c5ccccc54)cc4c5ccc6c7ccccc7oc6c5n(c34)c21 ; c1ccc2c(c1)oc1c2ccc2c3cc(-c4ncncn4)cc4c5ccc6c7ccccc7oc6c5n(c34)c21 ; c1cnc(-c2cc3c4ccc5c6ccccc6oc5c4n4c3c(c2)c2ccc3c5ccccc5oc3c24)nc1 ; c1ccc(-c2cc3c4ccc5c6ccccc6oc5c4n4c3c(c2)c2ccc3c5ccccc5oc3c24)nc1 ; c1ccc(-n2c(-c3cc4c5ccc6c7ccccc7oc6c5n5c4c(c3)c3ccc4c6ccccc6oc4c35)nc3ccccc32)cc1]

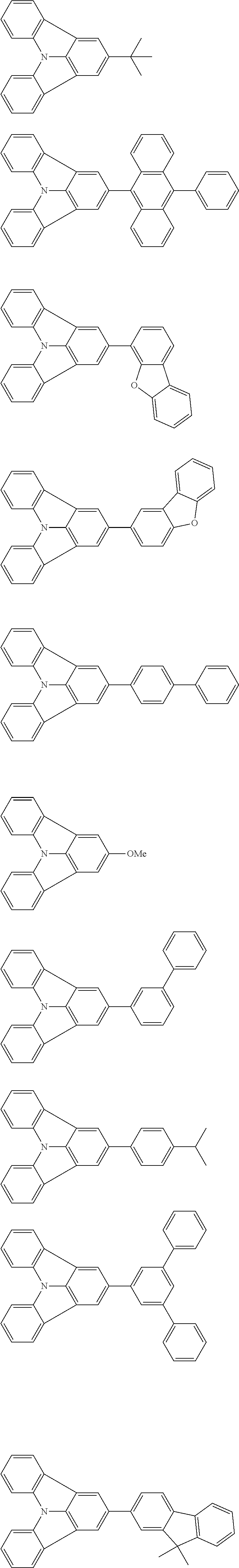

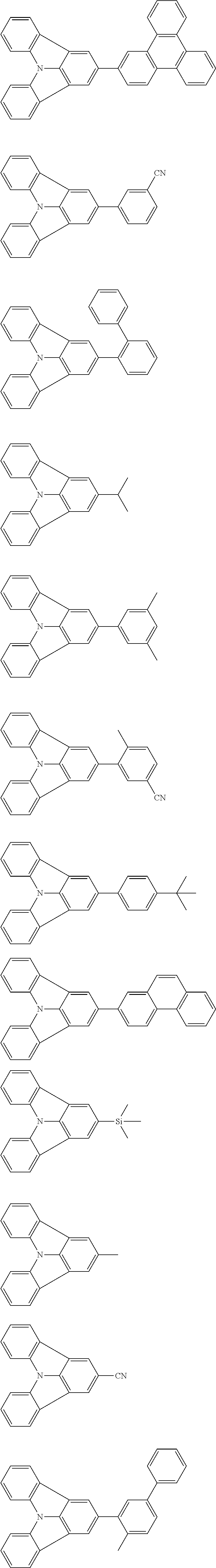

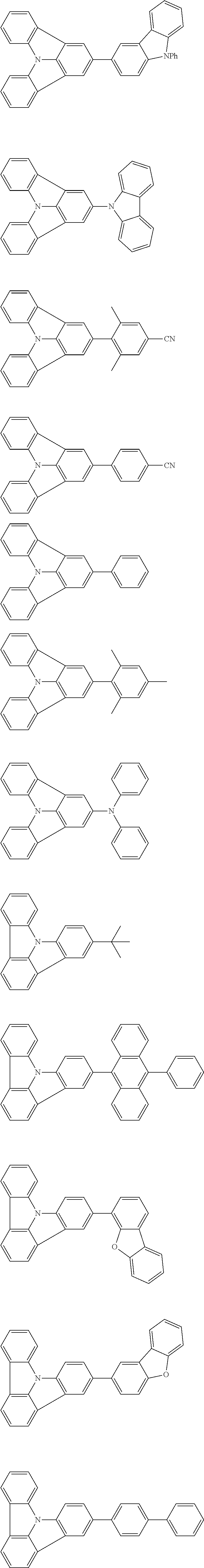

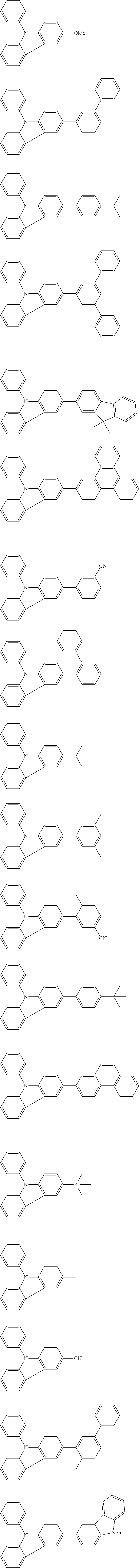

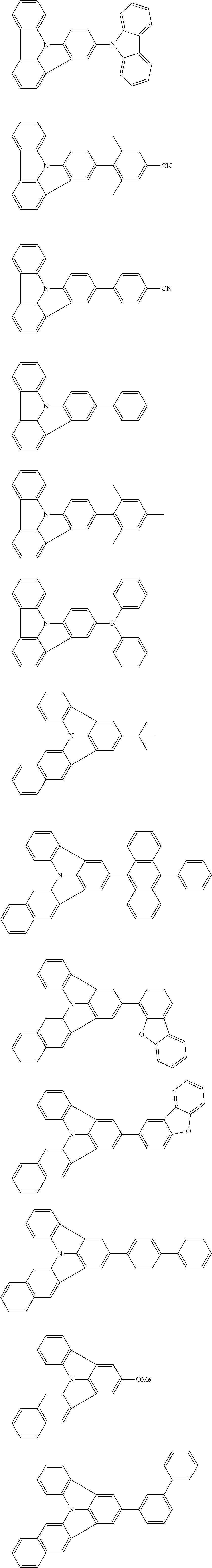

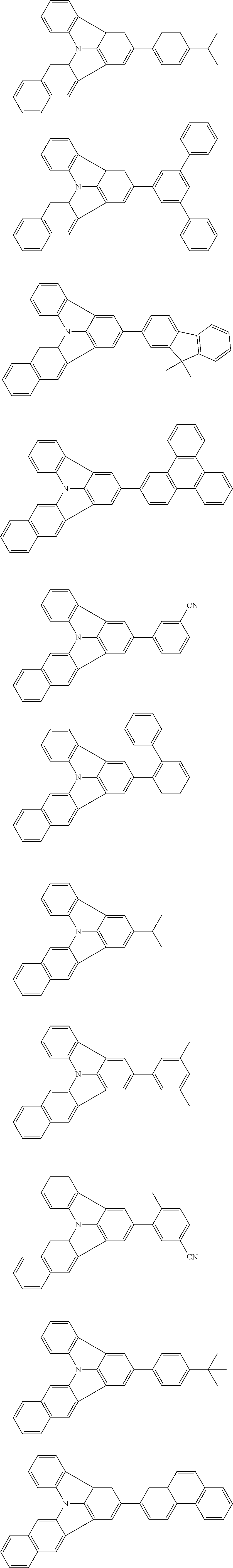

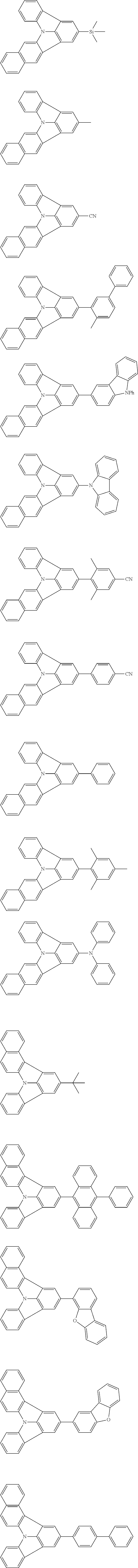

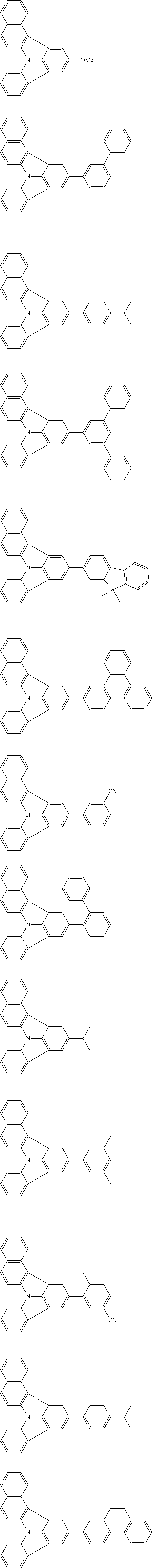

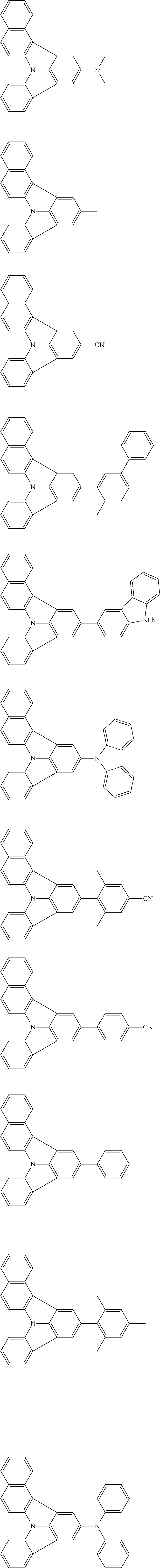

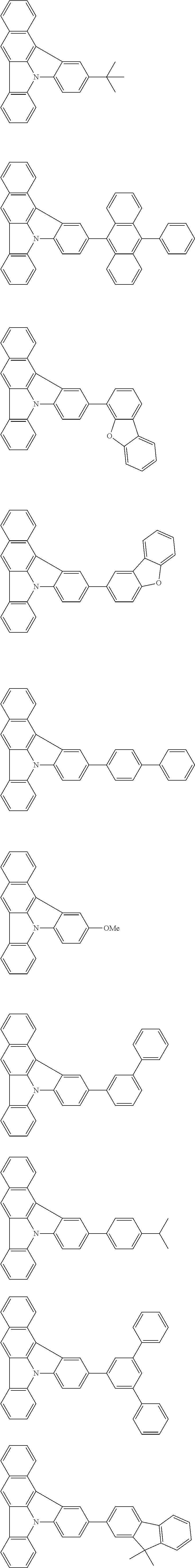

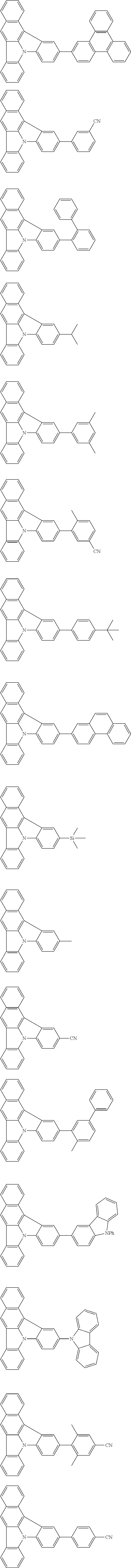

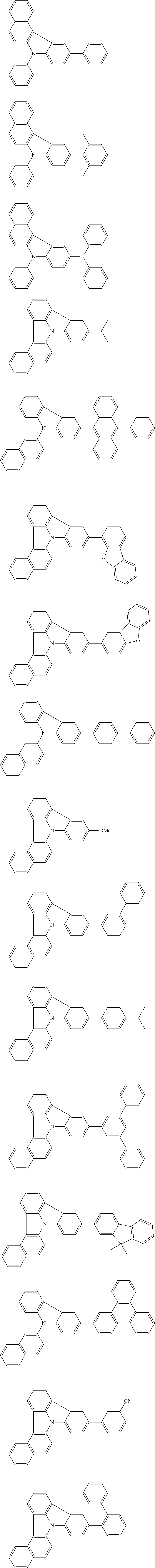

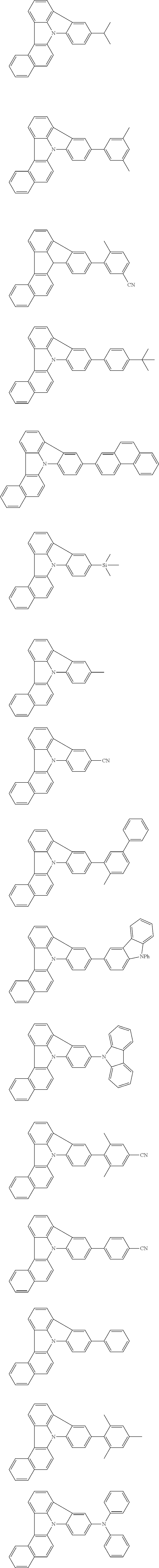

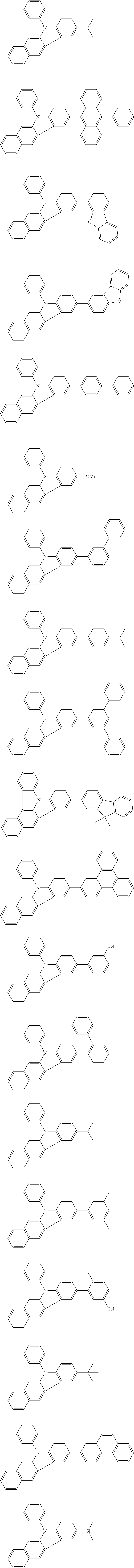

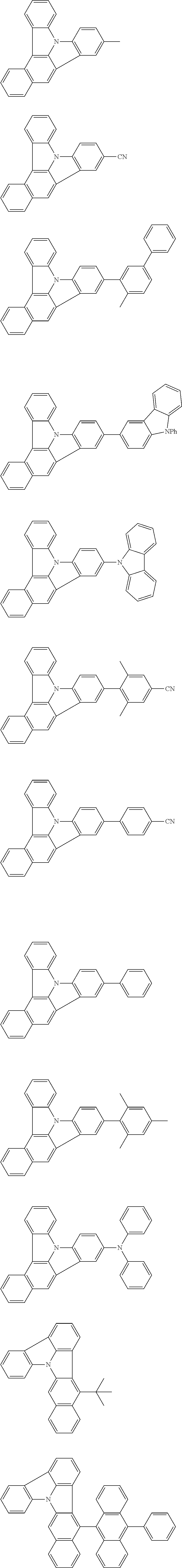

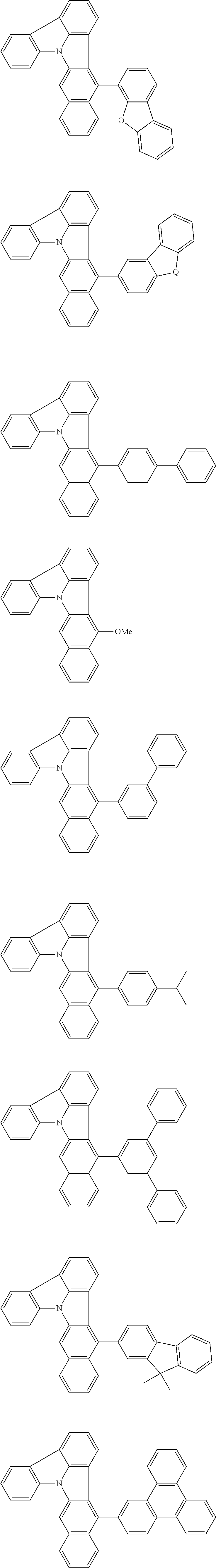

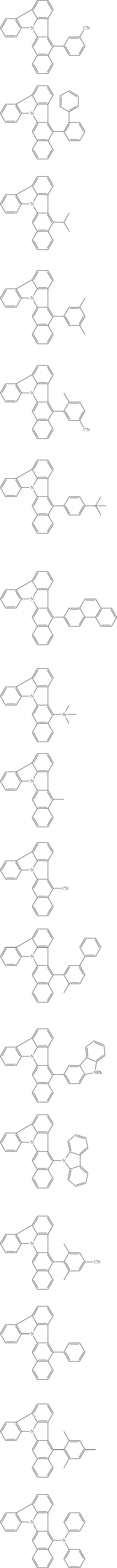

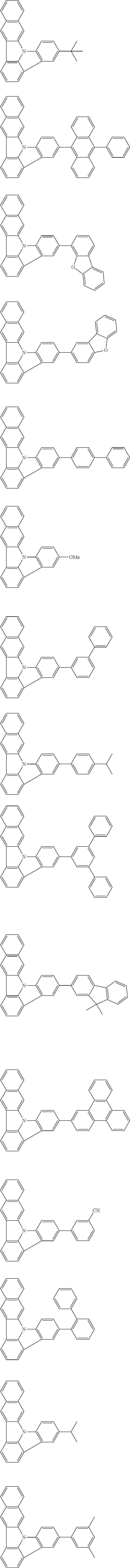

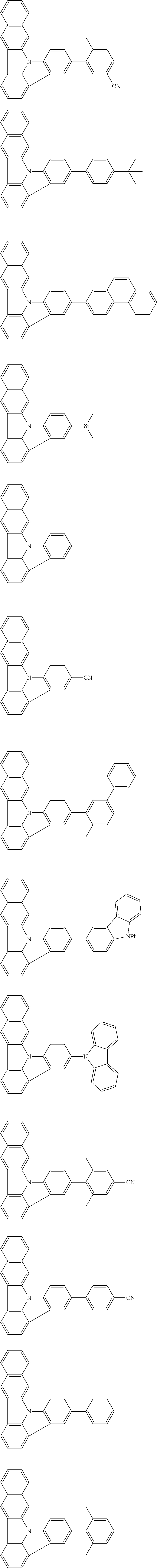

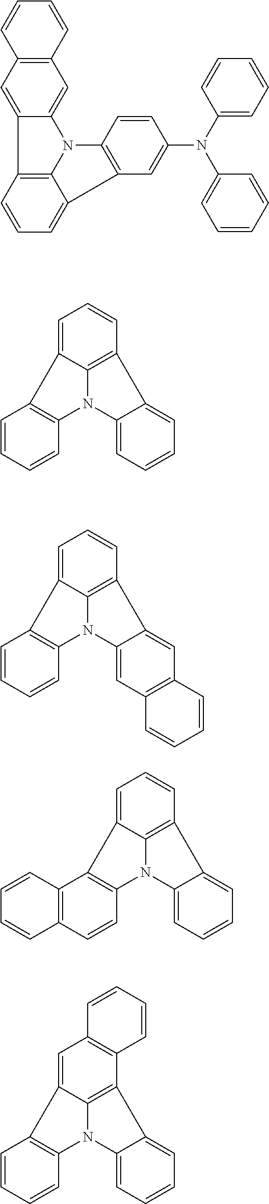

The dopant material 2 is a boron-containing compound represented by formula (D2):

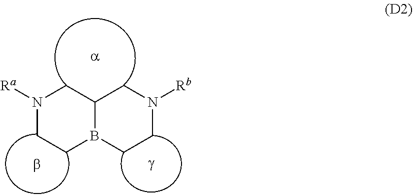

wherein

a ring α, a ring β, and a ring γ are each independently a substituted or unsubstituted aromatic hydrocarbon ring having 6 to 50 ring carbon atoms or a substituted or unsubstituted aromatic heterocyclic ring having 5 to 50 ring atoms;

R^{a} and R^{b} are each independently a substituted or unsubstituted aryl group having 6 to 50 ring carbon atoms, a substituted or unsubstituted heteroaryl group having 5 to 50 ring atoms, or a substituted or unsubstituted alkyl group having 1 to 20 carbon atoms;

R^{a} may be bonded to one or both of the ring α and the ring β directly or via a linker; and

R^{b} may be bonded to one or both of the ring α and the ring γ directly or via a linker.

Examples of the aromatic hydrocarbon ring having 6 to 50, preferably 6 to 30, more preferably 6 to 24, and still more preferably 6 to 18 ring carbon atoms include a benzene ring, a biphenyl ring, a naphthalene ring, a terphenyl ring (m-terphenyl ring, o-terphenyl ring, p-terphenyl ring), an anthracene ring, an acenaphthylene ring, a fluorene ring, a phenalene ring, a phenanthrene ring, a triphenylene ring, a fluoranthene ring, a pyrene ring, a naphthacene ring, a perylene ring, and a pentacene ring.

The aromatic heterocyclic ring having 5 to 50, preferably 5 to 30, more preferably 5 to 18, and still more preferably 5 to 13 ring atoms includes at least one, preferably 1 to 5 ring hetero atoms. The ring hetero atom is selected, for example, from a nitrogen atom, a sulfur atom, and an oxygen atom. Examples of the aromatic heterocyclic ring include a pyrrole ring, an oxazole ring, an isoxazole ring, a thiazole ring, an isothiazole ring, an imidazole ring, an oxadiazole ring, a thiadiazole ring, a triazole ring, a tetrazole ring, a pyrazole ring, a pyridine ring, a pyrimidine ring, a pyridazine ring, a pyrazine ring, a triazine ring, an indole ring, an isoindole ring, a 1H-indazole ring, a benzimidazole ring, a benzoxazole ring, a benzothiazole ring, a 1H-benzotriazole ring, a quinoline ring, an isoquinoline ring, a cinnoline ring, a quinazoline ring, a quinoxaline ring, a phthalazine ring, a naphthyridine ring, a purine ring, a pteridine ring, a carbazole ring, an acridine ring, a phenoxathiin ring, a phenoxazine ring, a phenothiazine ring, a phenazine ring, an indolizine ring, a furan ring, a benzofuran ring, an isobenzofuran ring, a dibenzofuran ring, a thiophene ring, a benzothiophene ring, a dibenzothiophene ring, a furazan ring, an oxadiazole ring, and a thianthrene ring.

The aromatic heterocyclic ring having 5 to 50, preferably 5 to 30, more preferably 5 to 18, and still more preferably 5 to 13 ring atoms includes at least one, preferably 1 to 5 ring hetero atoms. The ring hetero atom is selected, for example, from a nitrogen atom, a sulfur atom, and an oxygen atom. Examples of the aromatic heterocyclic ring include a pyrrole ring, an oxazole ring, an isoxazole ring, a thiazole ring, an isothiazole ring, an imidazole ring, an oxadiazole ring, a thiadiazole ring, a triazole ring, a tetrazole ring, a pyrazole ring, a pyridine ring, a pyrimidine ring, a pyridazine ring, a pyrazine ring, a triazine ring, an indole ring, an isoindole ring, a 1H-indazole ring, a benzimidazole ring, a benzoxazole ring, a benzothiazole ring, a 1H-benzotriazole ring, a quinoline ring, an isoquinoline ring, a cinnoline ring, a quinazoline ring, a quinoxaline ring, a phthalazine ring, a naphthyridine ring, a purine ring, a pteridine ring, a carbazole ring, an acridine ring, a phenoxathiin ring, a phenoxazine ring, a phenothiazine ring, a phenazine ring, an indolizine ring, a furan ring, a benzofuran ring, an isobenzofuran ring, a dibenzofuran ring, a thiophene ring, a benzothiophene ring, a dibenzothiophene ring, a furazan ring, an oxadiazole ring, and a thianthrene ring.

The optional substituent of the ring α, the ring β, and the ring γ is selected from a substituted or unsubstituted aryl group having 6 to 50, preferably 6 to 30, more preferably 6 to 24, and still more preferably 6 to 18 ring carbon atoms; a substituted or unsubstituted heteroaryl group having 5 to 50, preferably 5 to 30, more preferably 5 to 18, and still more preferably 5 to 13 ring atoms; a diarylamino group, a diheteroarylamino group, or an arylheteroarylamino group each having a substituent selected from a substituted or unsubstituted aryl group having 6 to 50, preferably 6 to 30, more preferably 6 to 24, and still more preferably 6 to 18 ring carbon atoms and a substituted or unsubstituted heteroaryl group having 5 to 50, preferably 5 to 30, more preferably 5 to 18, and still more preferably 5 to 13 ring atoms; a substituted or unsubstituted alkyl group having 1 to 20, preferably 1 to 10, and more preferably 1 to 6 carbon atoms; a substituted or unsubstituted alkoxy group having 1 to 20, preferably 1 to 10, and more preferably 1 to 6 carbon atoms; and a substituted or unsubstituted aryloxy group having 6 to 50, preferably 6 to 30, more preferably 6 to 24, and still more preferably 6 to 18 ring carbon atoms.

The optional substituent may be substituted with an aryl group having 6 to 50, preferably 6 to 30, more preferably 6 to 24, and still more preferably 6 to 18 ring carbon atoms; a heteroaryl group having 5 to 50, preferably 5 to 30, more preferably 5 to 18, and still more preferably 5 to 13 ring atoms; or an alkyl group having 1 to 20, preferably 1 to 10, and more preferably 1 to 6 carbon atoms.

Adjacent two on each of the ring α, the ring β, and the ring γ may be bonded to each other to form a substituted or unsubstituted aromatic hydrocarbon ring having 6 to 50, preferably 6 to 30, more preferably 6 to 24, and still more preferably 6 to 18 ring carbon atoms or a substituted or unsubstituted aromatic heterocyclic ring having 5 to 50, preferably 5 to 30, more preferably 5 to 18, and still more preferably 5 to 13 ring atoms. The details of the aromatic hydrocarbon ring and the aromatic heterocyclic ring are as described above with respect to the ring α, the ring β, and the ring γ.

The optional substituent of the ring thus formed is selected from an aryl group having 6 to 50, preferably 6 to 30, more preferably 6 to 24, and still more preferably 6 to 18 ring carbon atoms; a heteroaryl group having 5 to 50, preferably 5 to 30, more preferably 5 to 18, and still more preferably 5 to 13 ring atoms; and an alkyl group having 1 to 20, preferably 1 to 10, and more preferably 1 to 6 carbon atoms.

R^{a} and R^{b} are each independently a substituted or unsubstituted aryl group having 6 to 50, preferably 6 to 30, more preferably 6 to 24, and still more preferably 6 to 18 ring carbon atoms, a substituted or unsubstituted heteroaryl group having 5 to 50, preferably 5 to 30, more preferably 5 to 18, and still more preferably 5 to 13 ring atoms, or a substituted or unsubstituted alkyl group having 1 to 20, preferably 1 to 10, and more preferably 1 to 6 carbon atoms.

The details of the aryl group, the heteroaryl group, the alkyl group, the alkoxy group, and the aryloxy group mentioned with respect to the ring α, the ring β, and the ring γ and the details of the aryl group, the heteroaryl group, and the alkyl group of R^{a} and R^{b} are the same as those of corresponding groups described above with respect to R_{A}, R_{B}, and R_{C} of formula (D1).

The linker is —O—, —S—, or —CR^{c}R^{d}—. R^{c} and R^{d} are each independently a hydrogen atom or an alkyl group having 1 to 20, preferably 1 to 10, and more preferably 1 to 6 carbon atoms.

The details of the alkyl group are as described above with respect to the alkyl group of R_{A}, R_{B}, and R_{C} of formula (D1).

Formula (D2) is preferably represented by formula (D2a):

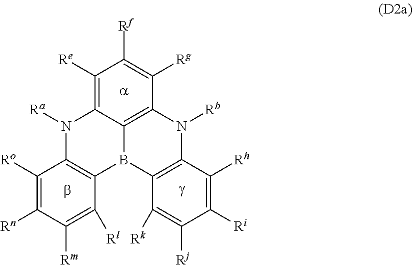

In formula (D2a), R^{a} and R^{b} are as defined above.

R^{e} to R^{o} are each independently a hydrogen atom or an optional substituent that is described above with respect to the ring α, the ring β, and the ring γ.

Adjacent two selected from R^{e} to R^{g}, adjacent two selected from R^{h} to R^{k}, and adjacent two selected from R^{l} to R^{o} may be bonded to each other to form a substituted or unsubstituted aromatic hydrocarbon ring having 6 to 50, preferably 6 to 30, more preferably 6 to 24, and still more preferably 6 to 18 ring carbon atoms or a substituted or unsubstituted aromatic heterocyclic ring having 5 to 50, preferably 5 to 30, more preferably 5 to 18, and still more preferably 5 to 13 ring atoms.

The details of the ring thus formed are as described above with respect to the ring formed by adjacent two bonded to each other on the ring α, the ring β, and the ring γ.

The dopant material 2 may be an oligomer, preferably a dimer to a hexamer, more preferably a dimer or a trimer, and still more preferably a dimer each comprising a unit structure represented by formula (D2) preferably formula (D2a). The oligomer may be a compound wherein two or more structural units are bonded to each other directly or via a linker, such as an alkylene group having 1 to 3 carbon atoms, a phenylene group and a naphthylene group; a compound wherein the ring α, the ring β, the ring γ, or the ring formed by the substituents on the ring α, the ring β, or the ring γ is commonly owned by two or more structural units; or a compound wherein the ring α, the ring β, the ring γ, or the ring formed by the substituents on the ring α, the ring β, or the ring γ in one structural unit is fused to any of the rings of another structural unit.

Examples of the oligomer having a ring commonly owned or the oligomer having a fused ring are shown below, wherein each R on the ring α, the ring β, or the ring γ is omitted for conciseness.

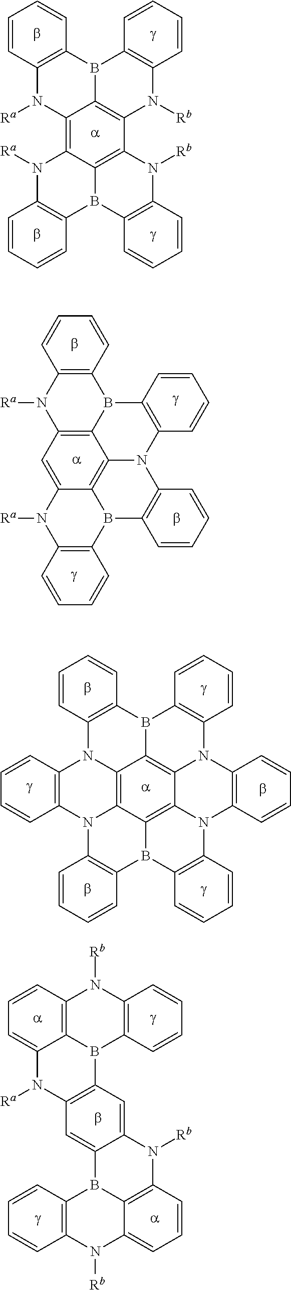

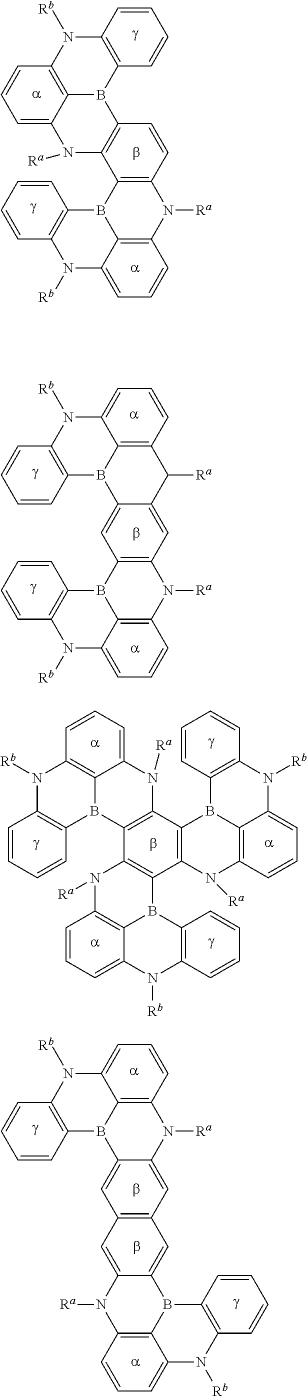

Examples of the compound represented by formula (D2) preferably formula (D2a) are shown below, although not limited thereto.

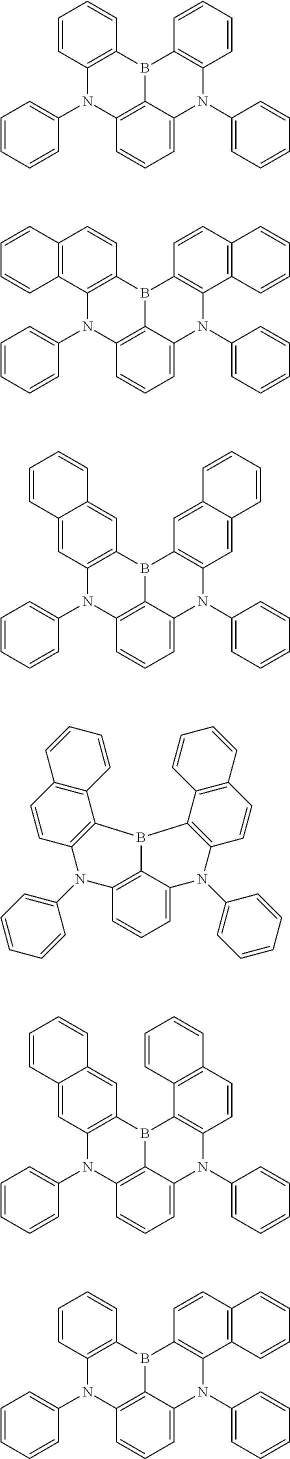 [SMILES: c1ccc(N2c3ccccc3B3c4ccccc4N(c4ccccc4)c4cccc2c43)cc1 ; c1ccc(N2c3cccc4c3B(c3ccc5ccccc5c32)c2ccc3ccccc3c2N4c2ccccc2)cc1 ; c1ccc(N2c3cc4ccccc4cc3B3c4cc5ccccc5cc4N(c4ccccc4)c4cccc2c43)cc1 ; c1ccc(N2c3cccc4c3B(c3c2ccc2ccccc32)c2c(ccc3ccccc23)N4c2ccccc2)cc1 ; c1ccc(N2c3cc4ccccc4cc3B3c4c2cccc4N(c2ccccc2)c2ccc4ccccc4c23)cc1 ; c1ccc(N2c3ccccc3B3c4ccc5ccccc5c4N(c4ccccc4)c4cccc2c43)cc1]

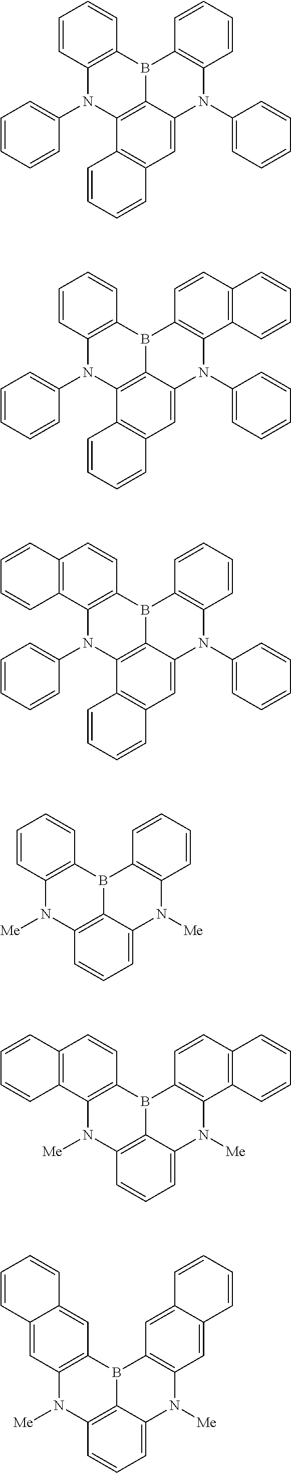 [SMILES: c1ccc(N2c3ccccc3B3c4ccccc4N(c4ccccc4)c4c3c2cc2ccccc42)cc1 ; c1ccc(N2c3cc4ccccc4c4c3B(c3ccccc3N4c3ccccc3)c3ccc4ccccc4c32)cc1 ; c1ccc(N2c3ccccc3B3c4ccc5ccccc5c4N(c4ccccc4)c4c3c2cc2ccccc42)cc1 ; CN1c2ccccc2B2c3ccccc3N(C)c3cccc1c32 ; CN1c2cccc3c2B(c2ccc4ccccc4c21)c1ccc2ccccc2c1N3C ; CN1c2cc3ccccc3cc2B2c3cc4ccccc4cc3N(C)c3cccc1c32]

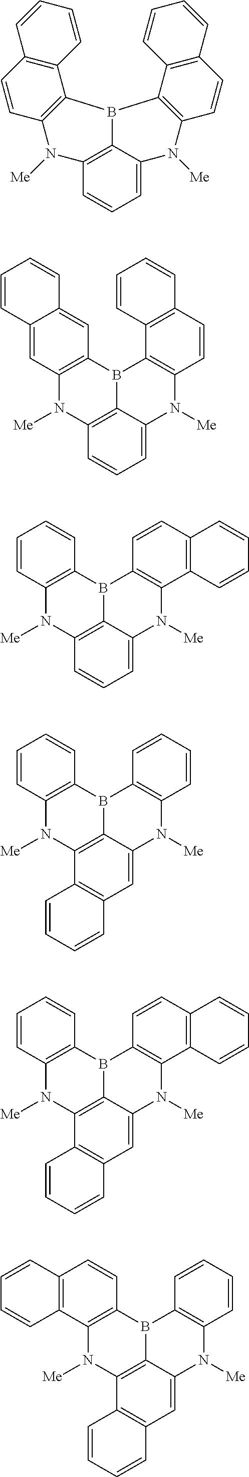 [SMILES: CN1c2cccc3c2B(c2c1ccc1ccccc21)c1c(ccc2ccccc12)N3C ; CN1c2cc3ccccc3cc2B2c3c1cccc3N(C)c1ccc3ccccc3c12 ; CN1c2ccccc2B2c3ccc4ccccc4c3N(C)c3cccc1c32 ; CN1c2ccccc2B2c3ccccc3N(C)c3c2c1cc1ccccc31 ; CN1c2cc3ccccc3c3c2B(c2ccccc2N3C)c2ccc3ccccc3c21 ; CN1c2ccccc2B2c3ccc4ccccc4c3N(C)c3c2c1cc1ccccc31]

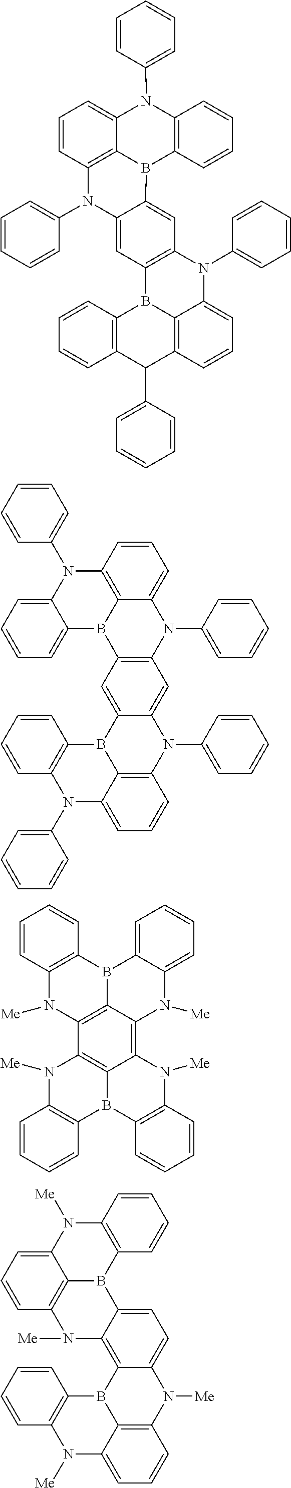

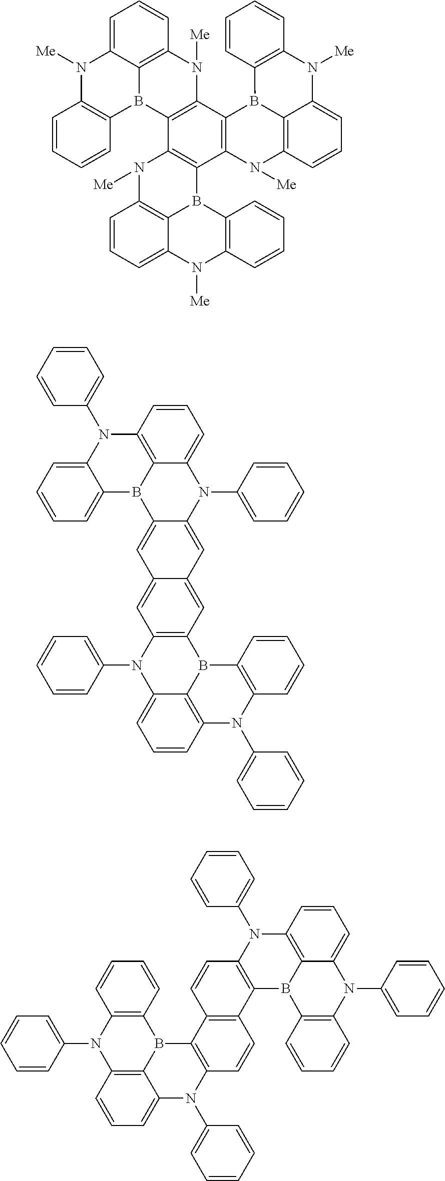

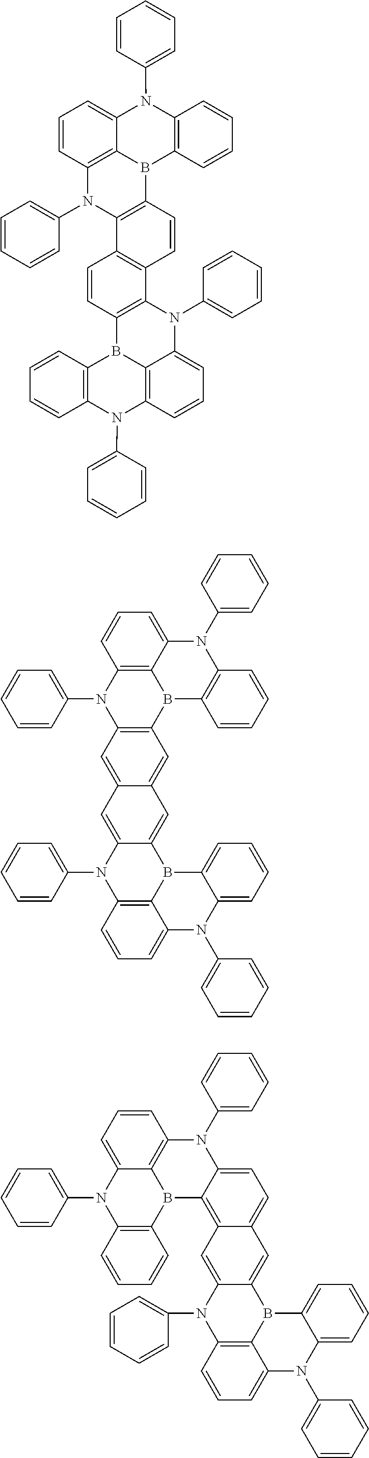

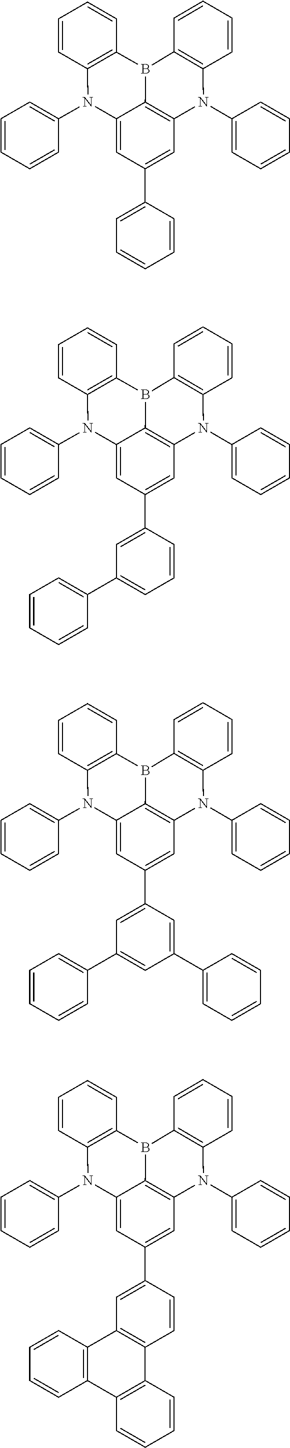 [SMILES: c1ccc(-c2cc3c4c(c2)N(c2ccccc2)c2ccccc2B4c2ccccc2N3c2ccccc2)cc1 ; c1ccc(-c2cccc(-c3cc4c5c(c3)N(c3ccccc3)c3ccccc3B5c3ccccc3N4c3ccccc3)c2)cc1 ; c1ccc(-c2cc(-c3ccccc3)cc(-c3cc4c5c(c3)N(c3ccccc3)c3ccccc3B5c3ccccc3N4c3ccccc3)c2)cc1 ; c1ccc(N2c3ccccc3B3c4ccccc4N(c4ccccc4)c4cc(-c5ccc6c7ccccc7c7ccccc7c6c5)cc2c43)cc1]

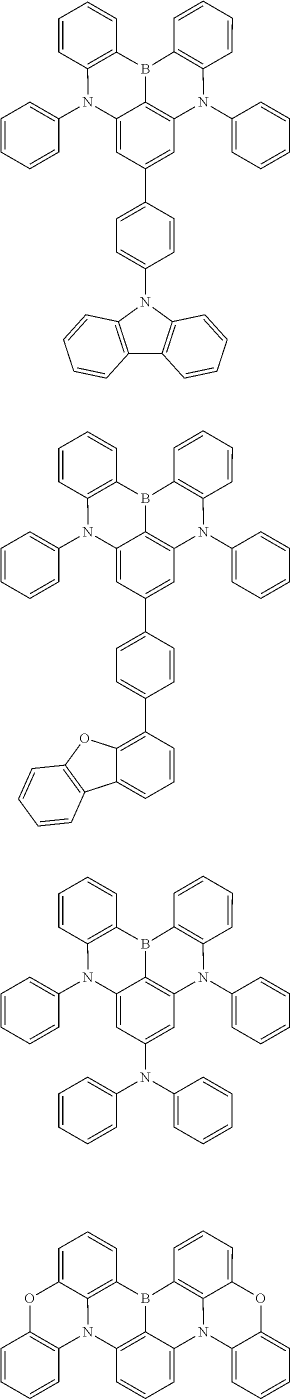 [SMILES: c1ccc(N2c3ccccc3B3c4ccccc4N(c4ccccc4)c4cc(-c5ccc(-n6c7ccccc7c7ccccc76)cc5)cc2c43)cc1 ; c1ccc(N2c3ccccc3B3c4ccccc4N(c4ccccc4)c4cc(-c5ccc(-c6cccc7c6oc6ccccc67)cc5)cc2c43)cc1 ; c1ccc(N(c2ccccc2)c2cc3c4c(c2)N(c2ccccc2)c2ccccc2B4c2ccccc2N3c2ccccc2)cc1 ; c1ccc2c(c1)Oc1cccc3c1N2c1cccc2c1B3c1cccc3c1N2c1ccccc1O3]

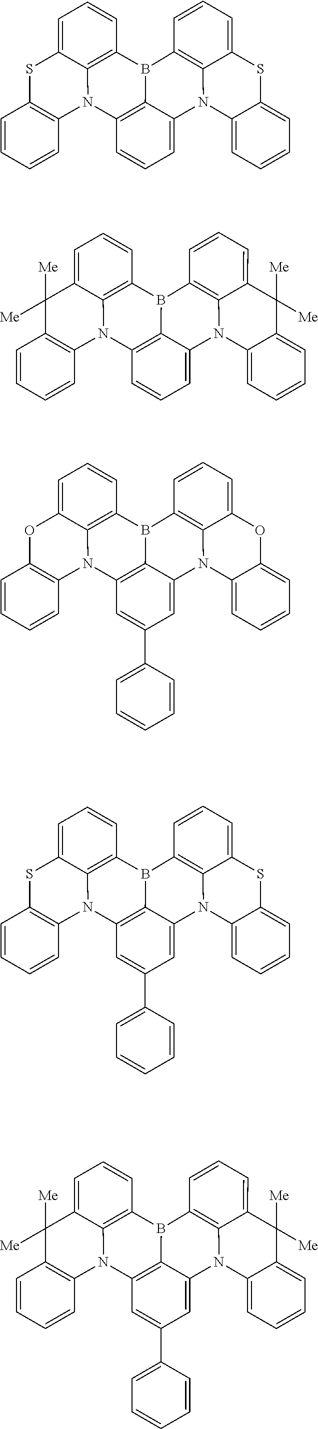 [SMILES: c1ccc2c(c1)Sc1cccc3c1N2c1cccc2c1B3c1cccc3c1N2c1ccccc1S3 ; CC1(C)c2ccccc2N2c3cccc4c3B(c3cccc1c32)c1cccc2c1N4c1ccccc1C2(C)C ; c1ccc(-c2cc3c4c(c2)N2c5ccccc5Oc5cccc(c52)B4c2cccc4c2N3c2ccccc2O4)cc1 ; c1ccc(-c2cc3c4c(c2)N2c5ccccc5Sc5cccc(c52)B4c2cccc4c2N3c2ccccc2S4)cc1 ; CC1(C)c2ccccc2N2c3cc(-c4ccccc4)cc4c3B(c3cccc1c32)c1cccc2c1N4c1ccccc1C2(C)C]

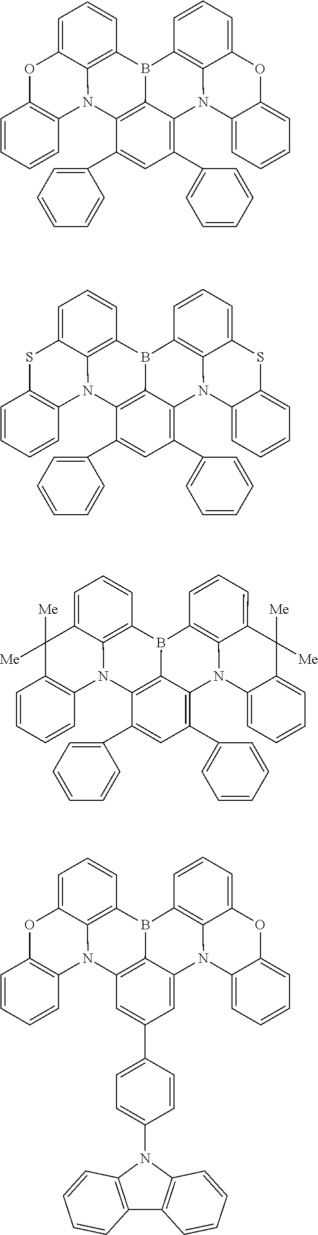 [SMILES: c1ccc(-c2cc(-c3ccccc3)c3c4c2N2c5ccccc5Oc5cccc(c52)B4c2cccc4c2N3c2ccccc2O4)cc1 ; c1ccc(-c2cc(-c3ccccc3)c3c4c2N2c5ccccc5Sc5cccc(c52)B4c2cccc4c2N3c2ccccc2S4)cc1 ; CC1(C)c2ccccc2N2c3c(cccc31)B1c3cccc4c3N(c3ccccc3C4(C)C)c3c(-c4ccccc4)cc(-c4ccccc4)c2c31 ; c1ccc2c(c1)Oc1cccc3c1N2c1cc(-c2ccc(-n4c5ccccc5c5ccccc54)cc2)cc2c1B3c1cccc3c1N2c1ccccc1O3]

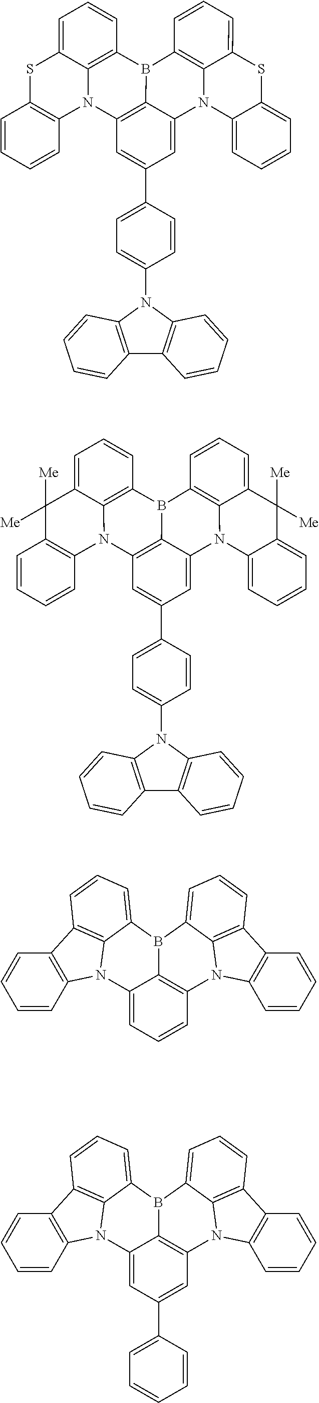 [SMILES: c1ccc2c(c1)Sc1cccc3c1N2c1cc(-c2ccc(-n4c5ccccc5c5ccccc54)cc2)cc2c1B3c1cccc3c1N2c1ccccc1S3 ; CC1(C)c2ccccc2N2c3cc(-c4ccc(-n5c6ccccc6c6ccccc65)cc4)cc4c3B(c3cccc1c32)c1cccc2c1N4c1ccccc1C2(C)C ; c1cc2c3c(c1)-n1c4ccccc4c4cccc(c41)B3c1cccc3c4ccccc4n-2c13 ; c1ccc(-c2cc3c4c(c2)-n2c5ccccc5c5cccc(c52)B4c2cccc4c5ccccc5n-3c24)cc1]

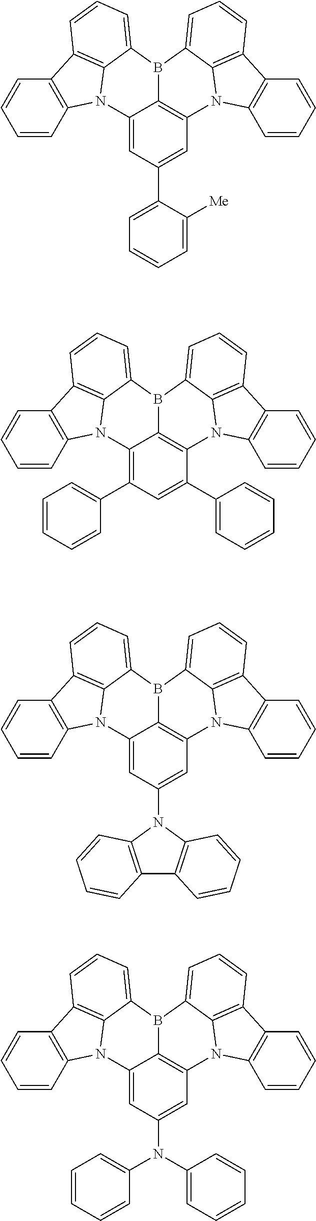 [SMILES: Cc1ccccc1-c1cc2c3c(c1)-n1c4ccccc4c4cccc(c41)B3c1cccc3c4ccccc4n-2c13 ; c1ccc(-c2cc(-c3ccccc3)c3c4c2-n2c5ccccc5c5cccc(c52)B4c2cccc4c5ccccc5n-3c24)cc1 ; c1ccc2c(c1)c1ccccc1n2-c1cc2c3c(c1)-n1c4ccccc4c4cccc(c41)B3c1cccc3c4ccccc4n-2c13 ; c1ccc(N(c2ccccc2)c2cc3c4c(c2)-n2c5ccccc5c5cccc(c52)B4c2cccc4c5ccccc5n-3c24)cc1]

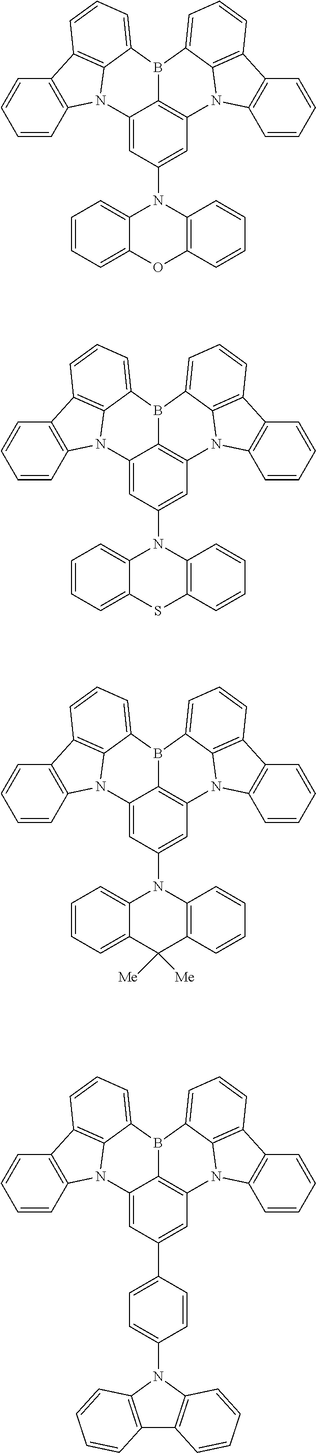 [SMILES: c1ccc2c(c1)Oc1ccccc1N2c1cc2c3c(c1)-n1c4ccccc4c4cccc(c41)B3c1cccc3c4ccccc4n-2c13 ; c1ccc2c(c1)Sc1ccccc1N2c1cc2c3c(c1)-n1c4ccccc4c4cccc(c41)B3c1cccc3c4ccccc4n-2c13 ; CC1(C)c2ccccc2N(c2cc3c4c(c2)-n2c5ccccc5c5cccc(c52)B4c2cccc4c5ccccc5n-3c24)c2ccccc21 ; c1ccc2c(c1)c1ccccc1n2-c1ccc(-c2cc3c4c(c2)-n2c5ccccc5c5cccc(c52)B4c2cccc4c5ccccc5n-3c24)cc1]

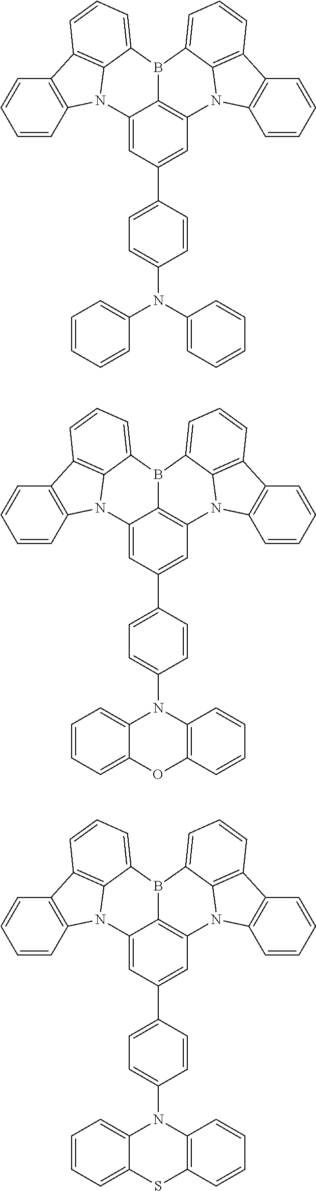

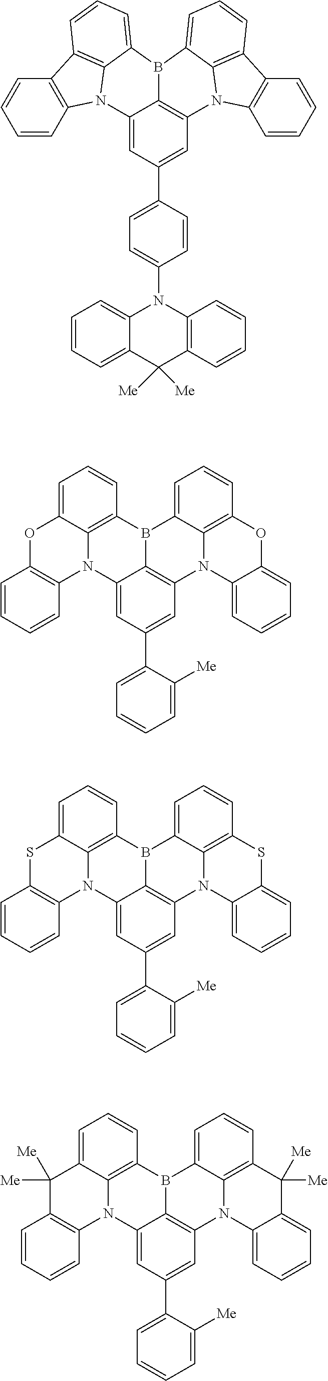 [SMILES: CC1(C)c2ccccc2N(c2ccc(-c3cc4c5c(c3)-n3c6ccccc6c6cccc(c63)B5c3cccc5c6ccccc6n-4c35)cc2)c2ccccc21 ; Cc1ccccc1-c1cc2c3c(c1)N1c4ccccc4Oc4cccc(c41)B3c1cccc3c1N2c1ccccc1O3 ; Cc1ccccc1-c1cc2c3c(c1)N1c4ccccc4Sc4cccc(c41)B3c1cccc3c1N2c1ccccc1S3 ; Cc1ccccc1-c1cc2c3c(c1)N1c4ccccc4C(C)(C)c4cccc(c41)B3c1cccc3c1N2c1ccccc1C3(C)C]

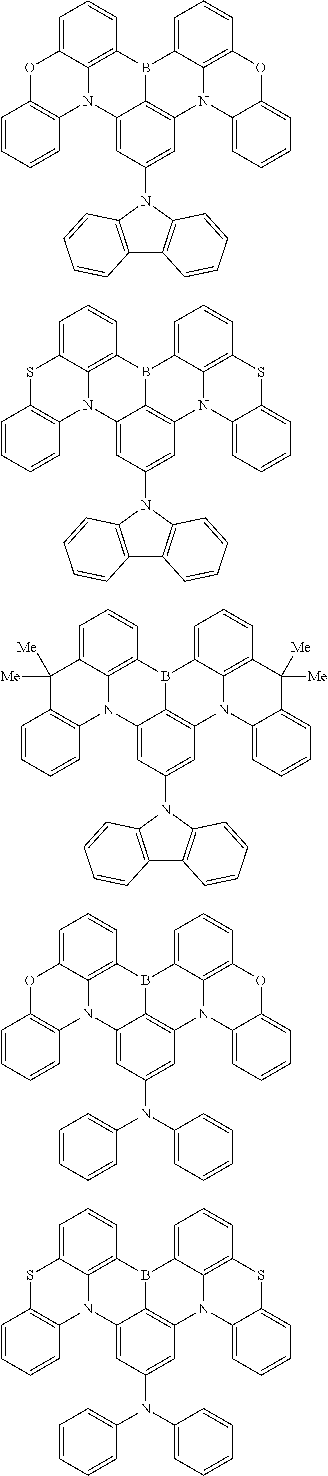

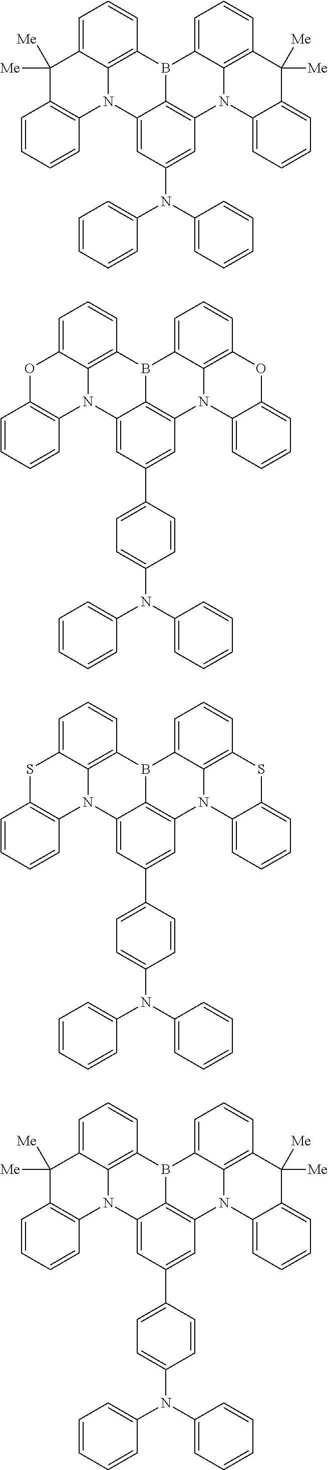

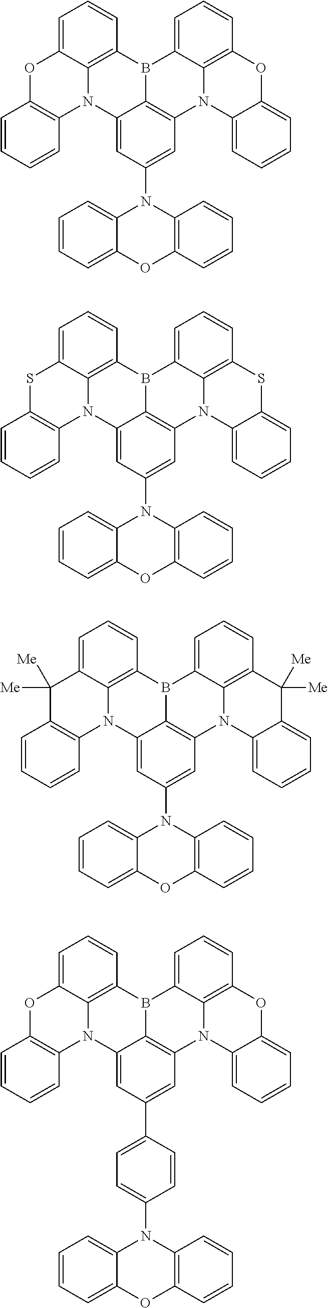

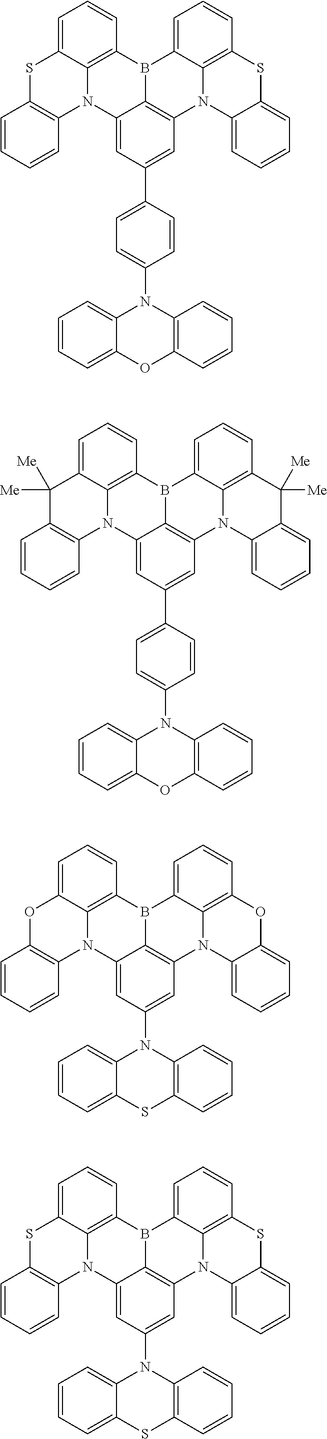 [SMILES: c1ccc2c(c1)Oc1ccccc1N2c1ccc(-c2cc3c4c(c2)N2c5ccccc5Sc5cccc(c52)B4c2cccc4c2N3c2ccccc2S4)cc1 ; CC1(C)c2ccccc2N2c3cc(-c4ccc(N5c6ccccc6Oc6ccccc65)cc4)cc4c3B(c3cccc1c32)c1cccc2c1N4c1ccccc1C2(C)C ; c1ccc2c(c1)Oc1cccc3c1N2c1cc(N2c4ccccc4Sc4ccccc42)cc2c1B3c1cccc3c1N2c1ccccc1O3 ; c1ccc2c(c1)Sc1ccccc1N2c1cc2c3c(c1)N1c4ccccc4Sc4cccc(c41)B3c1cccc3c1N2c1ccccc1S3]

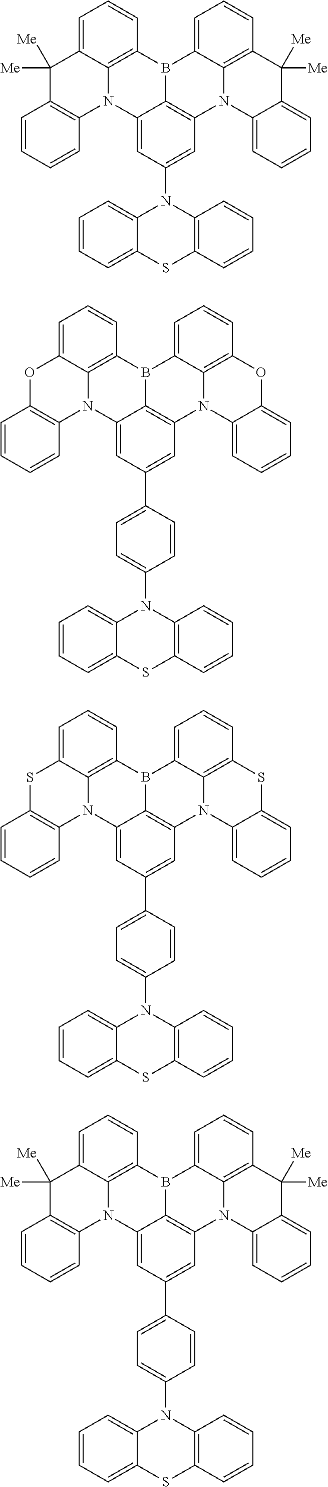

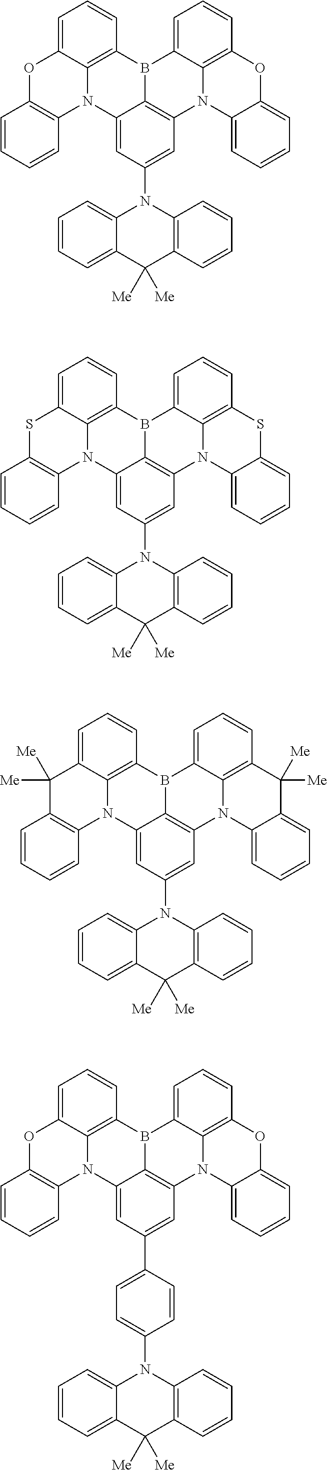 [SMILES: CC1(C)c2ccccc2N(c2cc3c4c(c2)N2c5ccccc5Oc5cccc(c52)B4c2cccc4c2N3c2ccccc2O4)c2ccccc21 ; CC1(C)c2ccccc2N(c2cc3c4c(c2)N2c5ccccc5Sc5cccc(c52)B4c2cccc4c2N3c2ccccc2S4)c2ccccc21 ; CC1(C)c2ccccc2N(c2cc3c4c(c2)N2c5ccccc5C(C)(C)c5cccc(c52)B4c2cccc4c2N3c2ccccc2C4(C)C)c2ccccc21 ; CC1(C)c2ccccc2N(c2ccc(-c3cc4c5c(c3)N3c6ccccc6Oc6cccc(c63)B5c3cccc5c3N4c3ccccc3O5)cc2)c2ccccc21]

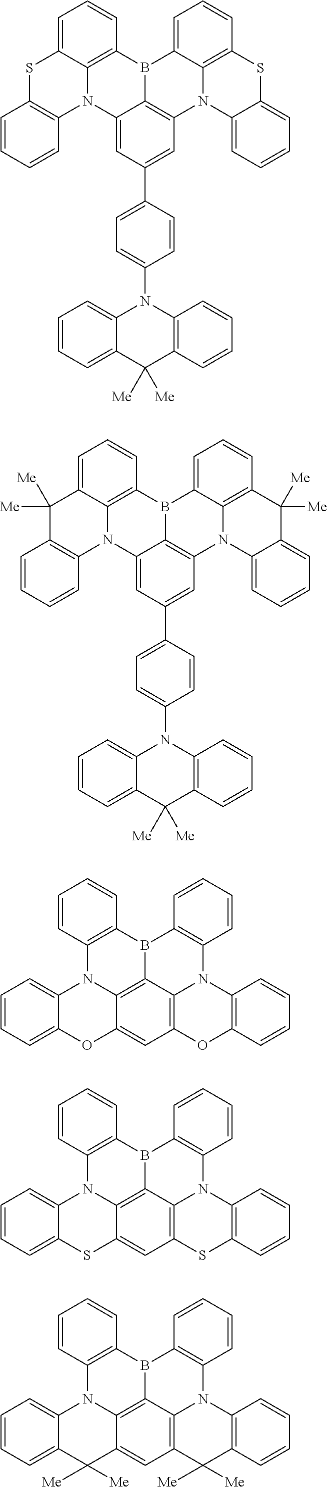

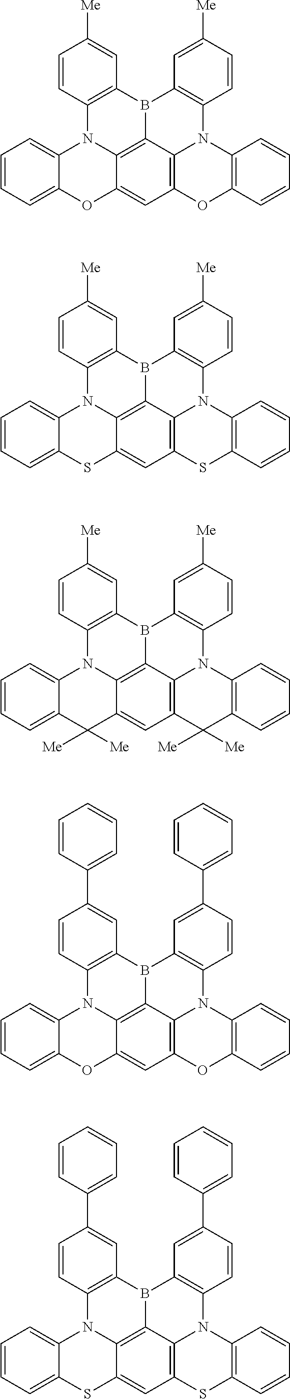 [SMILES: Cc1ccc2c(c1)B1c3cc(C)ccc3-n3c4ccccc4oc4cc5oc6ccccc6n-2c5c1c43 ; Cc1ccc2c(c1)B1c3cc(C)ccc3-n3c4ccccc4sc4cc5sc6ccccc6n-2c5c1c43 ; Cc1ccc2c(c1)B1c3cc(C)ccc3N3c4ccccc4C(C)(C)c4cc5c(c1c43)N2c1ccccc1C5(C)C ; c1ccc(-c2ccc3c(c2)B2c4cc(-c5ccccc5)ccc4-n4c5ccccc5oc5cc6oc7ccccc7n-3c6c2c54)cc1 ; c1ccc(-c2ccc3c(c2)B2c4cc(-c5ccccc5)ccc4-n4c5ccccc5sc5cc6sc7ccccc7n-3c6c2c54)cc1]

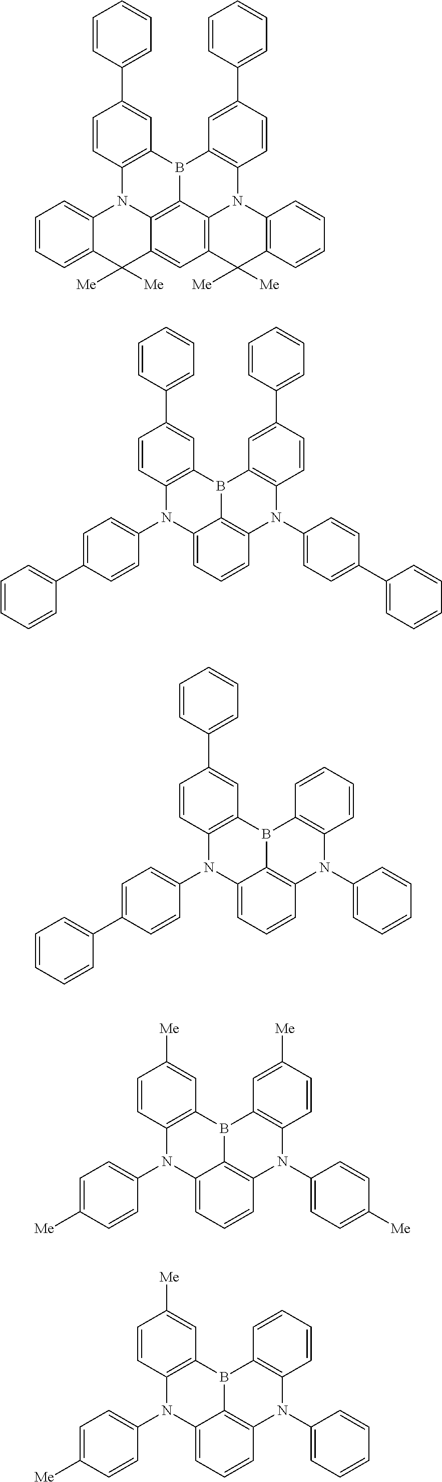

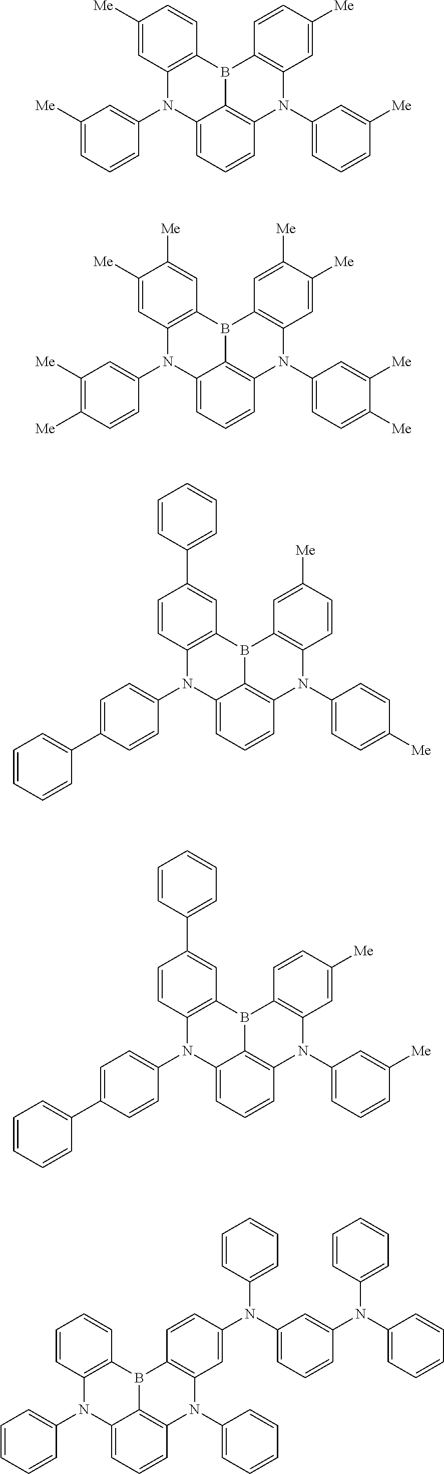 [SMILES: Cc1cccc(N2c3cc(C)ccc3B3c4ccc(C)cc4N(c4cccc(C)c4)c4cccc2c43)c1 ; Cc1ccc(N2c3cc(C)c(C)cc3B3c4cc(C)c(C)cc4N(c4ccc(C)c(C)c4)c4cccc2c43)cc1C ; Cc1ccc(N2c3ccc(C)cc3B3c4cc(-c5ccccc5)ccc4N(c4ccc(-c5ccccc5)cc4)c4cccc2c43)cc1 ; Cc1cccc(N2c3cc(C)ccc3B3c4cc(-c5ccccc5)ccc4N(c4ccc(-c5ccccc5)cc4)c4cccc2c43)c1 ; c1ccc(N(c2ccccc2)c2cccc(N(c3ccccc3)c3ccc4c(c3)N(c3ccccc3)c3cccc5c3B4c3ccccc3N5c3ccccc3)c2)cc1]

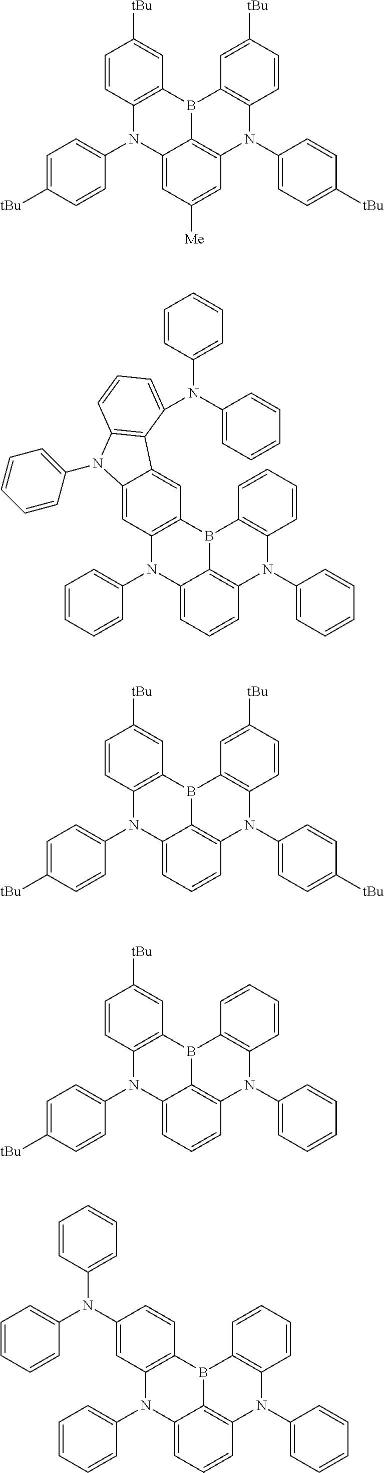 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1 ; c1ccc(N2c3ccccc3B3c4cc5c6c(N(c7ccccc7)c7ccccc7)cccc6n(-c6ccccc6)c5cc4N(c4ccccc4)c4cccc2c43)cc1 ; CC(C)(C)c1ccc(N2c3ccc(C(C)(C)C)cc3B3c4cc(C(C)(C)C)ccc4N(c4ccc(C(C)(C)C)cc4)c4cccc2c43)cc1 ; CC(C)(C)c1ccc(N2c3ccc(C(C)(C)C)cc3B3c4ccccc4N(c4ccccc4)c4cccc2c43)cc1 ; c1ccc(N(c2ccccc2)c2ccc3c(c2)N(c2ccccc2)c2cccc4c2B3c2ccccc2N4c2ccccc2)cc1]

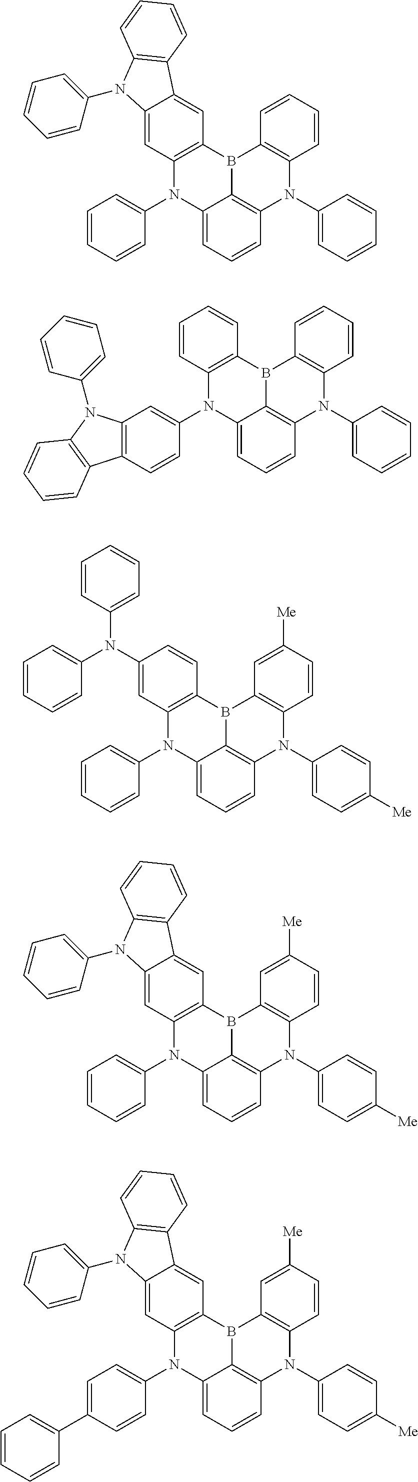 [SMILES: c1ccc(N2c3ccccc3B3c4cc5c6ccccc6n(-c6ccccc6)c5cc4N(c4ccccc4)c4cccc2c43)cc1 ; c1ccc(N2c3ccccc3B3c4ccccc4N(c4ccc5c6ccccc6n(-c6ccccc6)c5c4)c4cccc2c43)cc1 ; Cc1ccc(N2c3ccc(C)cc3B3c4ccc(N(c5ccccc5)c5ccccc5)cc4N(c4ccccc4)c4cccc2c43)cc1 ; Cc1ccc(N2c3ccc(C)cc3B3c4cc5c6ccccc6n(-c6ccccc6)c5cc4N(c4ccccc4)c4cccc2c43)cc1 ; Cc1ccc(N2c3ccc(C)cc3B3c4cc5c6ccccc6n(-c6ccccc6)c5cc4N(c4ccc(-c5ccccc5)cc4)c4cccc2c43)cc1]

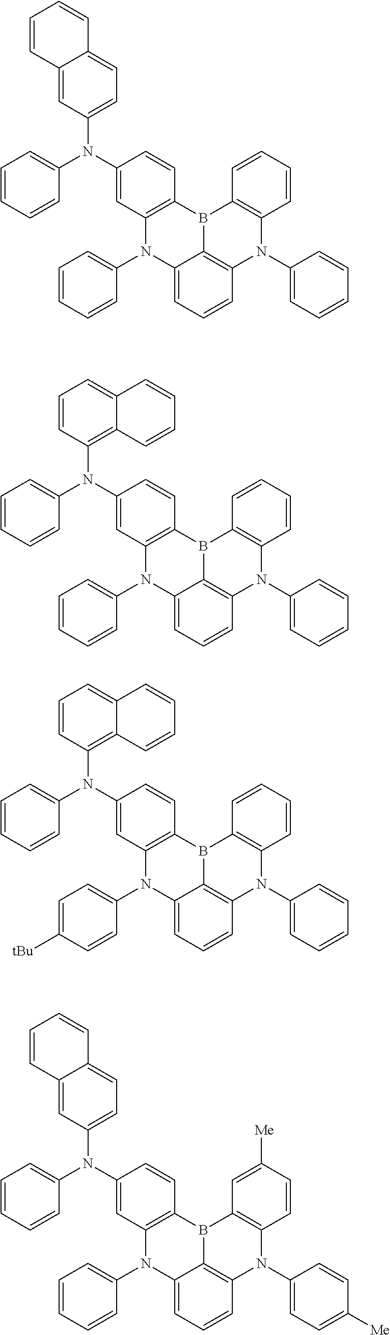

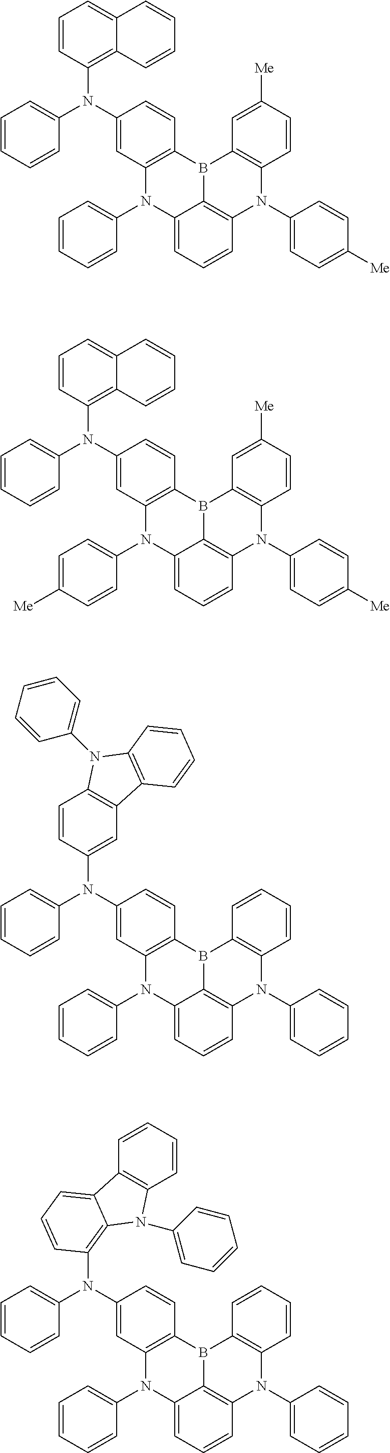 [SMILES: Cc1ccc(N2c3ccc(C)cc3B3c4ccc(N(c5ccccc5)c5cccc6ccccc56)cc4N(c4ccccc4)c4cccc2c43)cc1 ; Cc1ccc(N2c3ccc(C)cc3B3c4ccc(N(c5ccccc5)c5cccc6ccccc56)cc4N(c4ccc(C)cc4)c4cccc2c43)cc1 ; c1ccc(N(c2ccc3c(c2)N(c2ccccc2)c2cccc4c2B3c2ccccc2N4c2ccccc2)c2ccc3c(c2)c2ccccc2n3-c2ccccc2)cc1 ; c1ccc(N2c3ccccc3B3c4ccc(N(c5ccccc5)c5cccc6c7ccccc7n(-c7ccccc7)c56)cc4N(c4ccccc4)c4cccc2c43)cc1]

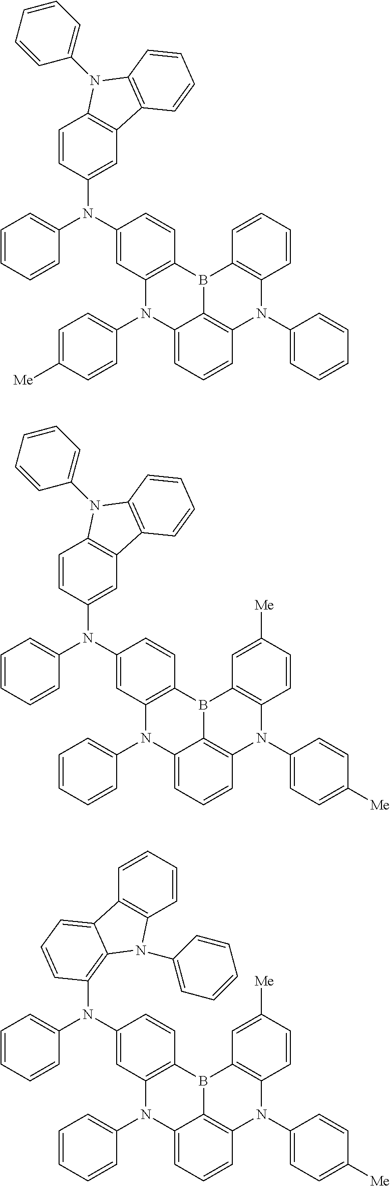 [SMILES: Cc1ccc(N2c3cc(N(c4ccccc4)c4ccc5c(c4)c4ccccc4n5-c4ccccc4)ccc3B3c4ccccc4N(c4ccccc4)c4cccc2c43)cc1 ; Cc1ccc(N2c3ccc(C)cc3B3c4ccc(N(c5ccccc5)c5ccc6c(c5)c5ccccc5n6-c5ccccc5)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccccc4)c4cccc2c43)cc1]

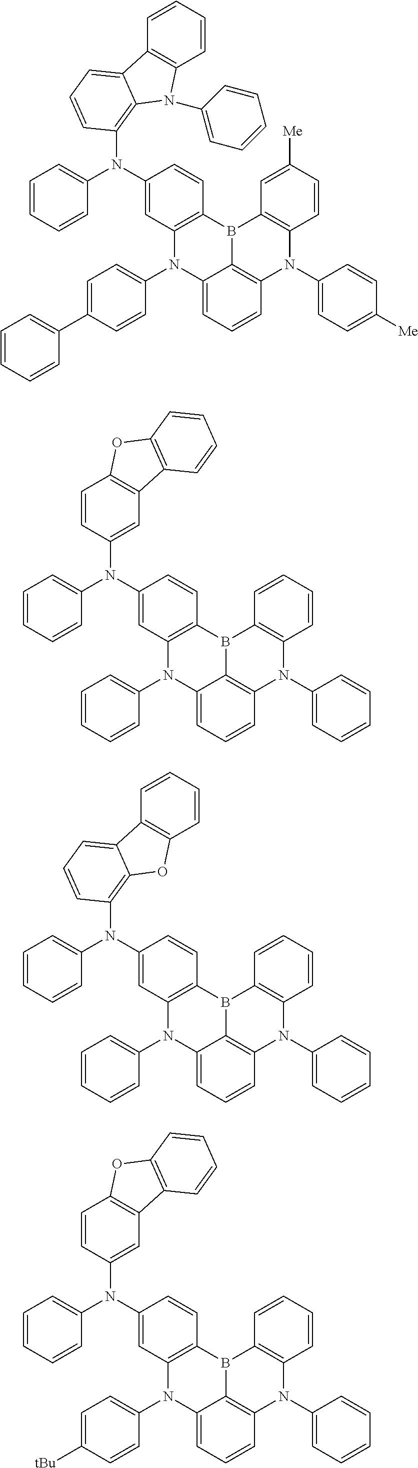

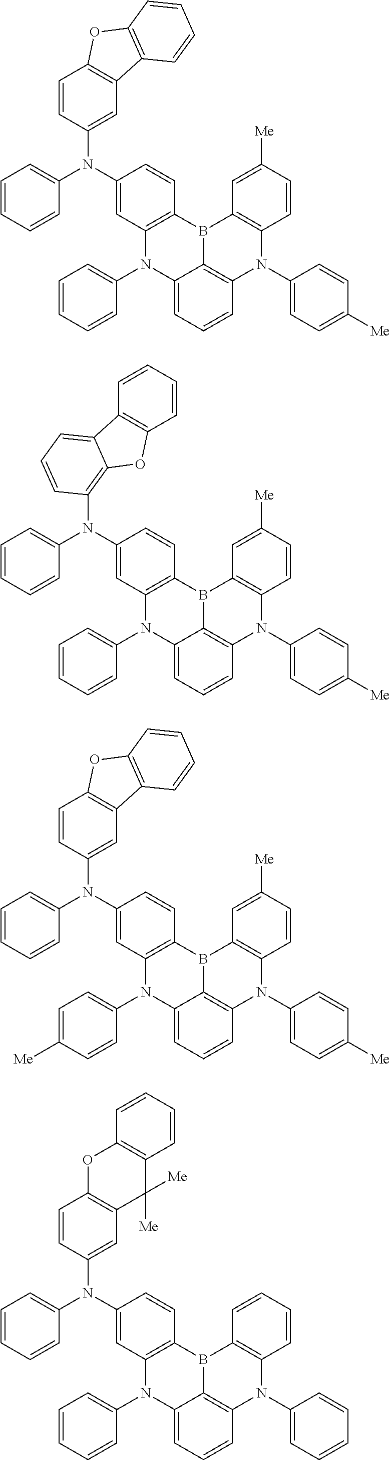

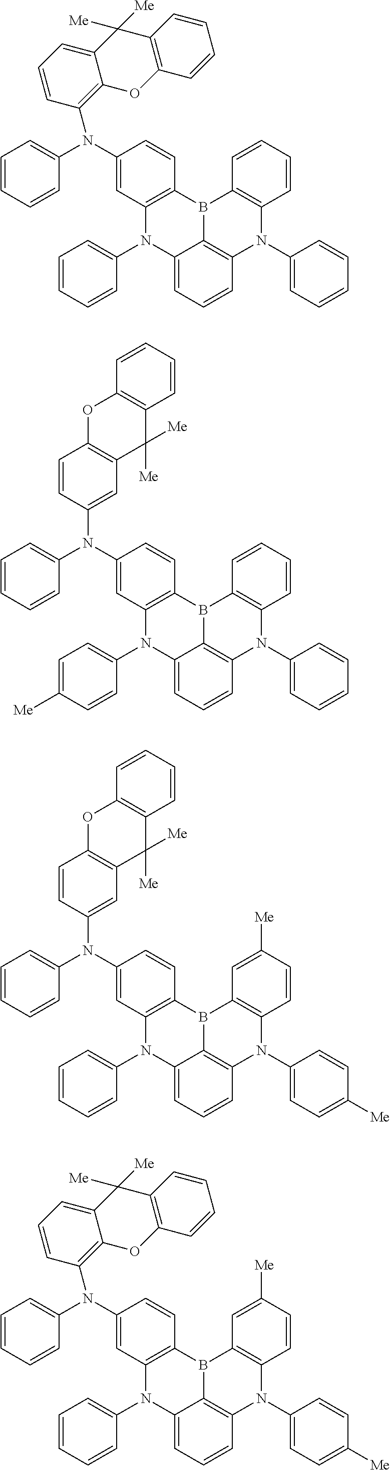 [SMILES: CC1(C)c2ccccc2Oc2c(N(c3ccccc3)c3ccc4c(c3)N(c3ccccc3)c3cccc5c3B4c3ccccc3N5c3ccccc3)cccc21 ; Cc1ccc(N2c3cc(N(c4ccccc4)c4ccc5c(c4)C(C)(C)c4ccccc4O5)ccc3B3c4ccccc4N(c4ccccc4)c4cccc2c43)cc1 ; Cc1ccc(N2c3ccc(C)cc3B3c4ccc(N(c5ccccc5)c5ccc6c(c5)C(C)(C)c5ccccc5O6)cc4N(c4ccccc4)c4cccc2c43)cc1 ; Cc1ccc(N2c3ccc(C)cc3B3c4ccc(N(c5ccccc5)c5cccc6c5Oc5ccccc5C6(C)C)cc4N(c4ccccc4)c4cccc2c43)cc1]

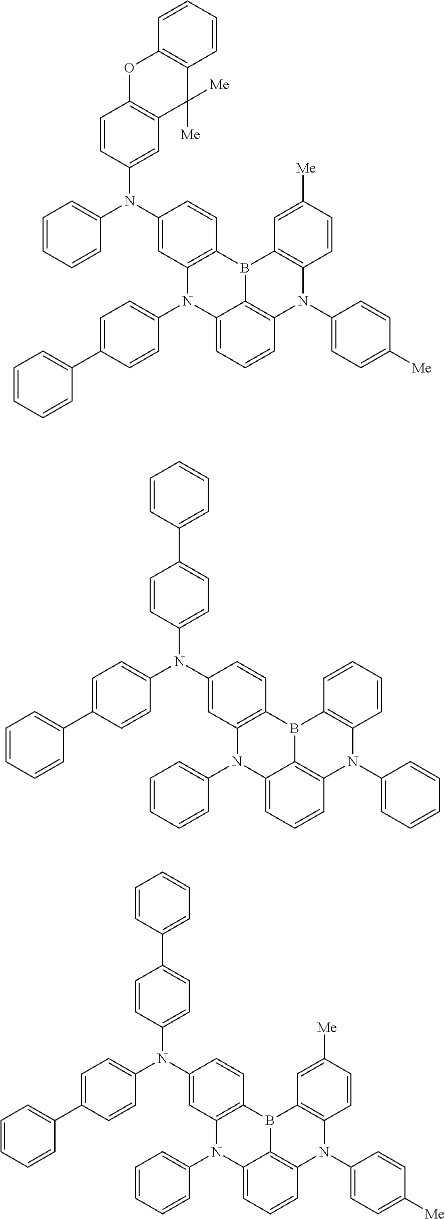 [SMILES: Cc1ccc(N2c3ccc(C)cc3B3c4ccc(N(c5ccccc5)c5ccc6c(c5)C(C)(C)c5ccccc5O6)cc4N(c4ccc(-c5ccccc5)cc4)c4cccc2c43)cc1 ; c1ccc(-c2ccc(N(c3ccc(-c4ccccc4)cc3)c3ccc4c(c3)N(c3ccccc3)c3cccc5c3B4c3ccccc3N5c3ccccc3)cc2)cc1 ; Cc1ccc(N2c3ccc(C)cc3B3c4ccc(N(c5ccc(-c6ccccc6)cc5)c5ccc(-c6ccccc6)cc5)cc4N(c4ccccc4)c4cccc2c43)cc1]

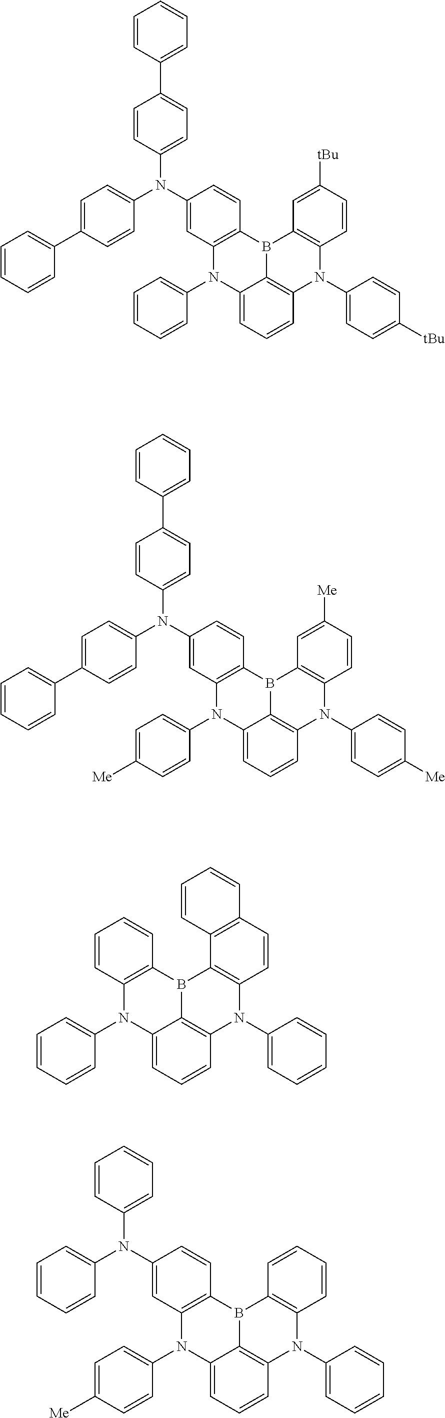 [SMILES: CC(C)(C)c1ccc(N2c3ccc(C(C)(C)C)cc3B3c4ccc(N(c5ccc(-c6ccccc6)cc5)c5ccc(-c6ccccc6)cc5)cc4N(c4ccccc4)c4cccc2c43)cc1 ; Cc1ccc(N2c3ccc(C)cc3B3c4ccc(N(c5ccc(-c6ccccc6)cc5)c5ccc(-c6ccccc6)cc5)cc4N(c4ccc(C)cc4)c4cccc2c43)cc1 ; c1ccc(N2c3ccccc3B3c4c2cccc4N(c2ccccc2)c2ccc4ccccc4c23)cc1 ; Cc1ccc(N2c3cc(N(c4ccccc4)c4ccccc4)ccc3B3c4ccccc4N(c4ccccc4)c4cccc2c43)cc1]

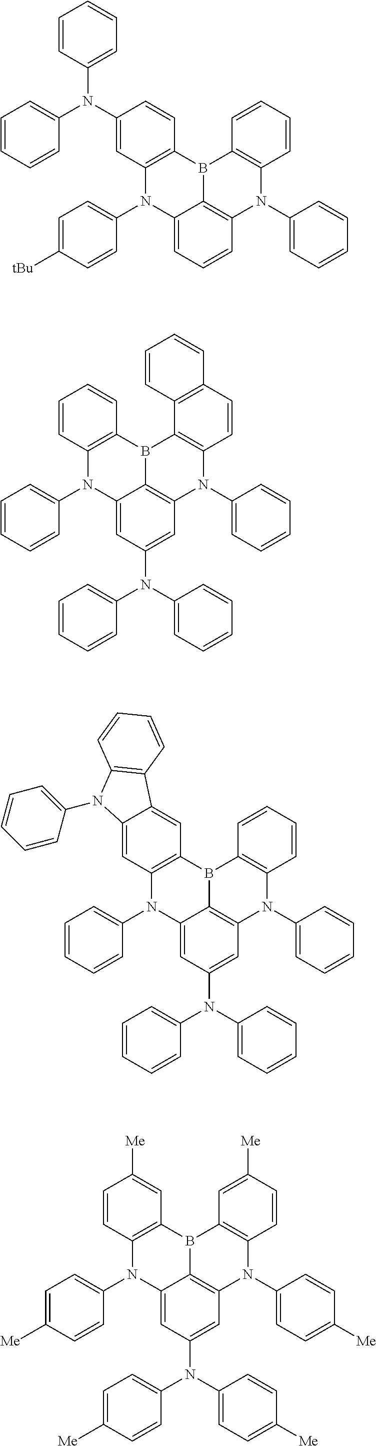 [SMILES: CC(C)(C)c1ccc(N2c3cc(N(c4ccccc4)c4ccccc4)ccc3B3c4ccccc4N(c4ccccc4)c4cccc2c43)cc1 ; c1ccc(N(c2ccccc2)c2cc3c4c(c2)N(c2ccccc2)c2ccc5ccccc5c2B4c2ccccc2N3c2ccccc2)cc1 ; c1ccc(N(c2ccccc2)c2cc3c4c(c2)N(c2ccccc2)c2cc5c(cc2B4c2ccccc2N3c2ccccc2)c2ccccc2n5-c2ccccc2)cc1 ; Cc1ccc(N(c2ccc(C)cc2)c2cc3c4c(c2)N(c2ccc(C)cc2)c2ccc(C)cc2B4c2cc(C)ccc2N3c2ccc(C)cc2)cc1]

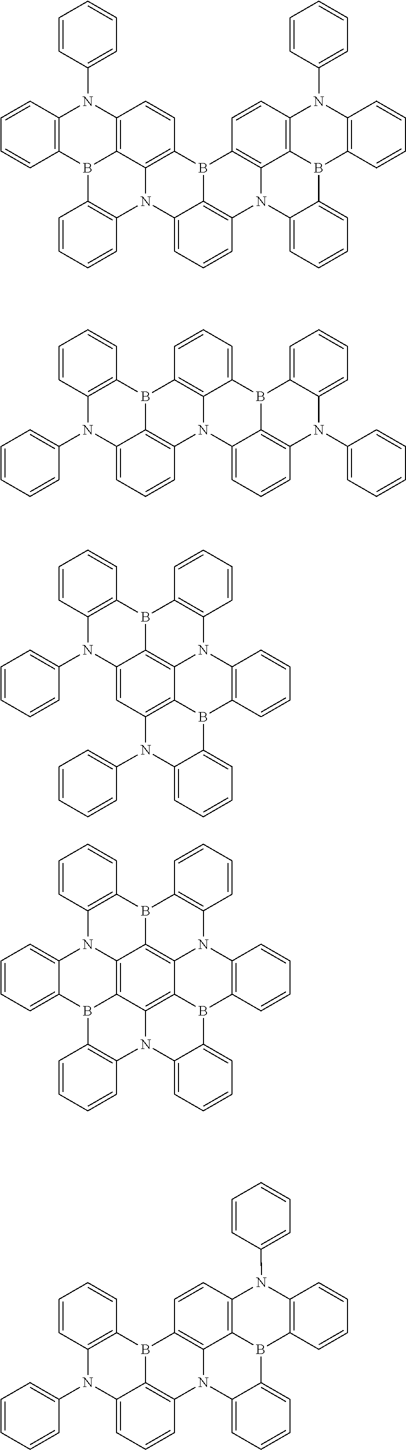

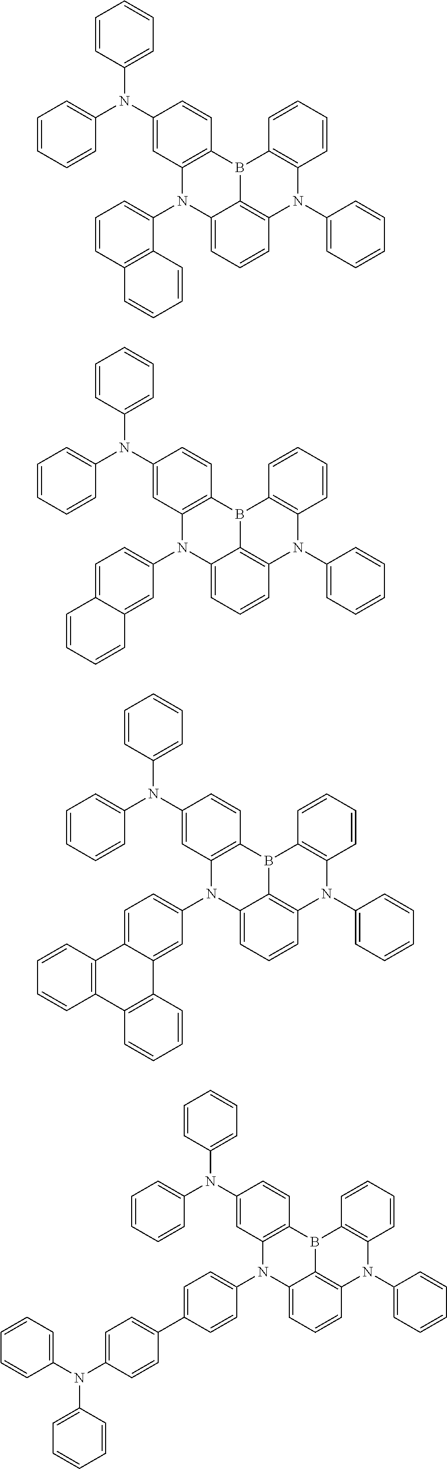

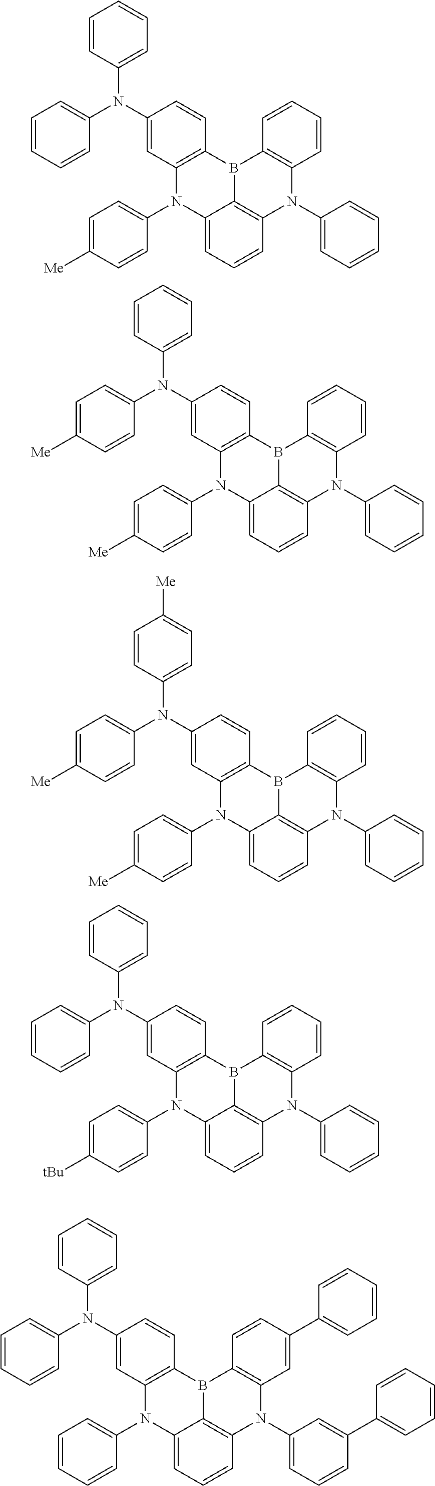

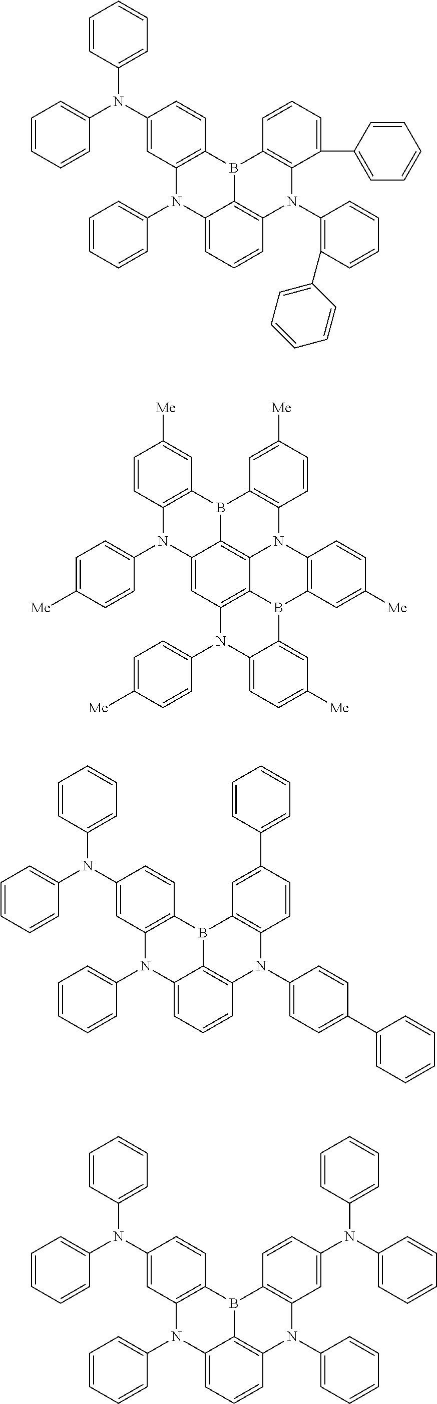 [SMILES: c1ccc(-c2ccccc2N2c3cccc4c3B(c3ccc(N(c5ccccc5)c5ccccc5)cc3N4c3ccccc3)c3cccc(-c4ccccc4)c32)cc1 ; Cc1ccc(N2c3ccc(C)cc3B3c4cc(C)ccc4N4c5ccc(C)cc5B5c6cc(C)ccc6N(c6ccc(C)cc6)c6cc2c3c4c65)cc1 ; c1ccc(-c2ccc(N3c4ccc(-c5ccccc5)cc4B4c5ccc(N(c6ccccc6)c6ccccc6)cc5N(c5ccccc5)c5cccc3c54)cc2)cc1 ; c1ccc(N(c2ccccc2)c2ccc3c(c2)N(c2ccccc2)c2cccc4c2B3c2ccc(N(c3ccccc3)c3ccccc3)cc2N4c2ccccc2)cc1]

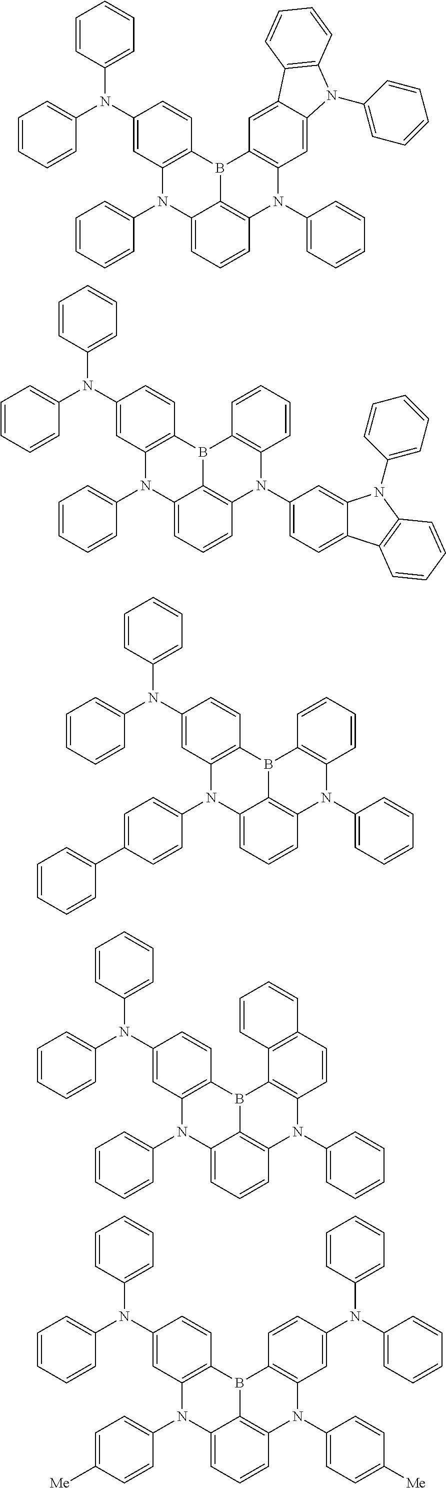

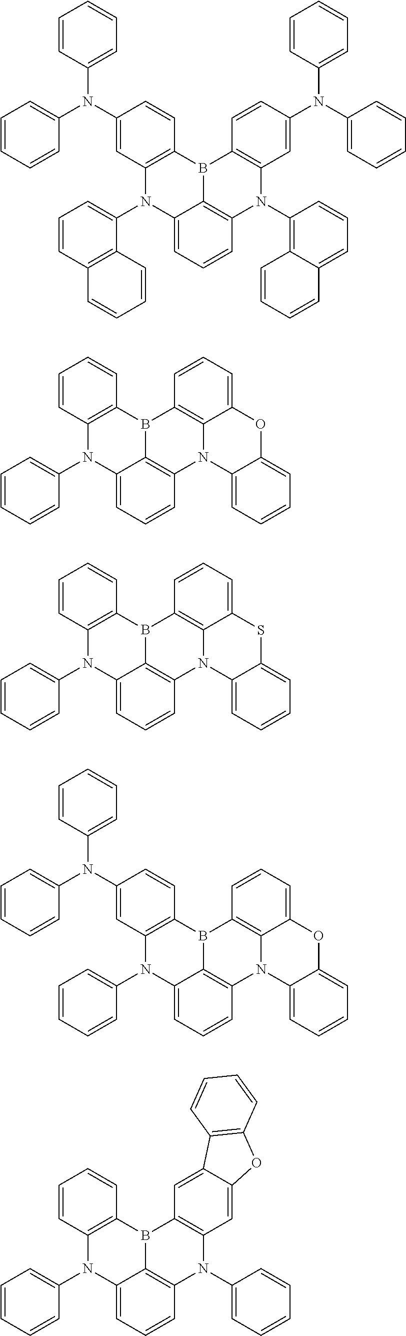

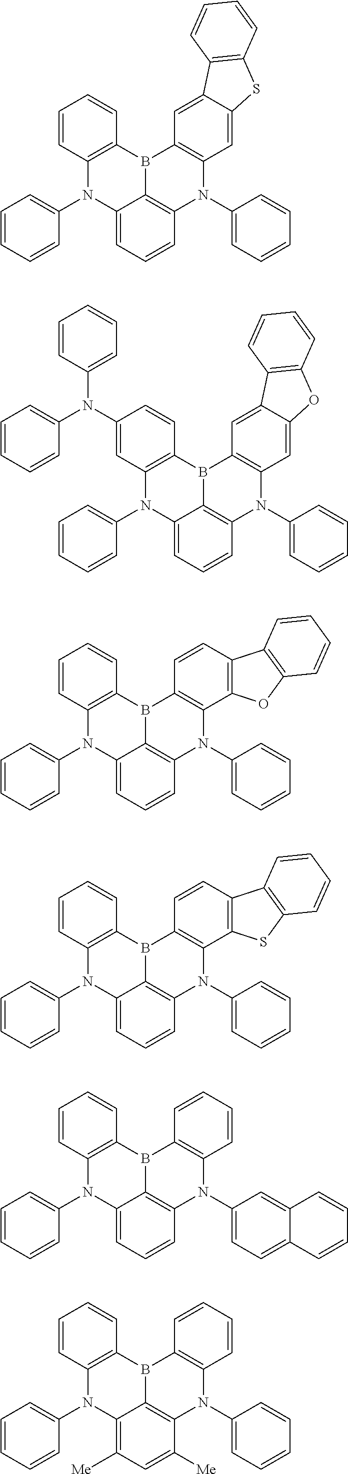

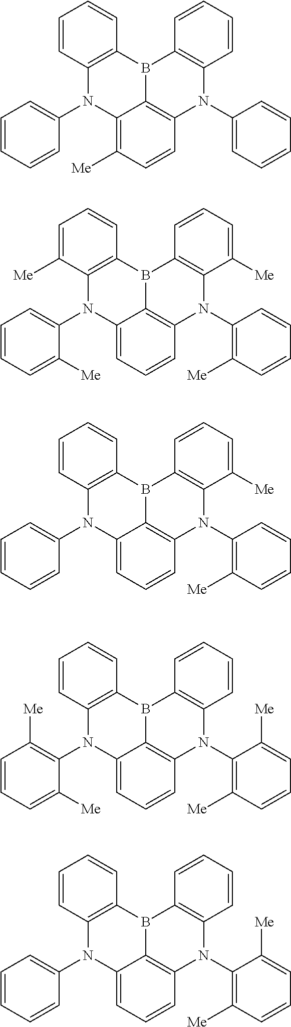

### First Compound

The first compound is used in the fluorescent emitting layer of the organic EL device of the invention together with the dopant material and the second compound and works as the host material (main host material) of the fluorescent emitting layer.

The first compound is at least one selected from an anthracene skeleton-containing compound represented by formula (19), a chrysene skeleton-containing compound represented by formula (21), a pyrene skeleton-containing compound represented by formula (22), and a fluorene skeleton-containing compound represented by formula (23), with the anthracene skeleton-containing compound being preferred.

An anthracene skeleton-containing compound represented by formula (19) is usable as the first compound:

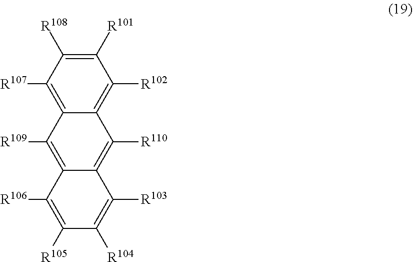

In formula (19), R¹⁰¹ to R¹¹⁰ are each independently a hydrogen atom, a substituent, or -L-Ar, provided that at least one of R¹⁰¹ to R¹¹⁰ is -L-Ar.

The details of the substituent are as described above with respect to the substituent of R_{A}, R_{B}, and R_{C}.

L is independently a single bond or a linker, wherein the linker is a substituted or unsubstituted arylene group having 6 to 50, preferably 6 to 30, more preferably 6 to 24, and still more preferably 6 to 18 ring carbon atoms or a substituted or unsubstituted heteroarylene group having 5 to 50, preferably 5 to 30, more preferably 5 to 18, and still more preferably 5 to 13 ring atoms.

Ar is independently a substituted or unsubstituted single ring group having 5 to 50, preferably 5 to 30, more preferably 5 to 24, and still more preferably 5 to 18 ring atoms, a substituted or unsubstituted fused ring group having 8 to 50, preferably 8 to 30, more preferably 8 to 24, and still more preferably 8 to 18 ring atoms, or a monovalent group wherein two or more selected from the single ring and the fused ring are bonded to each other via a single bond.

The single ring group having 5 to 50 ring atoms is a group having only a single ring structure and having no fused ring, for example, preferably an aryl group, such as a phenyl group, a biphenylyl group, a terphenylyl group, and a quaterphenylyl group, and a heteroaryl group, such as a pyridyl group, a pyrazinyl group, a pyrimidinyl group, a triazinyl group, a furyl group, and a thienyl group, and more preferably a phenyl group, a biphenylyl group, and a terphenylyl group.

The fused ring group having 8 to 50 ring atoms is a group having a fused ring structure wherein two or more rings are fused. Examples thereof are preferably a fused aryl group, such as a naphthyl group, a phenanthryl group, an anthryl group, a chrysenyl group, a benzanthryl group, a benzophenanthryl group, a triphenylenyl group, a benzochrysenyl group, an indenyl group, a fluorenyl group, a 9,9-dimethylfluorenyl group, a benzofluorenyl group, a dibenzofluorenyl group, a fluoranthenyl group, and a benzofluoranthenyl group, and a fused heteroaryl group, such as a benzofuranyl group, a benzothiophenyl group, an indolyl group, a dibenzofuranyl group, a dibenzothiophenyl group, a carbazolyl group, a quinolyl group, and a phenanthrolinyl group, with a naphthyl group, a phenanthryl group, an anthryl group, a 9,9-dimethylfluorenyl group, a fluoranthenyl group, a benzanthryl group, a dibenzothiophenyl group, a dibenzofuranyl group, and a carbazolyl group being more preferred.

The optional substituent of Ar is preferably the single ring group or the fused ring group each mentioned above.

The arylene group of the substituted or unsubstituted arylene group having 6 to 30 ring carbon atoms represented by L is a divalent group derived by removing two hydrogen atoms from an aromatic hydrocarbon compound selected from benzene, naphthylbenzene, biphenyl, terphenyl, naphthalene, acenaphthylene, anthracene, benzanthracene, aceanthracene, phenanthrene, benzo[c]phenanthrene, phenalene, fluorene, picene, pentaphene, pyrene, chrysene, benzo[g]chrysene, s-indacene, as-indacene, fluoranthene, benzo[k]fluoranthene, triphenylene, benzo[b]triphenylene, and perylene. Preferred are a phenylene group, a biphenyldiyl group, a terphenyldiyl group, and a naphthalenediyl group, with a phenylene group, a biphenyldiyl group, and a terphenyldiyl group being more preferred and a phenylene group being still more preferred.

The heteroarylene group of the substituted or unsubstituted heteroarylene group having 5 to 30 ring carbon atoms represented by L is a divalent group obtained by removing two hydrogen atoms from an aromatic heterocyclic ring having at least one and preferably 1 to 5 ring hetero atom, for example, a nitrogen atom, a sulfur atom, and an oxygen atom. Examples of the aromatic heterocyclic ring include pyrrole, furan, thiophene, pyridine, pyridazine, pyrimidine, pyrazine, triazine, imidazole, oxazole, thiazole, pyrazole, isoxazole, isothiazole, oxadiazole, thiadiazole, triazole, tetrazole, indole, isoindole, benzofuran, isobenzofuran, benzothiophene, isobenzothiophene, indolizine, quinolizine, quinoline, isoquinoline, cinnoline, phthalazine, quinazoline, quinoxaline, benzimidazole, benzoxazole, benzothiazole, indazole, benzisoxazole, benzoisothiazole, dibenzofuran, dibenzothiophene, carbazole, phenanthridine, acridine, phenanthroline, phenazine, phenothiazine, phenoxazine, and xanthene. Preferred examples of the heteroarylene group are divalent groups obtained by removing two hydrogen atoms from furan, thiophene, pyridine, pyridazine, pyrimidine, pyrazine, triazine, benzofuran, benzothiophene, dibenzofuran, and dibenzothiophene, with divalent groups obtained by removing two hydrogen atoms from benzofuran, benzothiophene, dibenzofuran, and dibenzothiophene being more preferred.

The compound of formula (19) is preferably an anthracene derivative represented by formula (20):

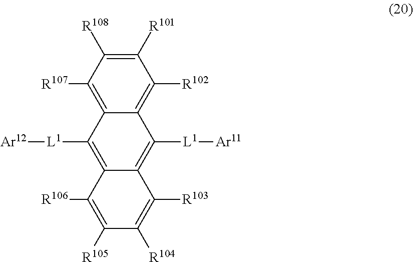

wherein:

R¹⁰¹ to R¹⁰⁸ are as defined in formula (19);

L¹ is as defined above with respect to L of formula (19); and

Ar¹¹ and Ar¹² are as defined above with respect to Ar of formula (19).

The anthracene derivative represented by formula (20) is preferably any of the anthracene derivatives (A), (B), and (C), which are selected according to the structure of the organic EL device and required properties.

### Anthracene Derivative (A)

The anthracene derivative (A) is a compound of formula (20), wherein Ar¹¹ and Ar¹² are independently a substituted or unsubstituted fused ring group having 8 to 50 ring atoms. Ar¹¹ and Ar¹² may be the same or different, preferably different.

Examples of the fused ring group having 8 to 50 ring atoms are as described above with respect to formula (19) and preferably a naphthyl group, a phenanthryl group, a benzanthryl group, a 9,9-dimethylfluorenyl group, and a dibenzofuranyl group.

### Anthracene Derivative (B)

The anthracene derivative (B) is a compound of formula (20), wherein one of Ar¹¹ and Ar¹² is a substituted or unsubstituted single ring group having 5 to 50 ring atoms and the other is a substituted or unsubstituted fused ring group having 8 to 50 ring atoms.

The details of the single ring group having 5 to 50 ring atoms and the fused ring group having 8 to 50 ring atoms are as described above with respect to formula (19).

In an embodiment of the invention, Ar¹² is preferably a naphthyl group, a phenanthryl group, a benzanthryl group, a 9,9-dimethylfluorenyl group, or a dibenzofuranyl group and Ar¹¹ is preferably an unsubstituted phenyl group or a phenyl group substituted with a single ring group or a fused ring group, for example, a phenyl group, a biphenyl group, a naphthyl group, a phenanthryl group, a 9,9-dimethylfluorenyl group, or a dibenzofuranyl group.

In another embodiment of the invention, Ar¹² is preferably a substituted or unsubstituted fused ring group having 8 to 50 ring atoms and Ar¹¹ is an unsubstituted phenyl group. The fused ring group is particularly preferably a phenanthryl group, a 9,9-dimethylfluorenyl group, a dibenzofuranyl group, or a benzanthryl group.

### Anthracene Derivative (C)

The anthracene derivative (C) is a compound of formula (20), wherein Ar¹¹ and Ar¹² are each independently a substituted or unsubstituted single ring group having 5 to 50 ring atoms.

Preferably, each of Ar¹¹ and Ar¹² is a substituted or unsubstituted phenyl group. More preferably, Ar¹¹ is an unsubstituted phenyl group and Ar¹² is phenyl group substituted with a single ring group or a fused ring group, or Ar¹¹ and Ar¹² are each independently a phenyl group substituted with a single ring group or a fused ring group.

The single ring group and the fused ring group as the optional substituent of Ar¹¹ and Ar¹² are as described above with respect to formula (19). The single ring group is preferably a phenyl group or a biphenyl group and the fused ring group is preferably a naphthyl group, a phenanthryl group, a 9,9-dimethylfluorenyl group, a dibenzofuranyl group, or a benzanthryl group.

Examples of the anthracene derivative represented by formula (19) or (20) are shown below.

In the following compounds, the six-membered rings are all benzene rings.

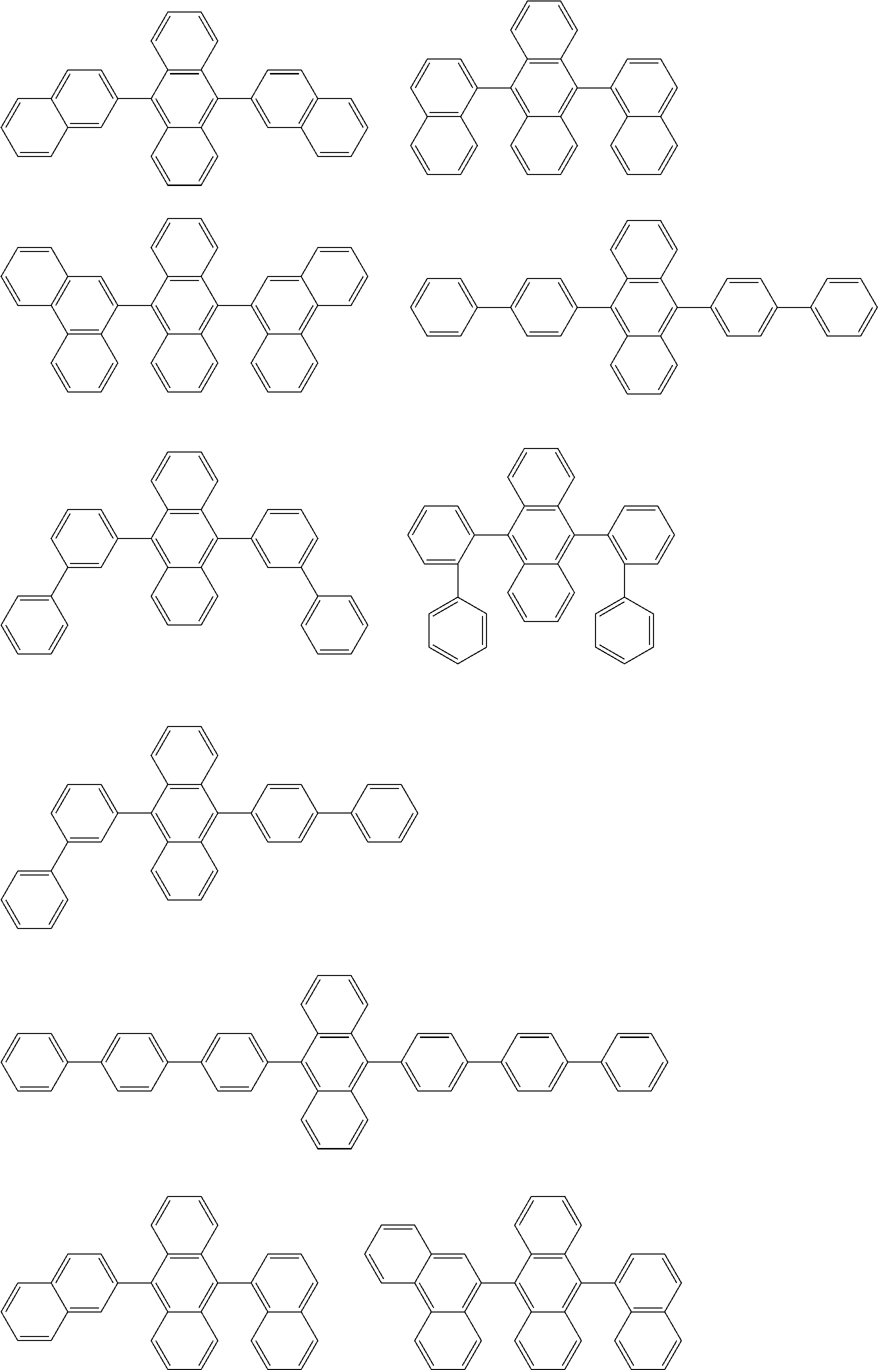

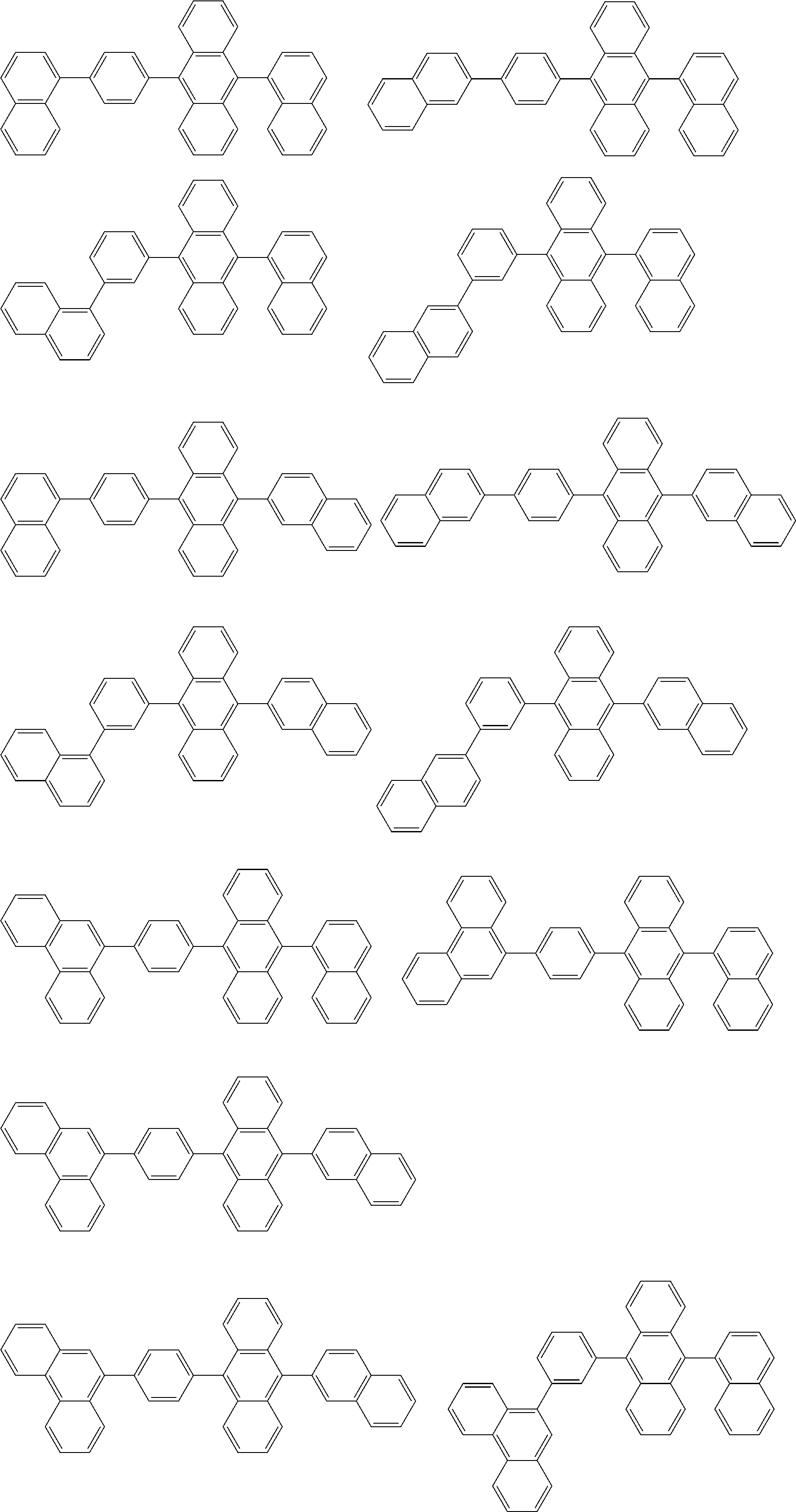

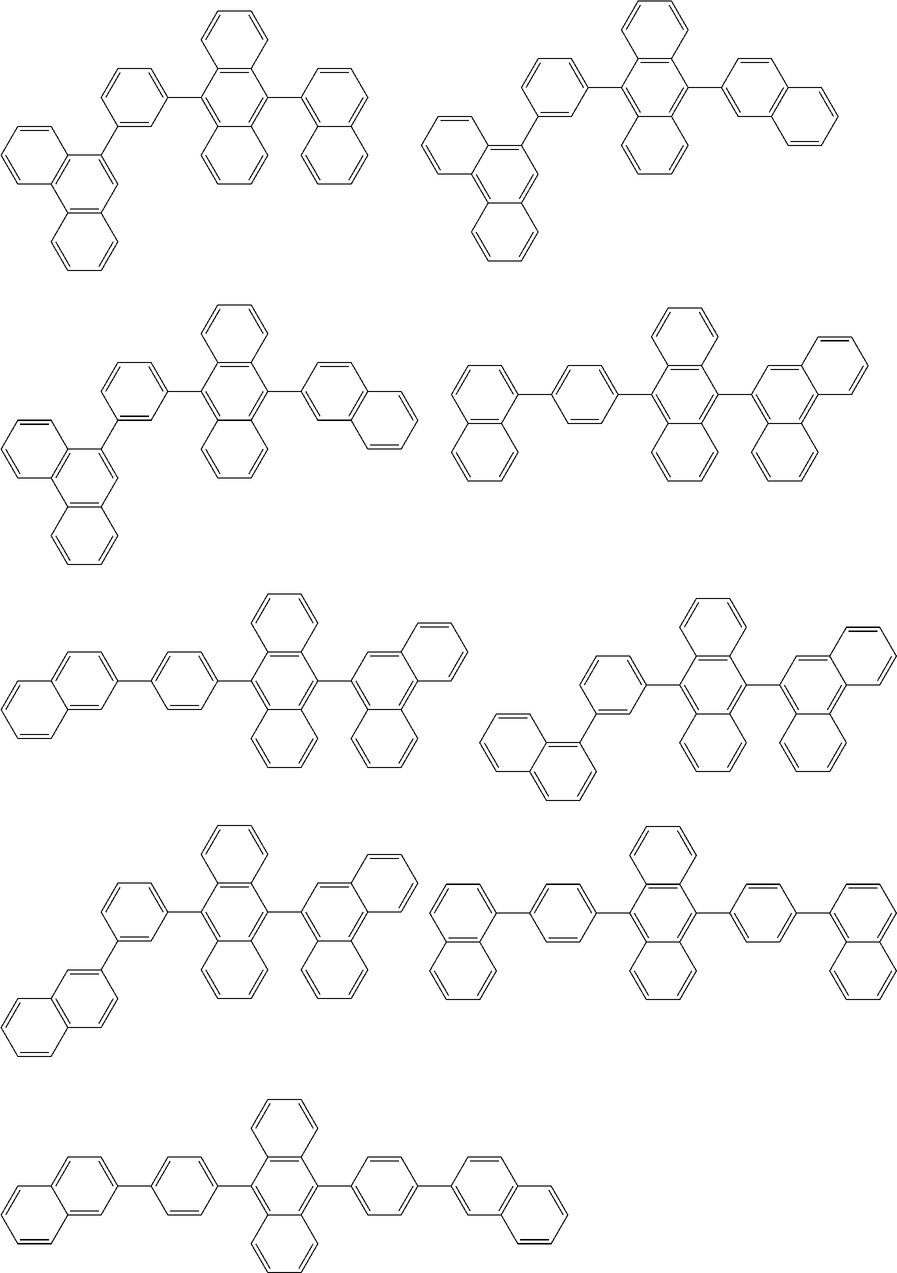

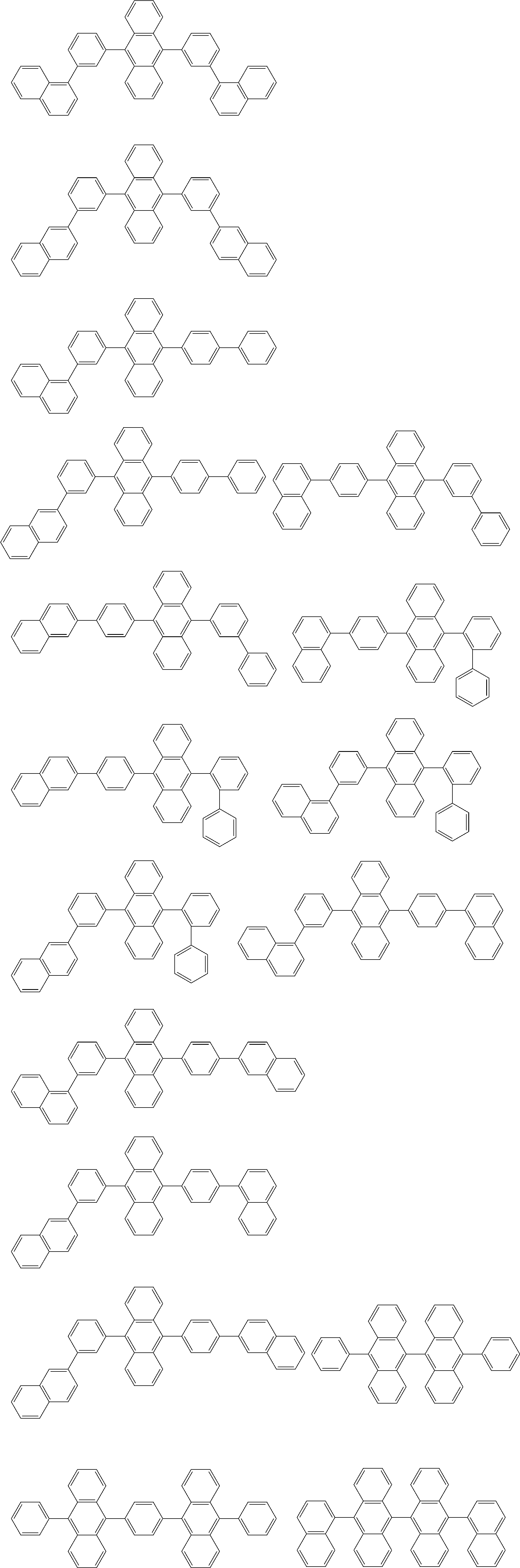

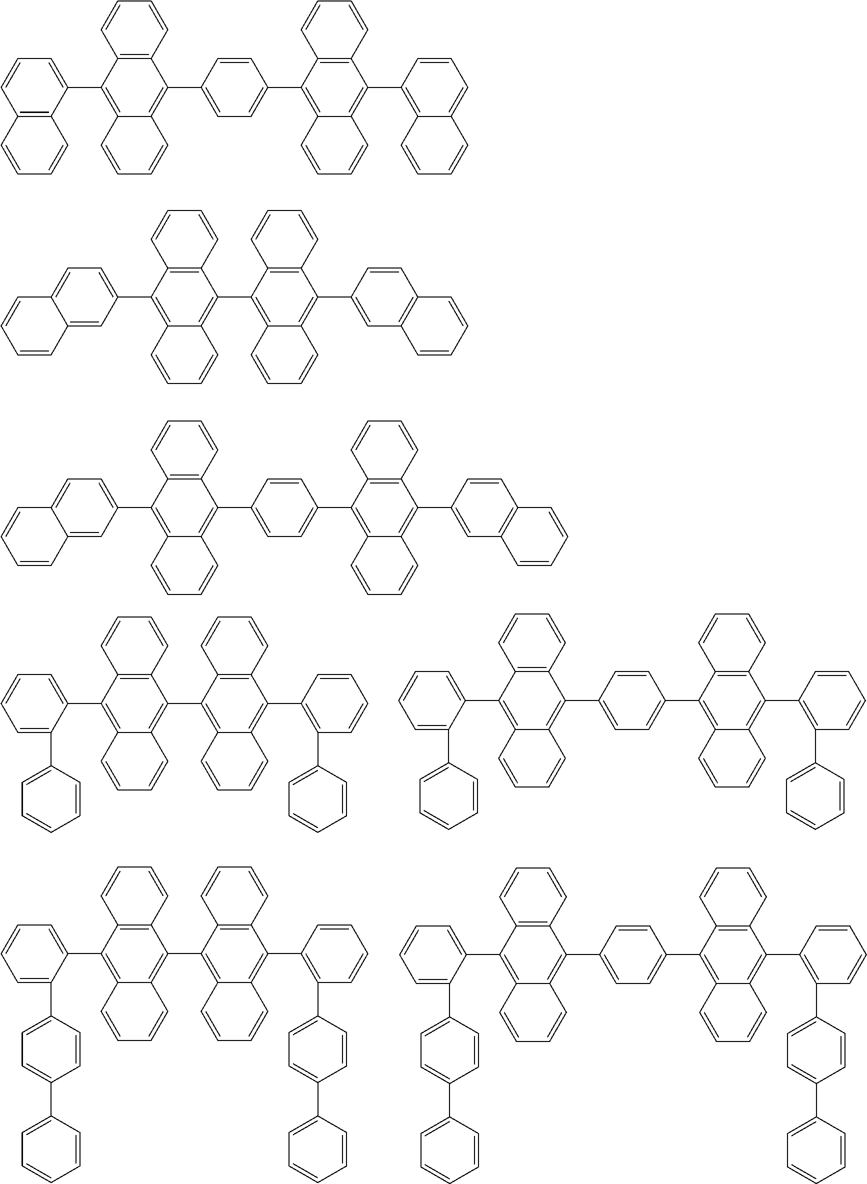

In the following compounds, the six-membered rings are all benzene rings.

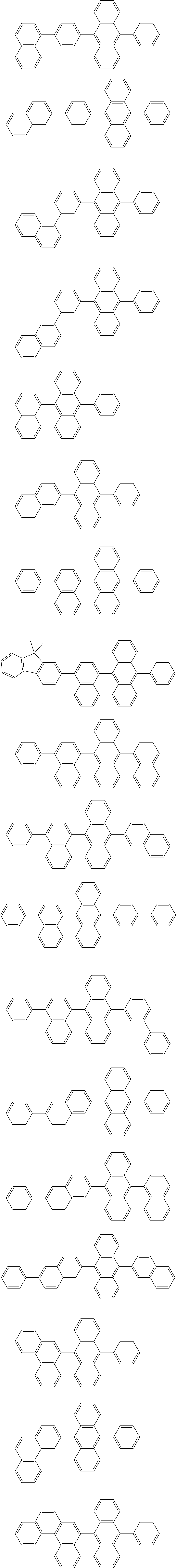

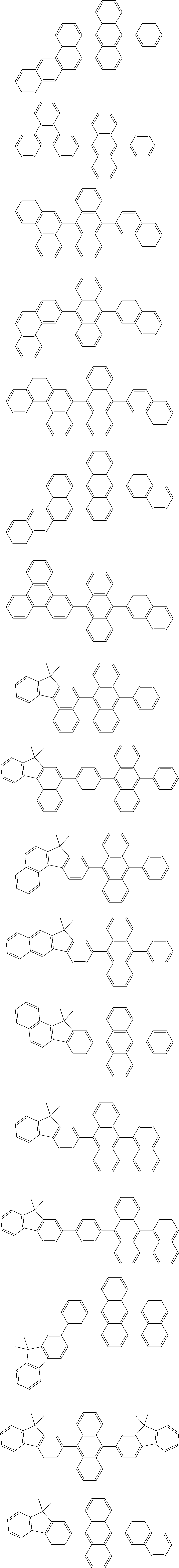

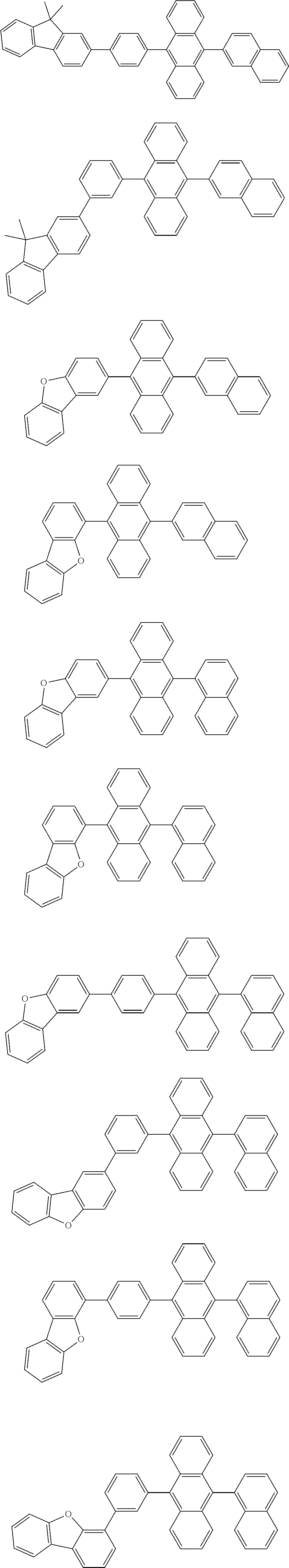

In the following compounds, the six-membered rings are all benzene rings.

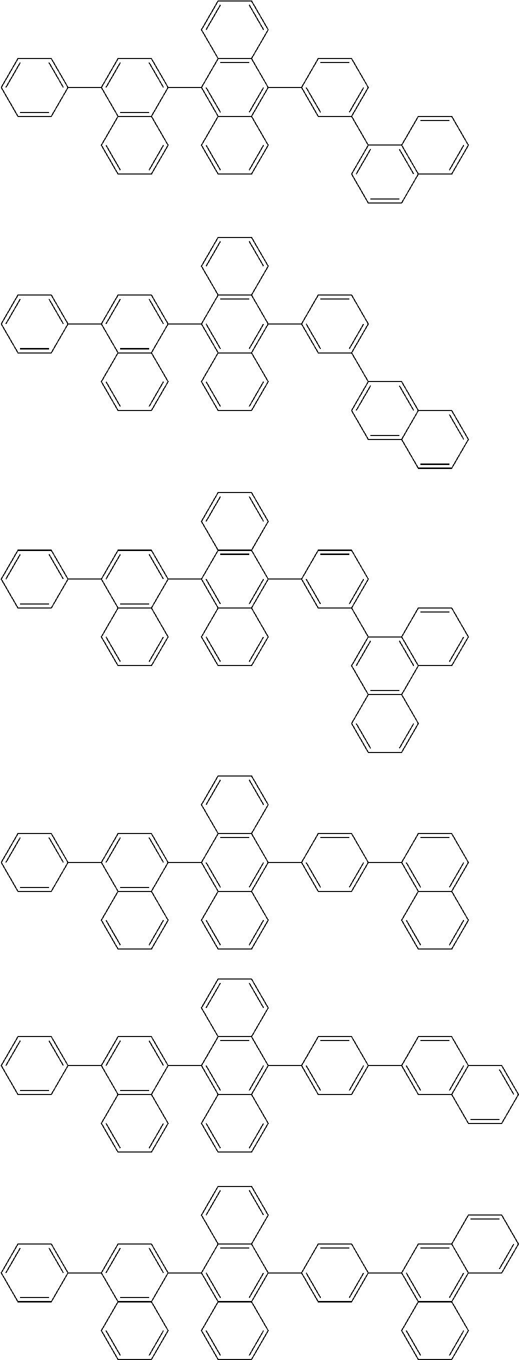

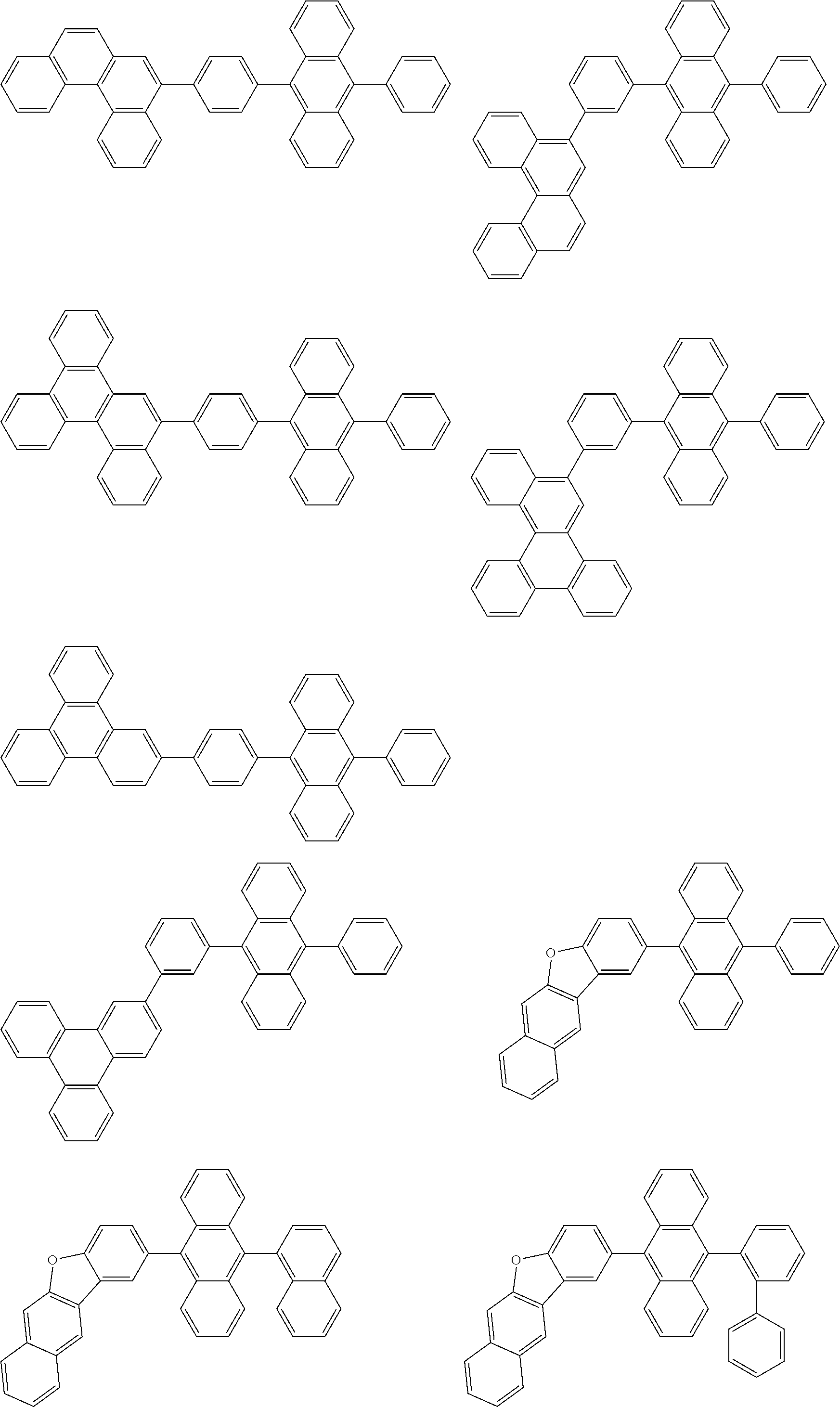

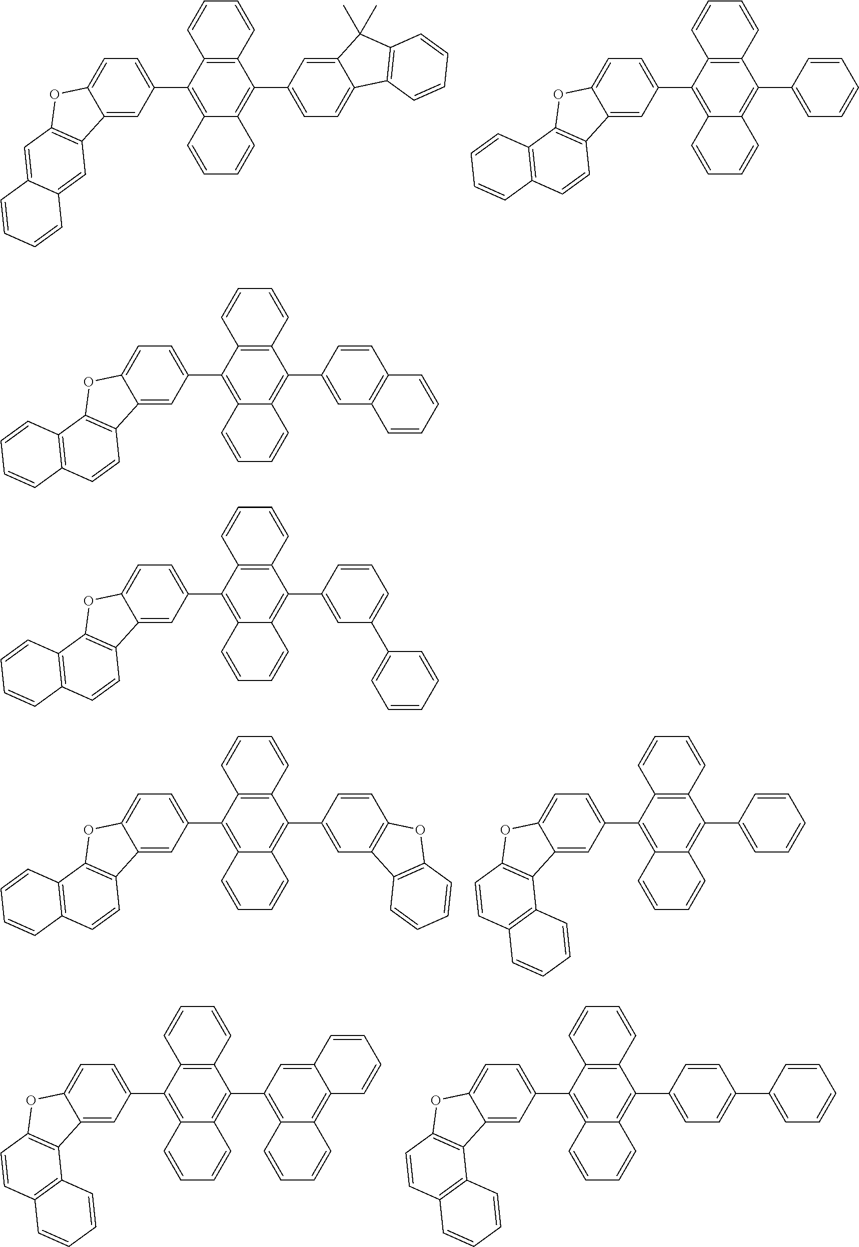

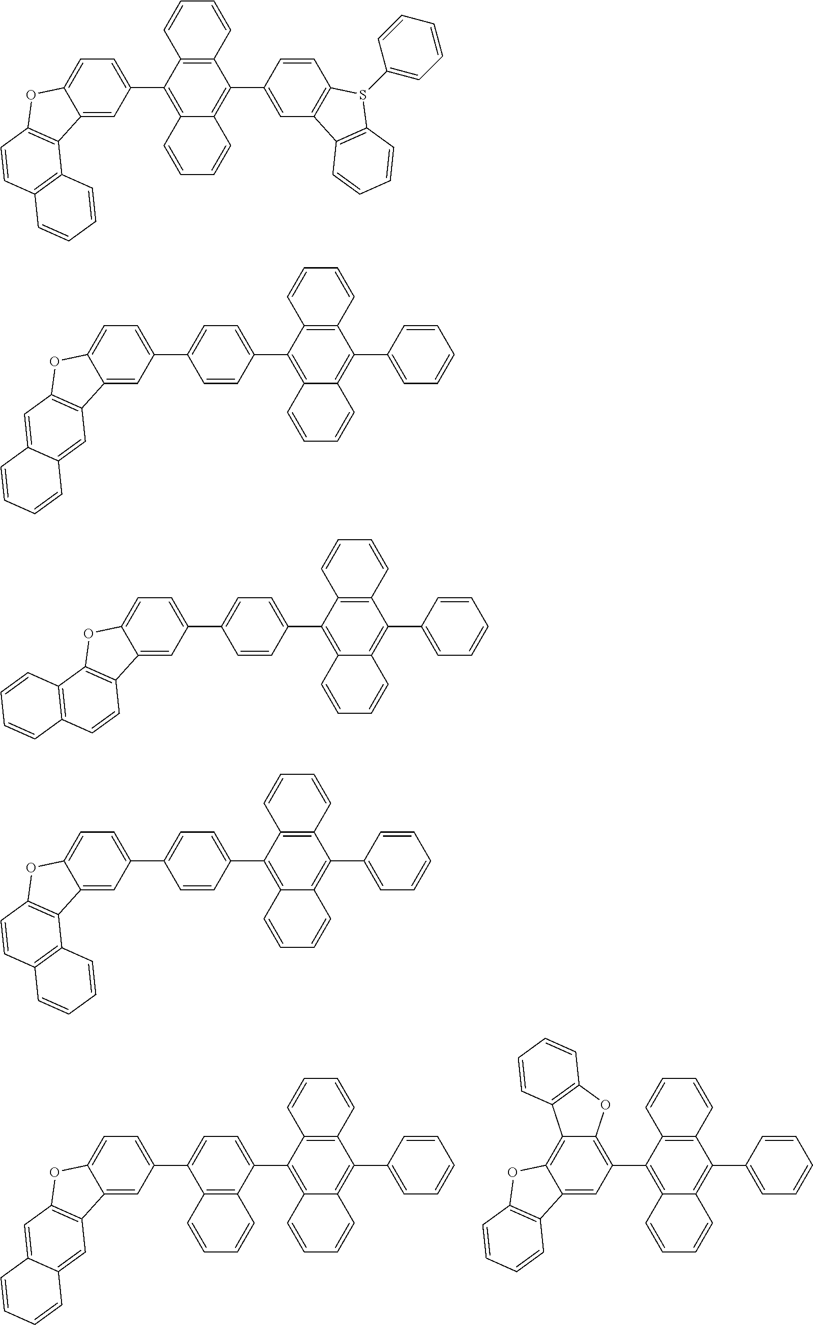

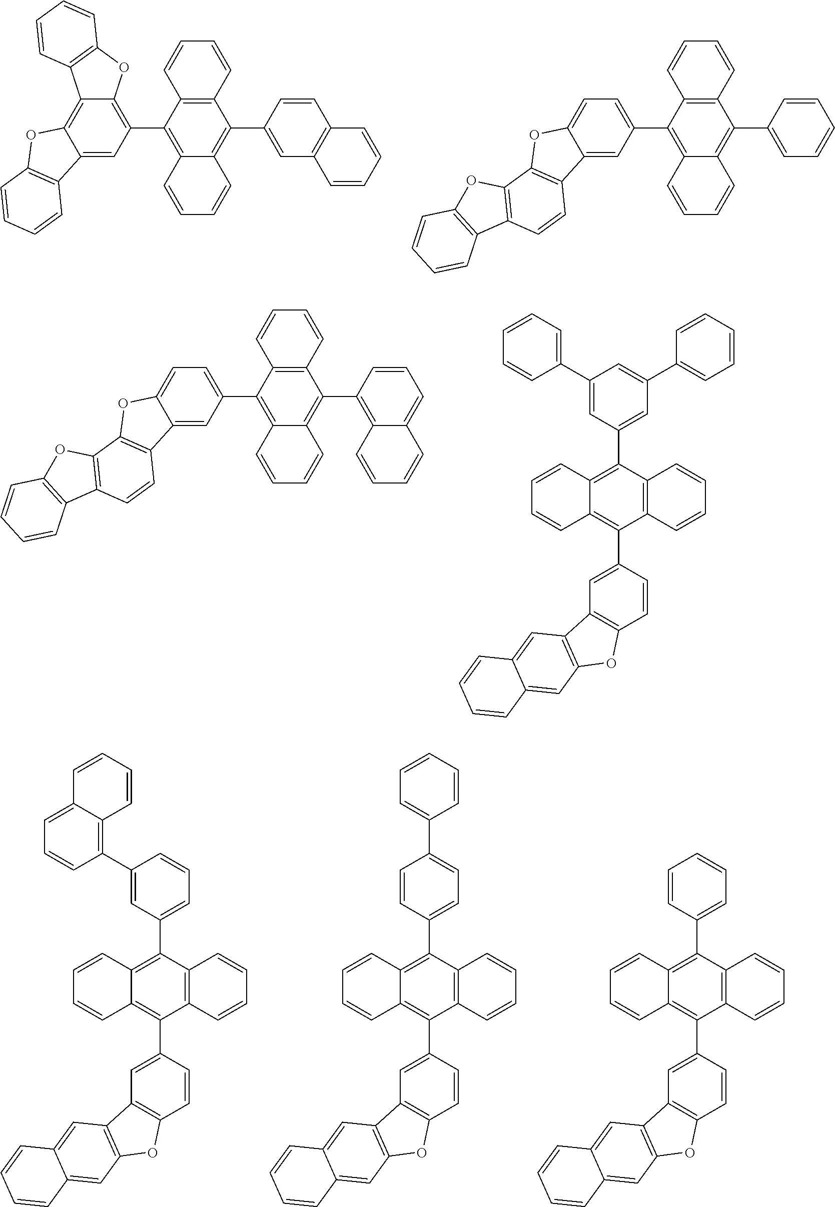

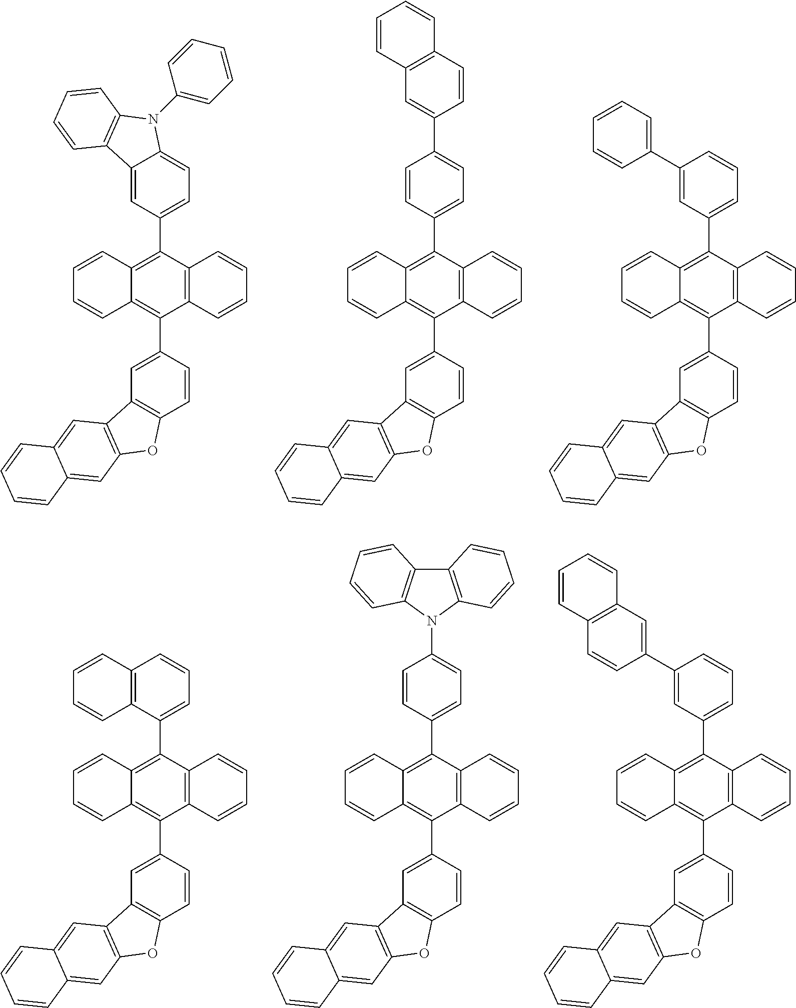 [SMILES: c1ccc(-n2c3ccccc3c3cc(-c4c5ccccc5c(-c5ccc6oc7cc8ccccc8cc7c6c5)c5ccccc45)ccc32)cc1 ; c1ccc2cc(-c3ccc(-c4c5ccccc5c(-c5ccc6oc7cc8ccccc8cc7c6c5)c5ccccc45)cc3)ccc2c1 ; c1ccc(-c2cccc(-c3c4ccccc4c(-c4ccc5oc6cc7ccccc7cc6c5c4)c4ccccc34)c2)cc1 ; c1ccc2cc3c(cc2c1)oc1ccc(-c2c4ccccc4c(-c4cccc5ccccc45)c4ccccc24)cc13 ; c1ccc2cc3c(cc2c1)oc1ccc(-c2c4ccccc4c(-c4ccc(-n5c6ccccc6c6ccccc65)cc4)c4ccccc24)cc13 ; c1cc(-c2ccc3ccccc3c2)cc(-c2c3ccccc3c(-c3ccc4oc5cc6ccccc6cc5c4c3)c3ccccc23)c1]

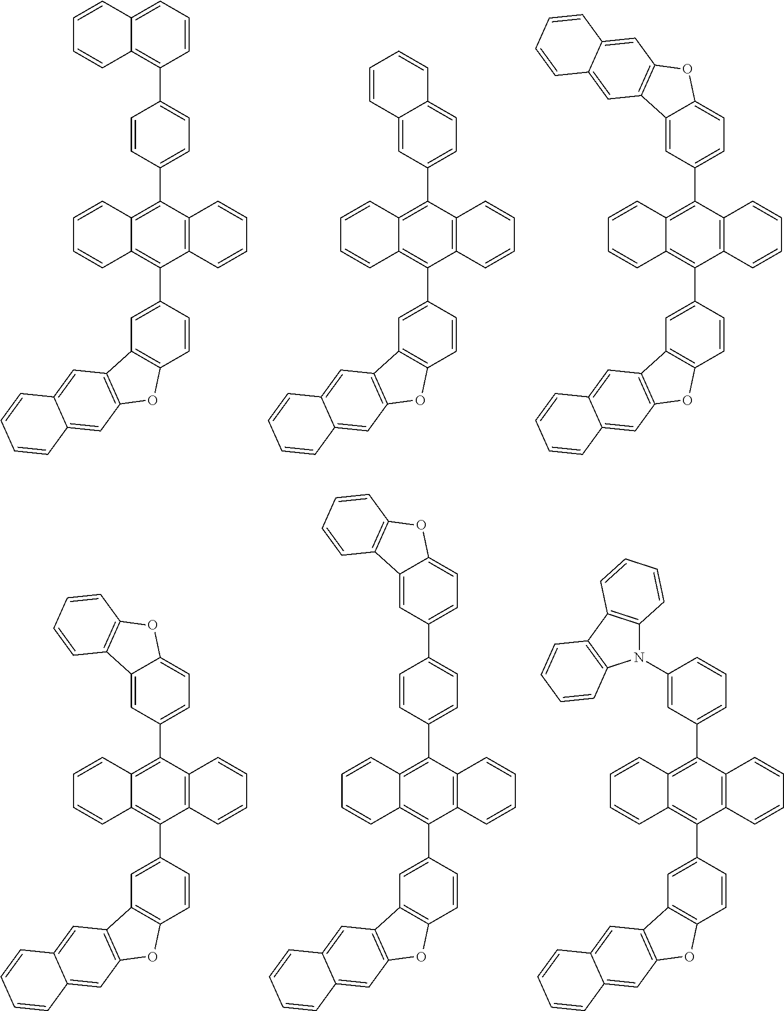 [SMILES: c1ccc2cc3c(cc2c1)oc1ccc(-c2c4ccccc4c(-c4ccc(-c5cccc6ccccc56)cc4)c4ccccc24)cc13 ; c1ccc2cc(-c3c4ccccc4c(-c4ccc5oc6cc7ccccc7cc6c5c4)c4ccccc34)ccc2c1 ; c1ccc2cc3c(cc2c1)oc1ccc(-c2c4ccccc4c(-c4ccc5oc6cc7ccccc7cc6c5c4)c4ccccc24)cc13 ; c1ccc2cc3c(cc2c1)oc1ccc(-c2c4ccccc4c(-c4ccc5oc6ccccc6c5c4)c4ccccc24)cc13 ; c1ccc2cc3c(cc2c1)oc1ccc(-c2c4ccccc4c(-c4ccc(-c5ccc6oc7ccccc7c6c5)cc4)c4ccccc24)cc13 ; c1cc(-c2c3ccccc3c(-c3ccc4oc5cc6ccccc6cc5c4c3)c3ccccc23)cc(-n2c3ccccc3c3ccccc32)c1]

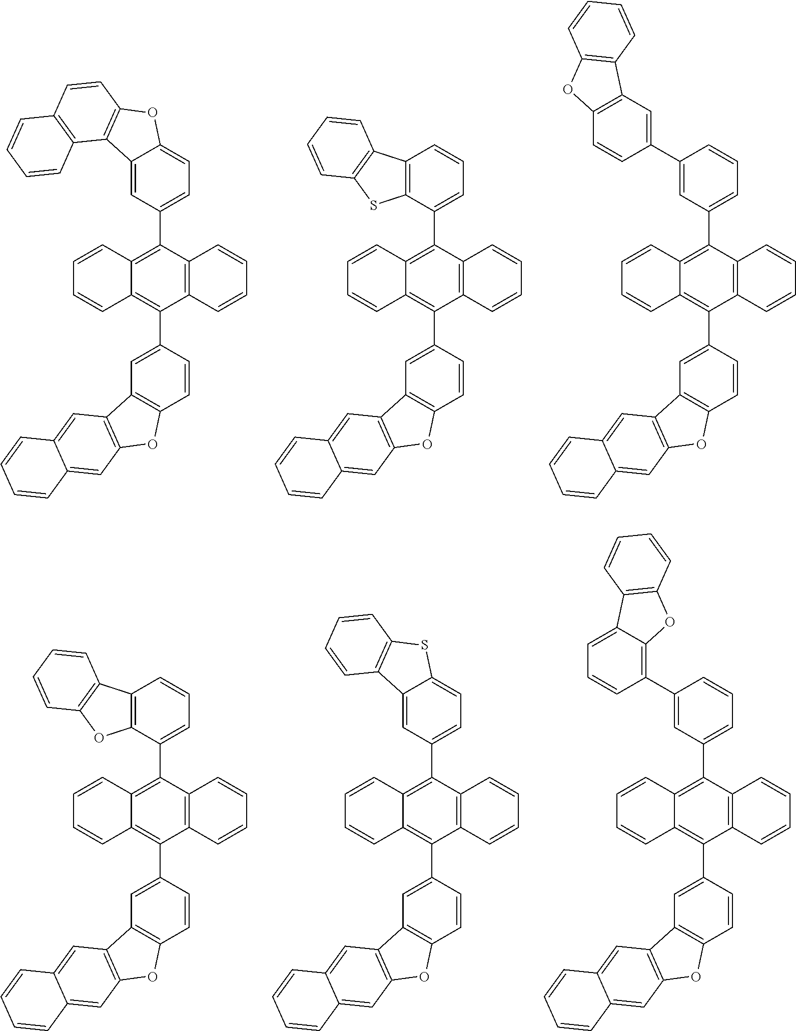

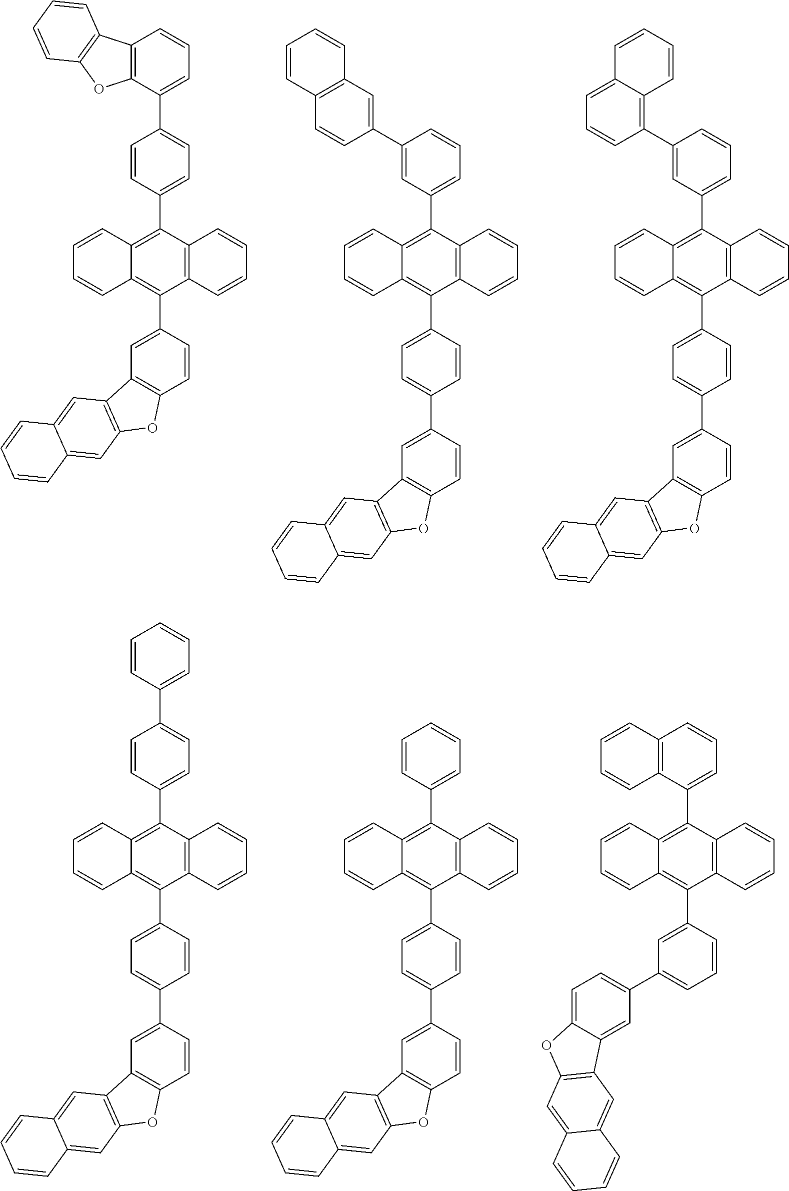 [SMILES: c1ccc2cc3c(cc2c1)oc1ccc(-c2c4ccccc4c(-c4ccc(-c5cccc6c5oc5ccccc56)cc4)c4ccccc24)cc13 ; c1cc(-c2ccc3ccccc3c2)cc(-c2c3ccccc3c(-c3ccc(-c4ccc5oc6cc7ccccc7cc6c5c4)cc3)c3ccccc23)c1 ; c1cc(-c2cccc3ccccc23)cc(-c2c3ccccc3c(-c3ccc(-c4ccc5oc6cc7ccccc7cc6c5c4)cc3)c3ccccc23)c1 ; c1ccc(-c2ccc(-c3c4ccccc4c(-c4ccc(-c5ccc6oc7cc8ccccc8cc7c6c5)cc4)c4ccccc34)cc2)cc1 ; c1ccc(-c2c3ccccc3c(-c3ccc(-c4ccc5oc6cc7ccccc7cc6c5c4)cc3)c3ccccc23)cc1 ; c1cc(-c2ccc3oc4cc5ccccc5cc4c3c2)cc(-c2c3ccccc3c(-c3cccc4ccccc34)c3ccccc23)c1]

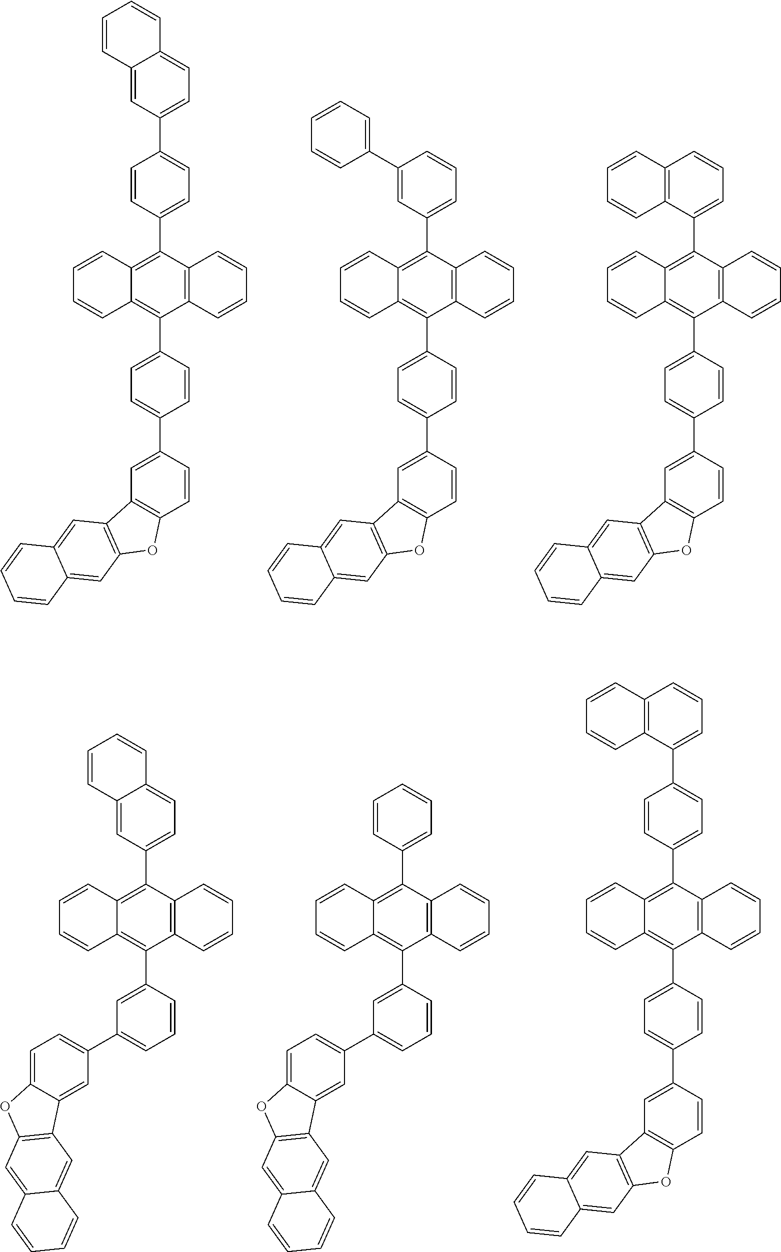 [SMILES: c1ccc2cc(-c3ccc(-c4c5ccccc5c(-c5ccc(-c6ccc7oc8cc9ccccc9cc8c7c6)cc5)c5ccccc45)cc3)ccc2c1 ; c1ccc(-c2cccc(-c3c4ccccc4c(-c4ccc(-c5ccc6oc7cc8ccccc8cc7c6c5)cc4)c4ccccc34)c2)cc1 ; c1ccc2cc3c(cc2c1)oc1ccc(-c2ccc(-c4c5ccccc5c(-c5cccc6ccccc56)c5ccccc45)cc2)cc13 ; c1cc(-c2ccc3oc4cc5ccccc5cc4c3c2)cc(-c2c3ccccc3c(-c3ccc4ccccc4c3)c3ccccc23)c1 ; c1ccc(-c2c3ccccc3c(-c3cccc(-c4ccc5oc6cc7ccccc7cc6c5c4)c3)c3ccccc23)cc1 ; c1ccc2cc3c(cc2c1)oc1ccc(-c2ccc(-c4c5ccccc5c(-c5ccc(-c6cccc7ccccc67)cc5)c5ccccc45)cc2)cc13]

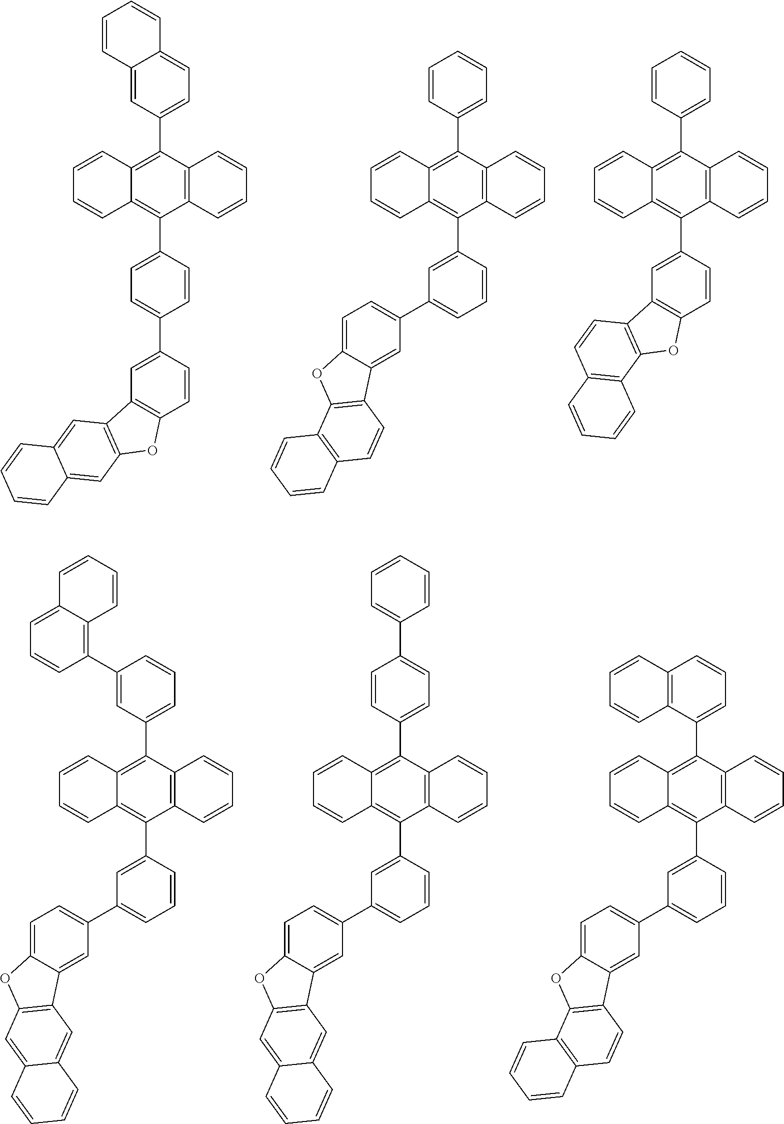 [SMILES: c1ccc2cc(-c3c4ccccc4c(-c4ccc(-c5ccc6oc7cc8ccccc8cc7c6c5)cc4)c4ccccc34)ccc2c1 ; c1ccc(-c2c3ccccc3c(-c3cccc(-c4ccc5oc6c7ccccc7ccc6c5c4)c3)c3ccccc23)cc1 ; c1ccc(-c2c3ccccc3c(-c3ccc4oc5c6ccccc6ccc5c4c3)c3ccccc23)cc1 ; c1cc(-c2ccc3oc4cc5ccccc5cc4c3c2)cc(-c2c3ccccc3c(-c3cccc(-c4cccc5ccccc45)c3)c3ccccc23)c1 ; c1ccc(-c2ccc(-c3c4ccccc4c(-c4cccc(-c5ccc6oc7cc8ccccc8cc7c6c5)c4)c4ccccc34)cc2)cc1 ; c1cc(-c2ccc3oc4c5ccccc5ccc4c3c2)cc(-c2c3ccccc3c(-c3cccc4ccccc34)c3ccccc23)c1]

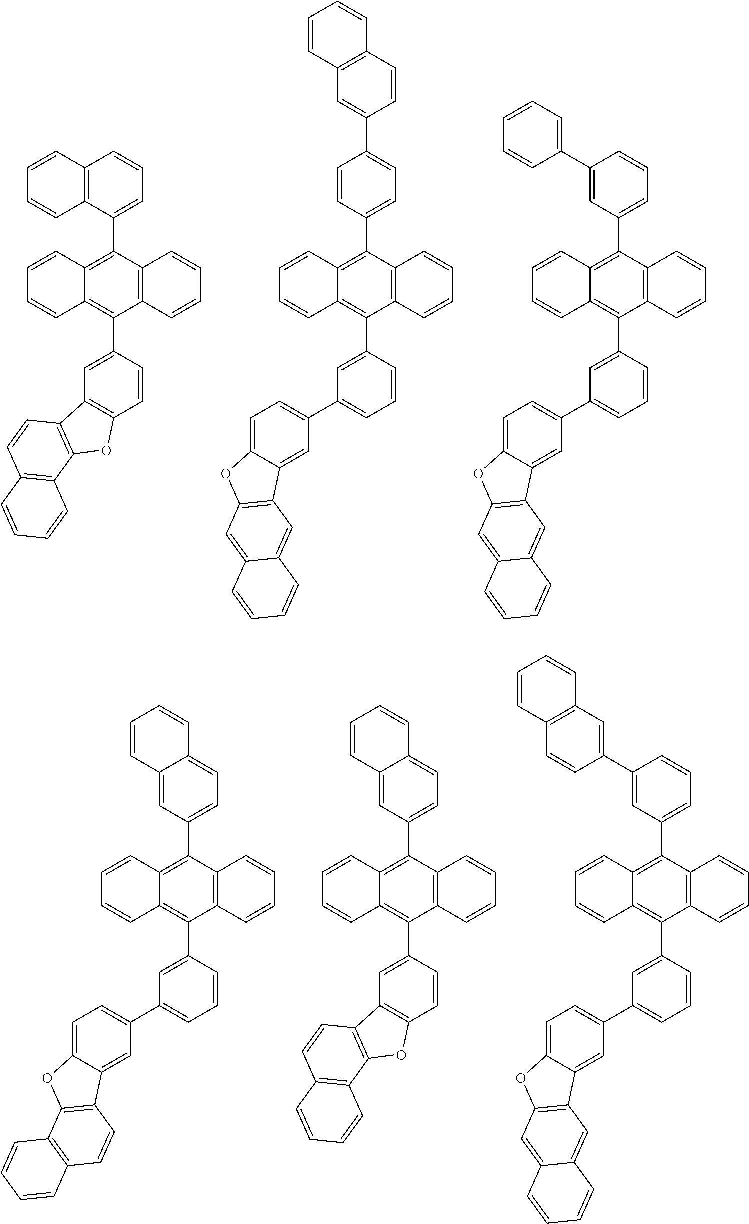

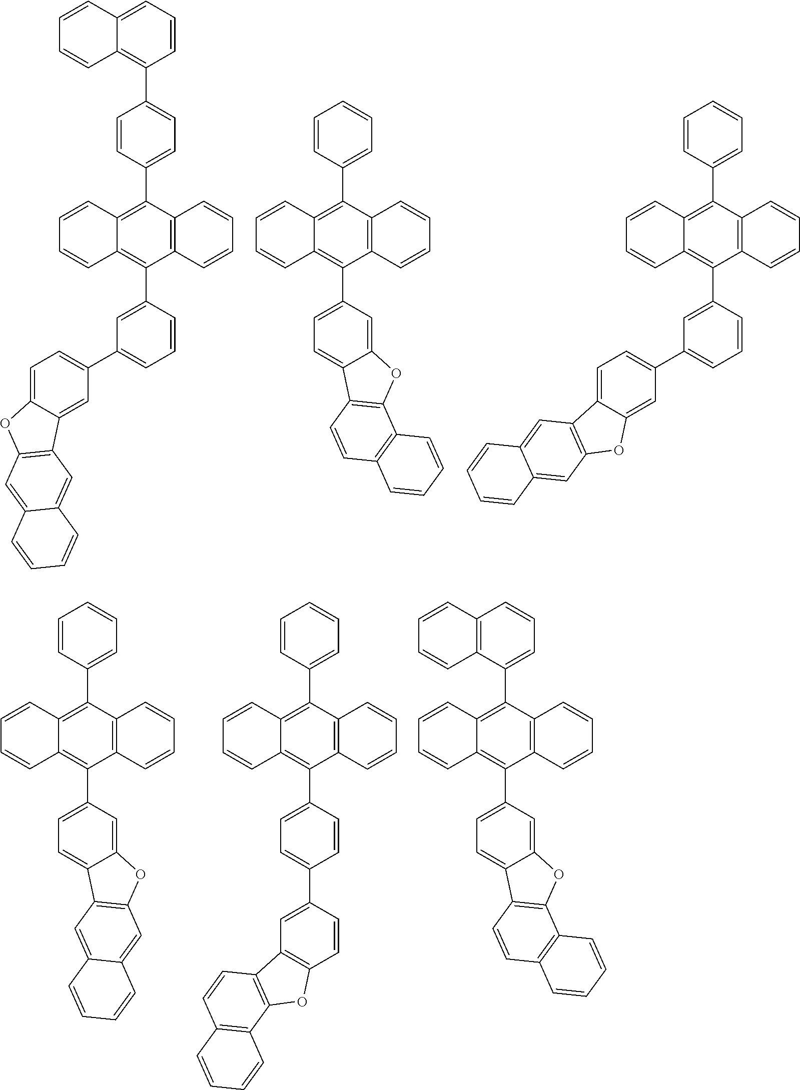

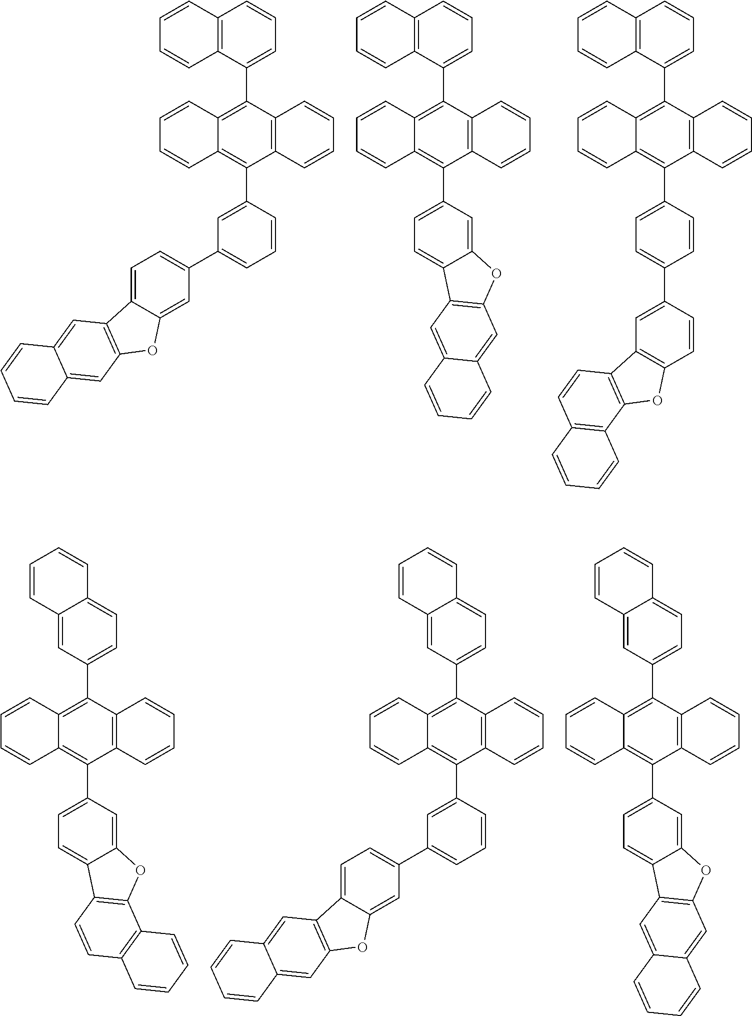

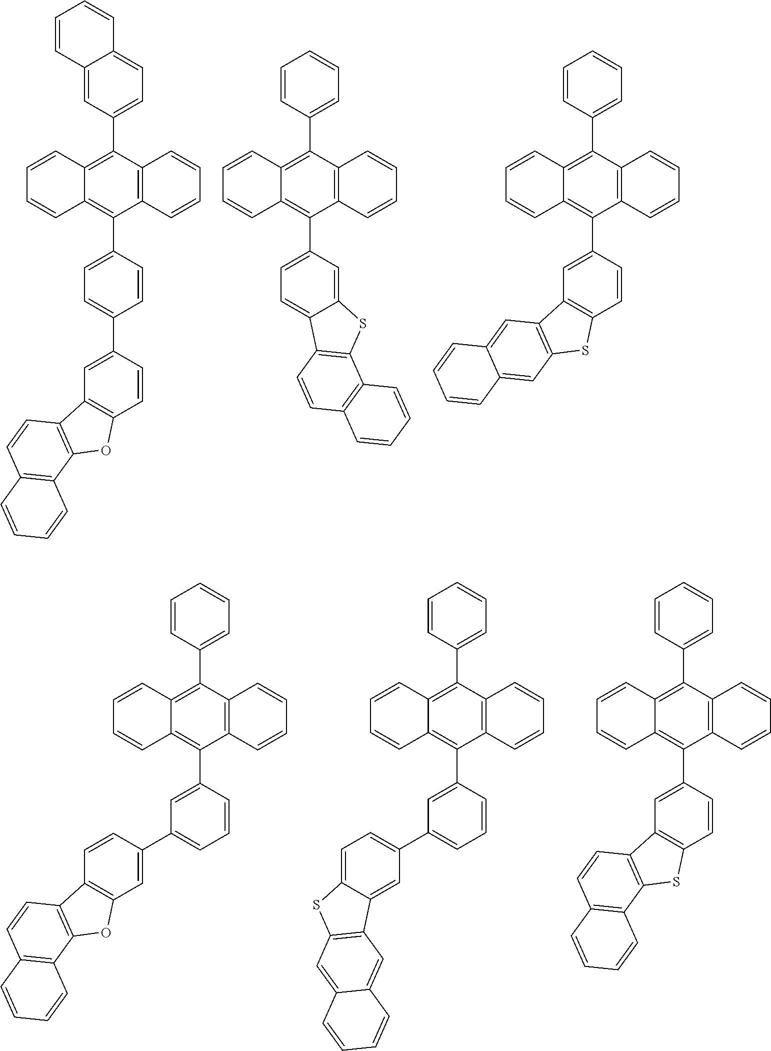

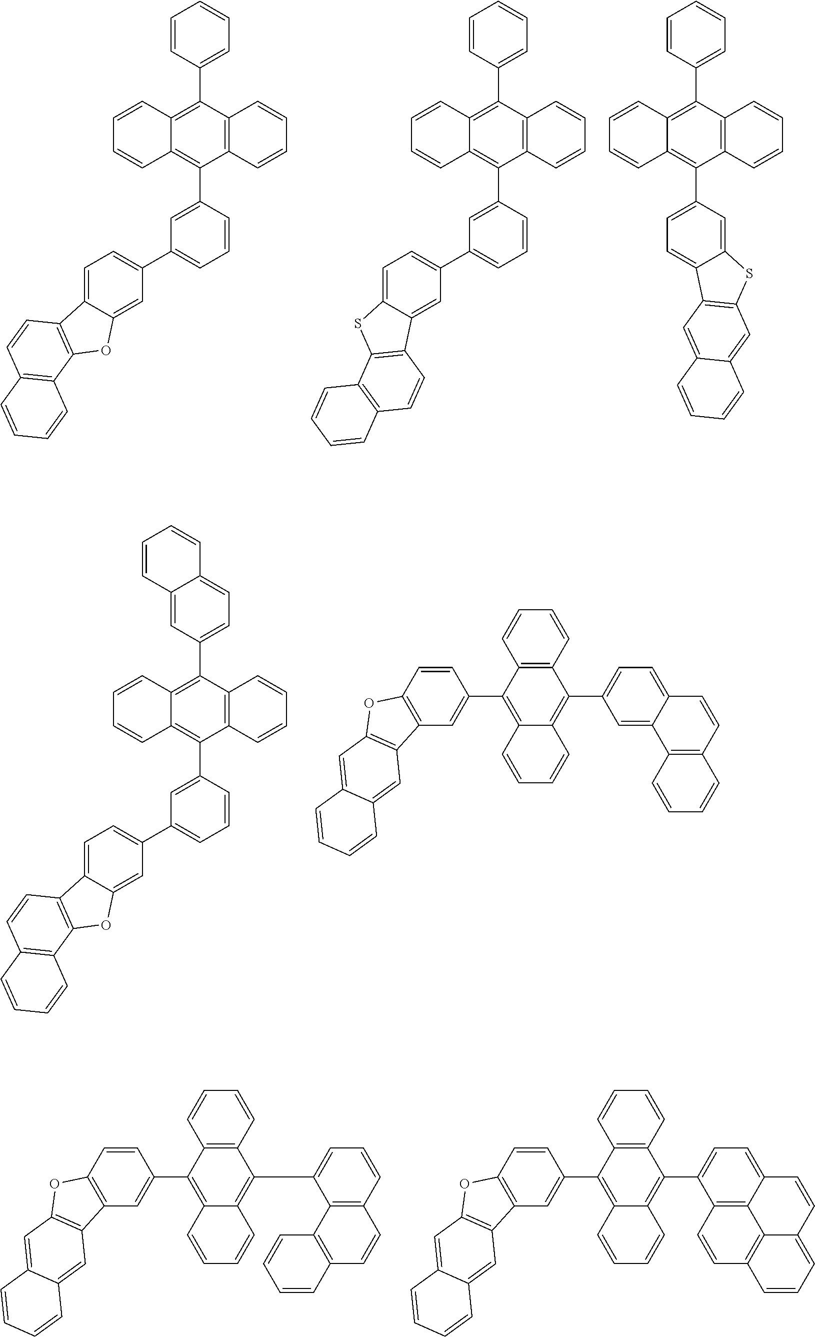

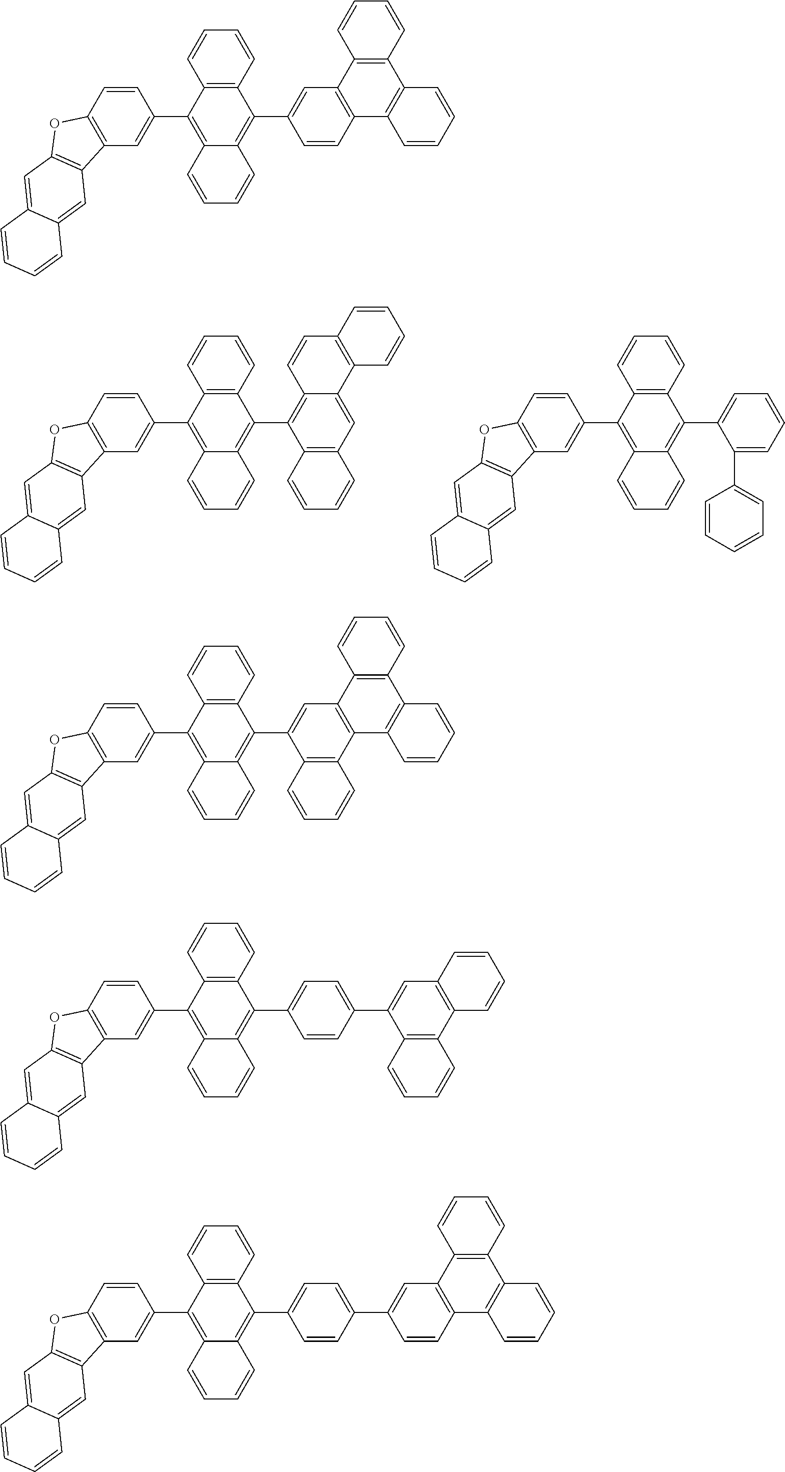 [SMILES: c1ccc2cc3c(cc2c1)oc1ccc(-c2c4ccccc4c(-c4ccc5c6ccccc6c6ccccc6c5c4)c4ccccc24)cc13 ; c1ccc2cc3c(cc2c1)oc1ccc(-c2c4ccccc4c(-c4c5ccccc5cc5c4ccc4ccccc45)c4ccccc24)cc13 ; c1ccc(-c2ccccc2-c2c3ccccc3c(-c3ccc4oc5cc6ccccc6cc5c4c3)c3ccccc23)cc1 ; c1ccc2cc3c(cc2c1)oc1ccc(-c2c4ccccc4c(-c4cc5c6ccccc6c6ccccc6c5c5ccccc45)c4ccccc24)cc13 ; c1ccc2cc3c(cc2c1)oc1ccc(-c2c4ccccc4c(-c4ccc(-c5cc6ccccc6c6ccccc56)cc4)c4ccccc24)cc13 ; c1ccc2cc3c(cc2c1)oc1ccc(-c2c4ccccc4c(-c4ccc(-c5ccc6c7ccccc7c7ccccc7c6c5)cc4)c4ccccc24)cc13]

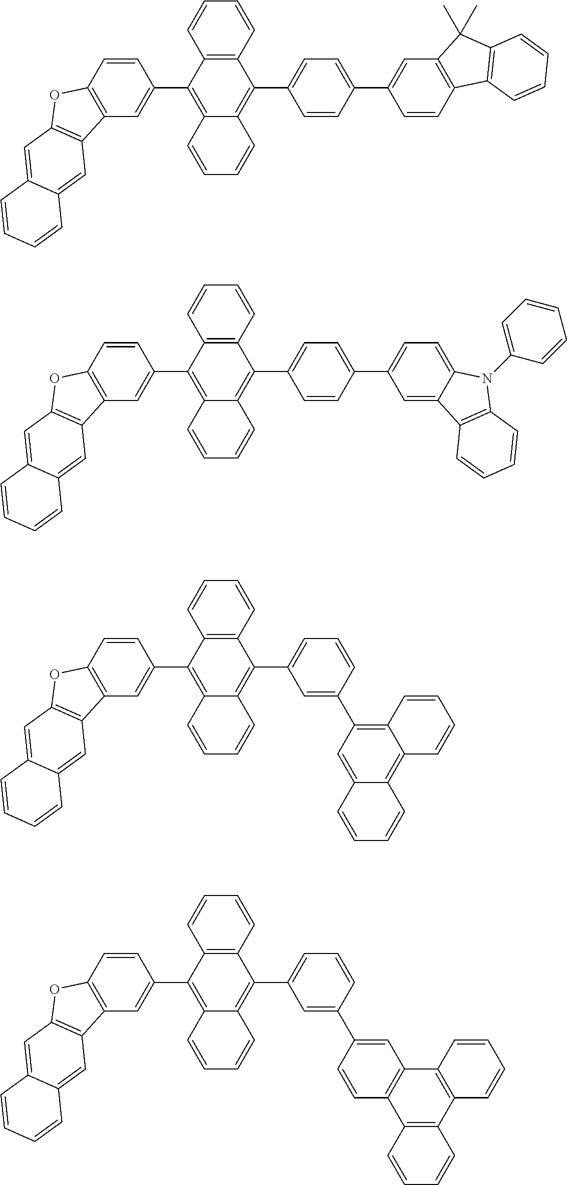 [SMILES: CC1(C)c2ccccc2-c2ccc(-c3ccc(-c4c5ccccc5c(-c5ccc6oc7cc8ccccc8cc7c6c5)c5ccccc45)cc3)cc21 ; c1ccc(-n2c3ccccc3c3cc(-c4ccc(-c5c6ccccc6c(-c6ccc7oc8cc9ccccc9cc8c7c6)c6ccccc56)cc4)ccc32)cc1 ; c1cc(-c2c3ccccc3c(-c3ccc4oc5cc6ccccc6cc5c4c3)c3ccccc23)cc(-c2cc3ccccc3c3ccccc23)c1 ; c1cc(-c2ccc3c4ccccc4c4ccccc4c3c2)cc(-c2c3ccccc3c(-c3ccc4oc5cc6ccccc6cc5c4c3)c3ccccc23)c1]

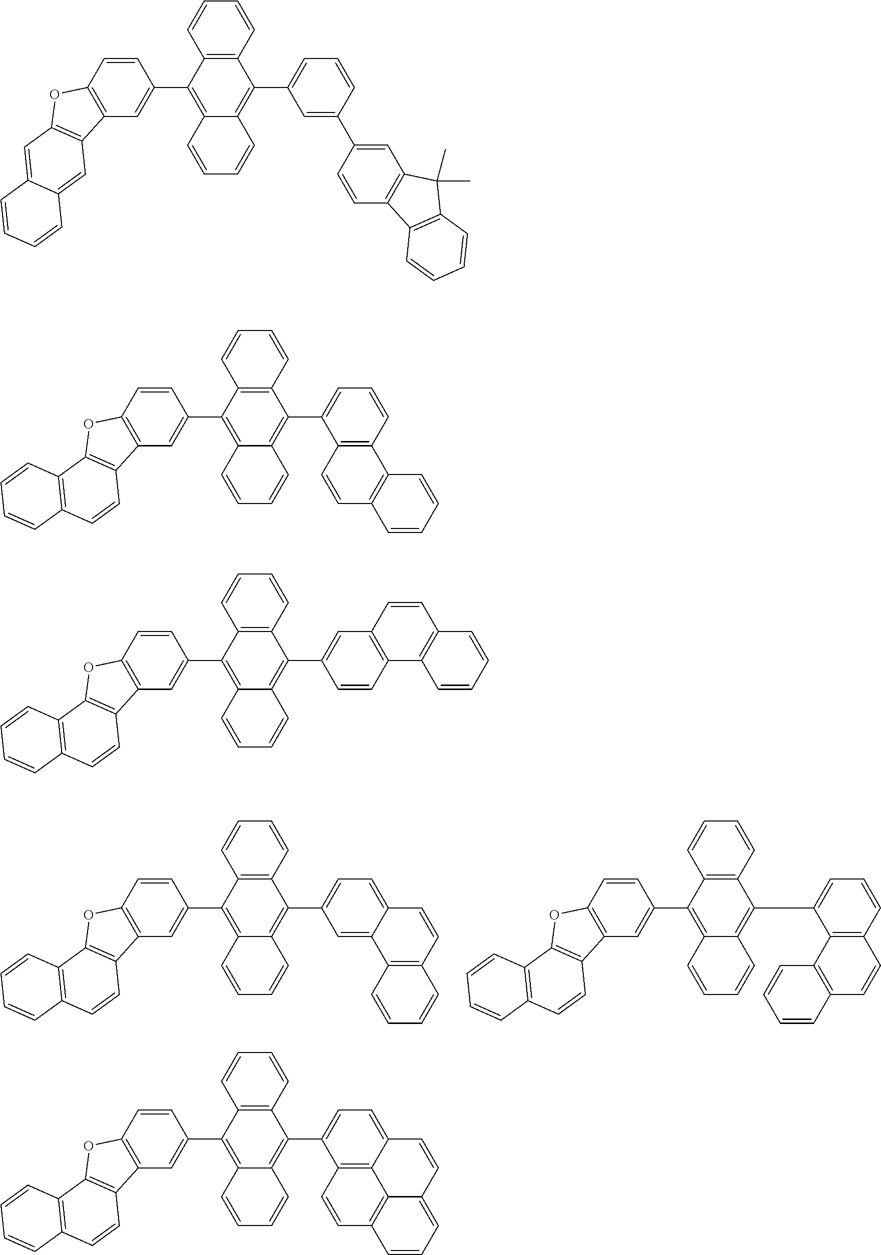

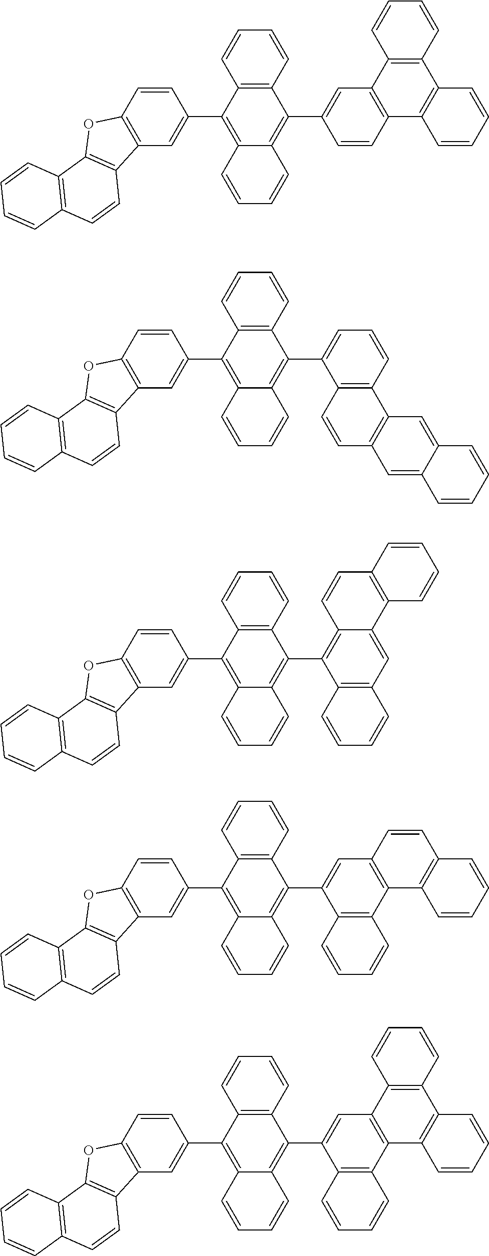

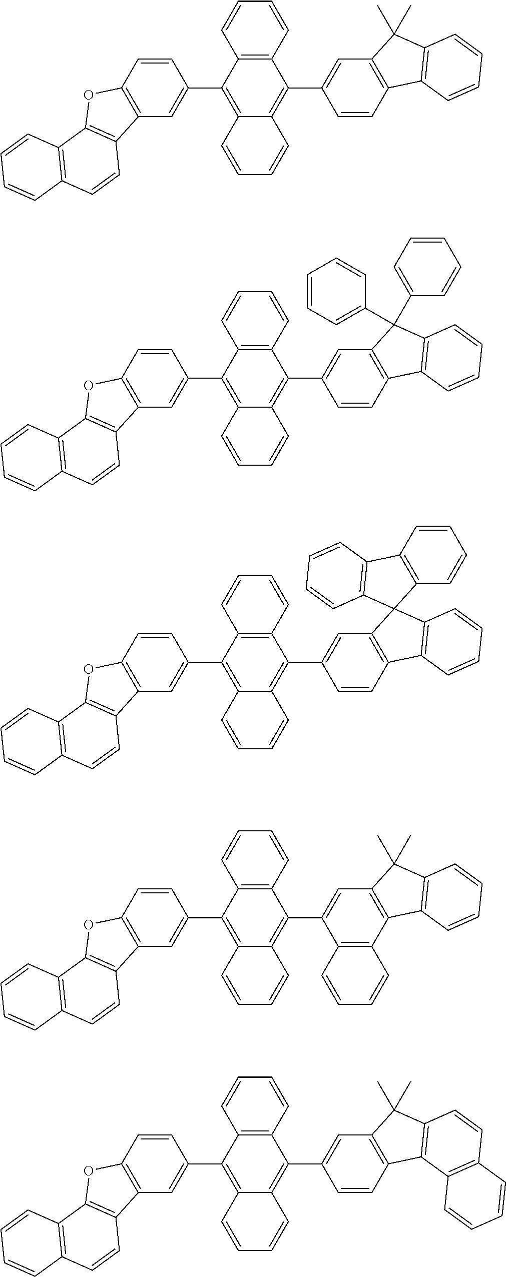 [SMILES: CC1(C)c2ccccc2-c2ccc(-c3c4ccccc4c(-c4ccc5oc6c7ccccc7ccc6c5c4)c4ccccc34)cc21 ; c1ccc(C2(c3ccccc3)c3ccccc3-c3ccc(-c4c5ccccc5c(-c5ccc6oc7c8ccccc8ccc7c6c5)c5ccccc45)cc32)cc1 ; c1ccc2c(c1)-c1ccccc1C21c2ccccc2-c2ccc(-c3c4ccccc4c(-c4ccc5oc6c7ccccc7ccc6c5c4)c4ccccc34)cc21 ; CC1(C)c2ccccc2-c2c1cc(-c1c3ccccc3c(-c3ccc4oc5c6ccccc6ccc5c4c3)c3ccccc13)c1ccccc21 ; CC1(C)c2cc(-c3c4ccccc4c(-c4ccc5oc6c7ccccc7ccc6c5c4)c4ccccc34)ccc2-c2c1ccc1ccccc21]

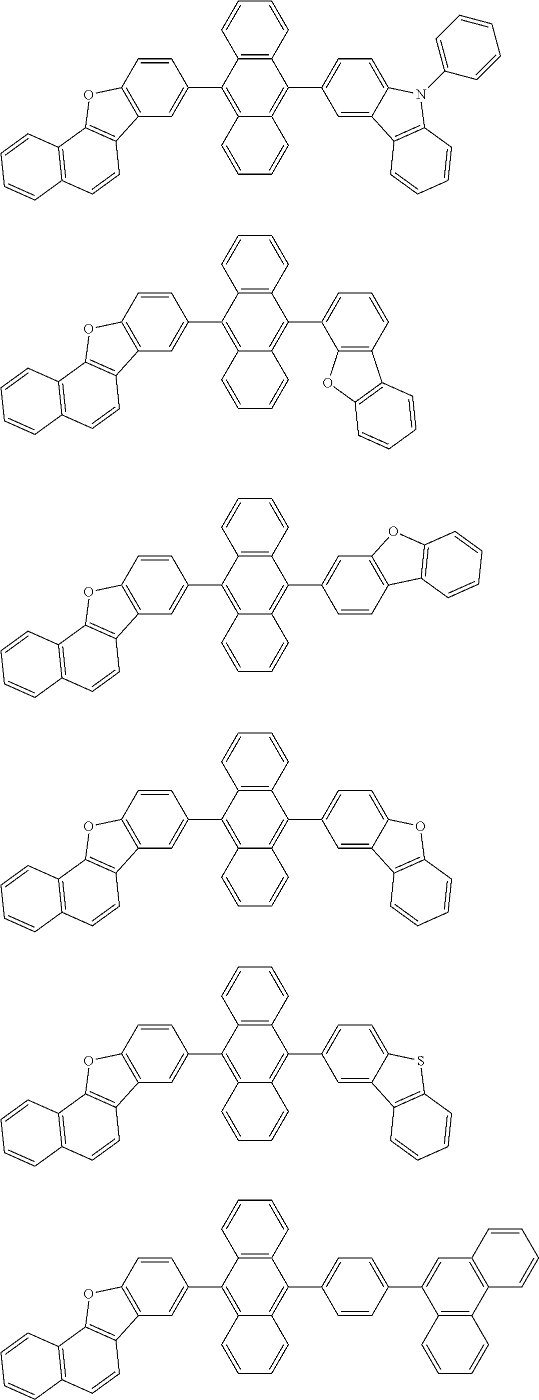 [SMILES: c1ccc(-n2c3ccccc3c3cc(-c4c5ccccc5c(-c5ccc6oc7c8ccccc8ccc7c6c5)c5ccccc45)ccc32)cc1 ; c1ccc2c(c1)ccc1c3cc(-c4c5ccccc5c(-c5cccc6c5oc5ccccc56)c5ccccc45)ccc3oc21 ; c1ccc2c(c1)ccc1c3cc(-c4c5ccccc5c(-c5ccc6c(c5)oc5ccccc56)c5ccccc45)ccc3oc21 ; c1ccc2c(c1)ccc1c3cc(-c4c5ccccc5c(-c5ccc6oc7ccccc7c6c5)c5ccccc45)ccc3oc21 ; c1ccc2c(c1)ccc1c3cc(-c4c5ccccc5c(-c5ccc6sc7ccccc7c6c5)c5ccccc45)ccc3oc21 ; c1ccc2c(c1)cc(-c1ccc(-c3c4ccccc4c(-c4ccc5oc6c7ccccc7ccc6c5c4)c4ccccc34)cc1)c1ccccc12]

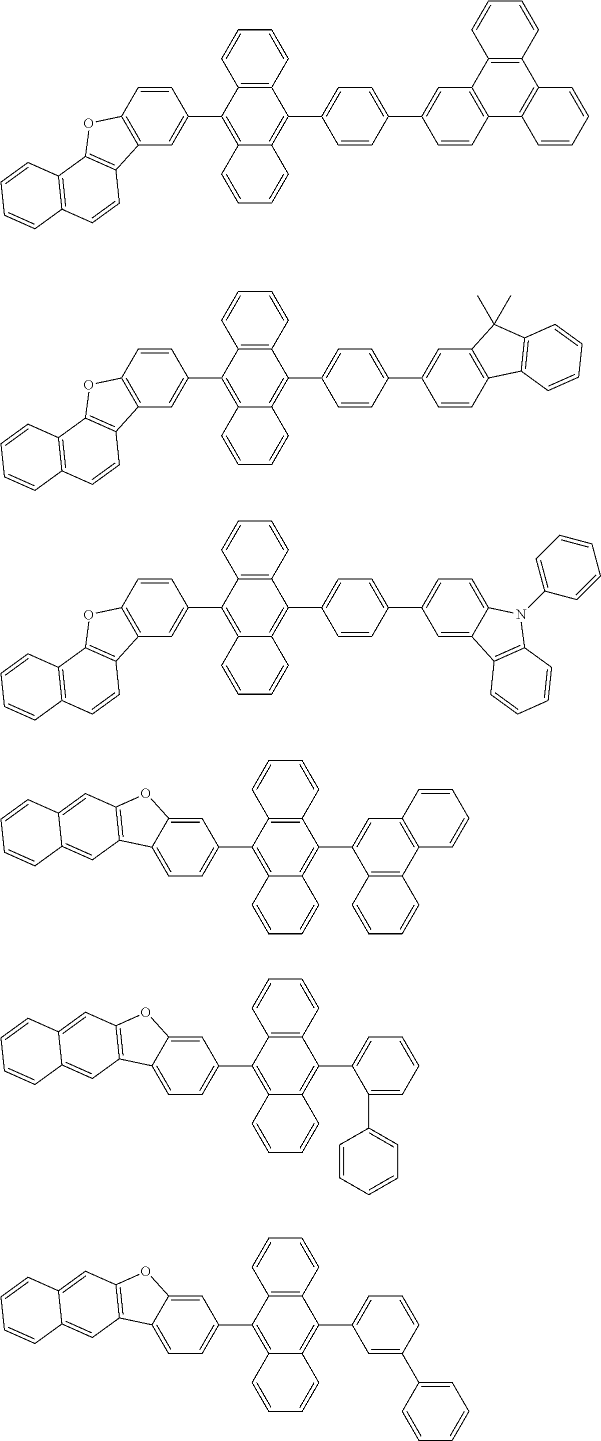

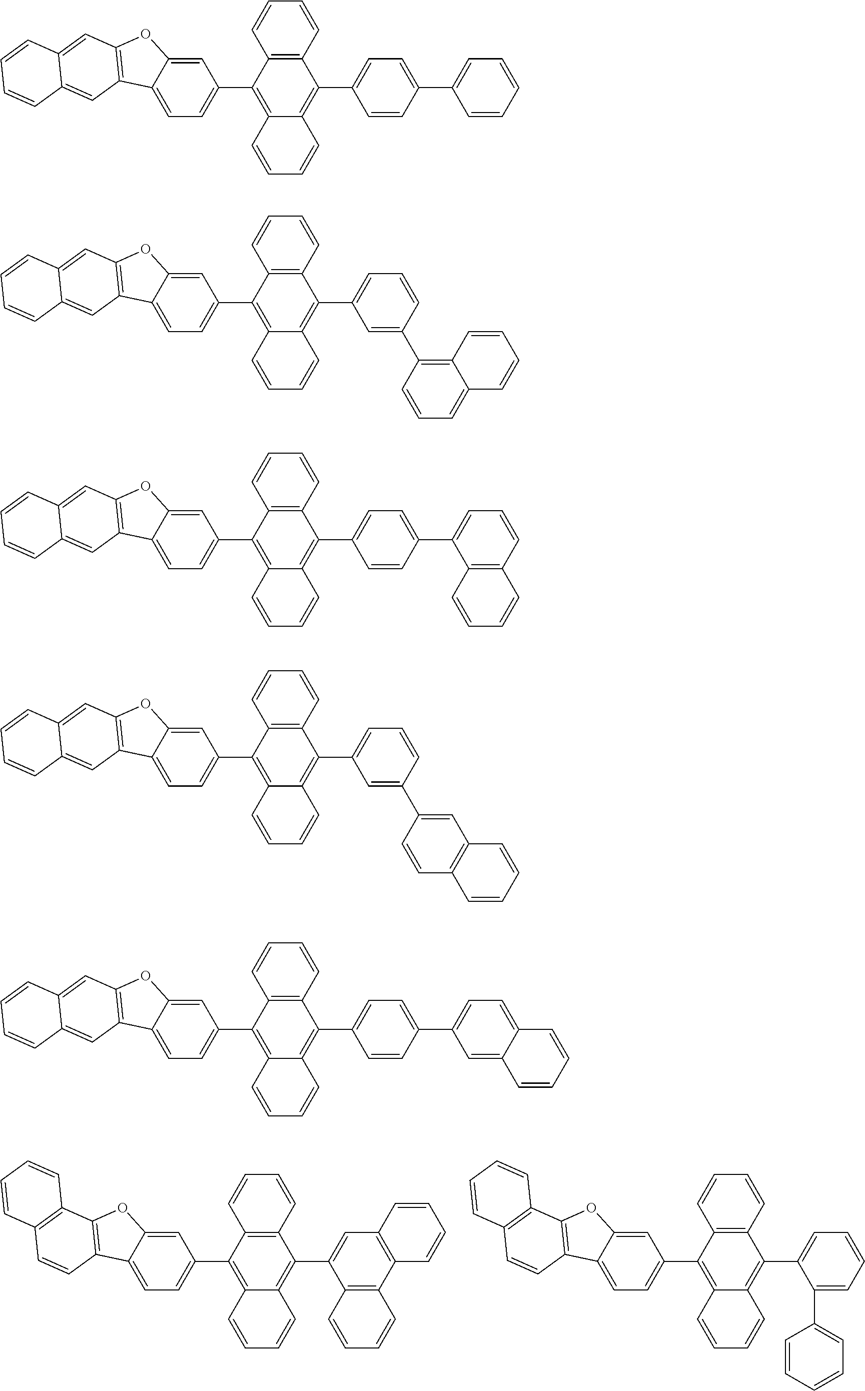

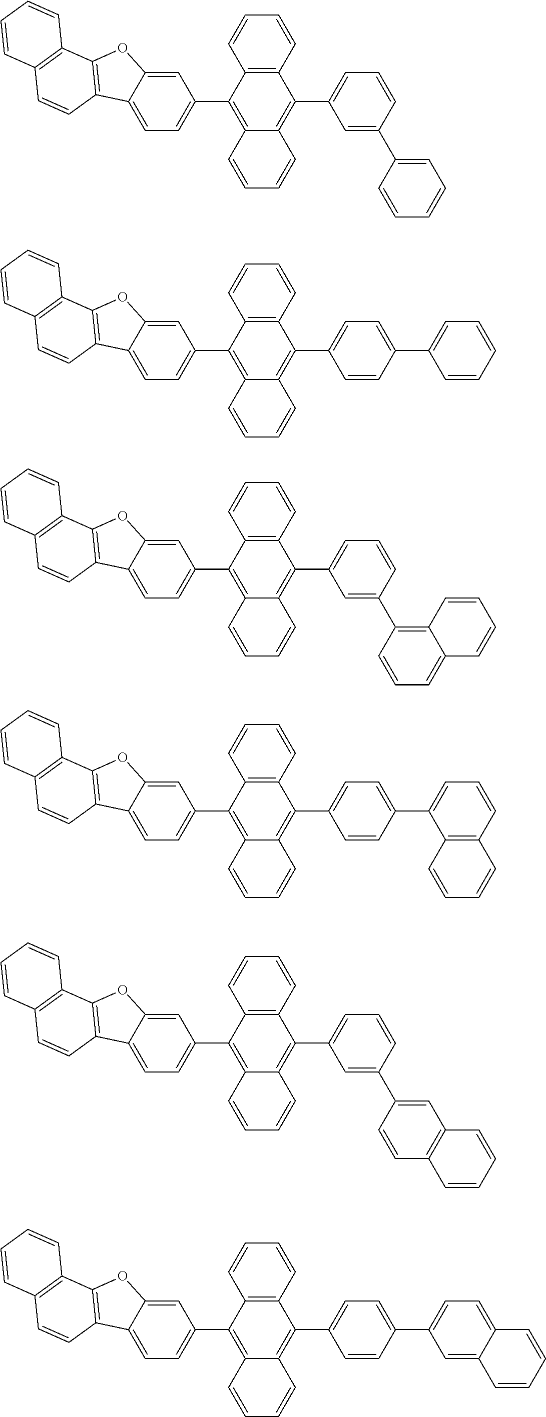

In the following compounds, the six-membered rings are all benzene rings.

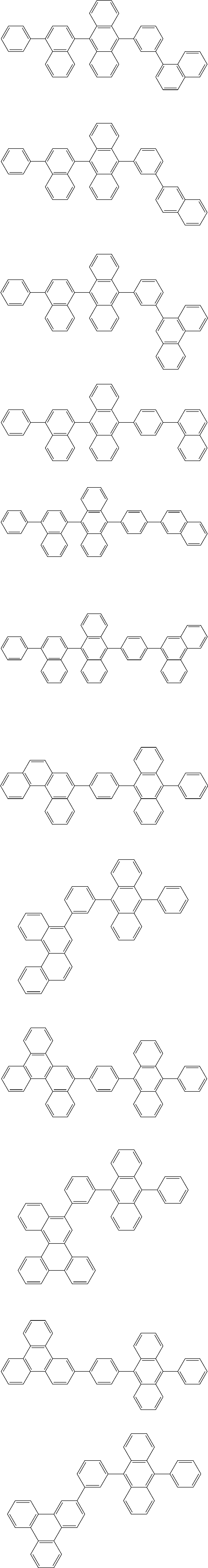

In the following compounds, the six-membered rings are all benzene rings.

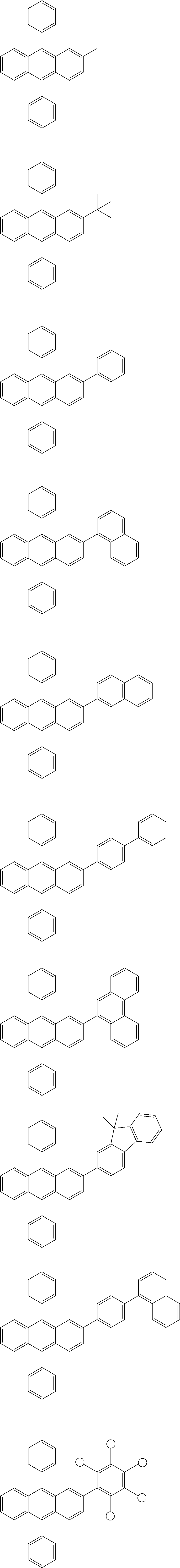

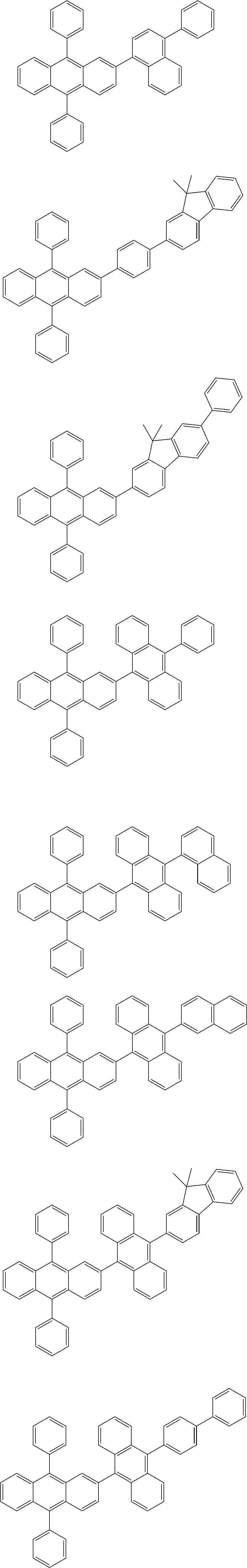

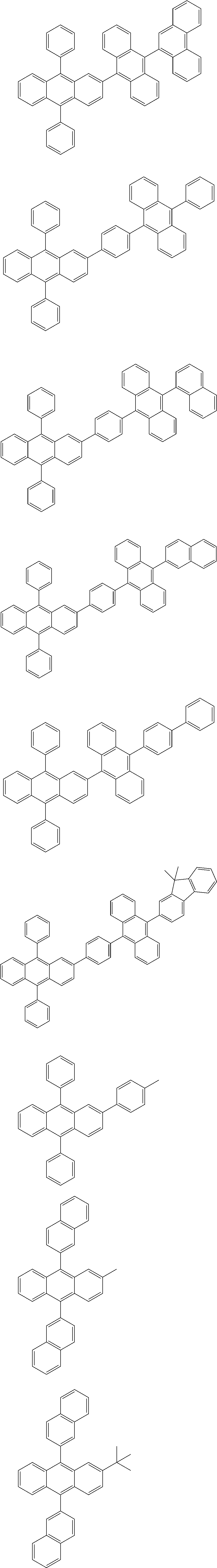

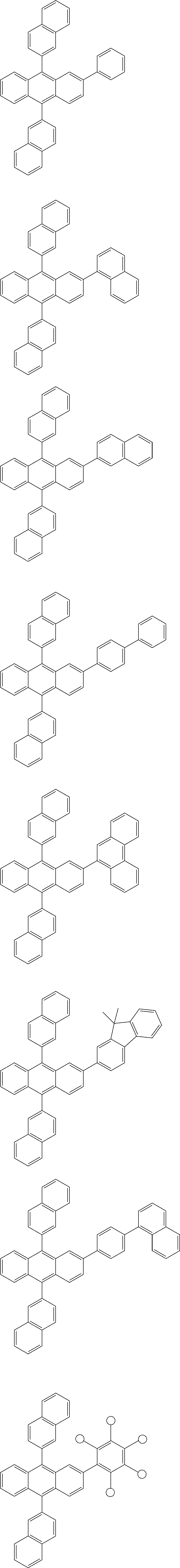

In the following compounds, the six-membered rings are all benzene rings.

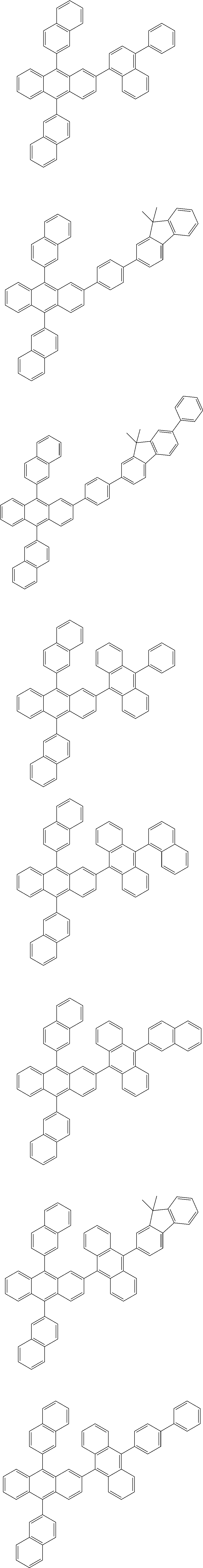

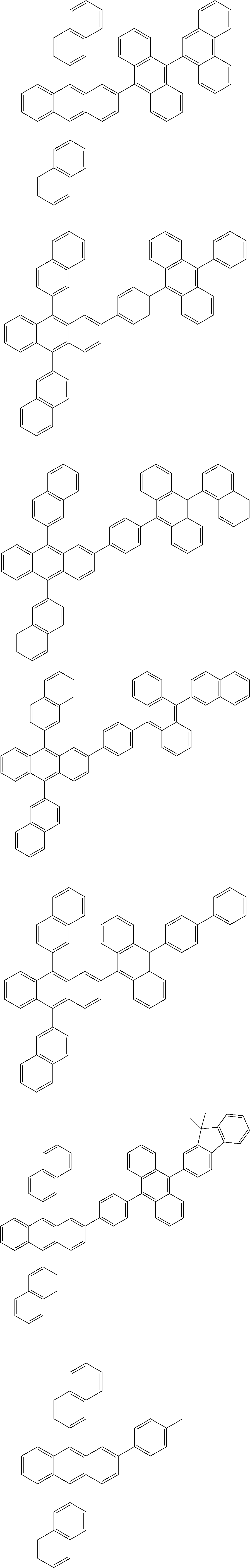

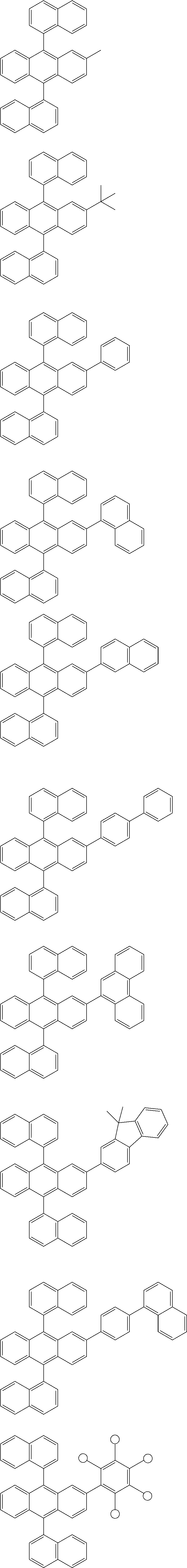

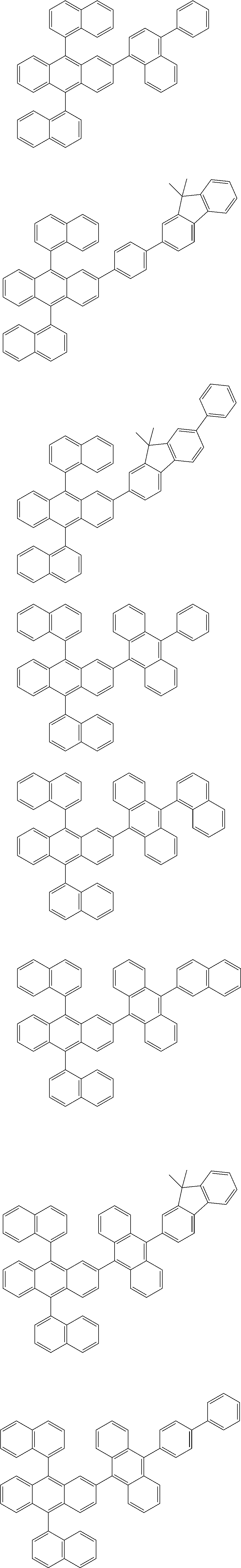

In the following compounds, the six-membered rings are all benzene rings.

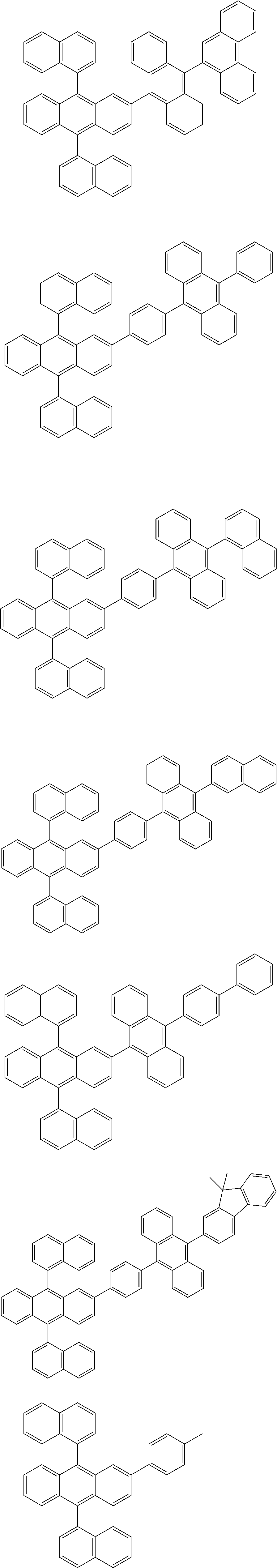

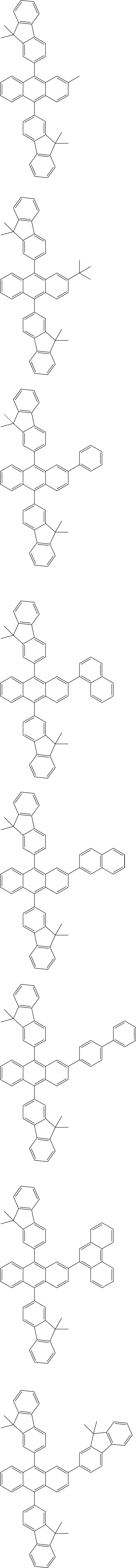

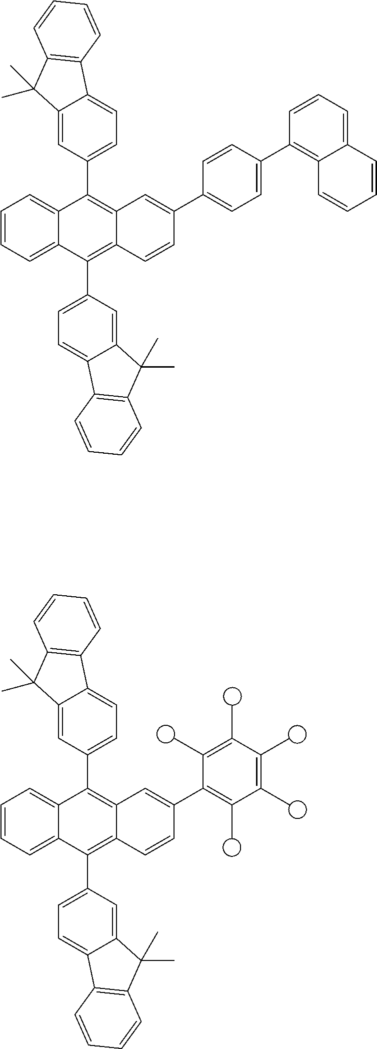

In the following compounds, the six-membered rings are all benzene rings.

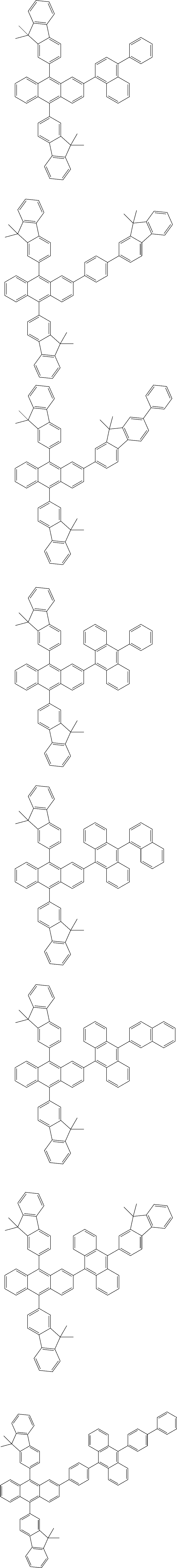

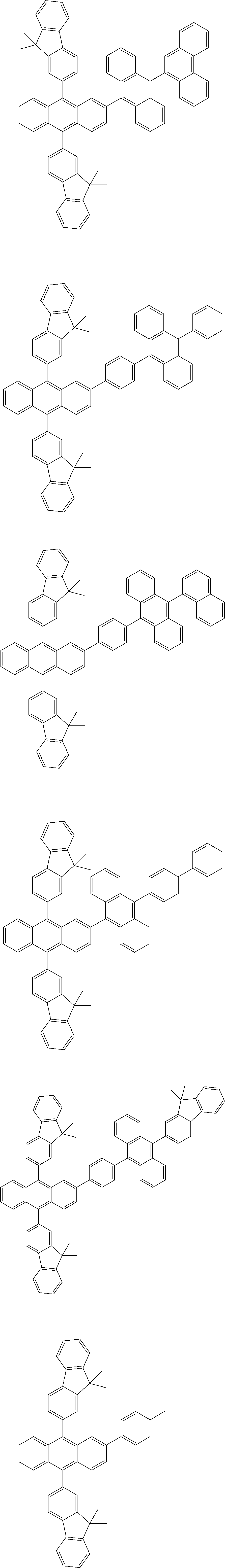

[chemical structure]

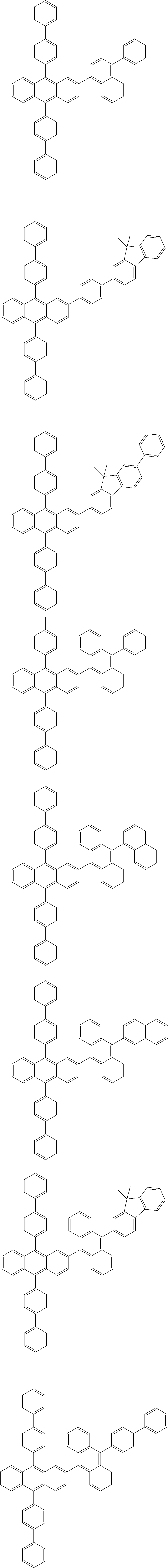

In the following compounds, the six-membered rings are all benzene rings.

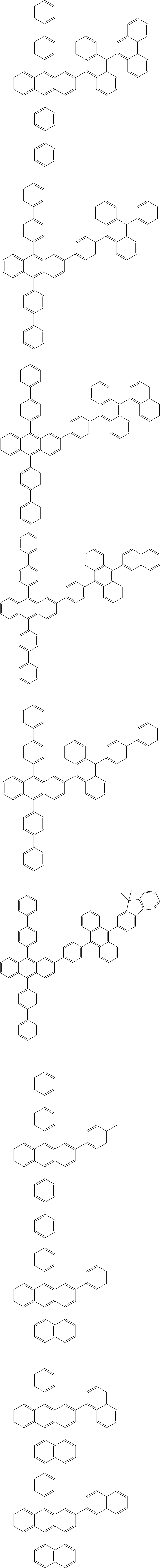

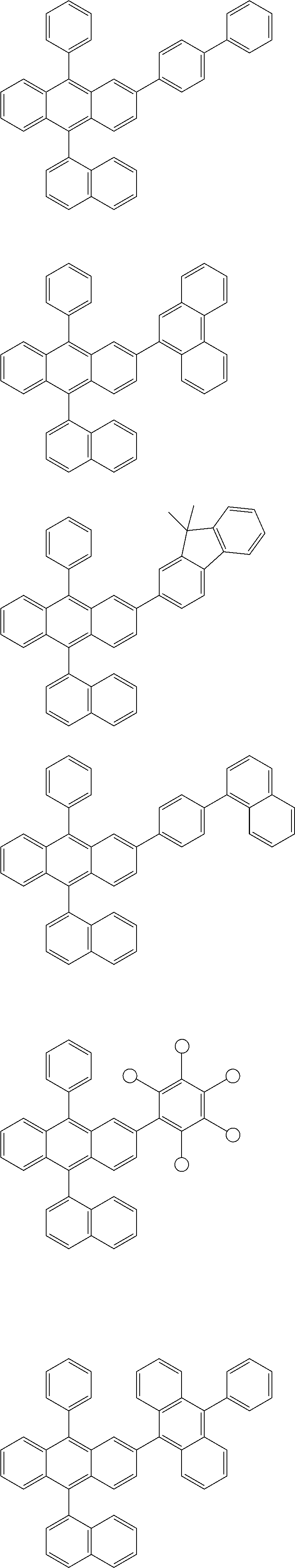

In the following compounds, the six-membered rings are all benzene rings.

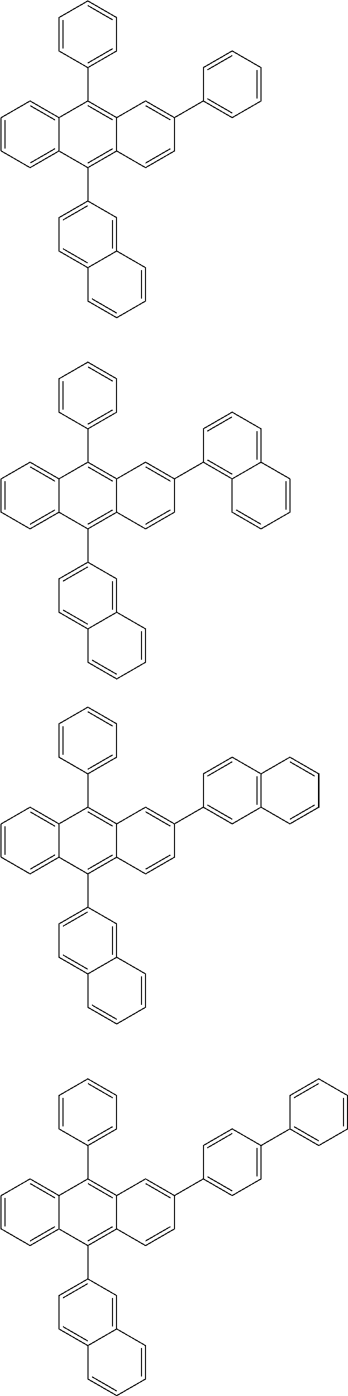

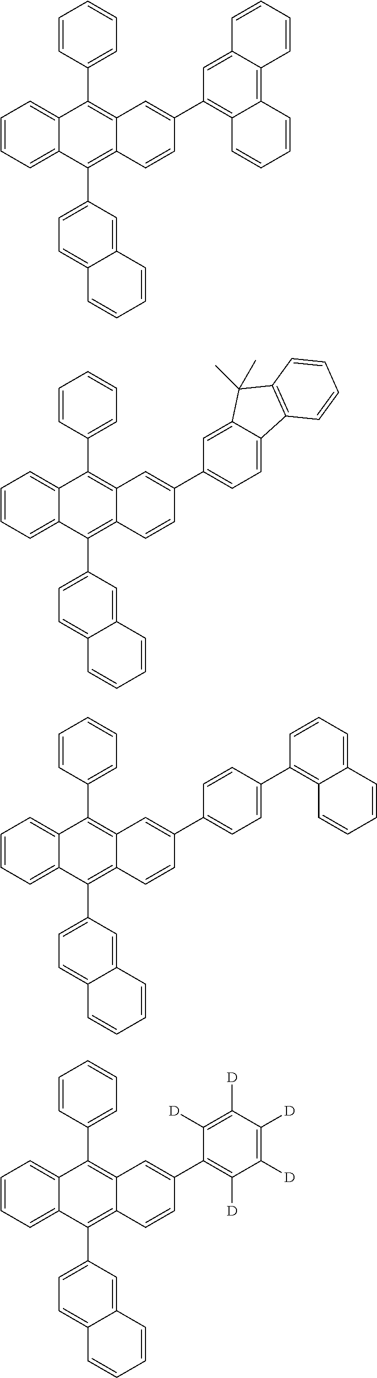

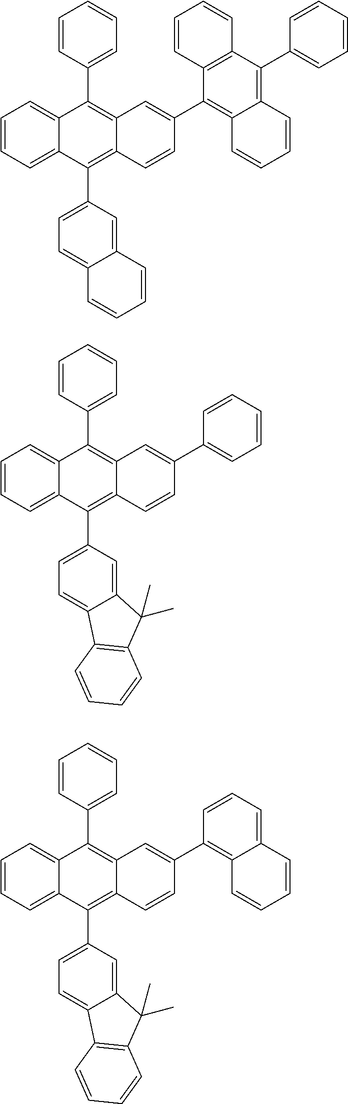

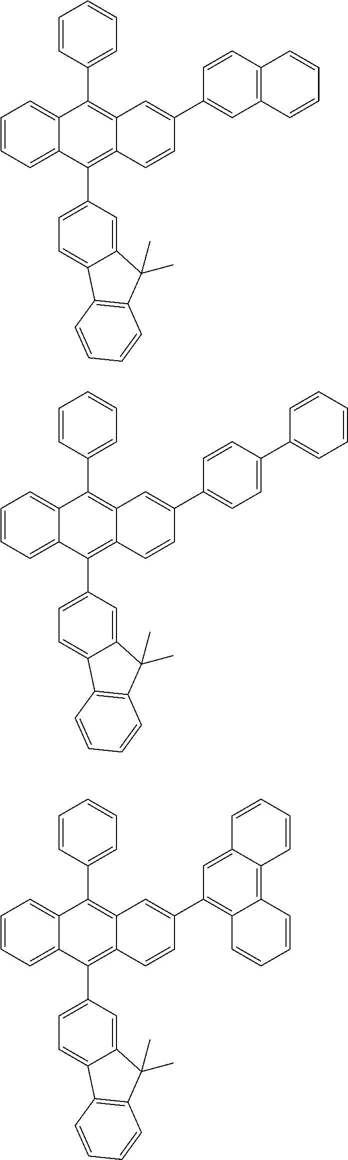

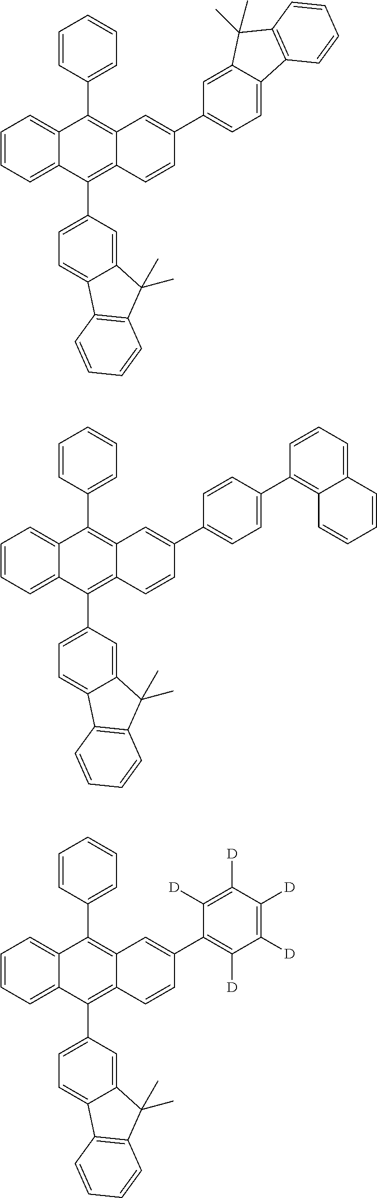

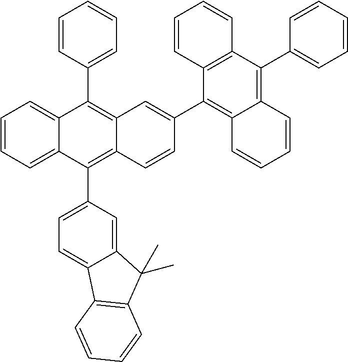 [SMILES: CC1(C)c2ccccc2-c2ccc(-c3c4ccccc4c(-c4ccccc4)c4cc(-c5c6ccccc6c(-c6ccccc6)c6ccccc56)ccc34)cc21]

In the following compounds, the six-membered rings are all benzene rings.

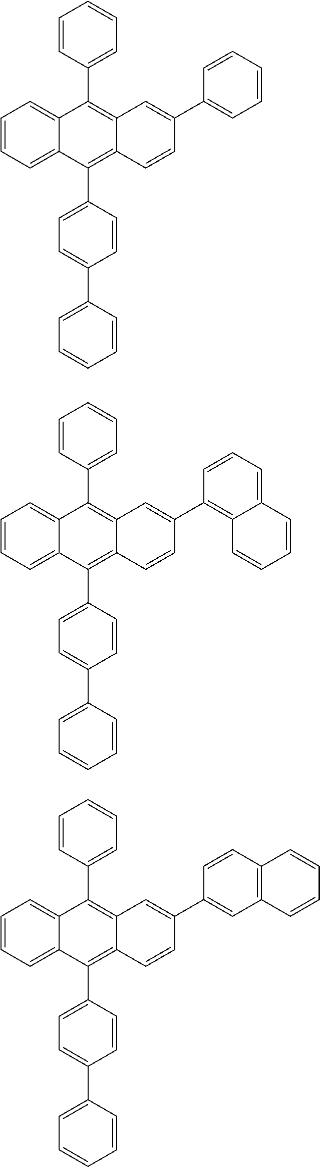

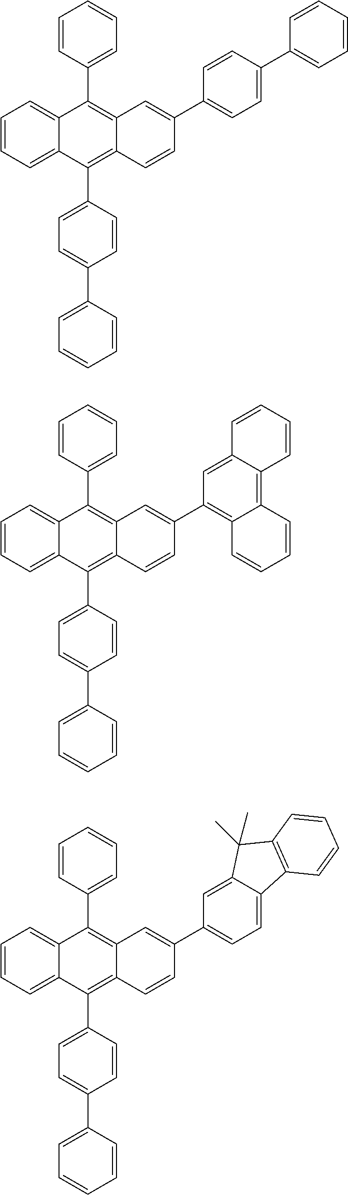

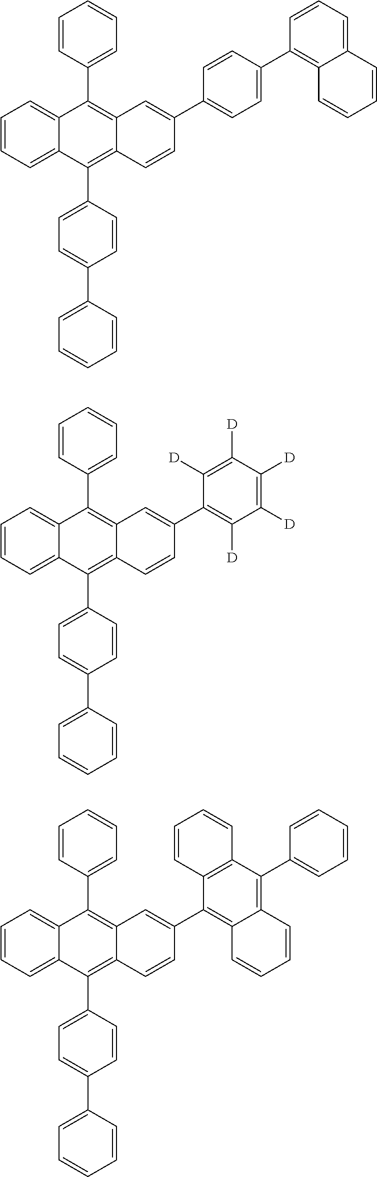

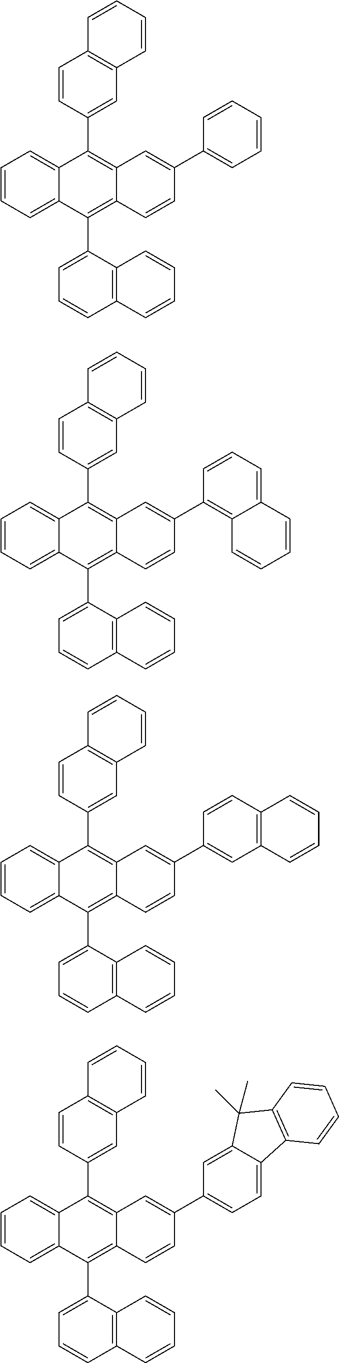

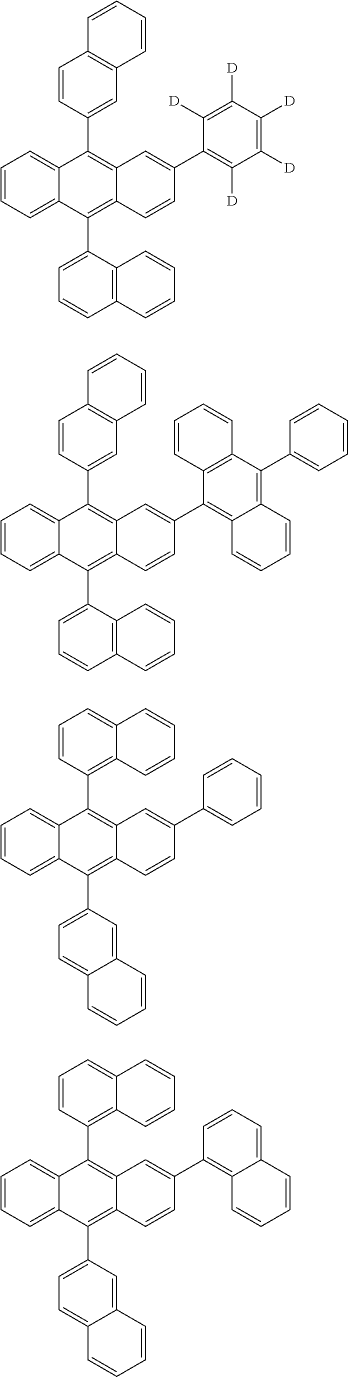

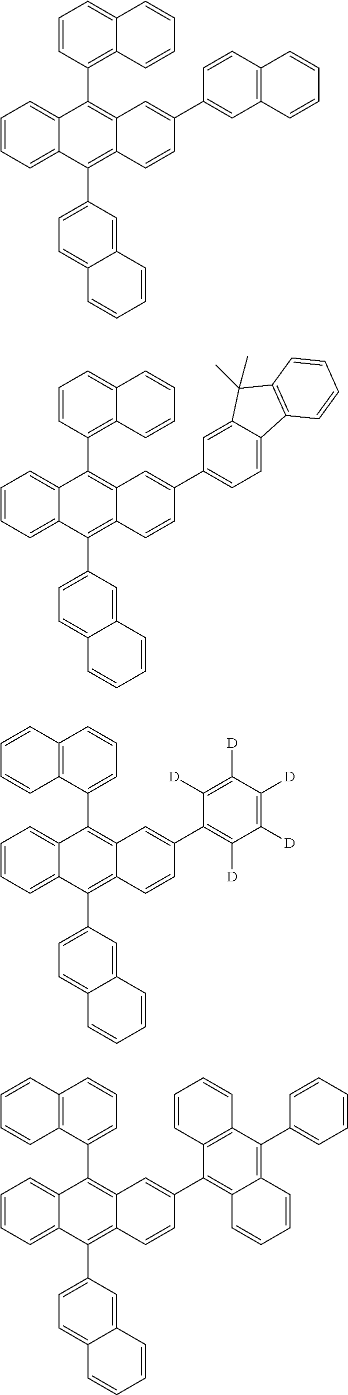

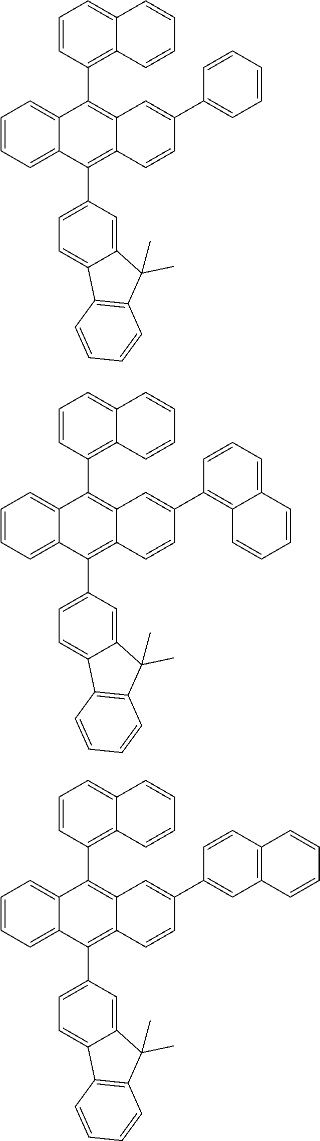

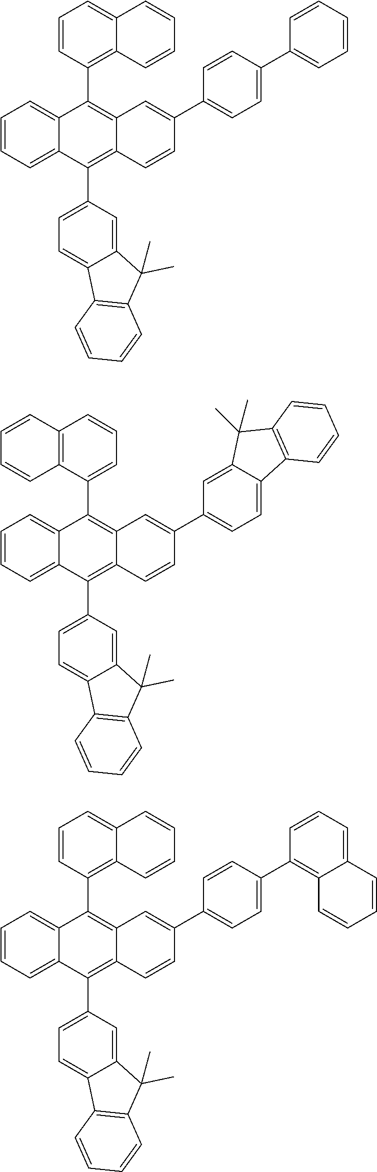

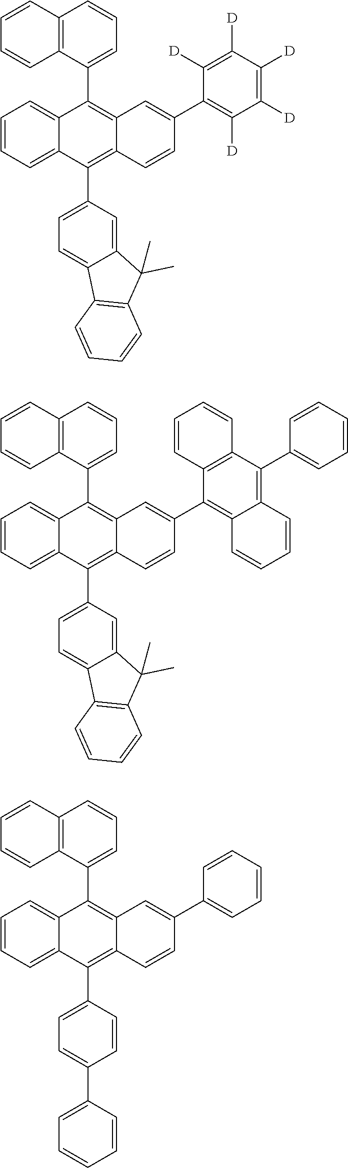

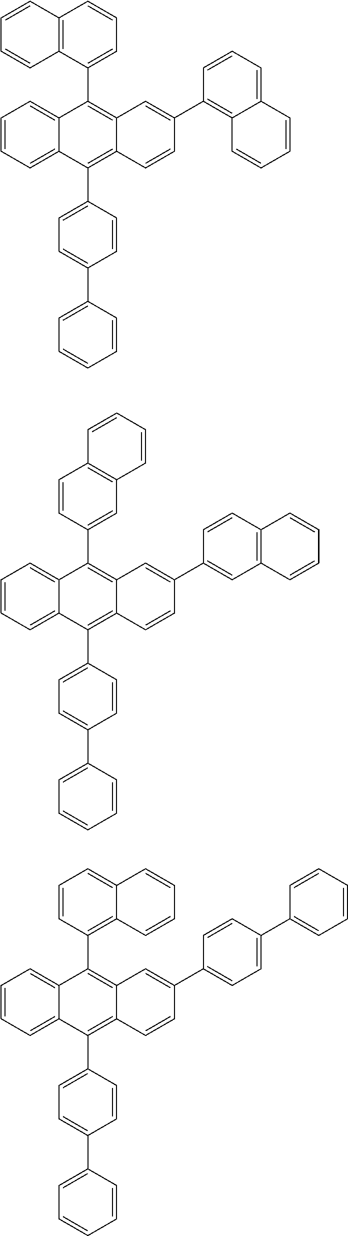

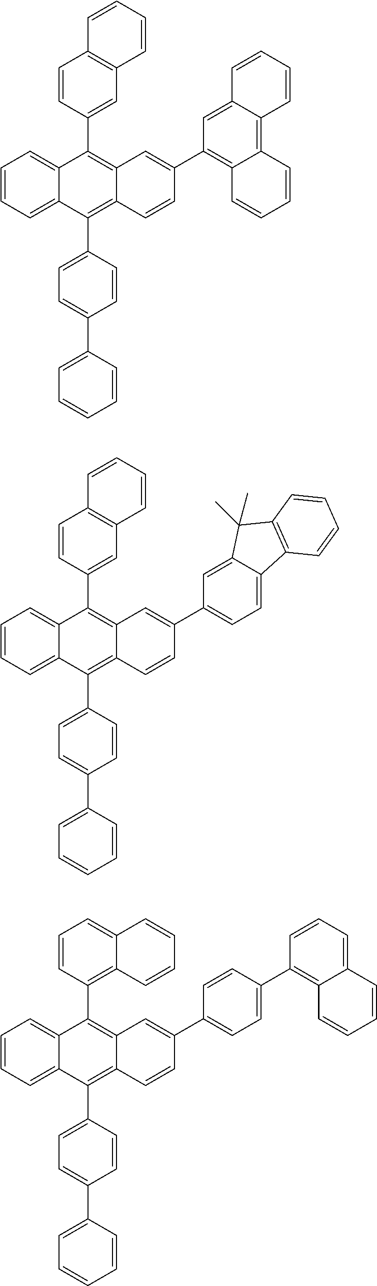

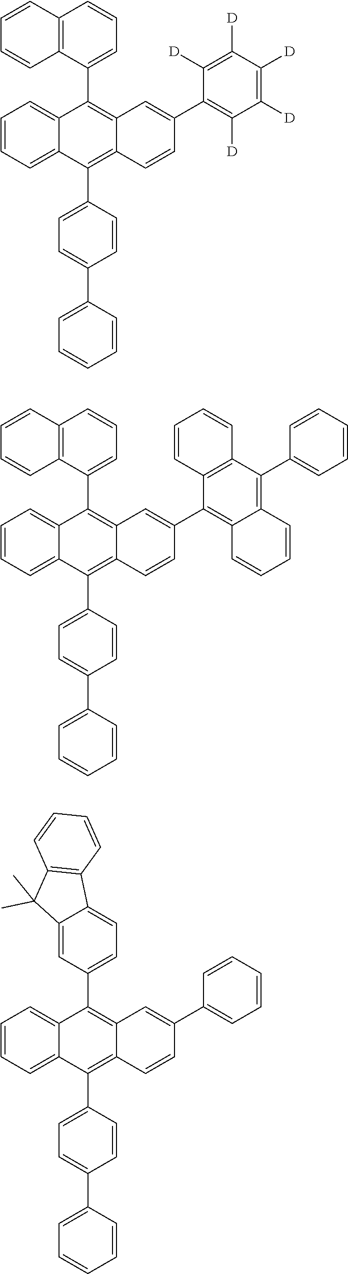

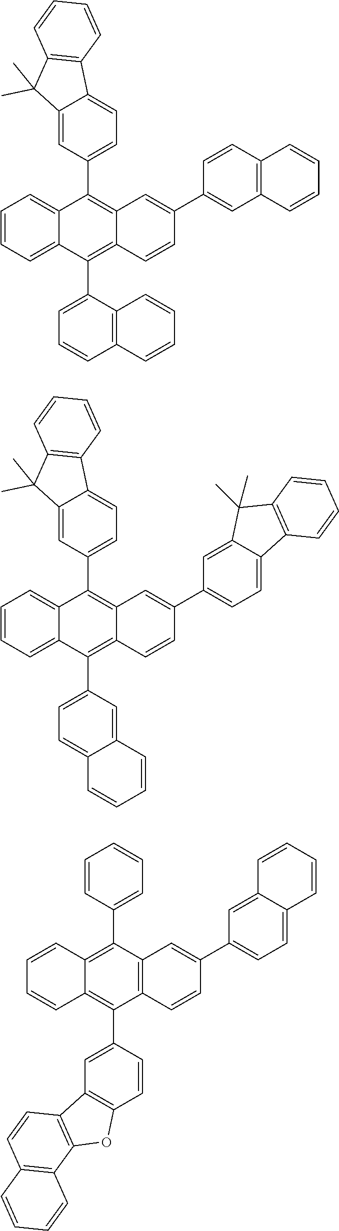

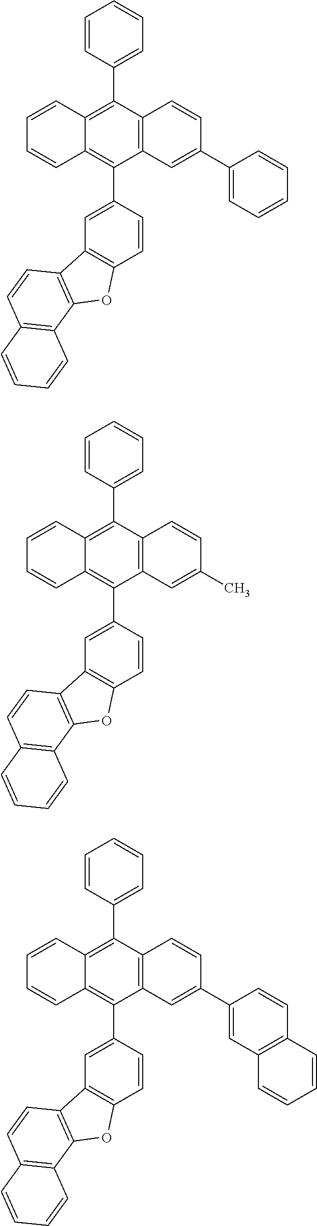 [SMILES: c1ccc(-c2ccc3c(-c4ccccc4)c4ccccc4c(-c4ccc5oc6c7ccccc7ccc6c5c4)c3c2)cc1 ; Cc1ccc2c(-c3ccccc3)c3ccccc3c(-c3ccc4oc5c6ccccc6ccc5c4c3)c2c1 ; c1ccc(-c2c3ccccc3c(-c3ccc4oc5c6ccccc6ccc5c4c3)c3cc(-c4ccc5ccccc5c4)ccc23)cc1]

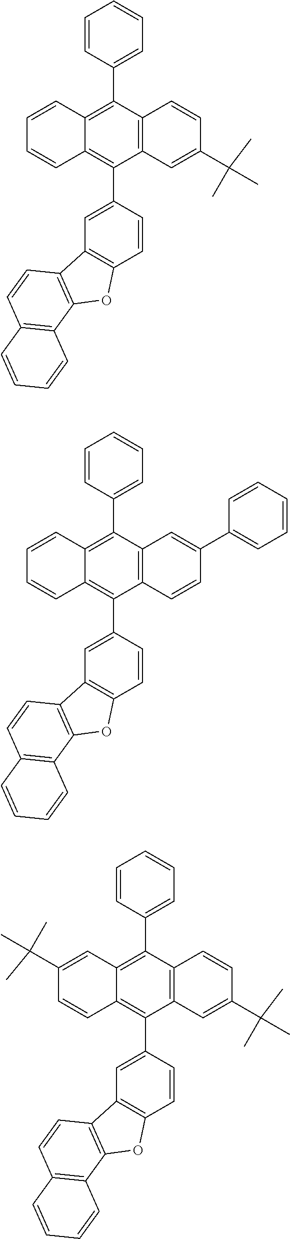

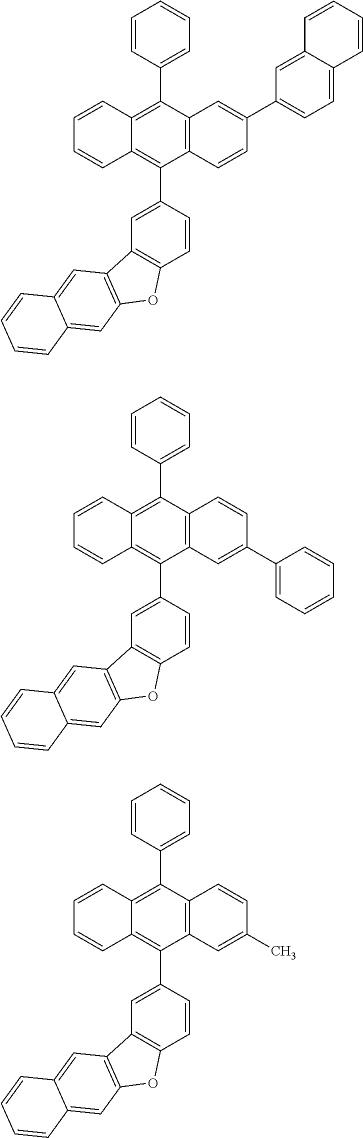 [SMILES: c1ccc(-c2c3ccccc3c(-c3ccc4oc5cc6ccccc6cc5c4c3)c3ccc(-c4ccc5ccccc5c4)cc23)cc1 ; c1ccc(-c2ccc3c(-c4ccccc4)c4ccccc4c(-c4ccc5oc6cc7ccccc7cc6c5c4)c3c2)cc1 ; Cc1ccc2c(-c3ccccc3)c3ccccc3c(-c3ccc4oc5cc6ccccc6cc5c4c3)c2c1]

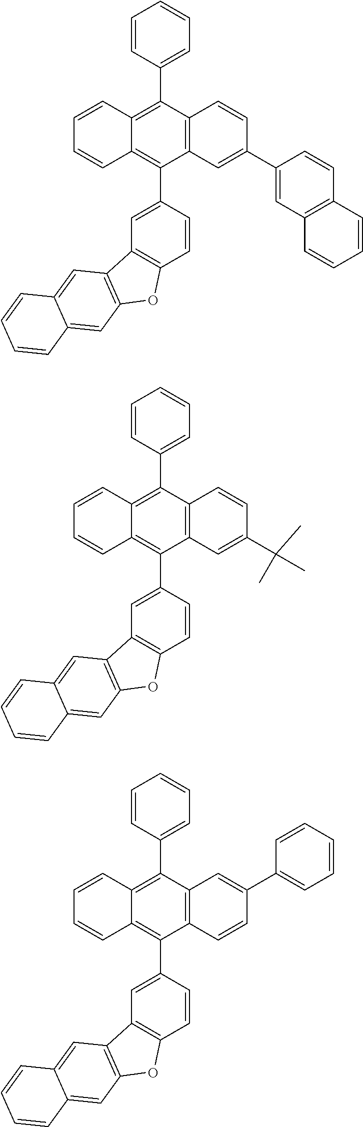 [SMILES: c1ccc(-c2c3ccccc3c(-c3ccc4oc5cc6ccccc6cc5c4c3)c3cc(-c4ccc5ccccc5c4)ccc23)cc1 ; CC(C)(C)c1ccc2c(-c3ccccc3)c3ccccc3c(-c3ccc4oc5cc6ccccc6cc5c4c3)c2c1 ; c1ccc(-c2ccc3c(-c4ccc5oc6cc7ccccc7cc6c5c4)c4ccccc4c(-c4ccccc4)c3c2)cc1]

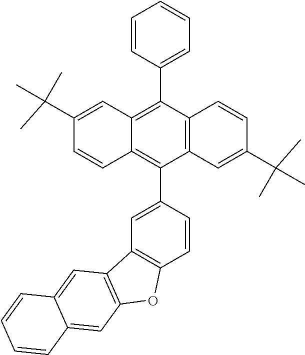 [SMILES: CC(C)(C)c1ccc2c(-c3ccc4oc5cc6ccccc6cc5c4c3)c3cc(C(C)(C)C)ccc3c(-c3ccccc3)c2c1]

A chrysene skeletal-containing compound represented by formula (21) is usable as the first compound:

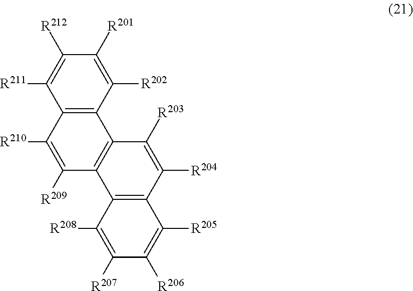

wherein:

R²⁰¹ to R²¹² are each independently a hydrogen atom, a substituent, or -L²-Ar²¹, provided that at least one of R²⁰¹ to R²¹² is -L²-Ar²¹;

the details of the substituent are as described above with respect to the substituent of R_{A}, R_{B}, and R_{C} of formula (D1),

the details of L² are Ar²¹ are as described above with respect to L and Ar of formula (19), respectively; and

one or both of R²⁰⁴ and R²¹⁰ are preferably -L²-Ar²¹.

Examples of the chrysene derivative represented by formula (21) are shown below, although not particularly limited thereto.

In the following compounds, the six-membered rings are all benzene rings.

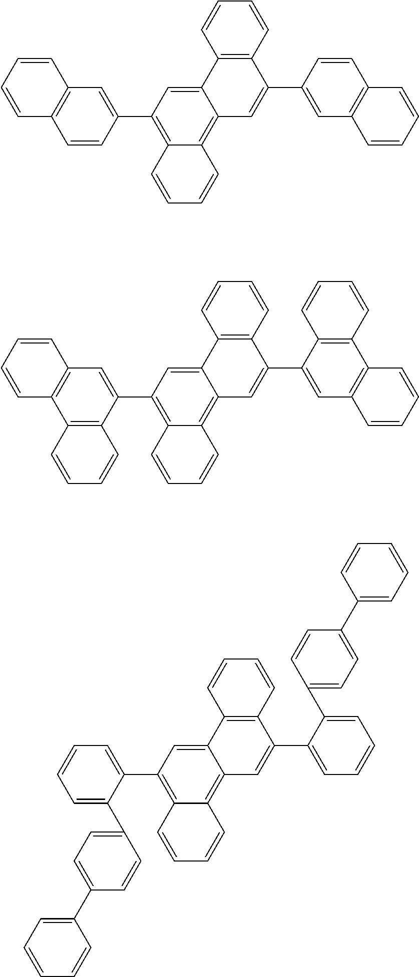

In the following compounds, the six-membered rings are all benzene rings.

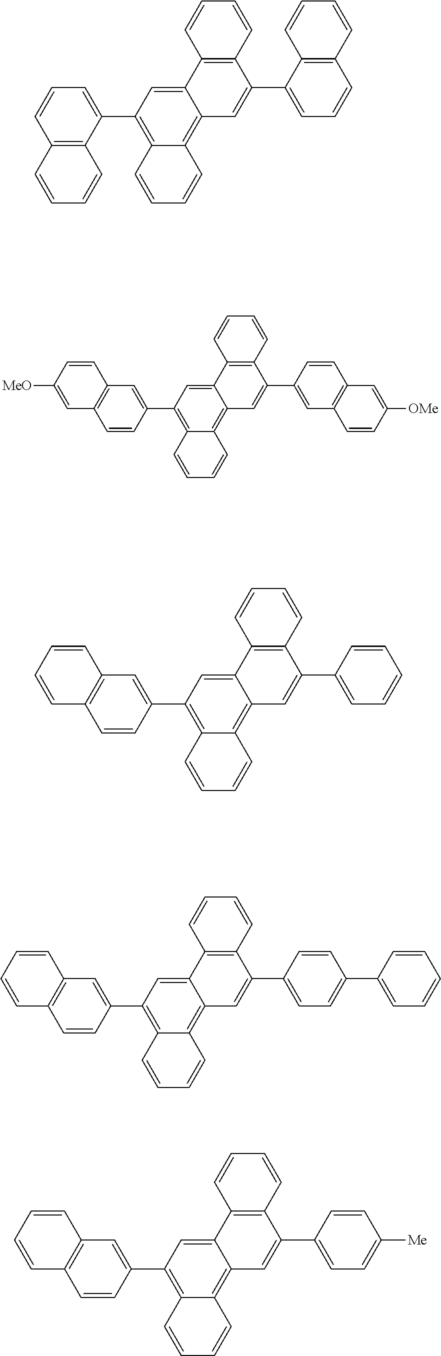

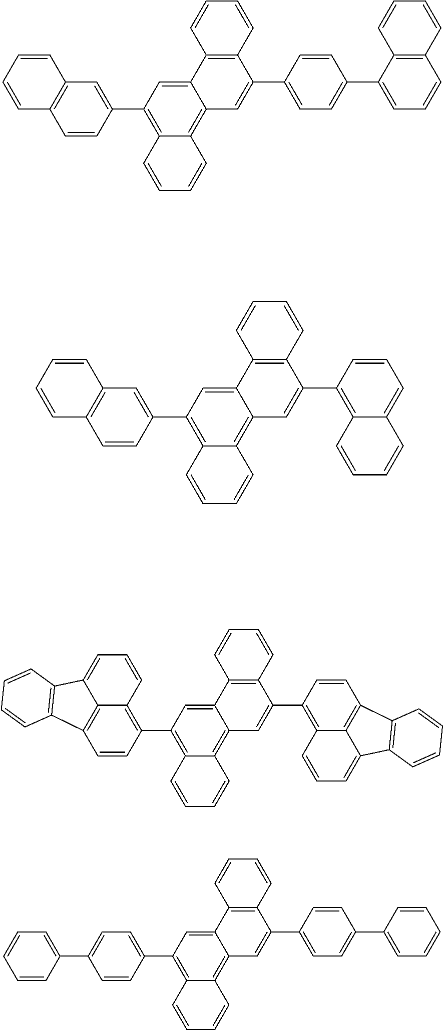

In the following compounds, the six-membered rings are all benzene rings.

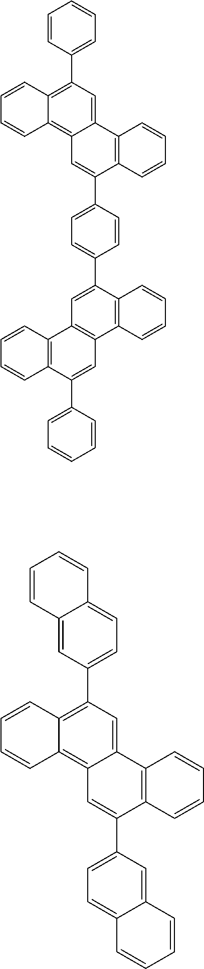

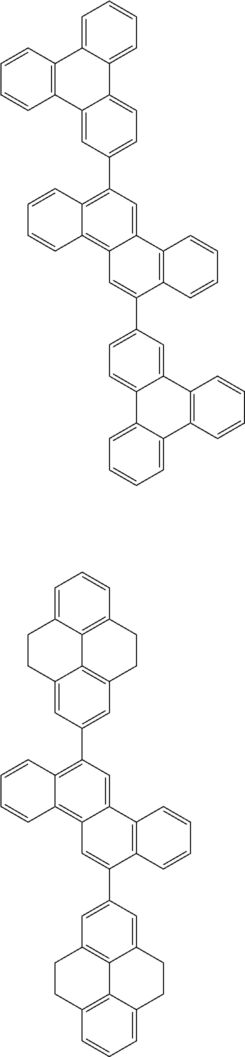

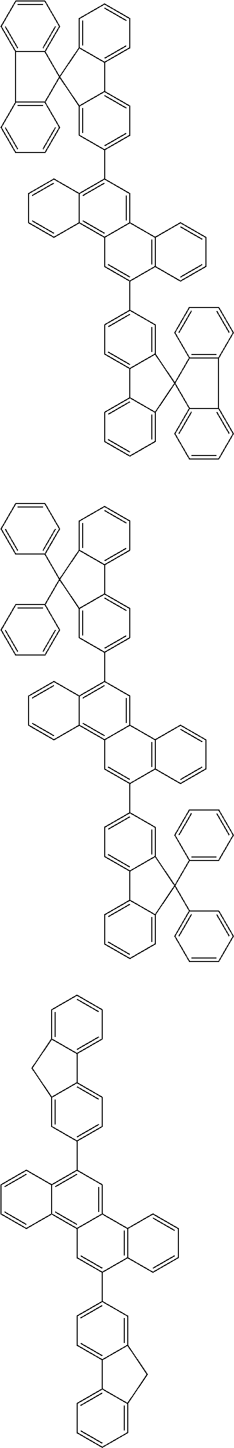

In the following compounds, the six-membered rings are all benzene rings.

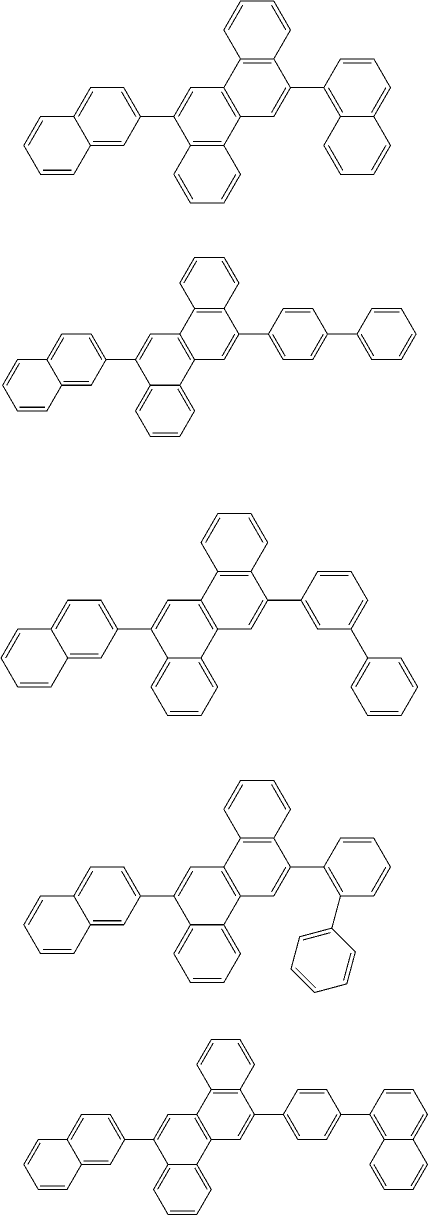

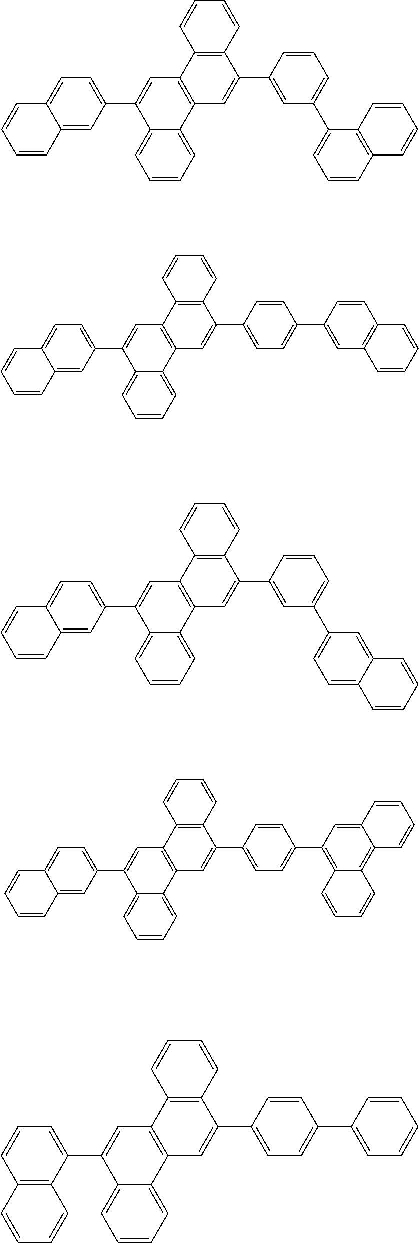

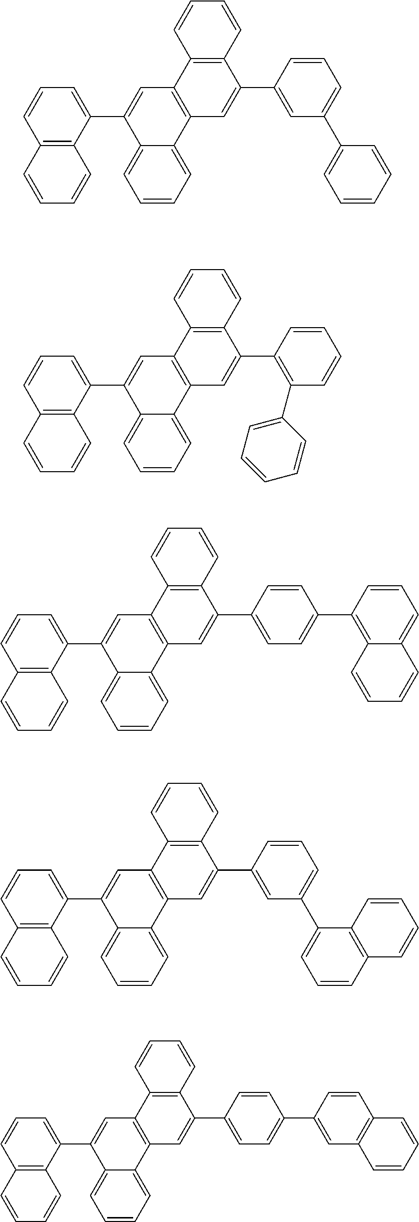

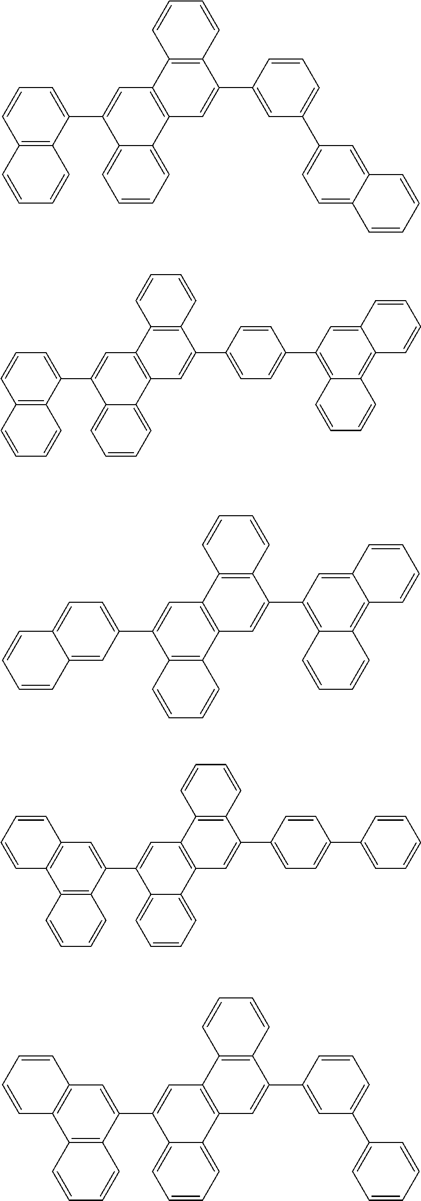

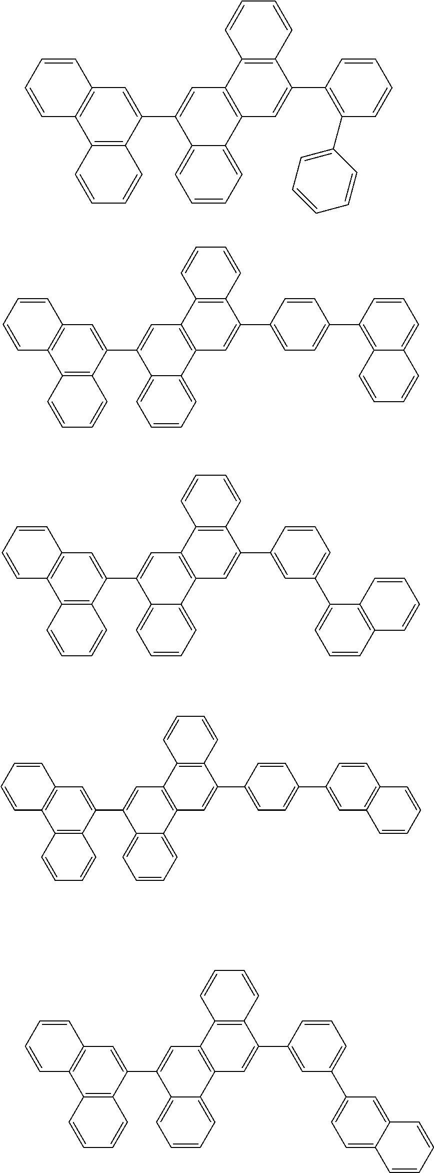

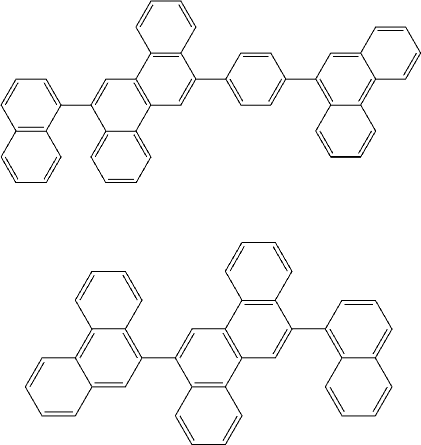

In the following compounds, the six-membered rings are all benzene rings.

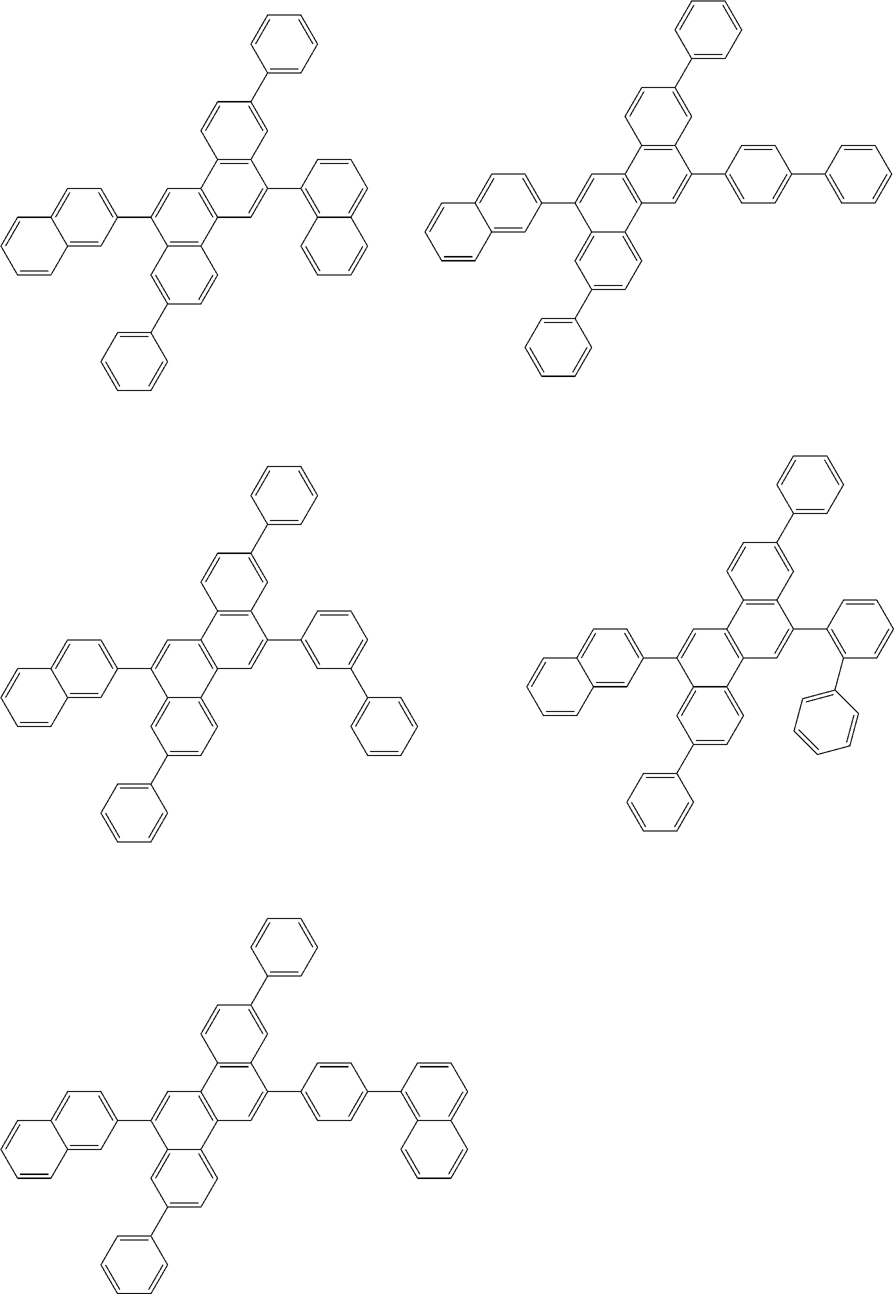

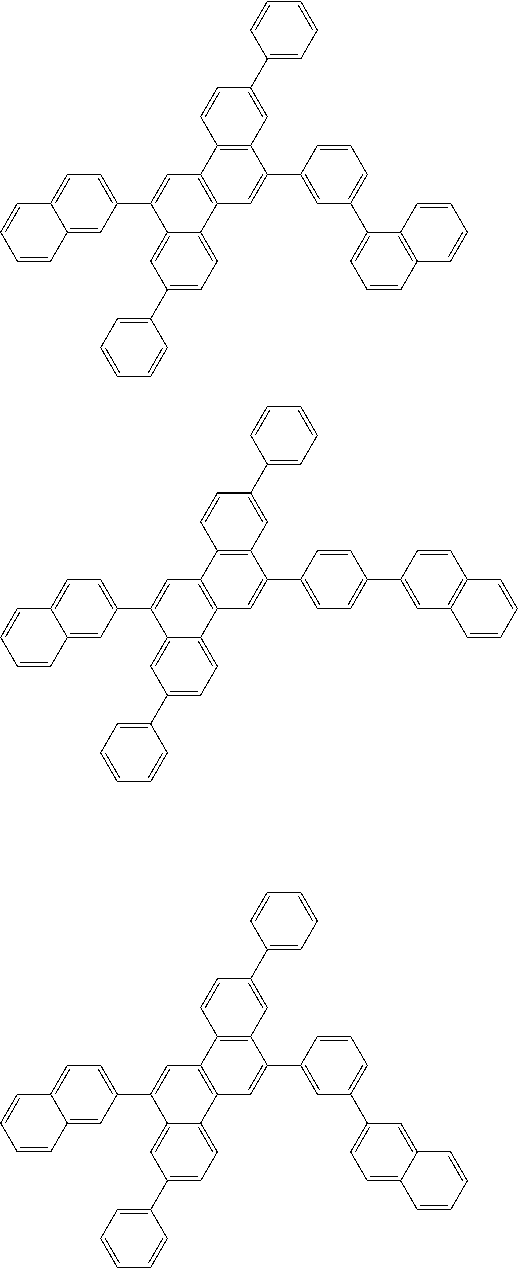

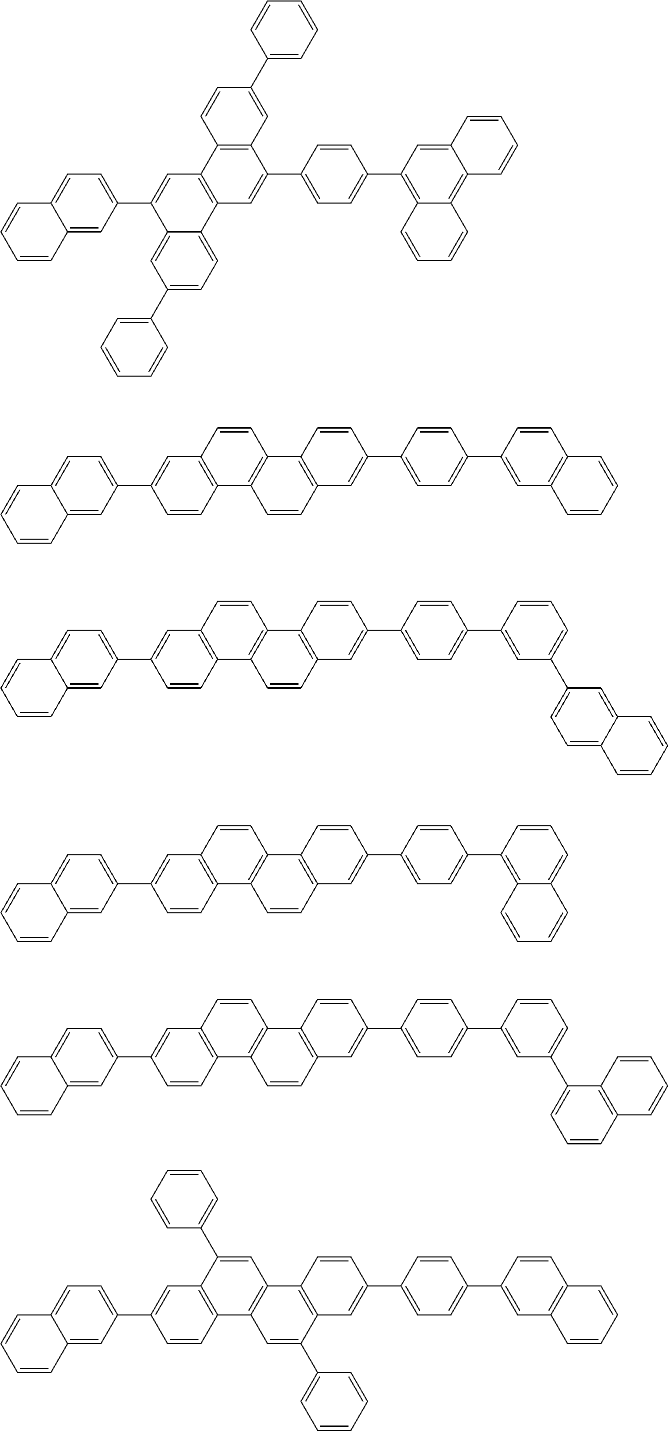

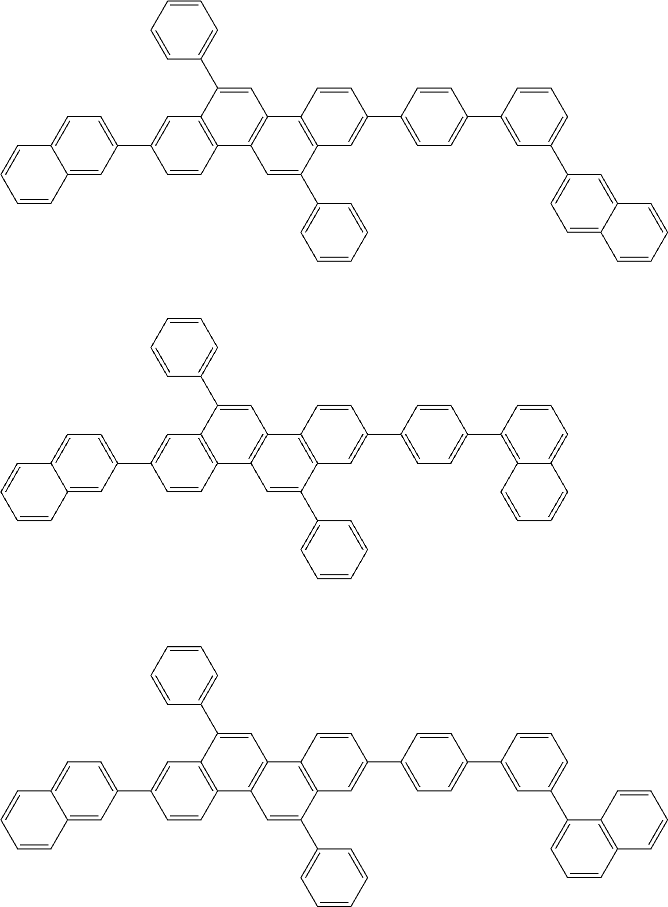

A pyrene skeleton-containing compound represented by formula (22) is usable as the first compound:

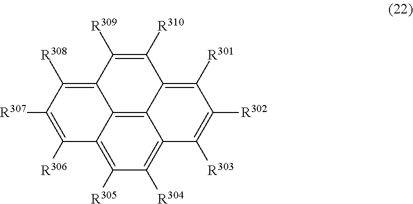

wherein:

R³⁰¹ to R³¹⁰ are each independently a hydrogen atom, a substituent, or -L³-Ar³¹, provided that at least one of R³⁰¹ to R³¹⁰ is -L³-Ar³¹;

the details of the substituent are as described above with respect to the substituent of R_{A}, R_{B}, and R_{C} of formula (D1);

the details of L³ and Ar³¹ are as described above with respect to L and Ar of formula (19), respectively; and

at least one of R³⁰¹, R³⁰³, R³⁰⁶, and R³⁰⁸ is preferably -L³-Ar³¹.

Examples of the pyrene derivative represented by formula (22) are shown below, although not particularly limited thereto.

In the following compounds, the six-membered rings are all benzene rings.

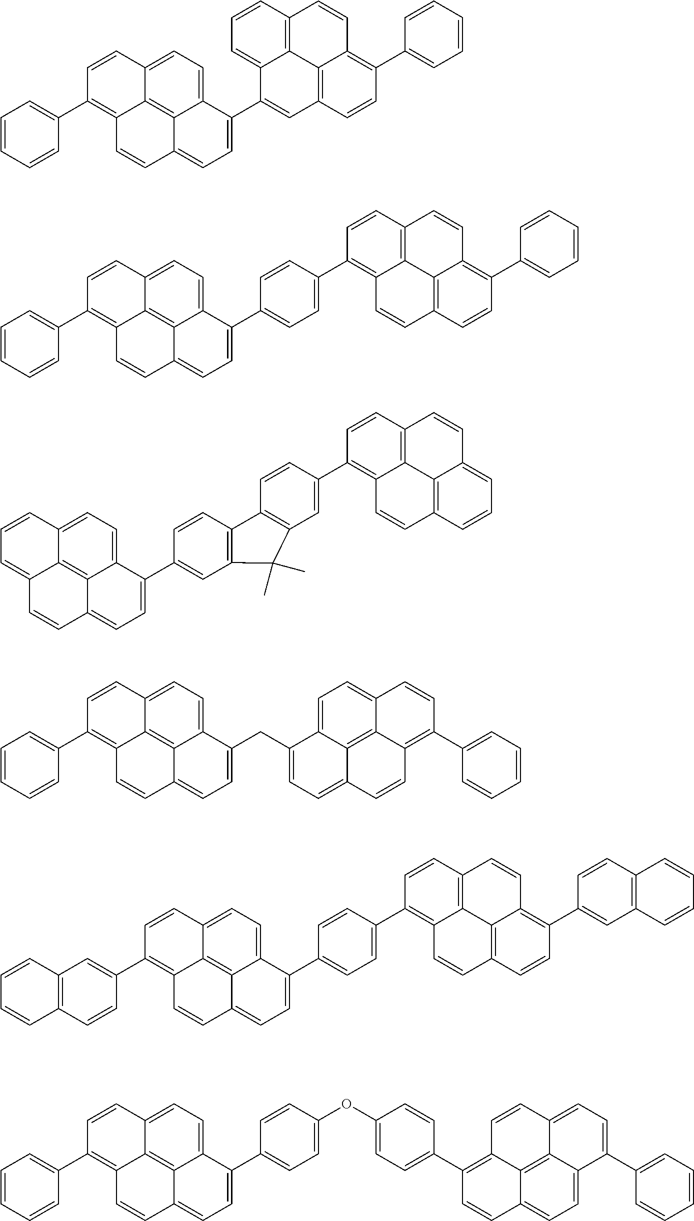

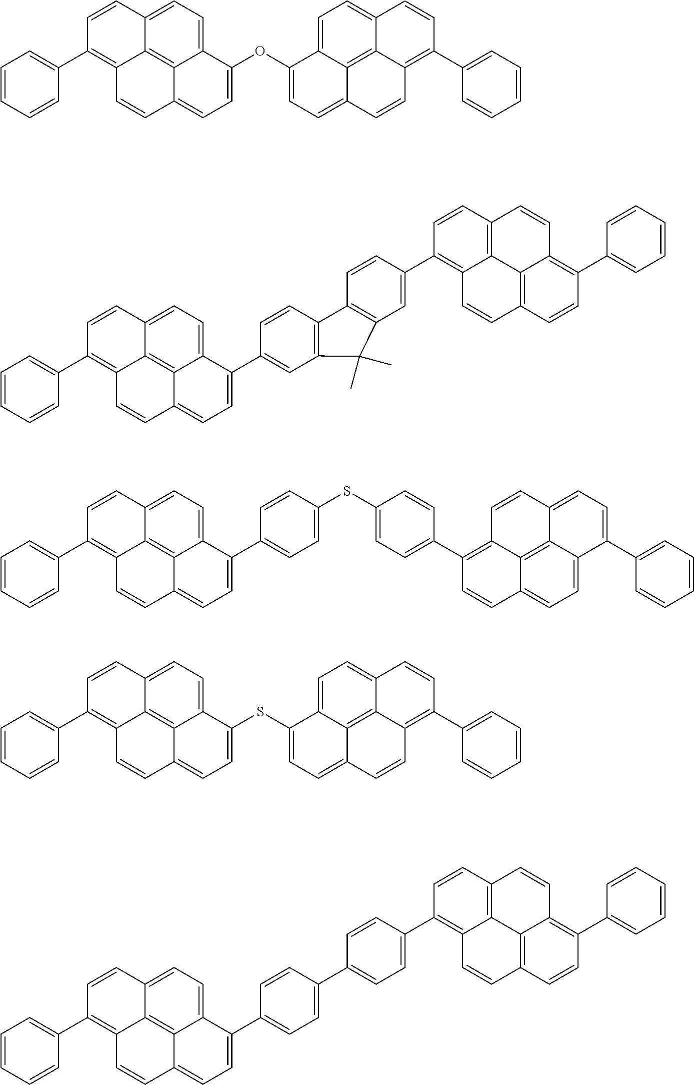

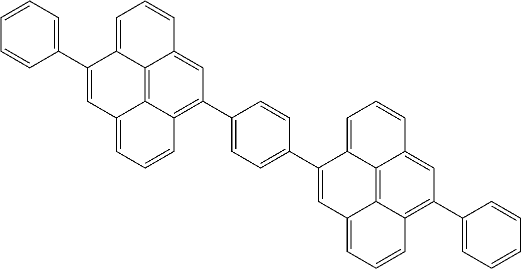 [SMILES: c1ccc(-c2cc3cccc4c(-c5ccc(-c6cc7cccc8c(-c9ccccc9)cc9cccc6c9c78)cc5)cc5cccc2c5c34)cc1]

In the following compounds, the six-membered rings are all benzene rings.

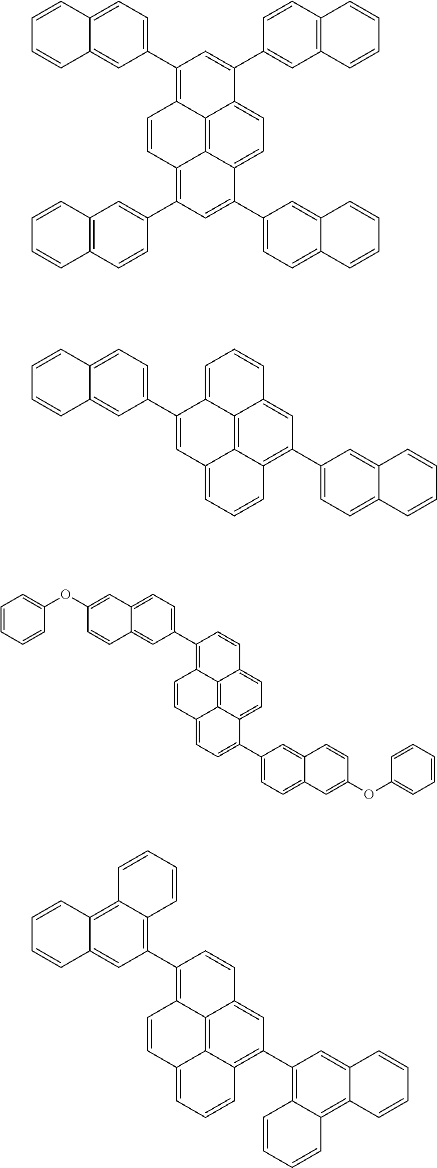

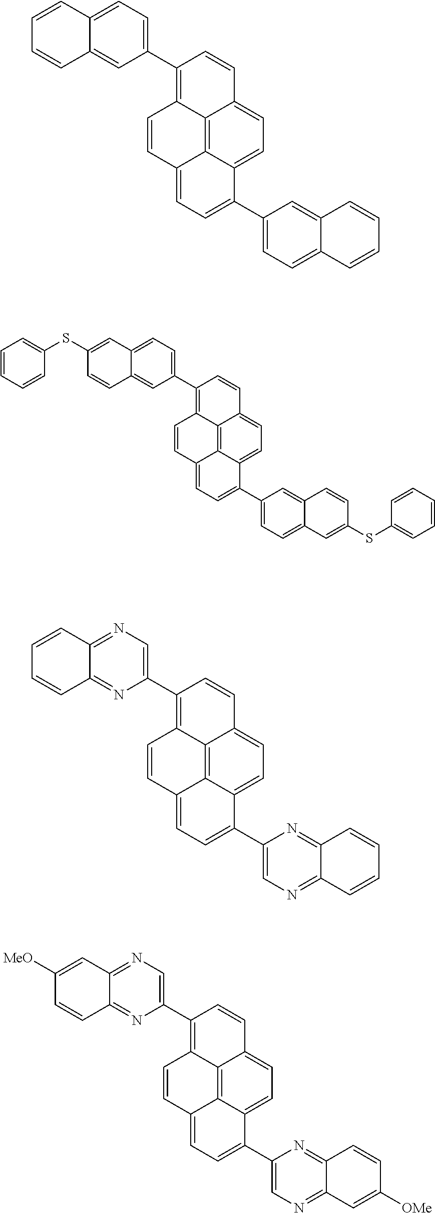

In the following compounds, the six-membered rings are all benzene rings.

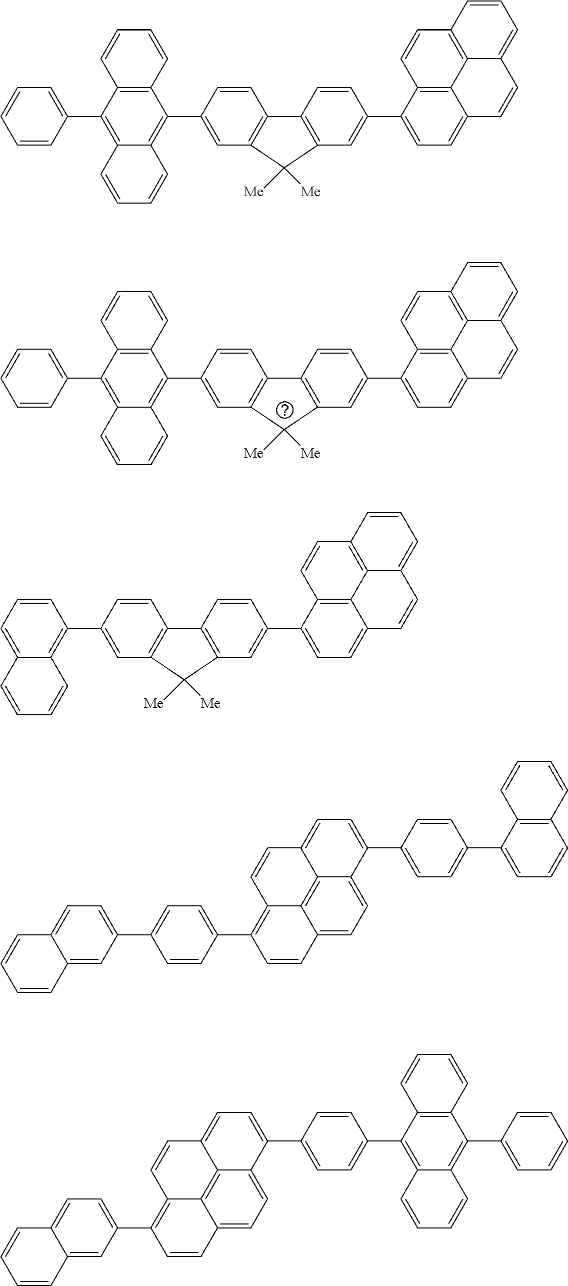

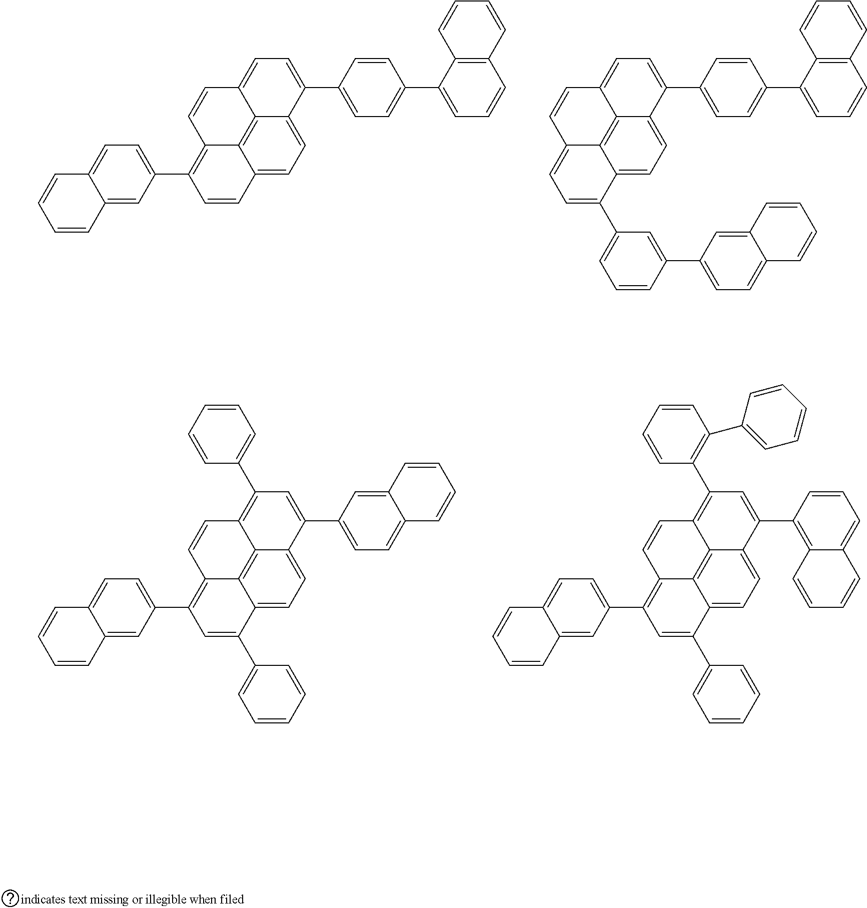

In the following compounds, the six-membered rings are all benzene rings.

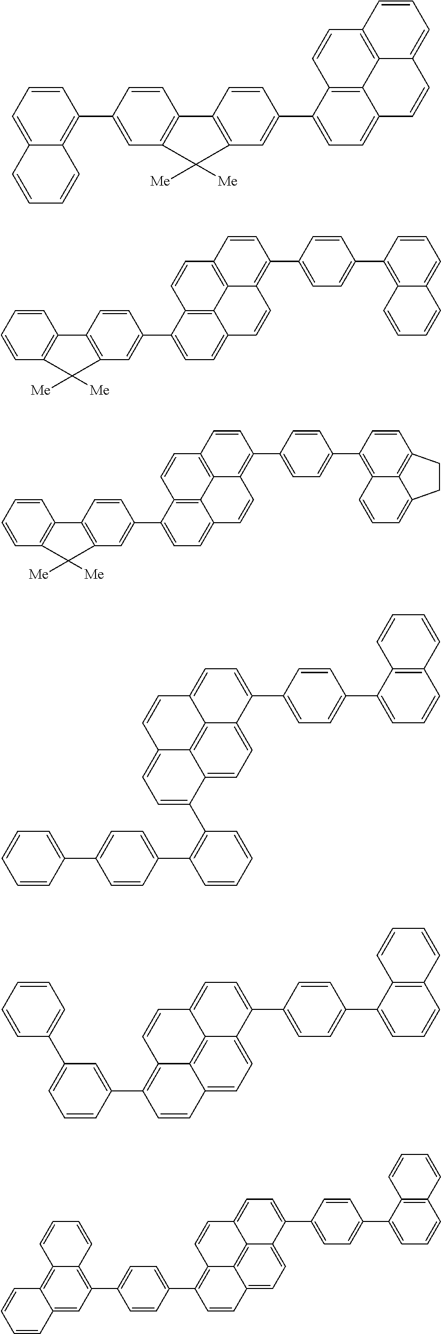

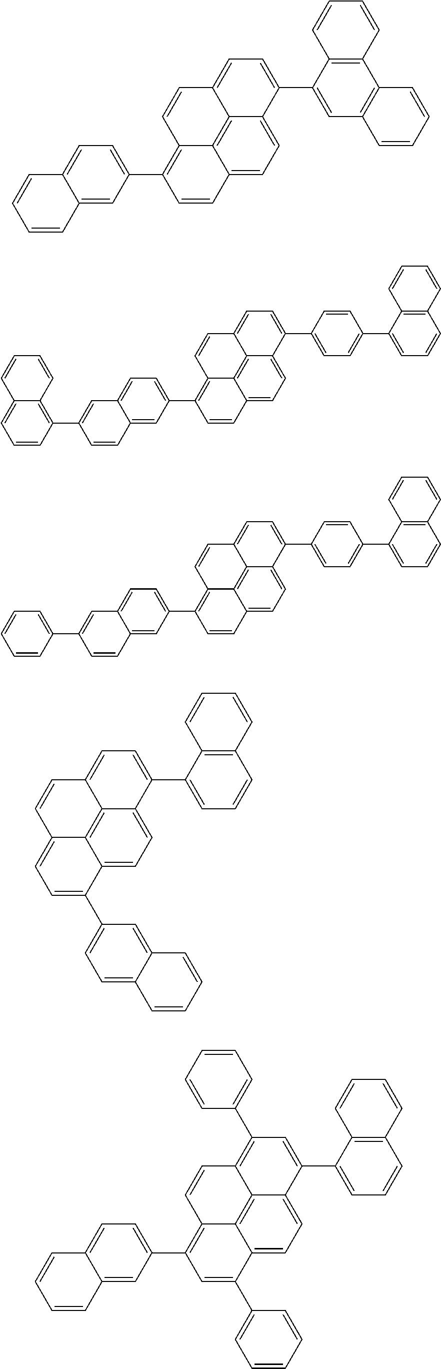

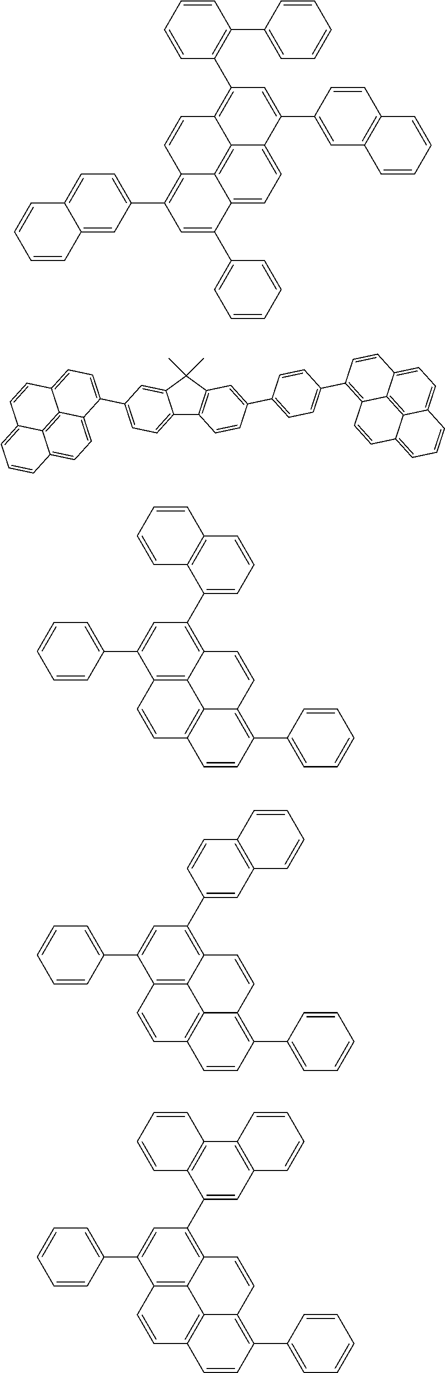

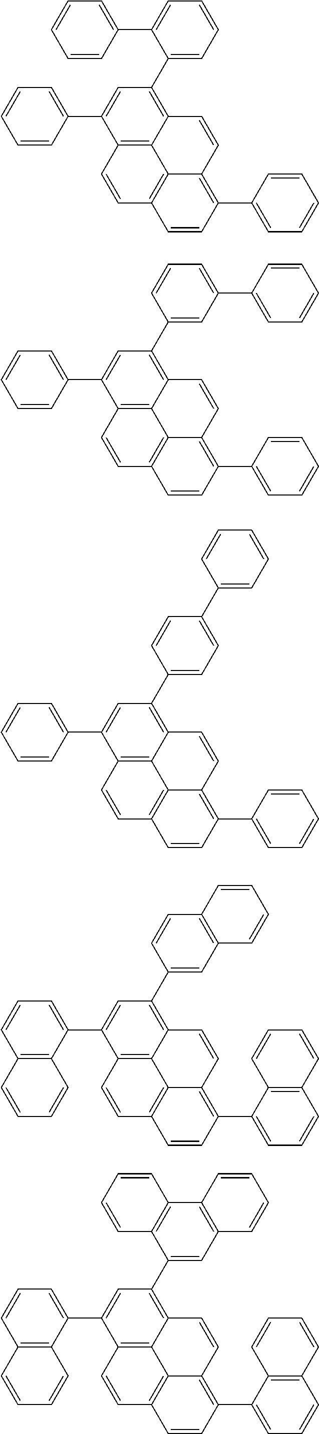

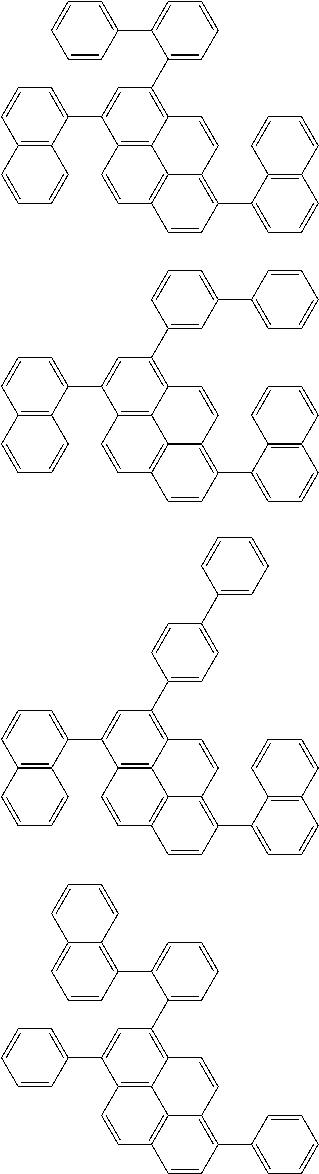

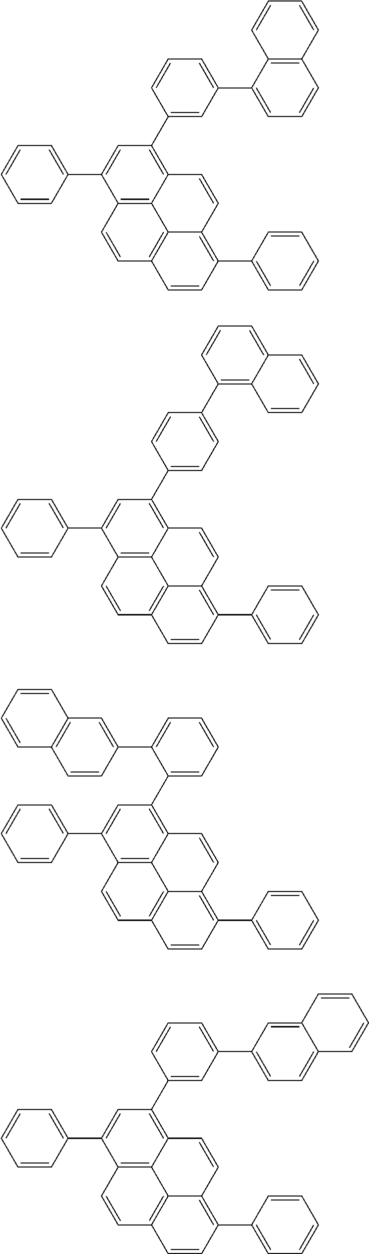

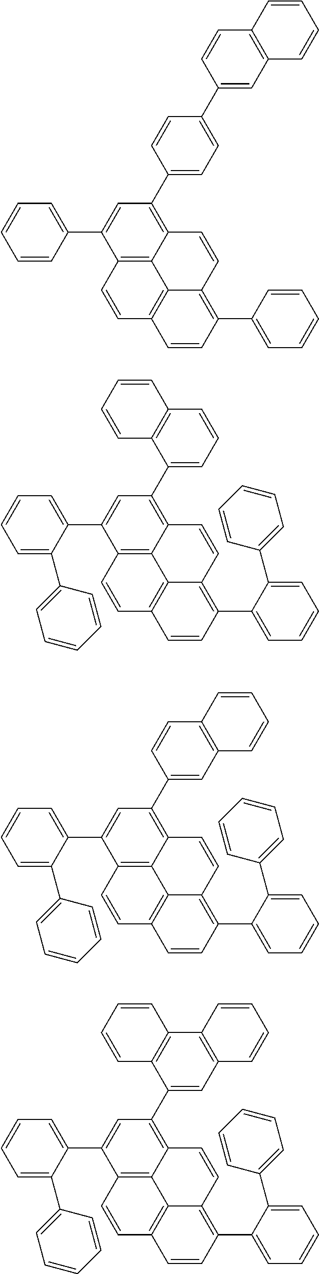

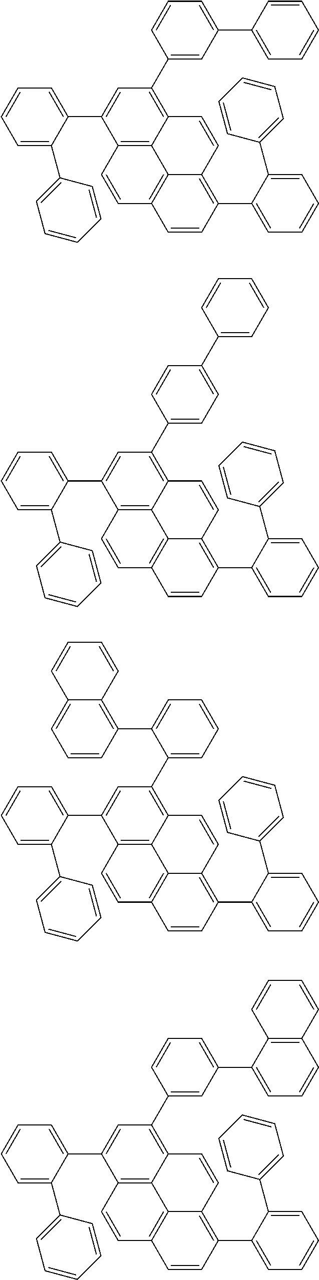

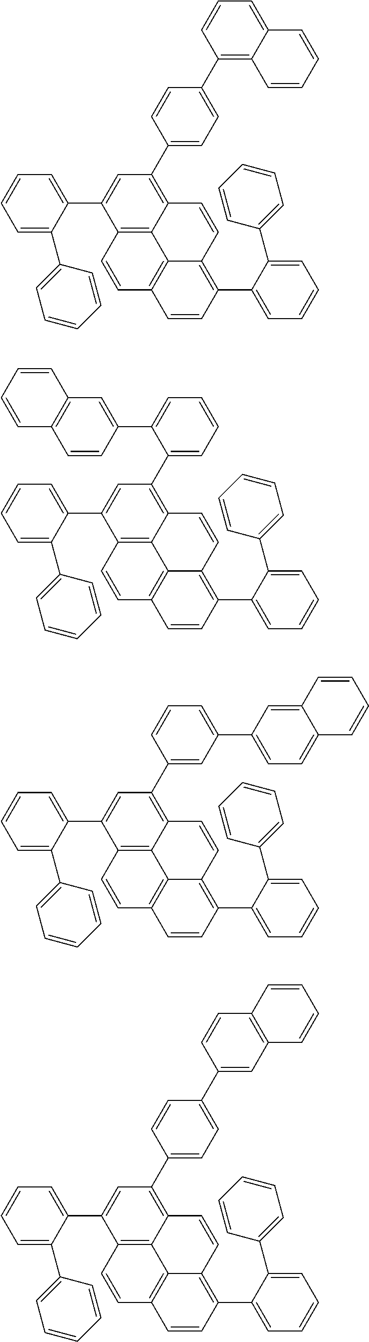

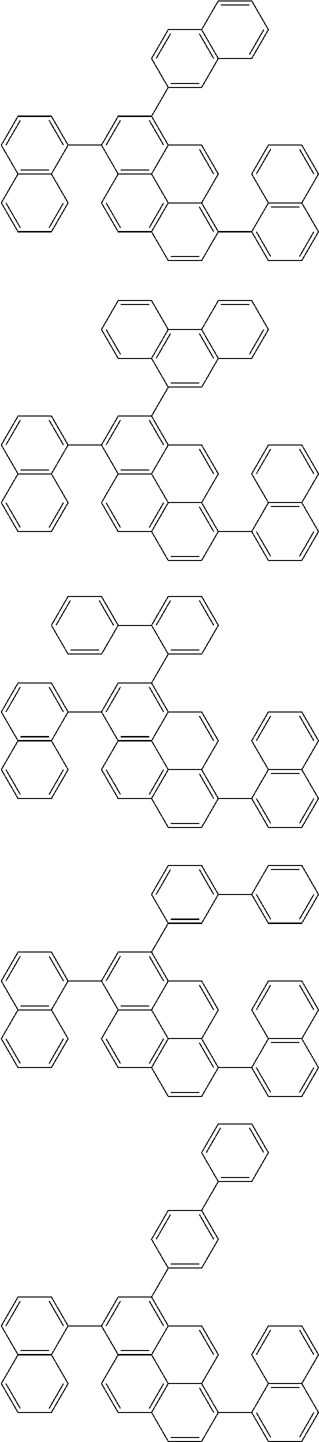

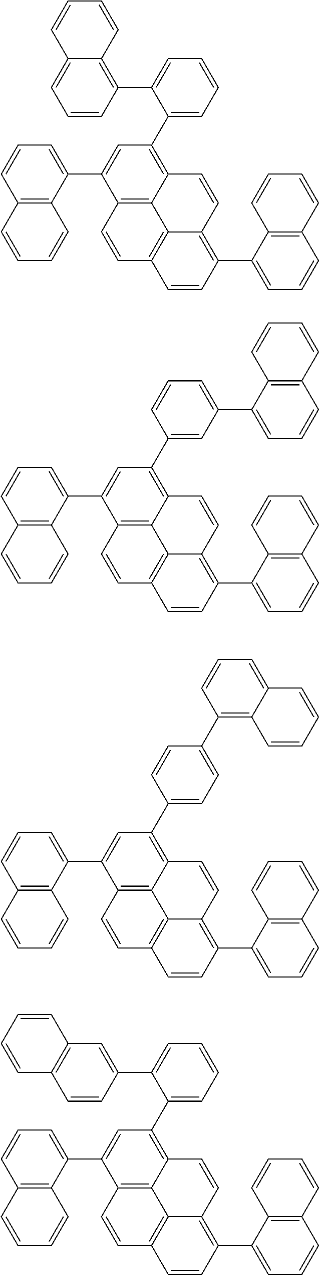

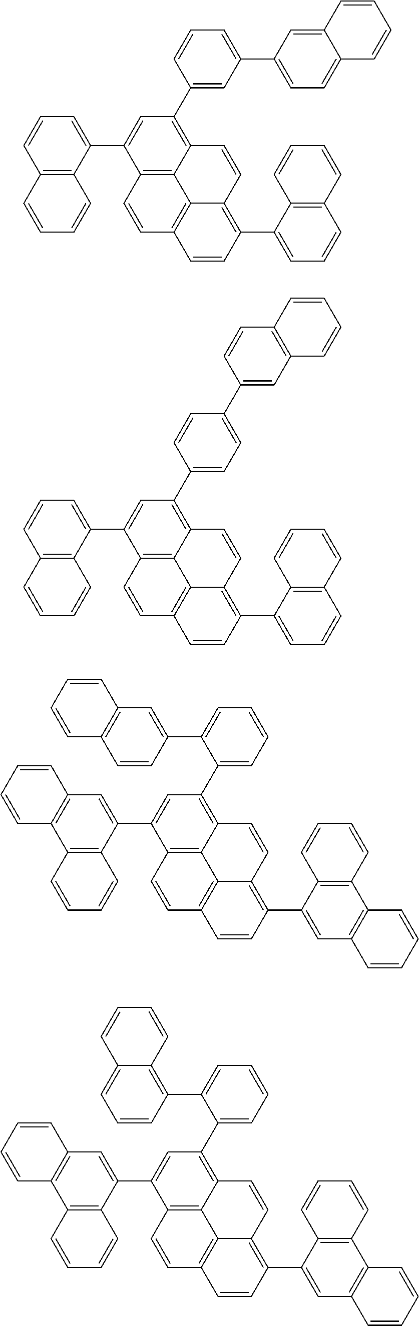

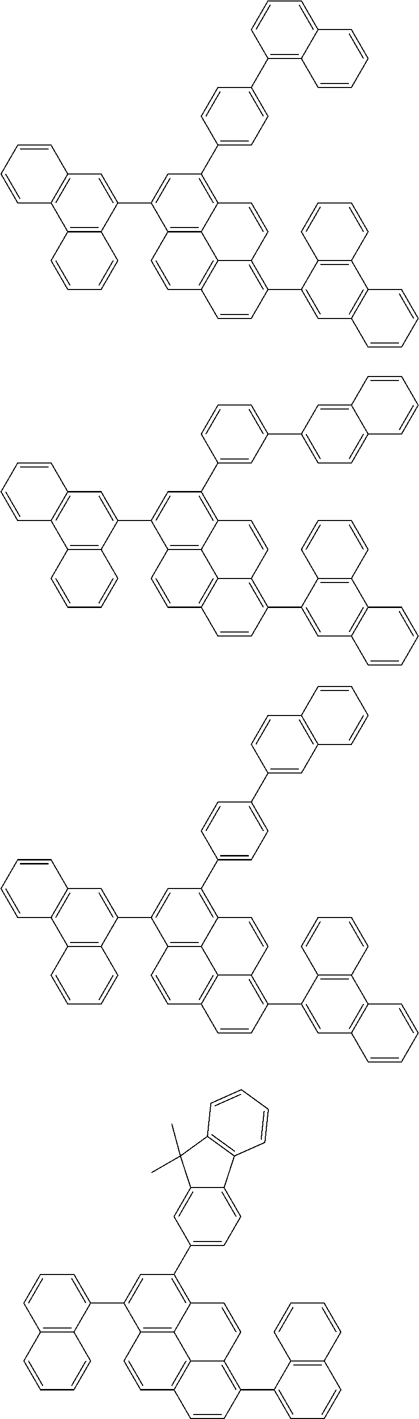

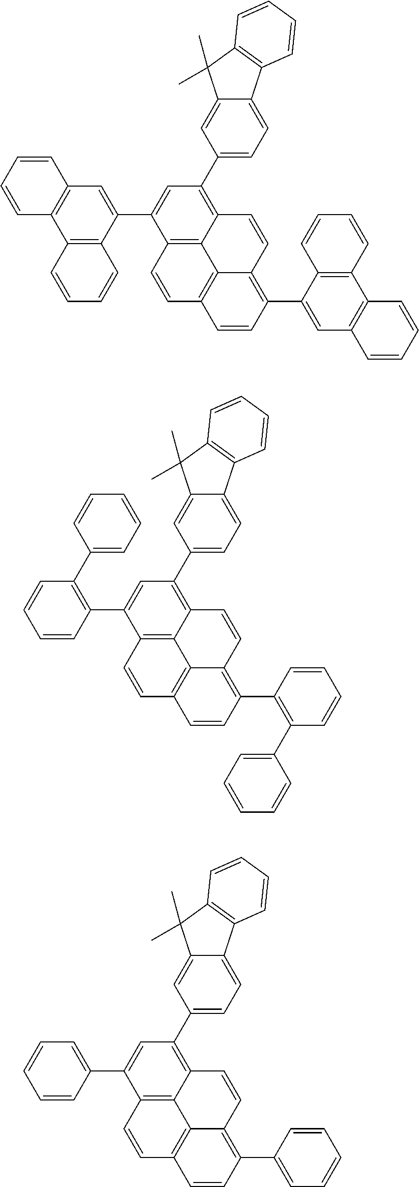

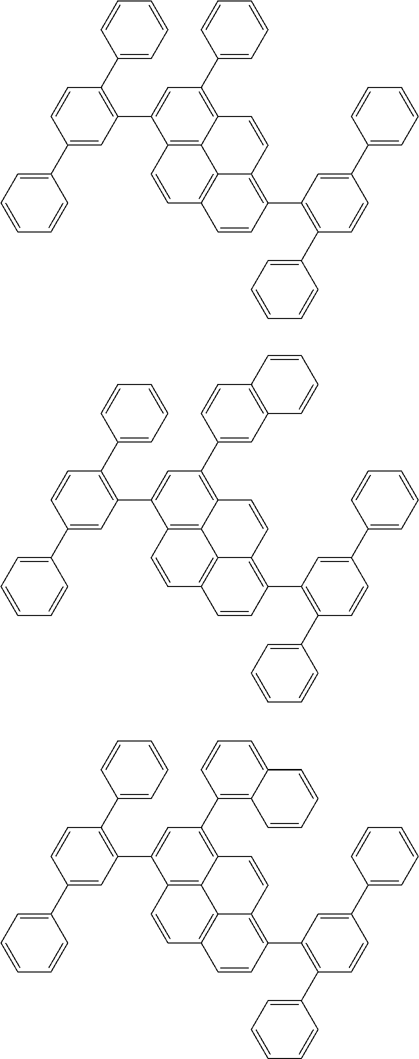

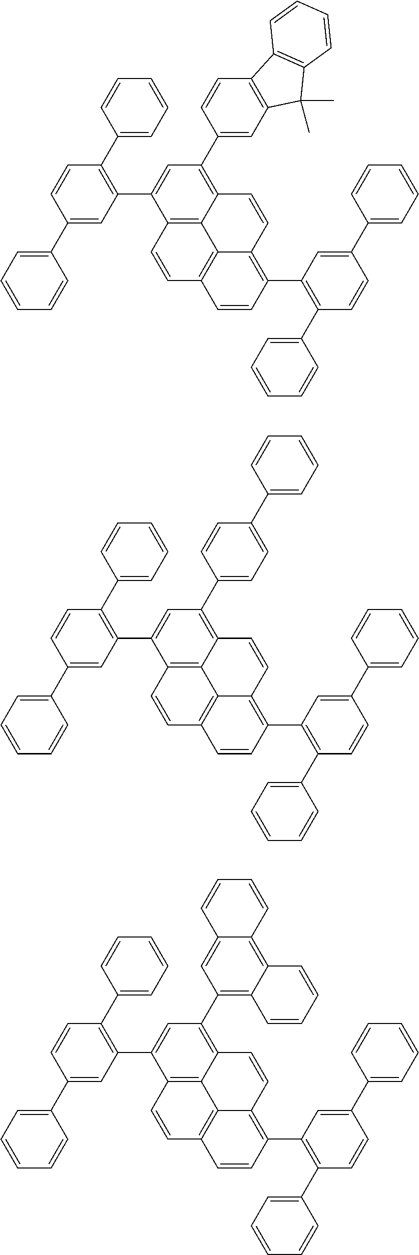

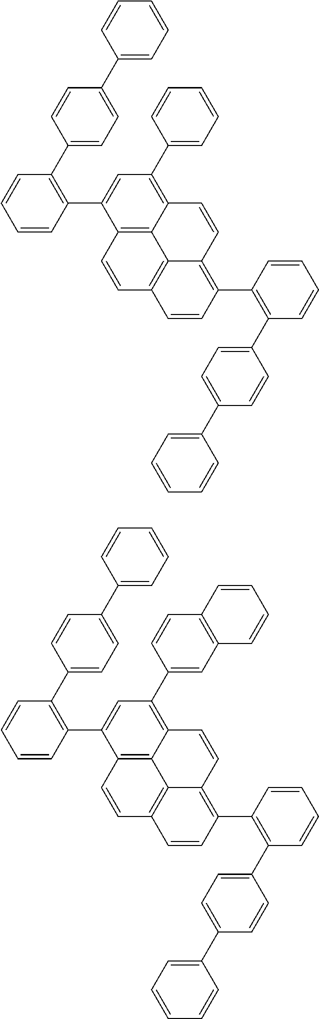

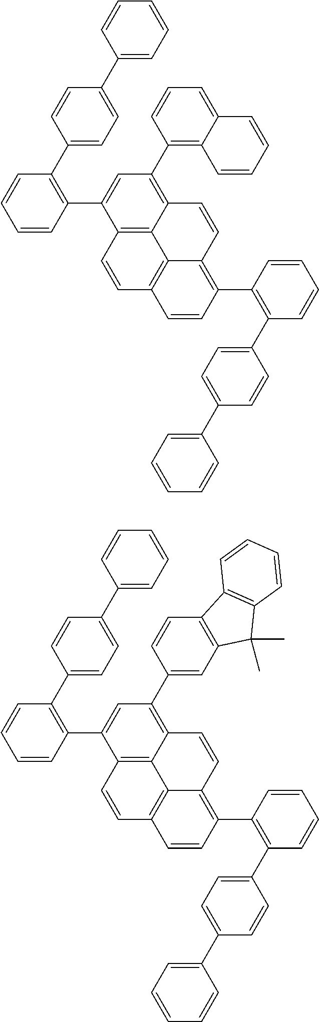

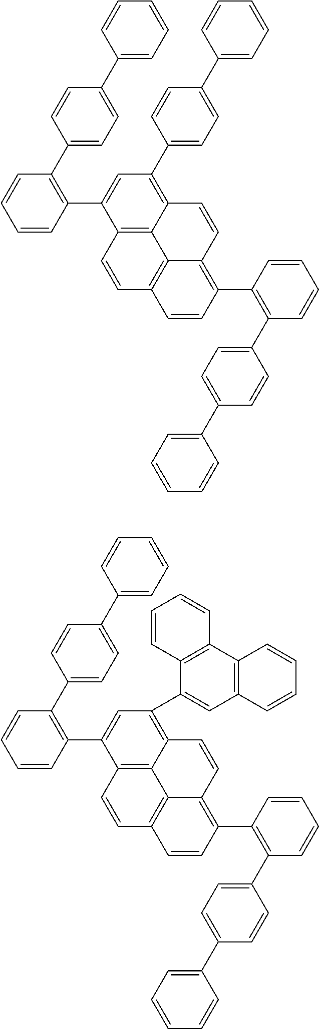

A fluorene skeleton-containing compound represented by formula (23) is usable as the first compound:

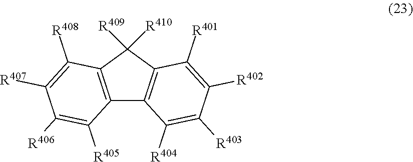

wherein:

R⁴⁰¹ to R⁴¹⁰ are each independently a hydrogen atom, a substituent, or -L⁴-Ar⁴¹, provided that at least one of R⁴⁰¹ to R⁴¹⁰ is -L⁴-Ar⁴¹;

the details of the substituent are as described above with respect to the substituent of R_{A}, R_{B}, and R_{C};

the details of L⁴ and Ar⁴¹ are as described above with respect to L and Ar of formula (19), respectively;

in at least one pair selected from R⁴⁰¹ and R⁴⁰², R⁴⁰² and R⁴⁰³, R⁴⁰³ and R⁴⁰⁴, R⁴⁰⁵ and R⁴⁰⁶, R⁴⁰⁶ and R⁴⁰⁷, and R⁴⁰⁷ and R⁴⁰⁸, adjacent two may be bonded to each other to form a substituted or unsubstituted ring structure;

each of R⁴⁰² and R⁴⁰⁷ is preferably -L⁴-Ar⁴¹;

each of R⁴⁰⁹ and R⁴¹⁰ is preferably a substituted or unsubstituted alkyl group having 1 to 20 carbon atoms or -L⁴-Ar⁴¹; and

the details of the alkyl group having 1 to 20 carbon atoms are as described above with respect to the alkyl group of R_{A}, R_{B}, and R_{C} of formula (D1).

Examples of the fluorene derivative represented by formula (23) are shown below, although not particularly limited.

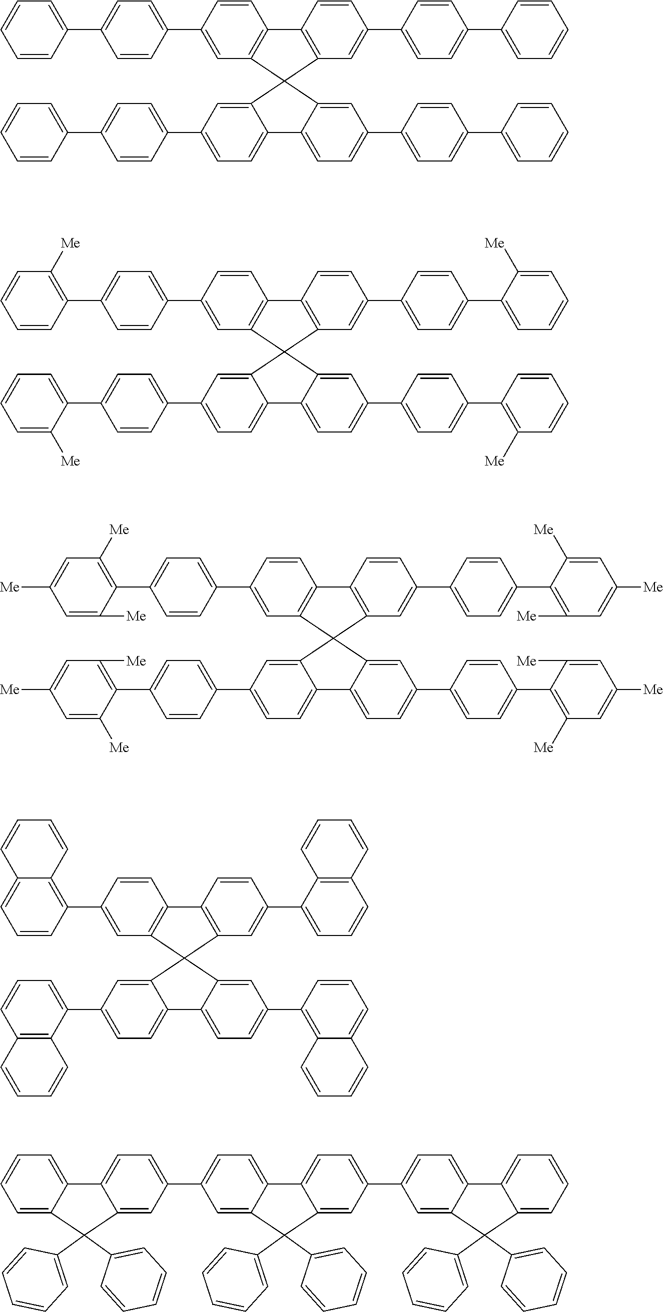

### Second Compound

The second compound is used in a fluorescent emitting layer of the organic EL device together with the dopant material and the first compound and works as a co-host material of the fluorescent emitting layer.

The second compound is at least one selected from the compounds represented by formula (3a):

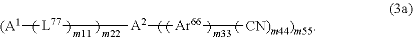

In formula (3a), L⁷⁷ is a substituted or unsubstituted arylene group having 6 to 50, preferably 6 to 30, more preferably 6 to 24, and still more preferably 6 to 18 ring carbon atoms or a substituted or unsubstituted heteroarylene group having 5 to 50, preferably 5 to 30, more preferably 5 to 18, and still more preferably 5 to 13 ring atoms.

The details of the arylene group having 6 to 50 ring carbon atoms and the details of the heteroarylene group having 5 to 50 ring atoms are as described above with respect to the corresponding groups of L in formula (19), respectively.

In formula (3a), Ar⁶⁶ is a di- to tetra-valent residue of an aromatic hydrocarbon ring having 6 to 50, preferably 6 to 30, more preferably 6 to 24, and still more preferably 6 to 18 ring carbon atoms or an aromatic heterocyclic ring having 5 to 50, preferably 5 to 30, more preferably 5 to 18, and still more preferably5 to 13 ring atoms, each optionally having a substituent.

The details of the aromatic hydrocarbon ring having 6 to 50 ring carbon atoms and the details of the aromatic heterocyclic ring having 5 to 50 ring atoms are as described above with respect to the corresponding rings of the ring π1 and the ring π2 in formula (D1), respectively.

In formula (3a), m11 is 0, 1, or 2 and preferably 0 or 1. When m11 is 0, L⁷⁷ is a single bond, and when m11 is 2, two L⁷⁷'s may be the same or different.

In formula (3a), m22 is 0 or 1. When m22 is 0, A¹-(L⁷⁷)ₘ₁₁- is not present and a hydrogen atom is bonded to A².

In formula (3a), m33 is 0, 1, 2, or 3, preferably 0, 1, or 2, and more preferably 0 or 1. When m33 is 0, Ar⁶⁶ is a single bond, and when m33 is 2 or 3, two or three Ar⁶⁶'s may be the same or different.

In formula (3a), m44 is 0, 1, 2, or 3, preferably 0, 1, or 2, and more preferably 0 or 1. When m44 is 0, CN is not present and a hydrogen atom is bonded to A⁶⁶.

In formula (3a), m55 is 1, 2, or 3 and preferably 1 or 2. When m55 is 2 or 3, two or three —(Ar⁶⁶)ₘ₃₃—(CN)ₘ₅₅ may be the same or different.

In formula (3a), A¹ is a monovalent group selected from formulae (A-1) to (A-12), and A² is a di- to tetra-valent group selected from formulae (A-1) to (A-12):

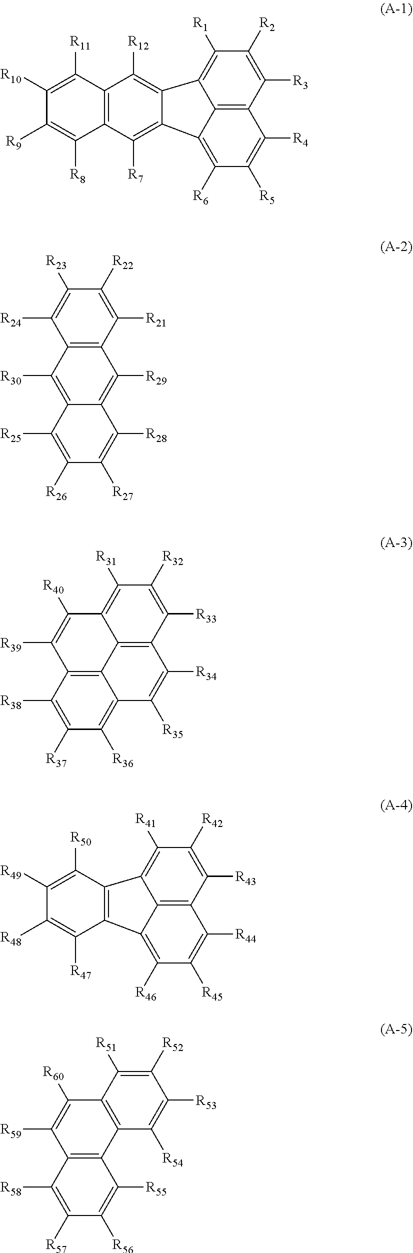

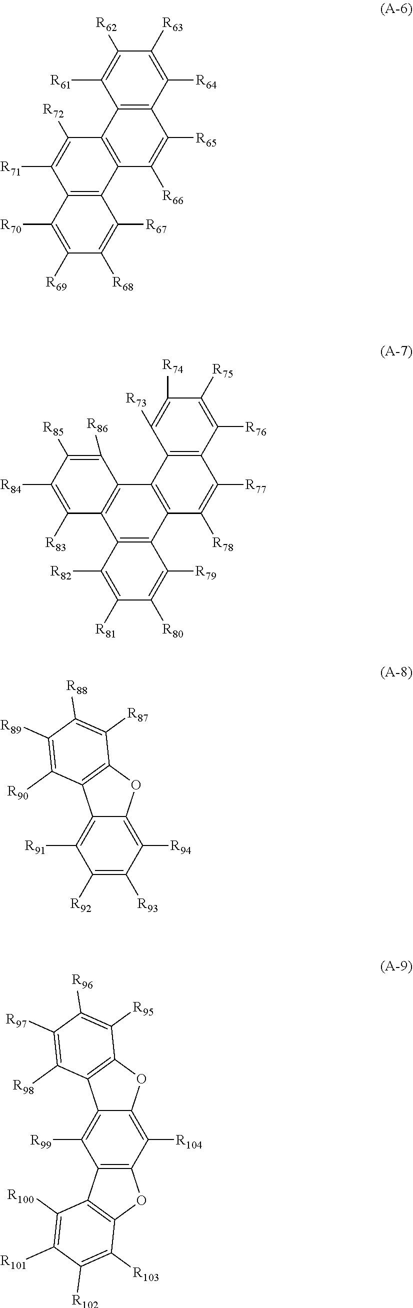

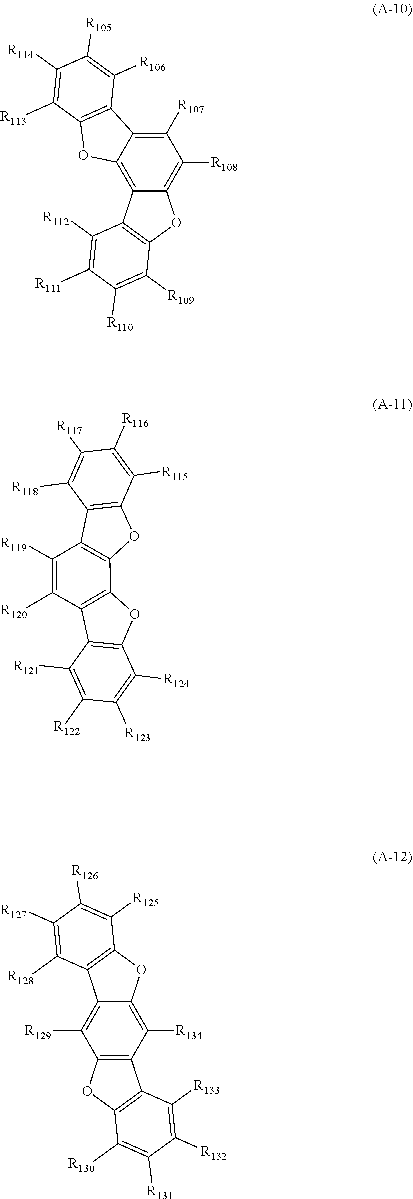

In formulae (A-1) to (A-12), one selected from R₁ to R₁₂, one selected from R₂₁ to R₃₀, one selected from R₃₁ to R₄₀, one selected from R₄₁ to R₅₀, one selected from R₅₁ to R₆₀, one selected from R₆₁ to R₇₂, one selected from R₇₃ to R₈₆, one selected from R₈₇ to R₉₄, one selected from R₉₅ to R₁₀₄, one selected from R₁₀₅ to R₁₁₄, one selected from R₁₁₅ to R₁₂₄, and one selected from R₁₂₅ to R₁₃₄ are single bonds each bonded to L⁷⁷;

or, two to four selected from R₁ to R₁₂, two to four selected from R₂₁ to R₃₀, two to four selected from R₃₁ to R₄₀, two to four selected from R₄₁ to R₅₀, two to four selected from R₅₁ to R₆₀, two to four selected from R₆₁ to R₇₂, two to four selected from R₇₃ to R₈₆, two to four selected from R₈₇ to R₉₄, two to four selected from R₉₅ to R₁₀₄, two to four selected from R₁₀₅ to R₁₁₄, two to four selected from R₁₁₅ to R₁₂₄, and two to four selected from R₁₂₅ to R₁₃₄ are single bonds, wherein one of the single bonds is bonded to L⁷⁷ and the other single bonds are bonded to Ar⁶⁶.

R₁ to R₁₂, R₂₁ to R₃₀, R₃₁ to R₄₀, R₄₁ to R₅₀, R₅₁ to R₆₀, R₆₁ to R₇₂, R₇₃ to R₈₆, R₈₇ to R₉₄, R₉₅ to R₁₀₄, R₁₀₅ to R₁₁₄, R₁₁₅ to R₁₂₄, and R₁₂₅ to R₁₃₄ each not the single bond are each independently a hydrogen atom, a halogen atom, a cyano group, a substituted or unsubstituted alkyl group having 1 to 20, preferably 1 to 10, and more preferably 1 to 6 carbon atoms, a substituted or unsubstituted cycloalkyl group having 3 to 20, preferably 3 to 6, and more preferably 5 or 6 ring carbon atoms, a group represented by —Si(R₁₀₁)(R₁₀₂)(R₁₀₃), or a substituted or unsubstituted aryl group having 6 to 50, preferably 6 to 30, more preferably 6 to 24, and still more preferably 6 to 18 ring carbon atoms.

The details of the alkyl group having 1 to 20 carbon atoms, the cycloalkyl group having 3 to 20 ring carbon atoms, the group represented by —Si(R₁₀₁)(R₁₀₂)(R₁₀₃) wherein R₁₀₁, R₁₀₂, and R₁₀₃ are as defined above, and the aryl group having 6 to 50 ring carbon atoms are as described above with respect to the corresponding groups of R_{A}, R_{B}, and R_{C} in formula (D1), respectively.

R₁ to R₁₂, R₂₁ to R₃₀, R₃₁ to R₄₀, R₄₁ to R₅₀, R₅₁ to R₆₀, R₆₁ to R₇₂, R₇₃ to R₈₆, R₈₇ to R₉₄, R₉₅ to R₁₀₁, R₁₀₅ to R₁₁₄, R₁₁₅ to R₁₂₄, and R₁₂₅ to R₁₃₄ each not the single bond may be all hydrogen atoms.

Adjacent two selected from R₁ to R₁₂, R₂₁ to R₃₀, R₃₁ to R₄₀, R₄₁ to R₅₀, R₅₁ to R₆₀, R₆₁ to R₇₂, R₇₃ to R₈₆, R₈₇ to R₉₄, R₉₅ to R₁₀₄, R₁₀₅ to R₁₁₄, R₁₁₅ to R₁₂₄, and R₁₂₅ to R₁₃₄ each not the single bond may be bonded to each other to form a substituted or unsubstituted ring structure.

The ring structure is selected, for example, from the aromatic hydrocarbon ring having 6 to 50 ring carbon atoms and the aromatic heterocyclic ring having 5 to 50 ring atoms each described above with respect to the ring π1 and the ring π2 of formula (D1), preferably selected from formulae (2) to (11) described above with respect to formula (1).

Examples of the compound represented by formula (3a) are shown below, although not limited thereto.

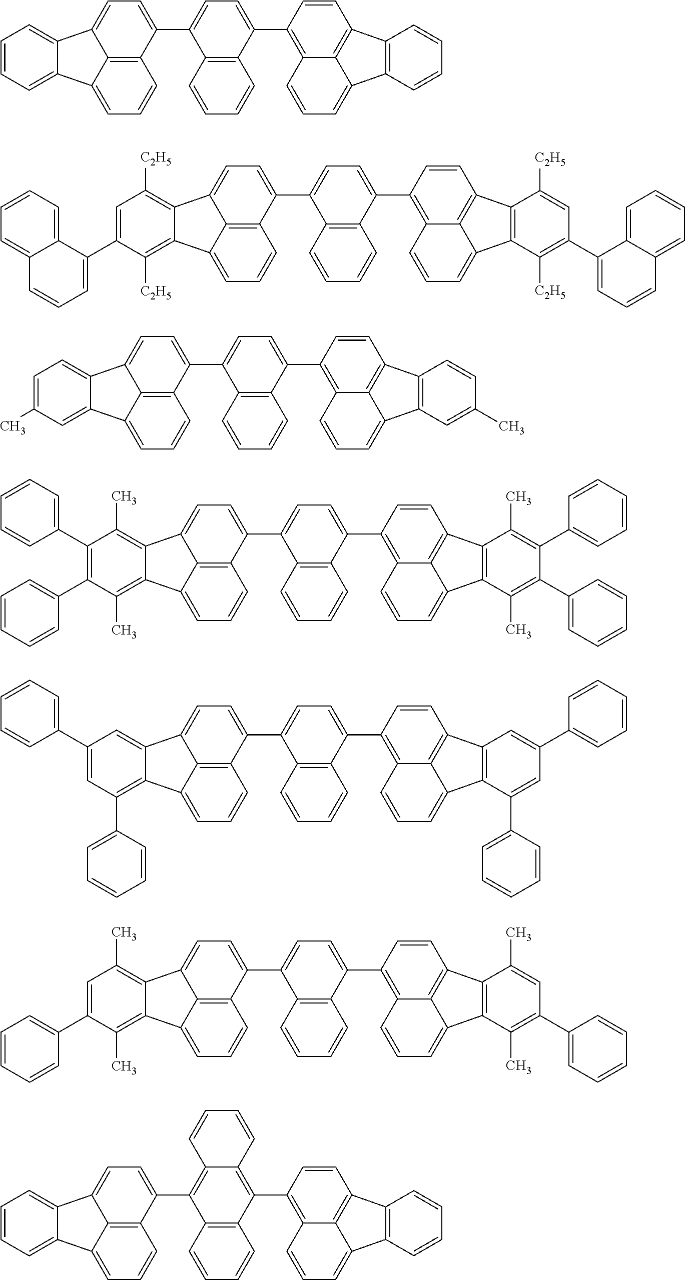

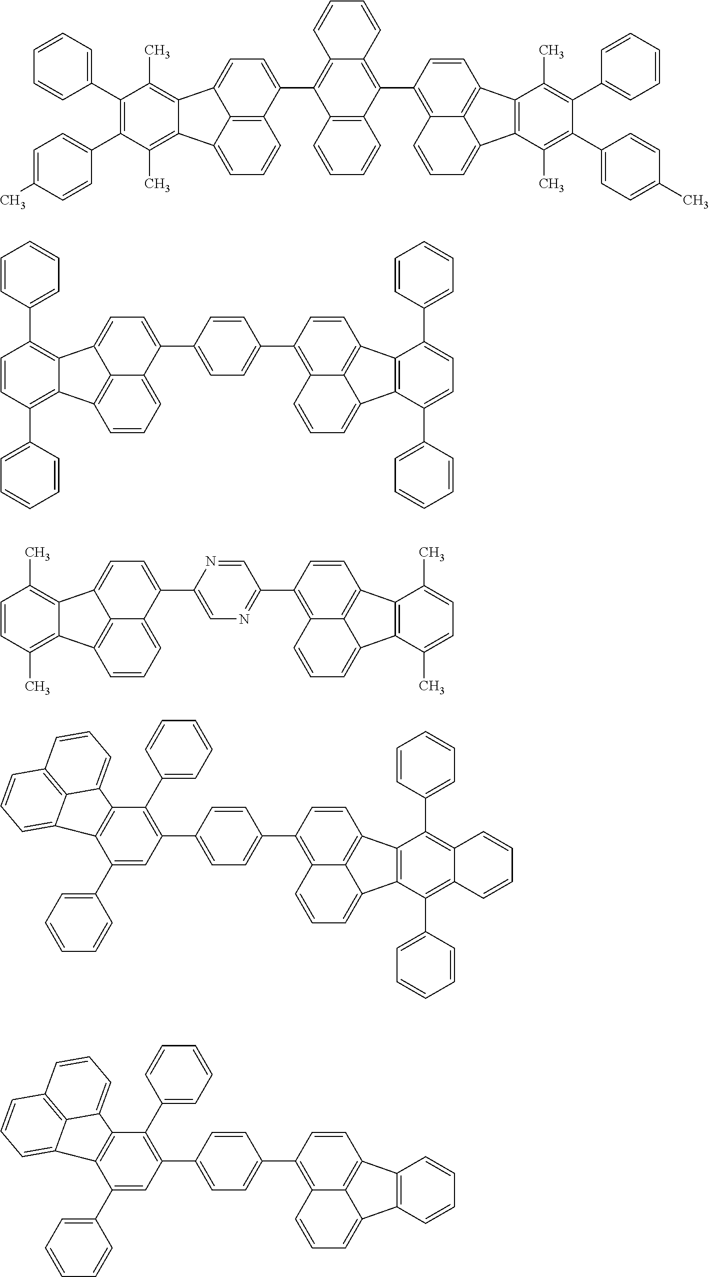

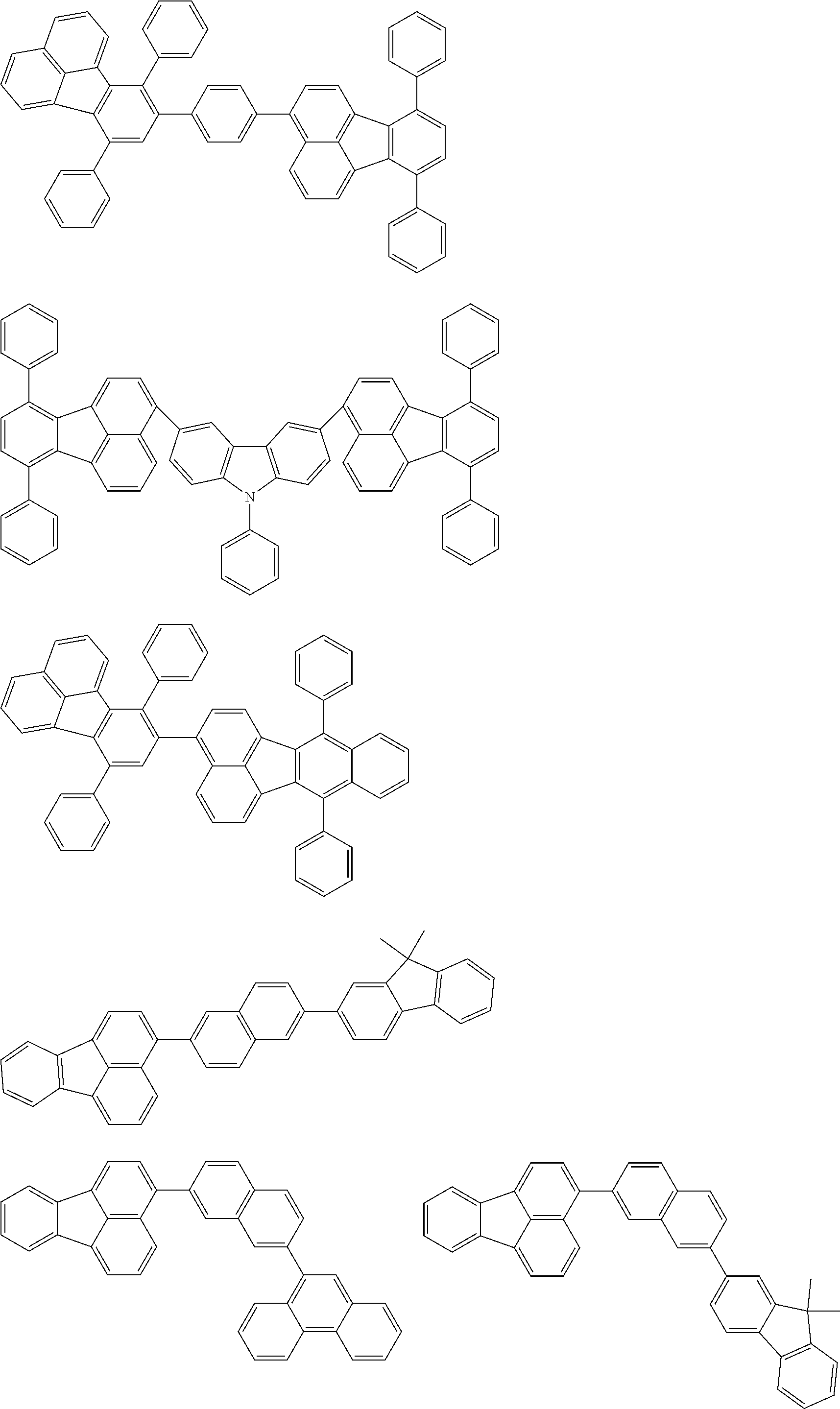

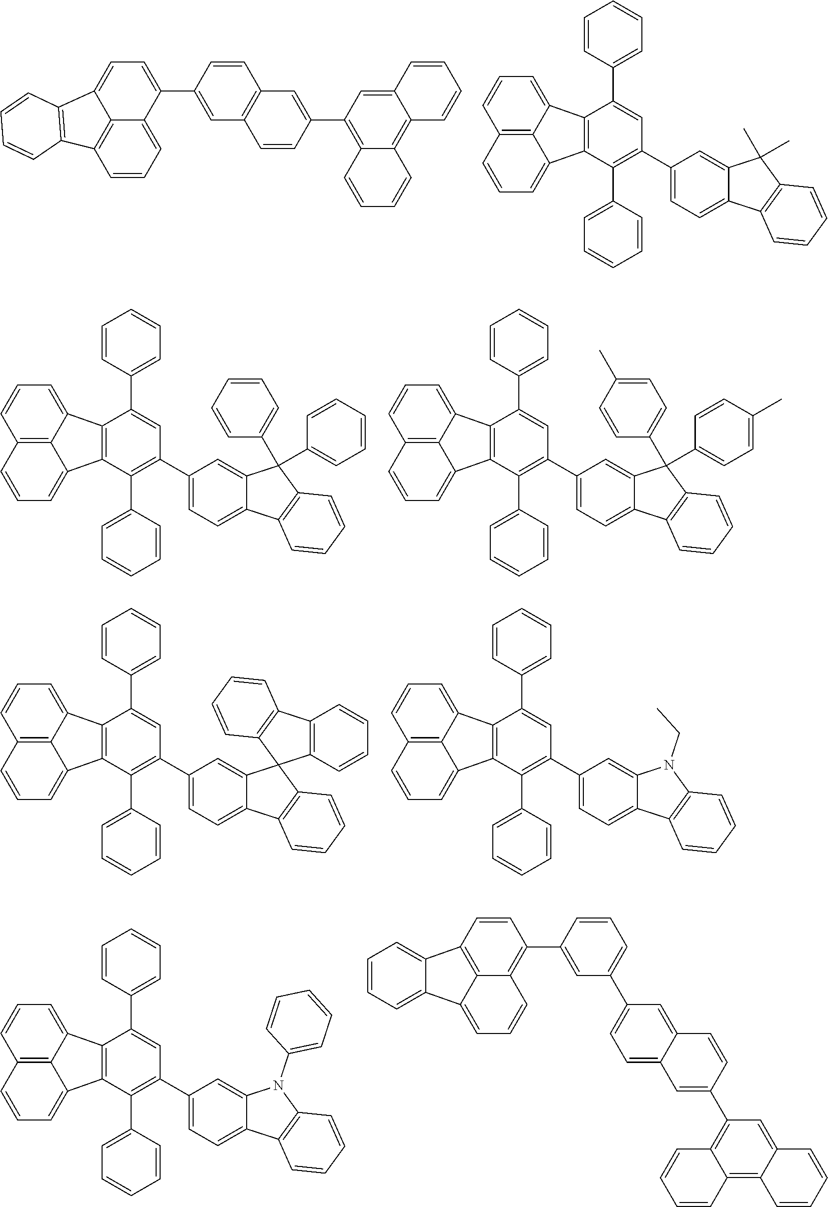

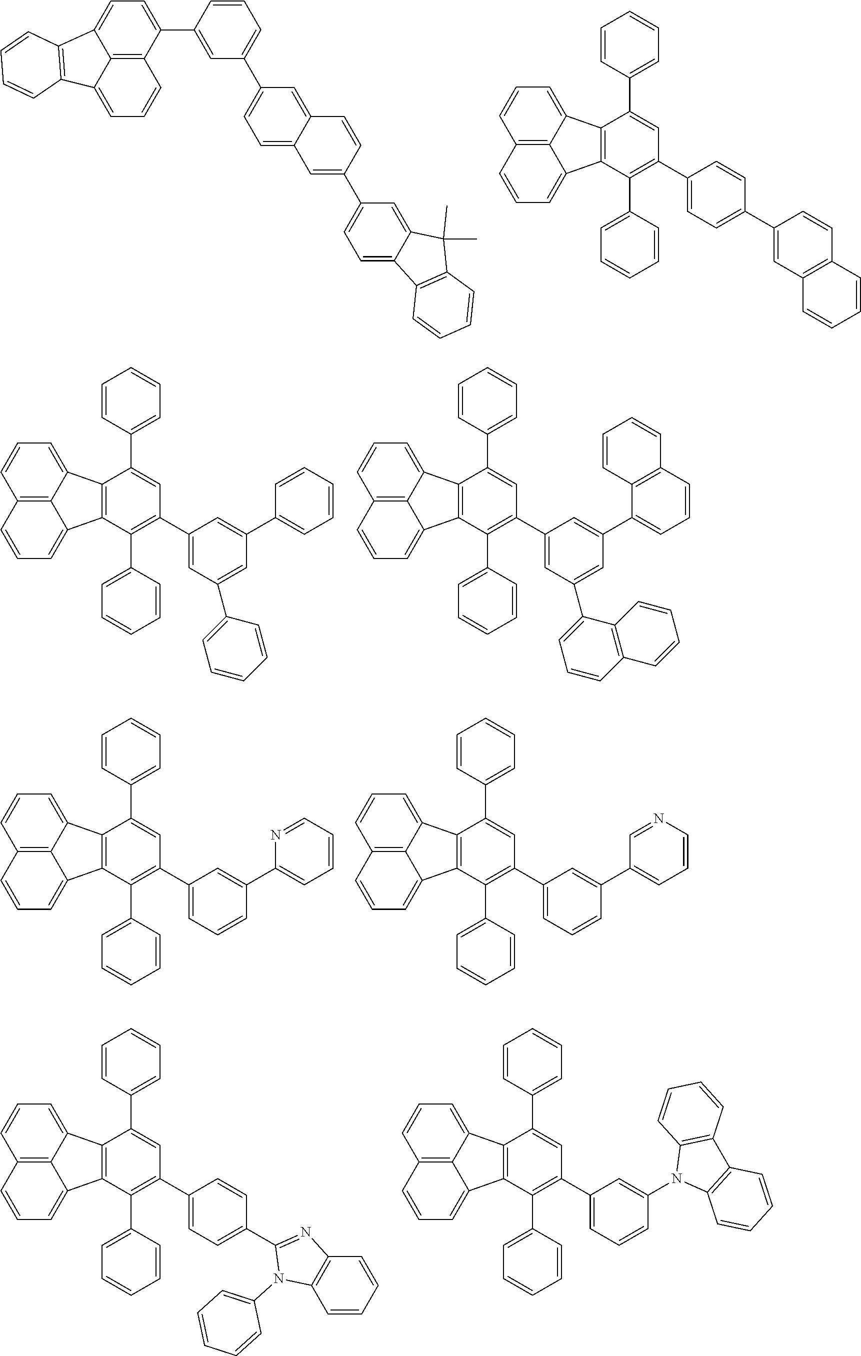

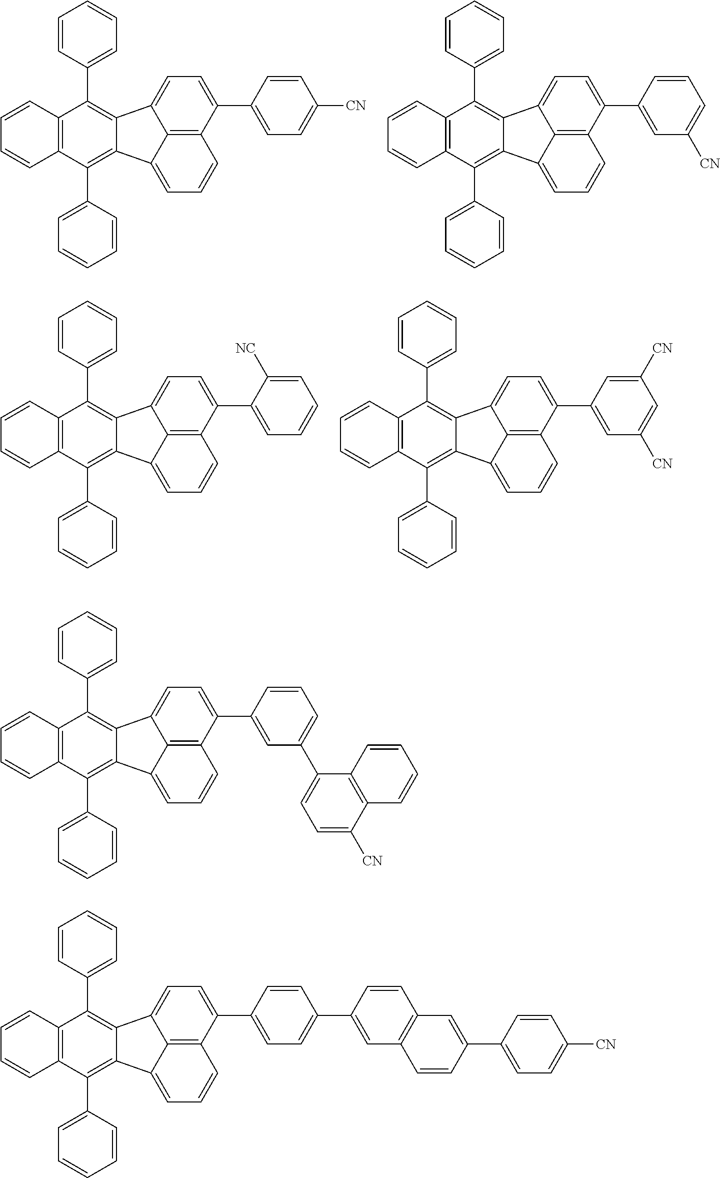

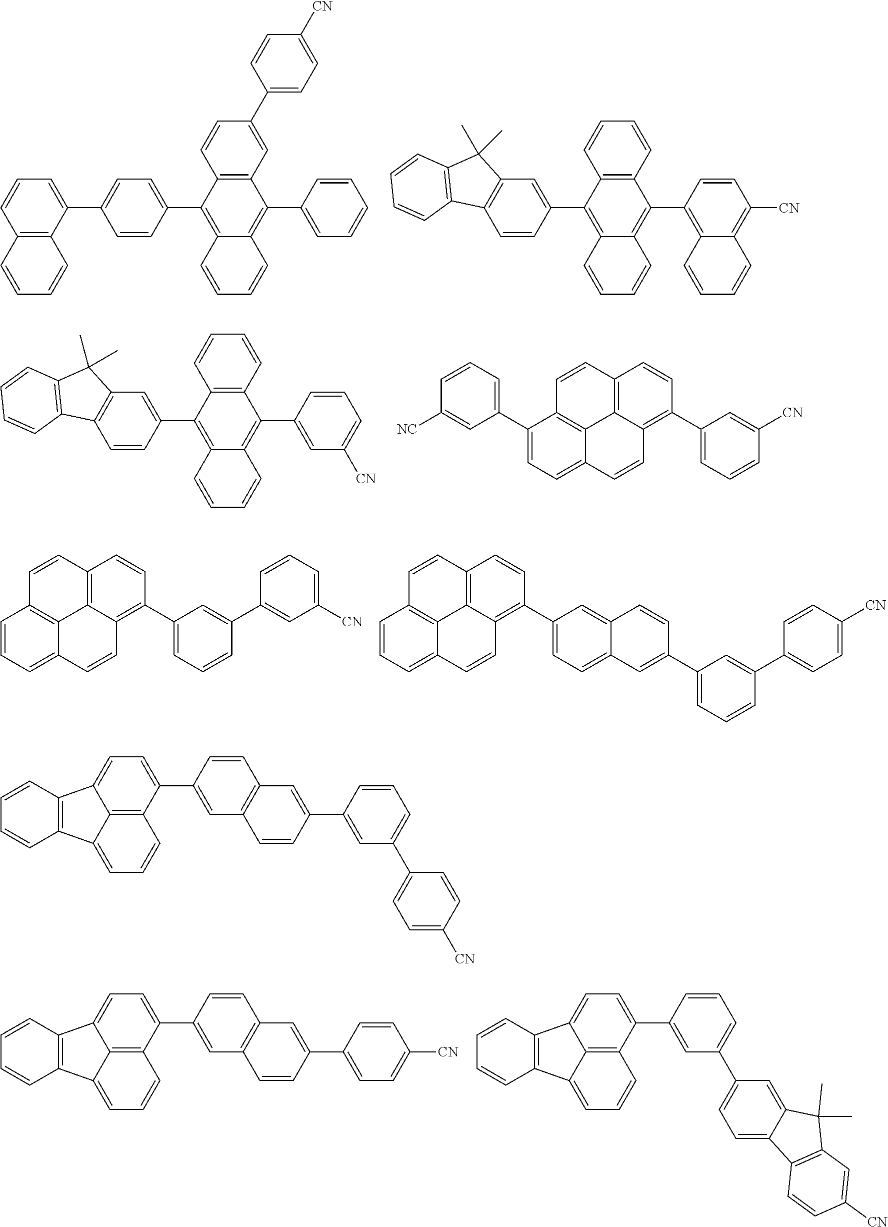

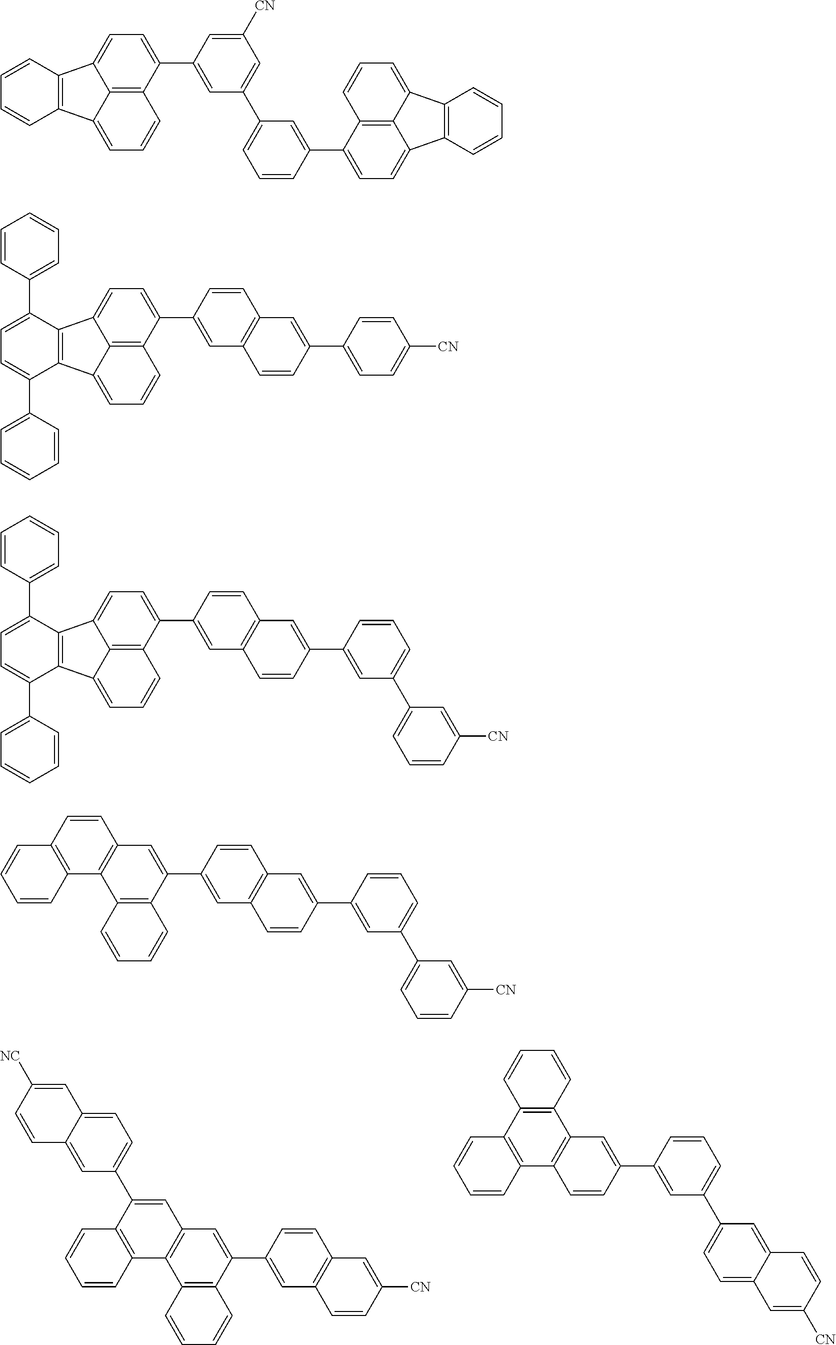

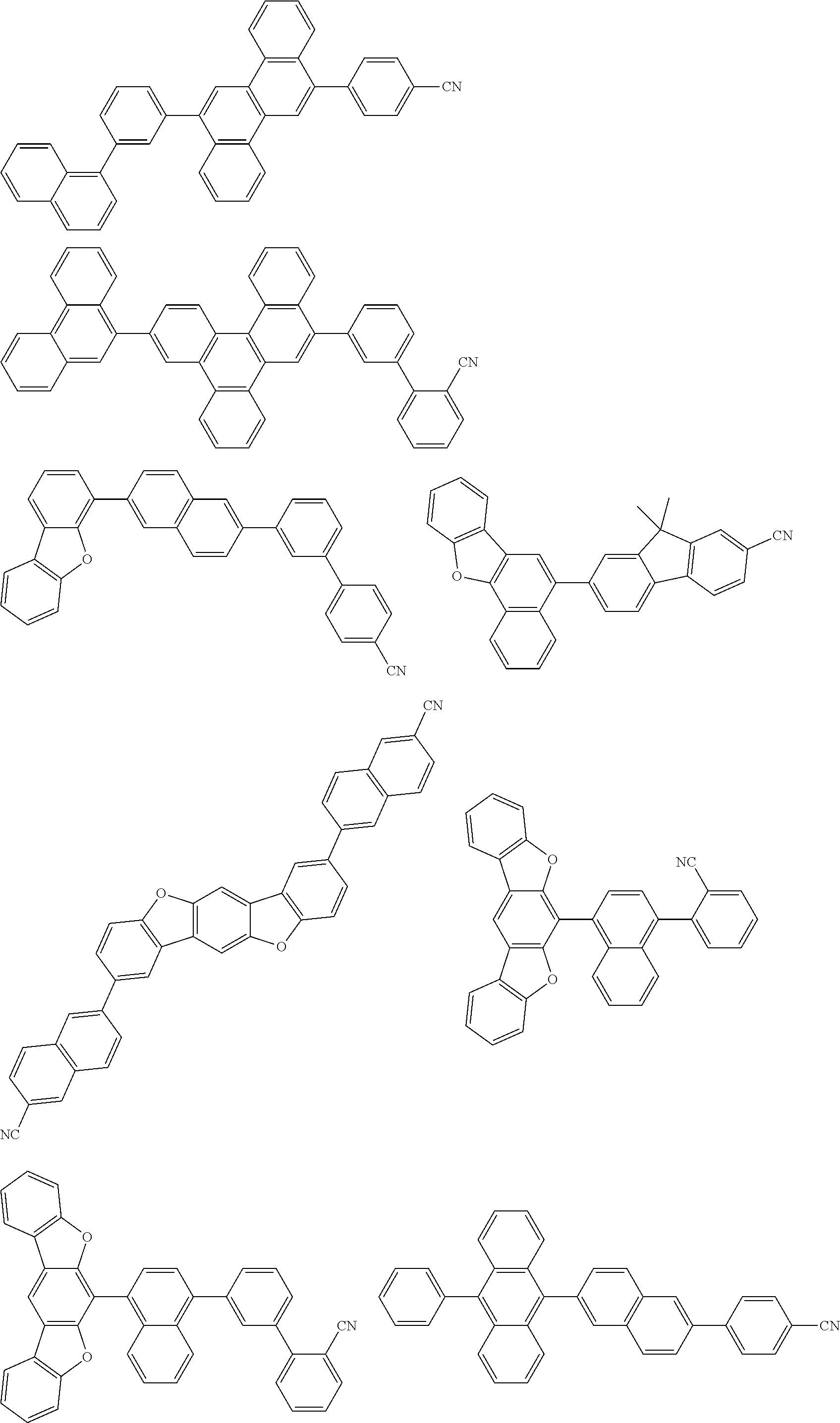

The substituent referred to by “substituent” or “substituted or unsubstituted” each mentioned above is, unless otherwise noted, preferably at least one selected from an alkyl group having 1 to 50, preferably 1 to 18, and more preferably 1 to 8 carbon atoms; a cycloalkyl group having 3 to 50, preferably 3 to 10, more preferably 3 to 8, and still more preferably 5 or 6 ring carbon atoms; an aryl group having 6 to 50, preferably 6 to 25, and more preferably 6 to 18 ring carbon atoms; an aralkyl group having 7 to 51, preferably 7 to 30, and more preferably 7 to 20 carbon atoms, which has an aryl group having 6 to 50, preferably 6 to 25, and more preferably 6 to 18 ring carbon atoms; an amino group; a mono- or di-substituted amino group having a substituent selected from an alkyl group having 1 to 50, preferably 1 to 18, and more preferably 1 to 8 carbon atoms and an aryl group having 6 to 50, preferably 6 to 25, and more preferably 6 to 18 ring carbon atoms; an alkoxy group having 1 to 50, preferably 1 to 18, and more preferably 1 to 8 carbon atoms; an aryloxy group having 6 to 50, preferably 6 to 25, and more preferably 6 to 18 ring carbon atoms; a mono-, di-, or tri-substituted silyl group having a substituent selected from an alkyl group having 1 to 50, preferably 1 to 18, and more preferably 1 to 8 carbon atoms and an aryl group having 6 to 50, preferably 6 to 25, and more preferably 6 to 18 ring carbon atoms; a heteroaryl group having 5 to 50, preferably 5 to 24, and more preferably 5 to 13 ring atoms; a haloalkyl group having 1 to 50, preferably 1 to 18, and more preferably 1 to 8 carbon atoms; a halogen atom; a cyano group; a nitro group; a sulfonyl group having a substituent selected from an alkyl group having 1 to 50, preferably 1 to 18, and more preferably 1 to 8 carbon atoms and an aryl group having 6 to 50, preferably 6 to 25, and more preferably 6 to 18 ring carbon atoms; a di-substituted phosphoryl group having a substituent selected from an alkyl group having 1 to 50, preferably 1 to 18, and more preferably 1 to 8 carbon atoms and an aryl group having 6 to 50, preferably 6 to 25, and more preferably 6 to 18 ring carbon atoms; an alkylsulfonyloxy group; an arylsulfonyloxy group; an alkylcarbonyloxy group; an arylcarbonyloxy group; a boron-containing group; a zinc-containing group; a tin-containing group; a silicon-containing group; a magnesium-containing group; a lithium-containing group; a hydroxyl group; an alkyl-substituted or aryl-substituted carbonyl group; a carboxyl group; a vinyl group; a (meth)acryloyl group; an epoxy group; and an oxetanyl group, although not particularly limited thereto.

The substituent may be further substituted with the optional substituent mentioned above and adjacent two substituents may be bonded to each other to form a ring structure.

The substituent is more preferably a substituted or unsubstituted an alkyl group having 1 to 50, preferably 1 to 18, and more preferably 1 to 8 carbon atoms; a substituted or unsubstituted a cycloalkyl group having 3 to 50, preferably 3 to 10, more preferably 3 to 8, and still more preferably 5 or 6 ring carbon atoms; a substituted or unsubstituted aryl group having 6 to 50, preferably 6 to 25, and more preferably 6 to 18 ring carbon atoms; a mono- or di-substituted amino group having a substituent selected from a substituted or unsubstituted alkyl group having 1 to 50, preferably 1 to 18, and more preferably 1 to 8 carbon atoms and a substituted or unsubstituted aryl group having 6 to 50, preferably 6 to 25, and more preferably 6 to 18 ring carbon atoms; a substituted or unsubstituted heteroaryl group having 5 to 50, preferably 5 to 24, and more preferably 5 to 13 ring atoms, a halogen atom, or a cyano group.

Examples of the alkyl group having 1 to 50 carbon atoms include a methyl group, an ethyl group, a n-propyl group, an isopropyl group, a n-butyl group, an isobutyl group, a s-butyl group, a t-butyl group, a pentyl group (inclusive of isomeric groups), a hexyl group (inclusive of isomeric groups), a heptyl group (inclusive of isomeric groups), an octyl group (inclusive of isomeric groups), a nonyl group (inclusive of isomeric groups), a decyl group (inclusive of isomeric groups), an undecyl group (inclusive of isomeric groups), and a dodecyl group (inclusive of isomeric groups). Preferred are a methyl group, an ethyl group, a n-propyl group, an isopropyl group, a n-butyl group, an isobutyl group, a s-butyl group, a t-butyl group, and a pentyl group (inclusive of isomeric groups), with a methyl group, an ethyl group, a n-propyl group, an isopropyl group, a n-butyl group, an isobutyl group, a s-butyl group, and a t-butyl group being more preferred and a methyl group, an ethyl group, an isopropyl group, and a t-butyl group being particularly preferred.

Examples of the cycloalkyl group having 3 to 50 ring carbon atoms include a cyclopropyl group, a cyclobutyl group, a cyclopentyl group, a cyclohexyl group, a cycloheptyl group, a cyclooctyl group, and an adamantyl group, with a cyclopentyl group and a cyclohexyl group being preferred.

Examples of the aryl group having 6 to 50 ring carbon atoms include a phenyl group, a biphenylyl group, a terphenylyl group, a naphthyl group, an acenaphthylenyl group, an anthryl group, a benzanthryl group, an aceanthryl group, a phenanthryl group, a benzo[c]phenanthryl group, a phenalenyl group, a fluorenyl group, a picenyl group, a pentaphenyl group, a pyrenyl group, a chrysenyl group, a benzo[g]chrysenyl group, a s-indacenyl group, an as-indacenyl group, a fluoranthenyl group, a benzo[k]fluoranthenyl group, a triphenylenyl group, a benzo[b]triphenylenyl group, and a perylenyl group. Preferred are a phenyl group, a biphenylyl group, a terphenylyl group, a naphthyl group, an anthryl group, a pyrenyl group, and a fluoranthenyl group, with a phenyl group, a biphenylyl group, and a terphenylyl group being more preferred and a phenyl group being still more preferred.

In the aralkyl group having 7 to 51 carbon atoms which includes an aryl group having 6 to 50 ring carbon atoms, the details of the aryl portion are as described above with respect to the aryl group having 6 to 50 ring carbon atoms and the details of the alkyl portion are as described above with respect to the alkyl group having 1 to 50 carbon atoms.

In the mono- or di-substituted amino group having a substituent selected from an alkyl group having 1 to 50 carbon atoms and an aryl group having 6 to 50 ring carbon atoms, the details of the aryl portion are as described above with respect to the aryl group having 6 to 50 ring carbon atoms and the details of the alkyl portion are as described above with respect to the alkyl group having 1 to 50 carbon atoms.

The details of the alkyl portion of the alkoxy group having 1 to 50 carbon atoms are as described above with respect to the alkyl group having 1 to 50 carbon atoms.

The details of the aryl portion of the aryloxy group having 6 to 50 ring carbon atoms are as described above with respect to the aryl group having 6 to 50 ring carbon atoms.

Examples of the mono-, di-, or tri-substituted silyl group having a substituent selected from an alkyl group having 1 to 50 carbon atoms and an aryl group having 6 to 50 ring carbon atoms include a monoalkylsilyl group, a dialkylsilyl group, a trialkylsilyl group, a monoarylsilyl group, a diarylsilyl group, a triarylsilyl group, a monoalkyldiarylsilyl group, and a dialkylmonoarylsilyl group. The details of the alkyl portion are as described above with respect to the alkyl group having 1 to 50 carbon atoms and the details of the aryl portion are as described above with respect to the aryl group having 6 to 50 ring carbon atoms.

Examples of the heteroaryl group having 5 to 50 ring atoms include a pyrrolyl group, a furyl group, a thienyl group, a pyridyl group, an imidazopyridyl group, a pyridazinyl group, a pyrimidinyl group, a pyrazinyl group, a triazinyl group, an imidazolyl group, an oxazolyl group, a thiazolyl group, a pyrazolyl group, an isoxazolyl group, an isothiazolyl group, an oxadiazolyl group, a thiadiazolyl group, a triazolyl group, a tetrazolyl group, an indolyl group, an isoindolyl group, a benzofuranyl group, an isobenzofuranyl group, a benzothiophenyl group, an isobenzothiophenyl group, an indolizinyl group, a quinolizinyl group, a quinolyl group, an isoquinolyl group, a cinnolyl group, a phthalazinyl group, a quinazolinyl group, a quinoxalinyl group, a benzimidazolyl group, a benzoxazolyl group, a benzothiazolyl group, an indazolyl group, a benzisoxazolyl group, a benzisothiazolyl group, a dibenzofuranyl group, a dibenzothiophenyl group, a carbazolyl group, a 9-phenylcarbazolyl group, a phenanthridinyl group, an acridinyl group, a phenanthrolinyl group, a phenazinyl group, a phenothiazinyl group, a phenoxazinyl group, and a xanthenyl group. Preferred are a pyridyl group, an imidazopyridyl group, a pyridazinyl group, a pyrimidinyl group, a pyrazinyl group, a triazinyl group, a benzimidazolyl group, a dibenzofuranyl group, a dibenzothiophenyl group, a carbazolyl group, a 9-phenylcarbazolyl group, a phenanthrolinyl group, and a quinazolinyl group.

The halogen atom is a fluorine atom, a chlorine atom, a bromine atom, and an iodine atom.

The haloalkyl group having 1 to 50 carbon atoms is a group derived from the alkyl group having 1 to 50 carbon atoms by replacing at least one hydrogen atom with a halogen atom.

The details of the aryl portion and the alkyl portion of the sulfonyl group having a substituent selected from an alkyl group having 1 to 50 carbon atoms and an aryl group having 6 to 50 ring carbon atoms, the di-substituted phosphoryl group having a substituent selected from an alkyl group having 1 to 50 carbon atoms and an aryl group having 6 to 50 ring carbon atoms, the alkylsulfonyloxy group, the arylsulfonyloxy group, the alkylcarbonyloxy group, the arylcarbonyloxy group, and the alkyl-substitute or aryl-substituted carbonyl group are as described above with respect to the aryl group having 6 to 50 ring carbon atoms and the alkyl group having 1 to 50 carbon atoms, respectively.

An embodiment wherein examples, preferred examples, more preferred examples, etc. of a substituent are combined with examples, preferred examples, more preferred examples, etc. of another substituent is included in the scope of the invention. The same applies to the compounds, the ranges of the number of carbon atoms, and the ranges of the number of atoms. In addition, the substituents, the compounds, the ranges of the number of carbon atoms, and the ranges of the number of atoms may be combined freely and such a combination is included in the scope of the invention.

The organic EL device of the invention is described below in detail. In the following, the “light emitting layer” means a fluorescent emitting layer and a phosphorescent emitting layer, unless otherwise noted.

As described above, the organic EL device of the invention comprises a cathode, an anode, and an organic layer disposed between the cathode and the anode, wherein the organic layer comprises a fluorescent emitting layer. The fluorescent emitting layer comprises at least one first compound selected from the compounds represented by formulae (19), (21), (22), and (23), a second compound selected from the compounds represented by formula (3a), and a dopant material selected from the compounds represented by formulae (D1) and (D2).

The fluorescent emitting layer may be a TADF-based (thermally activated delayed fluorescence-based) light emitting layer. The fluorescent emitting layer does not contain a phosphorescent heavy metal complex, for example, an iridium complex, a platinum complex, an osmium complex, a rhenium complex, and a ruthenium complex.

The organic EL device of the invention may be any of a single color emitting device using fluorescence or thermally activated delayed fluorescence; a white-emitting hybrid device comprising two or more single color emitting devices; an emitting device of a simple type having a single emission unit; and an emitting device of a tandem type having two or more emission units. The “emission unit” referred to herein is the smallest unit for emitting light by the recombination of injected holes and injected electrons, which comprises one or more organic layers wherein at least one layer is a light emitting layer.

Representative device structures of the simple-type organic EL device are shown below.

### (1) Anode/Emission Unit/Cathode

The emission unit described below includes at least one fluorescent emitting layer. The emission unit may be a layered structure comprising two or more light emitting layers selected from a phosphorescent light emitting layer, a fluorescent light emitting layer, and a thermally activated delayed fluorescence-based light emitting layer. A space layer may be disposed between two light emitting layers to prevent the diffusion of excitons generated in the phosphorescent emitting layer into the fluorescent emitting layer. Representative layered structures of the emission unit are shown below, wherein the layer in the parenthesis is optional:
(a) (Hole injecting layer/)Hole transporting layer/Fluorescent emitting layer(/Electron transporting layer/Electron injecting layer);
(b) (Hole injecting layer/)Hole transporting layer/First fluorescent emitting layer/Second fluorescent emitting layer(/Electron transporting layer/Electron injecting layer);
(c) (Hole injecting layer/)Hole transporting layer/Phosphorescent emitting layer/Space layer/Fluorescent emitting layer(/Electron transporting layer/Electron injecting layer);
(d) (Hole injecting layer/)Hole transporting layer/First phosphorescent emitting layer/Second phosphorescent emitting layer/Space layer/Fluorescent emitting layer(/Electron transporting layer/Electron injecting layer);
(e) (Hole injecting layer/)Hole transporting layer/First phosphorescent emitting layer/Space layer/Second phosphorescent emitting layer/Space layer/Fluorescent emitting layer(/Electron transporting layer/Electron injecting layer);
(f) (Hole injecting layer/)Hole transporting layer/Phosphorescent emitting layer/Space layer/First fluorescent emitting layer/Second fluorescent emitting layer(/Electron transporting layer/Electron injecting layer); and
(g) (Hole injecting layer/)First hole transporting layer/Second hole transporting layer/Fluorescent emitting layer/First electron transporting layer/Second electron transporting layer(/Electron injecting layer).

The emission colors of the phosphorescent emitting layers and the fluorescent emitting layer may be different. For example, the layered structure (d) may be Hole transporting layer/First phosphorescent emitting layer (red)/Second phosphorescent emitting layer (green)/Space layer/Fluorescent emitting layer (blue)/Electron transporting layer.

An electron blocking layer may be disposed between the light emitting layer and the hole transporting layer or between the light emitting layer and the space layer, if necessary. Also, a hole blocking layer may be disposed between the light emitting layer and the electron transporting layer, if necessary. With such an electron blocking layer or a hole blocking layer, electrons and holes are confined in the light emitting layer to facilitate the charge recombination in the light emitting layer, thereby improving the emission efficiency.

Representative device structure of the tandem-type organic EL device is shown below.

### (2) Anode/First Emission Unit/Intermediate Layer/Second Emission Unit/Cathode

The layered structure of the first emission unit and the second emission unit may be independently selected from those described above with respect to the emission unit.

Generally, the intermediate layer is also called an intermediate electrode, an intermediate conductive layer, a charge generation layer, an electron withdrawing layer, a connecting layer, or an intermediate insulating layer. The intermediate layer may be formed by known materials so as to supply electrons to the first emission unit and holes to the second emission unit.

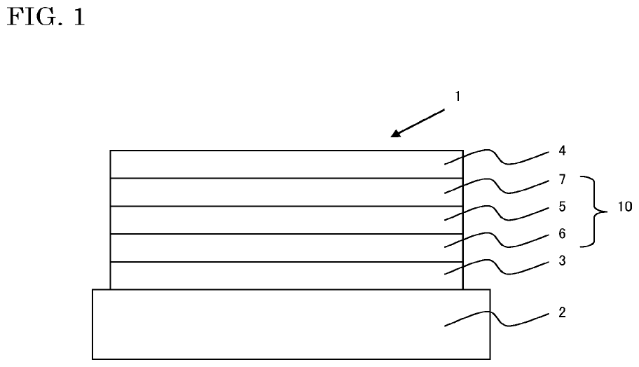

A schematic structure of an example of the organic EL device of the invention is shown in FIG. 1 wherein the organic EL device 1 comprises a substrate 2, an anode 3, a cathode 4, and an emission unit (organic layer) 10 disposed between the anode 3 and the cathode 4. The emission unit 10 comprises a fluorescent emitting layer 5. A hole injecting layer/hole transporting layer 6 may be disposed between the fluorescent emitting layer 5 and the anode 3, and an electron injecting layer/electron transporting layer 7 may be disposed between the fluorescent emitting layer 5 and the cathode 4. An electron blocking layer may be disposed on the anode 3 side of the fluorescent emitting layer 5, and a hole blocking layer may be disposed on the cathode 4 side of the fluorescent emitting layer 5. With these blocking layers, electrons and holes are confined in the fluorescent emitting layer 5 to facilitate the exciton generation in the fluorescent emitting layer 5.

In the present invention, a host material is referred to as a fluorescent host material when combinedly used with a fluorescent dopant material and as a phosphorescent host material when combinedly used with a phosphorescent dopant material. Therefore, the fluorescent host material and the phosphorescent host material are not distinguished from each other merely by the difference in their molecular structures. Namely, in the present invention, the term “fluorescent host material” means a material for constituting a fluorescent emitting layer which contains a fluorescent dopant material and does not mean a material that cannot be used as a material for a phosphorescent emitting layer. The same applies to the phosphorescent host material.

### Substrate

The organic EL device of the invention is formed on a light-transmissive substrate. The light-transmissive substrate serves as a support for the organic EL device and preferably a flat substrate having a transmittance of 50% or more to 400 to 700 nm visible light. Examples of the substrate include a glass plate and a polymer plate. The glass plate may include a plate made of soda-lime glass, barium-strontium-containing glass, lead glass, aluminosilicate glass, borosilicate glass, barium borosilicate glass, or quartz. The polymer plate may include a plate made of polycarbonate, acryl, polyethylene terephthalate, polyether sulfide, or polysulfone.

### Anode

The anode of the organic EL device injects holes to the hole transporting layer or the light emitting layer, and an anode having a work function of 4.5 eV or more is effective. Examples of the material for anode include an indium tin oxide alloy (ITO), tin oxide (NESA), an indium zinc oxide alloy, gold, silver, platinum, and copper. The anode is formed by making the electrode material into a thin film by a method, such as a vapor deposition method or a sputtering method. When getting the light emitted from the light emitting layer through the anode, the transmittance of anode to visible light is preferably 10% or more. The sheet resistance of anode is preferably several hundreds Ω/□ or less. The film thickness of anode depends upon the kind of material and generally 10 nm to 1 μm, preferably 10 to 200 nm.

### Cathode

The cathode injects electrons to the electron injecting layer, the electron transporting layer or the light emitting layer, and is formed preferably by a material having a small work function. Examples of the material for cathode include, but not limited to, indium, aluminum, magnesium, a magnesium-indium alloy, a magnesium-aluminum alloy, an aluminum-lithium alloy, an aluminum-scandium-lithium alloy, and a magnesium-silver alloy. Like the anode, the cathode is formed by making the material into a thin film by a method, such as the vapor deposition method and the sputtering method. The light emitted from a light emitting layer may be taken through the cathode, if necessary.

### Hole Injecting Layer

The hole injecting layer comprises a material having a high hole injecting ability (hole injecting material).

Examples of the hole injecting material include an aromatic amine compound, molybdenum oxide, titanium oxide, vanadium oxide, rhenium oxide, ruthenium oxide, chromium oxide, zirconium oxide, hafnium oxide, tantalum oxide, silver oxide, tungsten oxide, and manganese oxide.

### Hole Transporting Layer

The hole transporting layer is an organic layer formed between the light emitting layer and the anode and transports holes from the anode to the light emitting layer. When the hole transporting layer is formed by two or more layers, the layer closer to the anode may be defined as a hole injecting layer in some cases. The hole injecting layer injects holes from the anode to the organic layer unit efficiently.

An aromatic amine compound, for example, the aromatic amine derivative represented by formula (I) is preferably used as a material for the hole transporting layer:

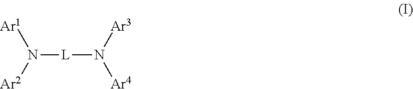

wherein:

Ar¹ to Ar⁴ are each independently a substituted or unsubstituted non-fused aryl group having 6 to 50, preferably 6 to 30, more preferably 6 to 20, and still more preferably 6 to 12 ring carbon atoms, a substituted or unsubstituted fused aryl group having 6 to 50, preferably 6 to 30, more preferably 6 to 20, and still more preferably 6 to 12 ring carbon atoms, a substituted or unsubstituted non-fused heteroaryl group having 5 to 50, preferably 5 to 30, more preferably 5 to 20, and still more preferably 5 to 12 ring atoms, a substituted or unsubstituted fused heteroaryl group having 5 to 50, preferably 5 to 30, more preferably 5 to 20, and still more preferably 5 to 12 ring atoms, or a group wherein the non-fused aryl group or the fused aryl group is bonded to the non-fused heteroaryl group or the fused heteroaryl group;

Ar¹ and Ar², and Ar³ and Ar⁴ may be bonded to each other to form a ring; and

L represents a substituted or unsubstituted non-fused arylene group having 6 to 50, preferably 6 to 30, more preferably 6 to 20, and still more preferably 6 to 12 ring carbon atoms, a substituted or unsubstituted fused arylene group having 6 to 50, preferably 6 to 30, more preferably 6 to 20, and still more preferably 6 to 12 ring carbon atoms, a substituted or unsubstituted non-fused heteroarylene group having 5 to 50, preferably 5 to 30, more preferably 5 to 20, and still more preferably 5 to 12 ring atoms, or a substituted or unsubstituted fused heteroarylene group having 5 to 50, preferably 5 to 30, more preferably 5 to 20, and still more preferably 5 to 12 ring atoms.

Examples of the compound represented by formula (I) are shown below.

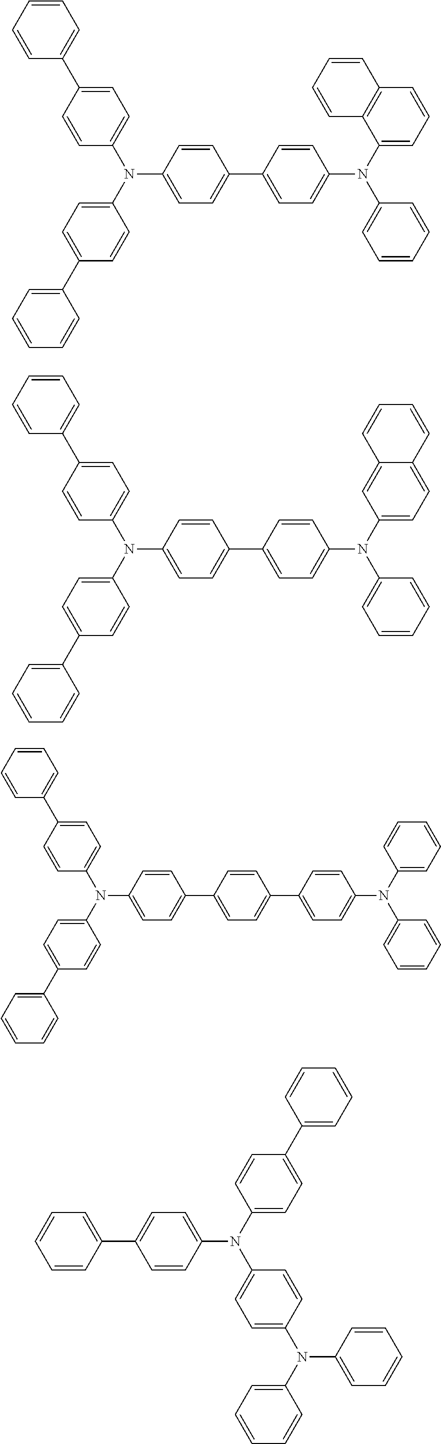

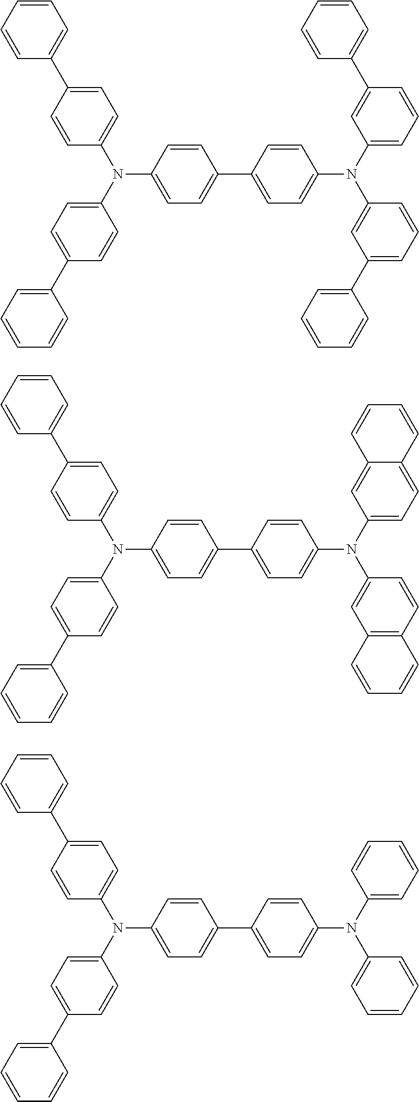

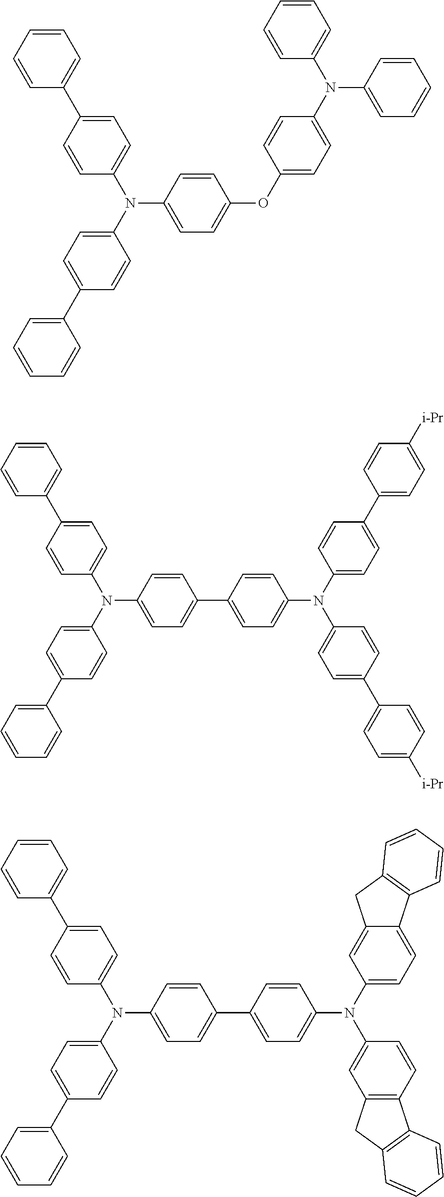

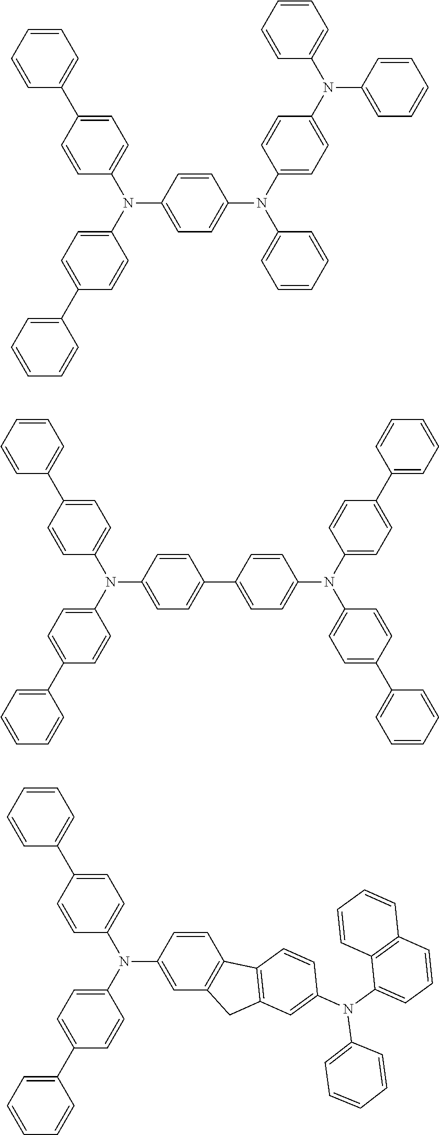

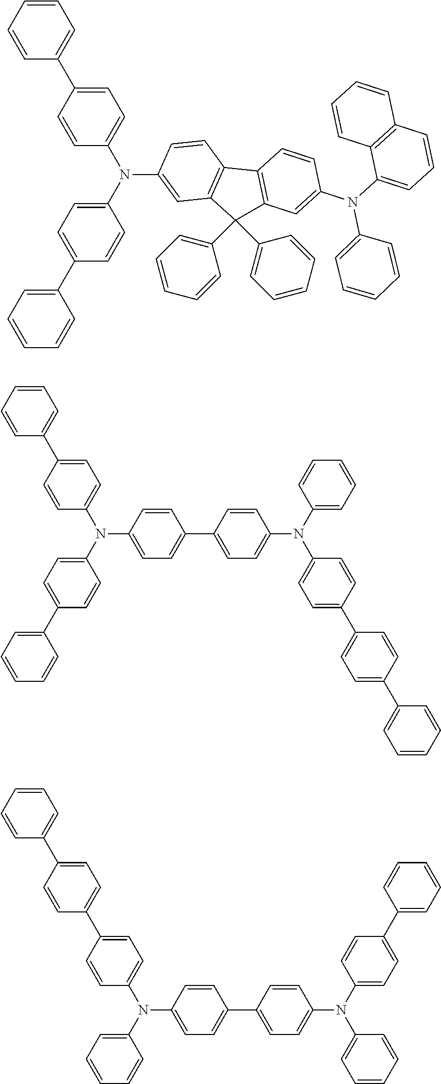 [SMILES: c1ccc(-c2ccc(N(c3ccc(-c4ccccc4)cc3)c3ccc4c(c3)C(c3ccccc3)(c3ccccc3)c3cc(N(c5ccccc5)c5cccc6ccccc56)ccc3-4)cc2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(-c2ccc(-c3ccc(N(c4ccccc4)c4ccc(-c5ccc(N(c6ccccc6)c6ccc(-c7ccccc7)cc6)cc5)cc4)cc3)cc2)cc1]

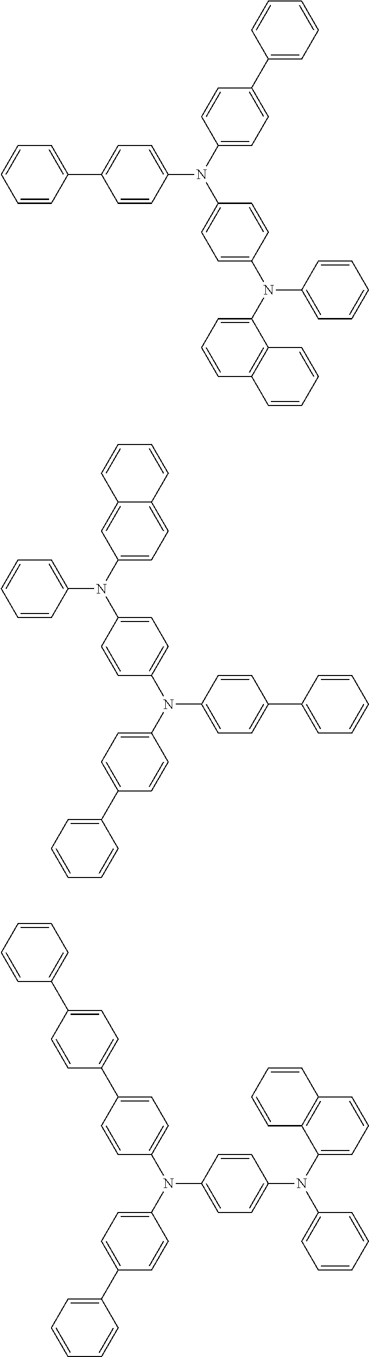 [SMILES: c1ccc(-c2ccc(N(c3ccc(-c4ccccc4)cc3)c3ccc(N(c4ccccc4)c4cccc5ccccc45)cc3)cc2)cc1 ; c1ccc(-c2ccc(N(c3ccc(-c4ccccc4)cc3)c3ccc(N(c4ccccc4)c4ccc5ccccc5c4)cc3)cc2)cc1 ; c1ccc(-c2ccc(-c3ccc(N(c4ccc(-c5ccccc5)cc4)c4ccc(N(c5ccccc5)c5cccc6ccccc56)cc4)cc3)cc2)cc1]

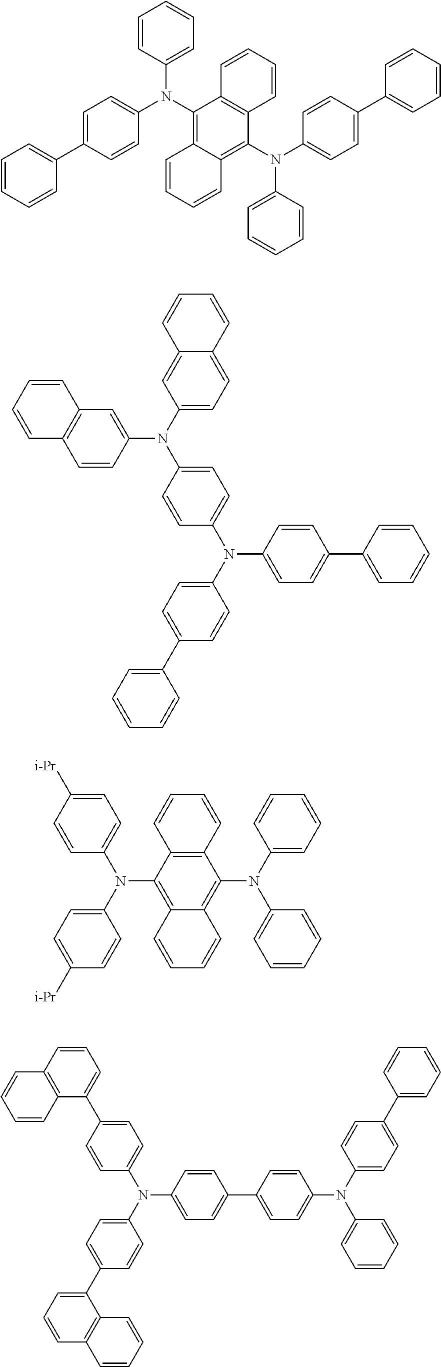 [SMILES: c1ccc(-c2ccc(N(c3ccccc3)c3c4ccccc4c(N(c4ccccc4)c4ccc(-c5ccccc5)cc4)c4ccccc34)cc2)cc1 ; c1ccc(-c2ccc(N(c3ccc(-c4ccccc4)cc3)c3ccc(N(c4ccc5ccccc5c4)c4ccc5ccccc5c4)cc3)cc2)cc1 ; CC(C)c1ccc(N(c2ccc(C(C)C)cc2)c2c3ccccc3c(N(c3ccccc3)c3ccccc3)c3ccccc23)cc1 ; c1ccc(-c2ccc(N(c3ccccc3)c3ccc(-c4ccc(N(c5ccc(-c6cccc7ccccc67)cc5)c5ccc(-c6cccc7ccccc67)cc5)cc4)cc3)cc2)cc1]

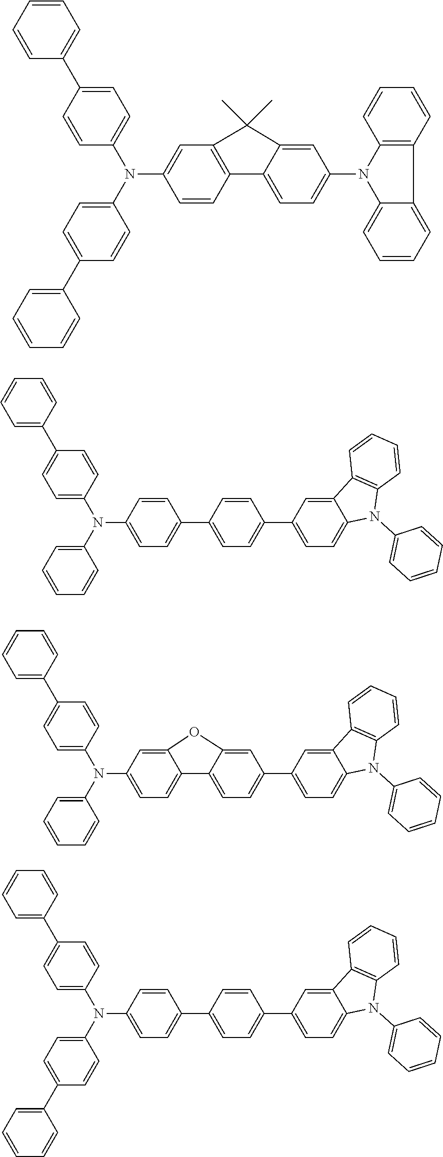

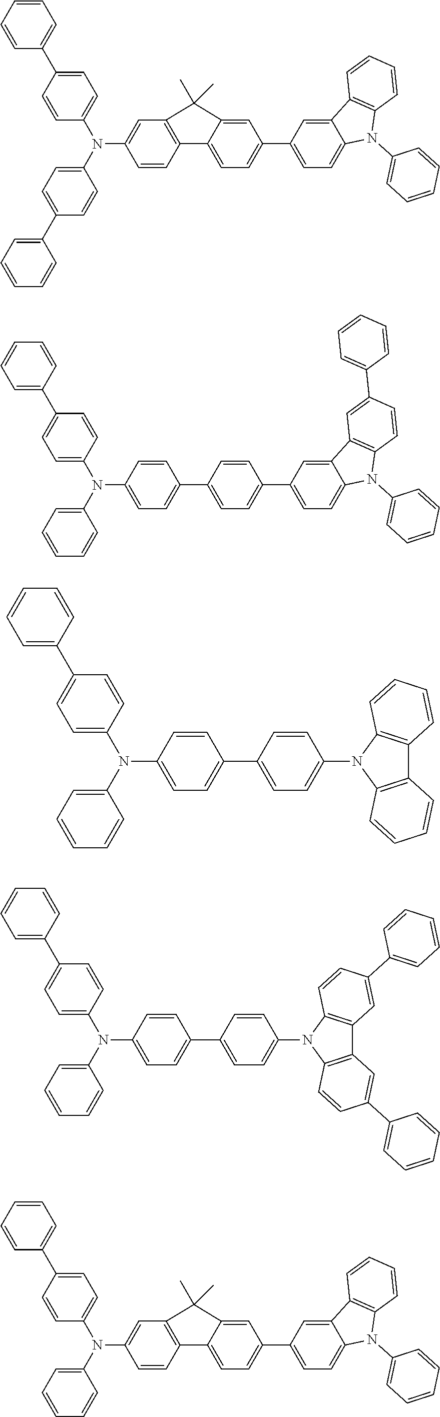

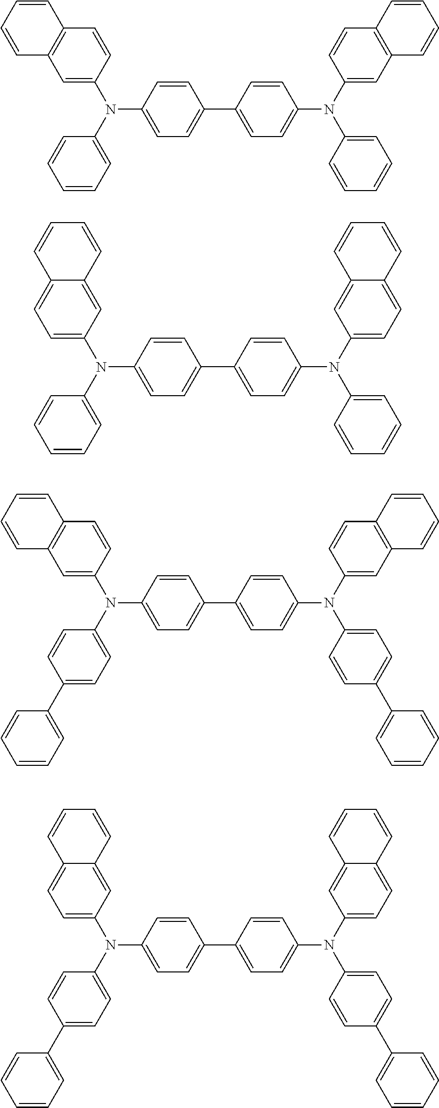

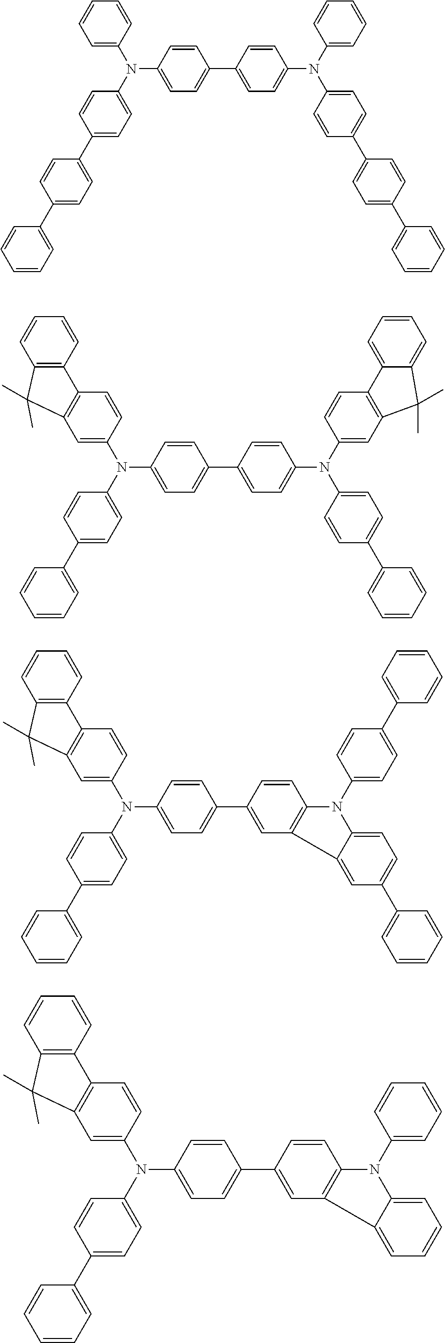

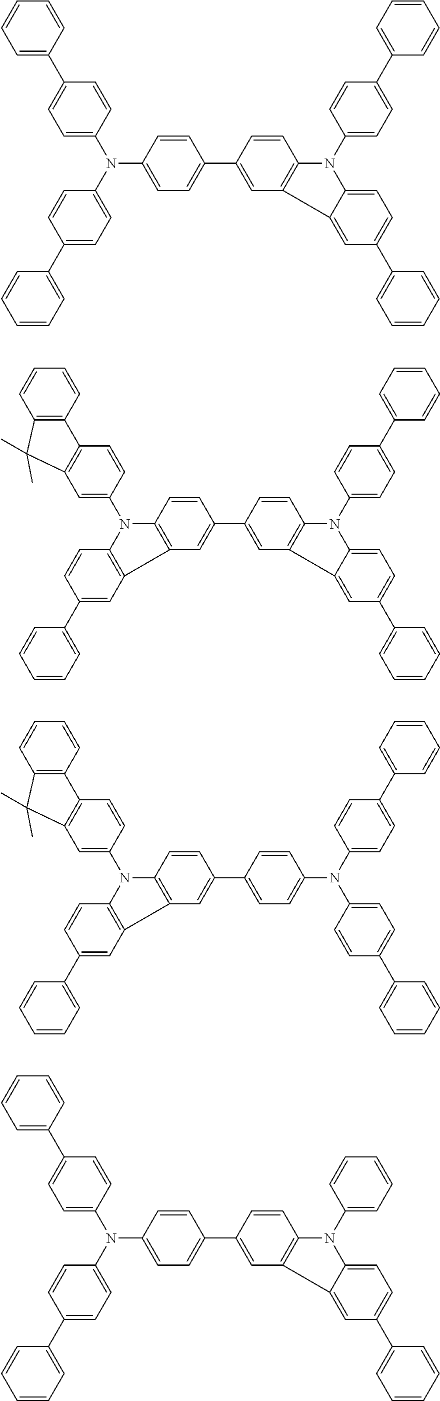

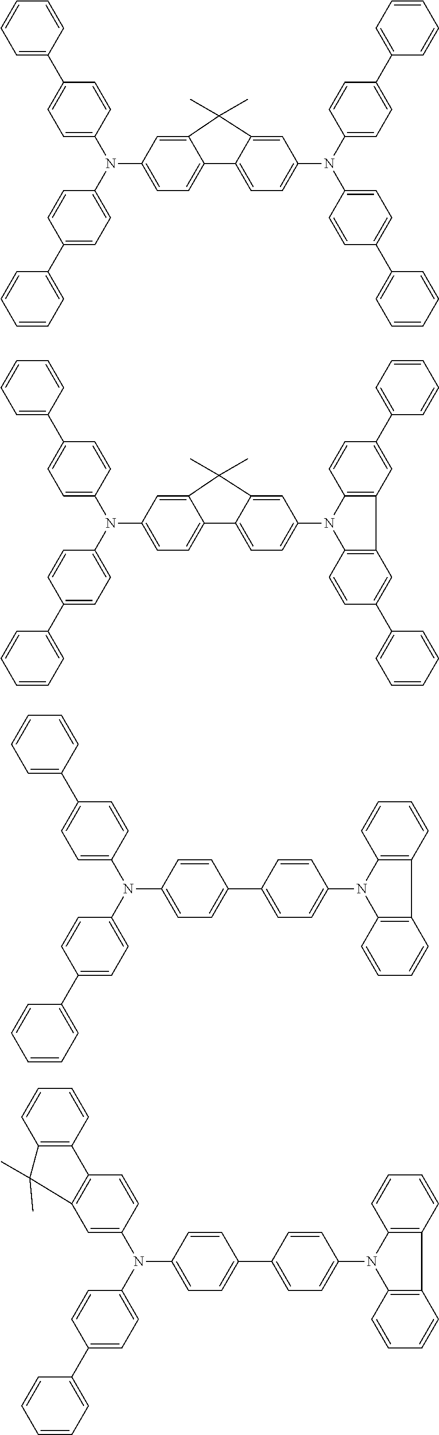

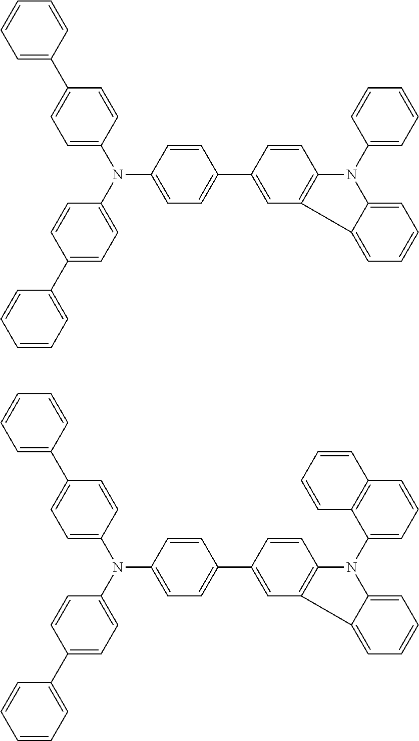

An aromatic amine represented by formula (II) is also preferred as the hole transporting layer material:

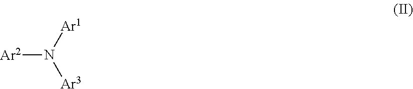

wherein Ar¹ to Ar³ are as defined above with respect to Ar¹ to Ar⁴ of formula (I).

Examples of the compound represented by formula (II) are shown below, although not limited thereto.

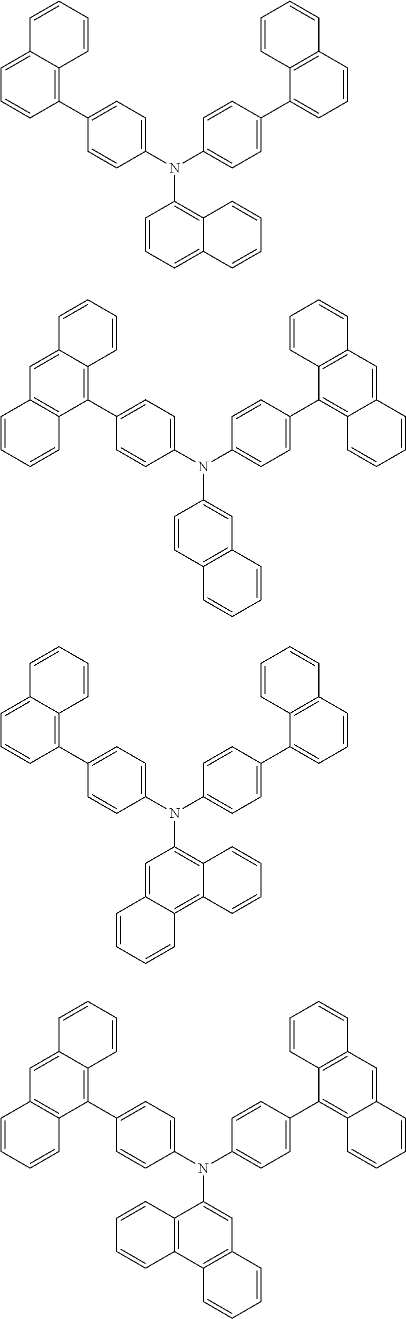 [SMILES: c1ccc2c(-c3ccc(N(c4ccc(-c5cccc6ccccc56)cc4)c4cccc5ccccc45)cc3)cccc2c1 ; c1ccc2cc(N(c3ccc(-c4c5ccccc5cc5ccccc45)cc3)c3ccc(-c4c5ccccc5cc5ccccc45)cc3)ccc2c1 ; c1ccc2c(-c3ccc(N(c4ccc(-c5cccc6ccccc56)cc4)c4cc5ccccc5c5ccccc45)cc3)cccc2c1 ; c1ccc2c(-c3ccc(N(c4ccc(-c5c6ccccc6cc6ccccc56)cc4)c4cc5ccccc5c5ccccc45)cc3)c3ccccc3cc2c1]

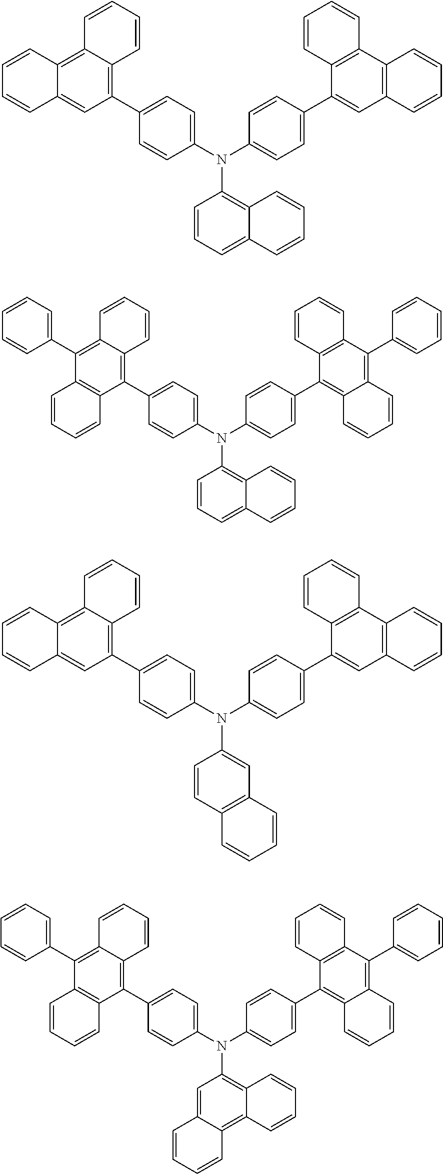 [SMILES: c1ccc2c(N(c3ccc(-c4cc5ccccc5c5ccccc45)cc3)c3ccc(-c4cc5ccccc5c5ccccc45)cc3)cccc2c1 ; c1ccc(-c2c3ccccc3c(-c3ccc(N(c4ccc(-c5c6ccccc6c(-c6ccccc6)c6ccccc56)cc4)c4cccc5ccccc45)cc3)c3ccccc23)cc1 ; c1ccc2cc(N(c3ccc(-c4cc5ccccc5c5ccccc45)cc3)c3ccc(-c4cc5ccccc5c5ccccc45)cc3)ccc2c1 ; c1ccc(-c2c3ccccc3c(-c3ccc(N(c4ccc(-c5c6ccccc6c(-c6ccccc6)c6ccccc56)cc4)c4cc5ccccc5c5ccccc45)cc3)c3ccccc23)cc1]

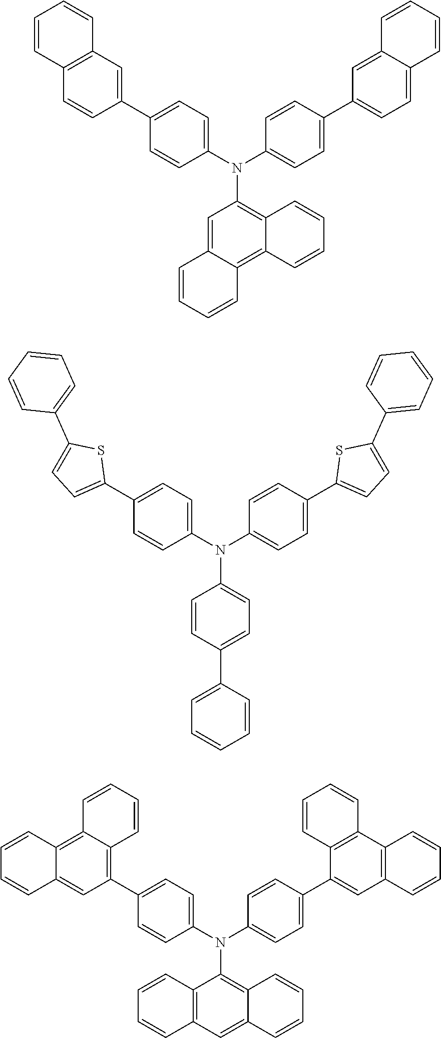 [SMILES: c1ccc2cc(-c3ccc(N(c4ccc(-c5ccc6ccccc6c5)cc4)c4cc5ccccc5c5ccccc45)cc3)ccc2c1 ; c1ccc(-c2ccc(N(c3ccc(-c4ccc(-c5ccccc5)s4)cc3)c3ccc(-c4ccc(-c5ccccc5)s4)cc3)cc2)cc1 ; c1ccc2c(N(c3ccc(-c4cc5ccccc5c5ccccc45)cc3)c3ccc(-c4cc5ccccc5c5ccccc45)cc3)c3ccccc3cc2c1]

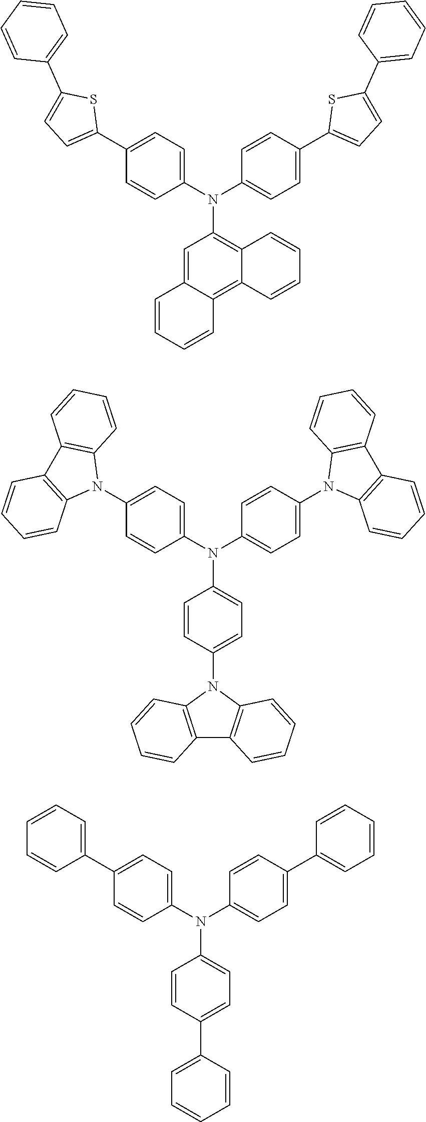 [SMILES: c1ccc(-c2ccc(-c3ccc(N(c4ccc(-c5ccc(-c6ccccc6)s5)cc4)c4cc5ccccc5c5ccccc45)cc3)s2)cc1 ; c1ccc2c(c1)c1ccccc1n2-c1ccc(N(c2ccc(-n3c4ccccc4c4ccccc43)cc2)c2ccc(-n3c4ccccc4c4ccccc43)cc2)cc1 ; c1ccc(-c2ccc(N(c3ccc(-c4ccccc4)cc3)c3ccc(-c4ccccc4)cc3)cc2)cc1]

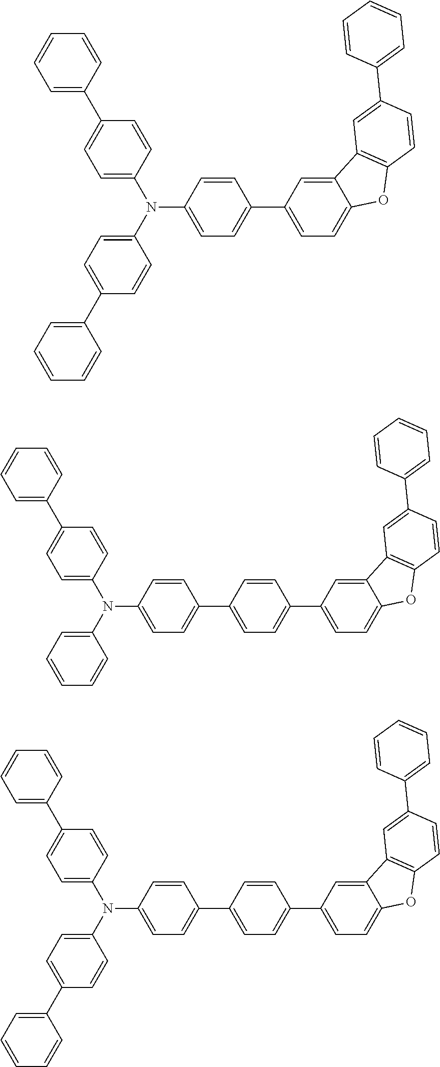 [SMILES: c1ccc(-c2ccc(N(c3ccc(-c4ccccc4)cc3)c3ccc(-c4ccc5oc6ccc(-c7ccccc7)cc6c5c4)cc3)cc2)cc1 ; c1ccc(-c2ccc(N(c3ccccc3)c3ccc(-c4ccc(-c5ccc6oc7ccc(-c8ccccc8)cc7c6c5)cc4)cc3)cc2)cc1 ; c1ccc(-c2ccc(N(c3ccc(-c4ccccc4)cc3)c3ccc(-c4ccc(-c5ccc6oc7ccc(-c8ccccc8)cc7c6c5)cc4)cc3)cc2)cc1]

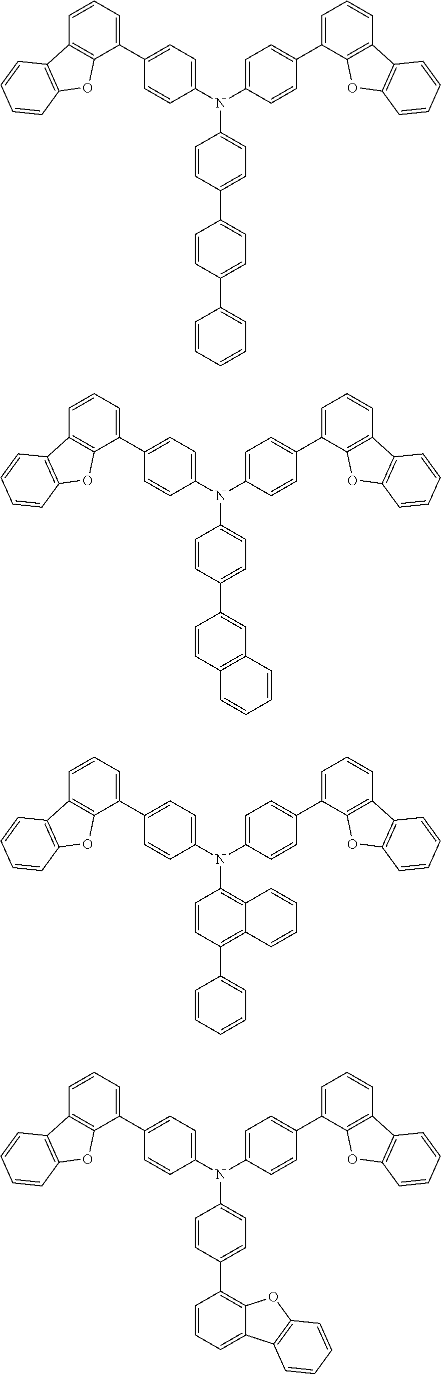 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc2cc(-c3ccc(N(c4ccc(-c5cccc6c5oc5ccccc56)cc4)c4ccc(-c5cccc6c5oc5ccccc56)cc4)cc3)ccc2c1 ; c1ccc(-c2ccc(N(c3ccc(-c4cccc5c4oc4ccccc45)cc3)c3ccc(-c4cccc5c4oc4ccccc45)cc3)c3ccccc23)cc1 ; c1ccc2c(c1)oc1c(-c3ccc(N(c4ccc(-c5cccc6c5oc5ccccc56)cc4)c4ccc(-c5cccc6c5oc5ccccc56)cc4)cc3)cccc12]

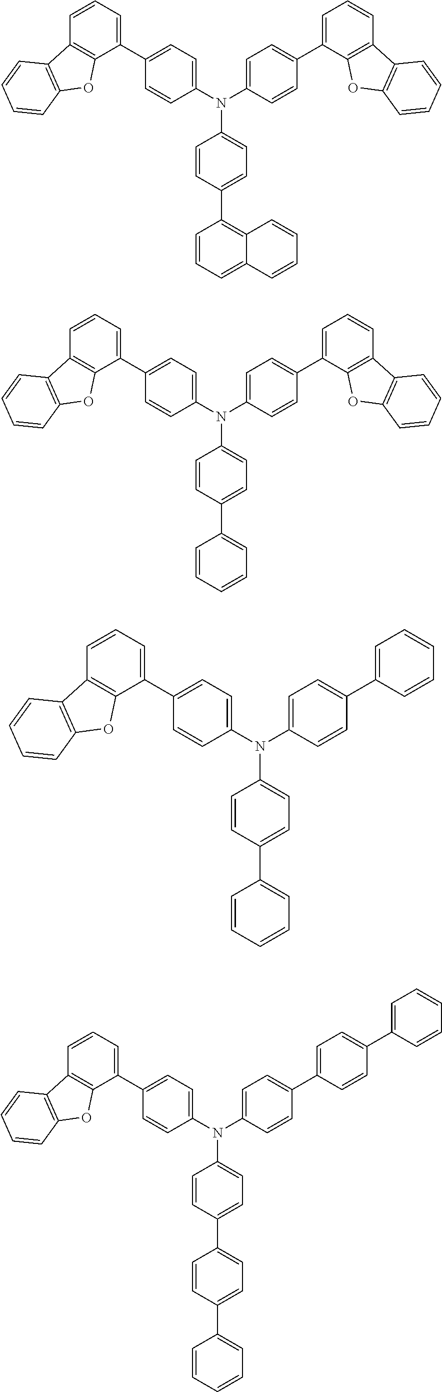 [SMILES: c1ccc2c(-c3ccc(N(c4ccc(-c5cccc6c5oc5ccccc56)cc4)c4ccc(-c5cccc6c5oc5ccccc56)cc4)cc3)cccc2c1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccccc4)cc3)c3ccc(-c4cccc5c4oc4ccccc45)cc3)cc2)cc1 ; c1ccc(-c2ccc(-c3ccc(N(c4ccc(-c5ccc(-c6ccccc6)cc5)cc4)c4ccc(-c5cccc6c5oc5ccccc56)cc4)cc3)cc2)cc1]

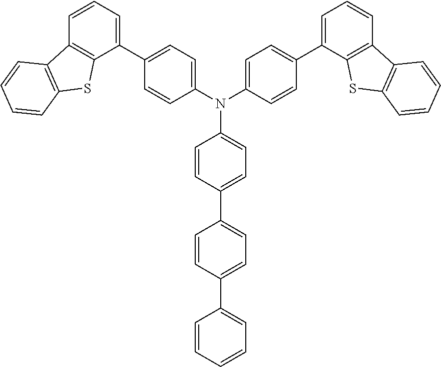 [SMILES: c1ccc(-c2ccc(-c3ccc(N(c4ccc(-c5cccc6c5sc5ccccc56)cc4)c4ccc(-c5cccc6c5sc5ccccc56)cc4)cc3)cc2)cc1]

The hole transporting layer may be made into two-layered structure of a first hole transporting layer (anode side) and a second hole transporting layer (cathode side).

The thickness of the hole transporting layer is preferably 10 to 200 nm, although not particularly limited thereto. If the hole transporting layer is of a two-layered structure of a first hole transporting layer (anode side) and a second hole transporting layer (cathode side), the thickness is preferably 50 to 150 nm and more preferably 50 to 110 nm for the first hole transporting layer, and preferably 5 to 50 nm and more preferably 5 to 30 nm for the second hole transporting layer.

A layer comprising an acceptor material may be disposed in contact with the anode side of the hole transporting layer or the first hole transporting layer. With such a layer, it is expected that the driving voltage is lowered and the production cost is reduced.

The acceptor material is preferably a compound represented by the following formula:

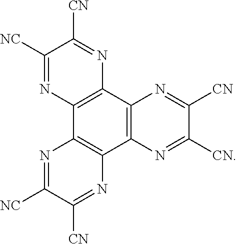 [SMILES: N#Cc1nc2c3nc(C#N)c(C#N)nc3c3nc(C#N)c(C#N)nc3c2nc1C#N]

The thickness of the layer comprising the acceptor material is preferably 5 to 20 nm, although not particularly limited thereto.

### Light Emitting Layer

The light emitting layer is an organic layer having a light emitting function and contains a host material and a dopant material when a doping system is employed. The major function of the host material is to promote the recombination of electrons and holes and confine excitons in the light emitting layer. The dopant material causes the excitons generated by recombination to emit light efficiently.

In case of a phosphorescent device, the major function of the host material is to confine the excitons generated on the dopant in the light emitting layer.

The light emitting layer may be made into a double dopant layer, in which two or more kinds of dopant materials having high quantum yield are combinedly used and each dopant material emits light with its own color. For example, a yellow-emitting layer is obtained by co-depositing a host material, a red-emitting dopant material and a green-emitting dopant material into a single emitting layer.

The easiness of hole injection to the light emitting layer and the easiness of electron injection to the light emitting layer may be different from each other. Also, the hole transporting ability expressed by hole mobility and the electron transporting ability expressed by electron mobility in the light emitting layer may be different from each other.

The light emitting layer is formed, for example, by a known method, such as a vapor deposition method, a spin coating method, and LB method. The light emitting layer may be also formed by making a solution of a binder, such as resin, and a material for the light emitting layer into a thin film by a method such as spin coating.

The light emitting layer is preferably a molecular deposit film. The molecular deposit film is a thin film formed by depositing a vaporized material or a film formed by solidifying a material in the form of solution or liquid. The molecular deposit film can be distinguished from a thin film formed by LB method (molecular build-up film) by the differences in the assembly structures and higher order structures and the functional difference due to the structural differences.

The thickness of the light emitting layer is preferably 5 to 50 nm, more preferably 7 to 50 nm, and still more preferably 10 to 50 nm. If being 5 nm or more, the light emitting layer is formed easily. If being 50 nm or less, the driving voltage is prevented from increasing.

### Dopant Material

The fluorescent dopant material (fluorescent emitting material) is a compound emitting light by releasing the energy of excited singlet state. A fluorescent dopant material other than the compounds represented by formulae (D1) and (D2) may be used. Such a fluorescent dopant material is not particularly limited as long as emitting light by releasing the energy of excited singlet state. Examples thereof include a fluoranthene derivative, a styrylarylene derivative, a pyrene derivative, an arylacetylene derivative, a fluorene derivative, a boron complex, a perylene derivative, an oxadiazole derivative, an anthracene derivative, a styrylamine derivative, and an arylamine derivative, with an anthracene derivative, a fluoranthene derivative, a styrylamine derivative, an arylamine derivative, a styrylarylene derivative, a pyrene derivative, and a boron complex being preferred, and an anthracene derivative, a fluoranthene derivative, a styrylamine derivative, an arylamine derivative, and a boron complex compound being more preferred.

The phosphorescent dopant material (phosphorescent emitting material) is a compound emitting light by releasing the energy of excited triplet state. Examples of the phosphorescent dopant material include a metal complex, such as an iridium complex, a platinum complex, an osmium complex, a rhenium complex, and a ruthenium complex.

### Host Material

In an embodiment of the invention, the fluorescent emitting layer comprises at least one first compound selected from the compounds represented by formulae (19), (21), (22), and (23) as the host material (main host material) and the second compound selected from the compounds represented by formula (3a) as the co-host material.

Another host material usable in the light emitting layer may include, for example, a metal complex, such as an aluminum complex, a beryllium complex, and a zinc complex; a heterocyclic compound, such as an oxadiazole derivative, a benzimidazole derivative, and a phenanthroline derivative; a fused aromatic compound, such as a carbazole derivative, an anthracene derivative, a phenanthrene derivative, a pyrene derivative, a chrysene derivative, and a fluorene derivative; and an aromatic amine compound, such as a triarylamine derivative and a fused aromatic polycyclic amine derivative.

### Electron Transporting Layer

The electron transporting layer is an organic layer disposed between the light emitting layer and the cathode and transports electrons from the cathode to the light emitting layer.

An aromatic heterocyclic compound having one or more hetero atoms in its molecule is preferably used as an electron transporting material used in the electron transporting layer, and a nitrogen-containing ring derivative is particularly preferred. In addition, the nitrogen-containing ring derivative is preferably an aromatic heterocyclic compound having a nitrogen-containing, 6- or 5-membered ring, or a fused aromatic heterocyclic compound having a nitrogen-containing, 6- or 5-membered ring.

The nitrogen-containing ring derivative is preferably, for example, a metal chelate complex of a nitrogen-containing ring represented by formula (A):

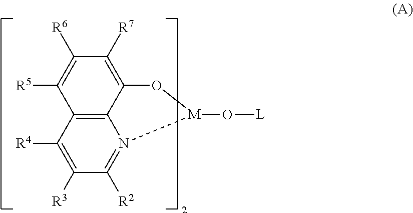

wherein:

each of R² to R⁷ independently represents a hydrogen atom, a halogen atom, a hydroxyl group, an amino group, a hydrocarbon group having 1 to 40, preferably 1 to 20, more preferably 1 to 10, and still more preferably 1 to 6 carbon atoms, an alkoxy group having 1 to 40, preferably 1 to 20, more preferably 1 to 10, and still more preferably 1 to 6 carbon atoms, an aryloxy group having 6 to 40, preferably 6 to 20, and more preferably 6 to 12 ring carbon atoms, an alkoxycarbonyl group having 2 to 40, preferably 2 to 20, more preferably 2 to 10, and still more preferably 2 to 5 carbon atoms, or an aromatic heterocyclic group having 9 to 40, preferably 9 to 30, and more preferably 9 to 20 ring atoms, each optionally having a substituent;

M is aluminum, gallium, or indium, with In being preferred; and

L is a group represented by formula (A′) or (A″):

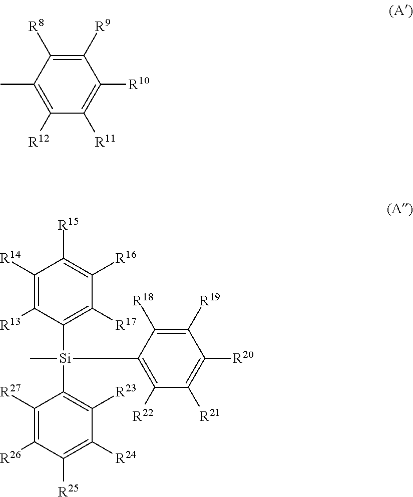

wherein:

each R⁸ to R¹² in formula (A′) independently represents a hydrogen atom or a substituted or unsubstituted hydrocarbon group having 1 to 40, preferably 1 to 20, more preferably 1 to 10, and still more preferably 1 to 6 carbon atoms and adjacent two may form a ring structure;

each of R¹³ to R²⁷ in formula (A″) independently represents a hydrogen atom or a substituted or unsubstituted hydrocarbon group having 1 to 40, preferably 1 to 20, more preferably 1 to 10, and still more preferably 1 to 6 carbon atoms and adjacent two may form a ring structure.

Examples of the divalent group formed by adjacent two of R⁸ to R¹² and R¹³ to R²⁷ which completes the ring structure include a tetramethylene group, a pentamethylene group, a hexamethylene group, a diphenylmethane-2,2′-diyl group, a diphenylethane-3,3′-diyl group, and a diphenylpropane-4,4′-diyl group.

A metal complex including 8-hydroxyquinoline or its derivative, an oxadiazole derivative, and a nitrogen-containing heterocyclic derivative are also preferably as the electron transporting material for used in the electron transporting layer.

An electron transporting material having a good thin film-forming property is preferably used. Examples of the electron transporting compound are shown below.

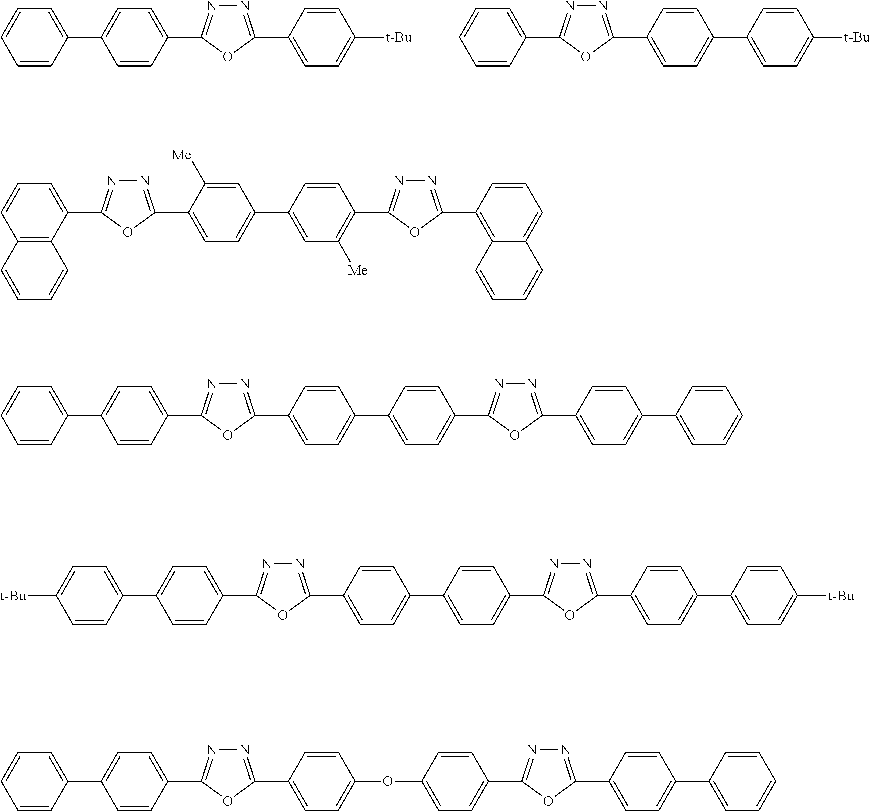 [SMILES: CC(C)(C)c1ccc(-c2nnc(-c3ccc(-c4ccccc4)cc3)o2)cc1 ; CC(C)(C)c1ccc(-c2ccc(-c3nnc(-c4ccccc4)o3)cc2)cc1 ; Cc1cc(-c2ccc(-c3nnc(-c4cccc5ccccc45)o3)c(C)c2)ccc1-c1nnc(-c2cccc3ccccc23)o1 ; c1ccc(-c2ccc(-c3nnc(-c4ccc(-c5ccc(-c6nnc(-c7ccc(-c8ccccc8)cc7)o6)cc5)cc4)o3)cc2)cc1 ; CC(C)(C)c1ccc(-c2ccc(-c3nnc(-c4ccc(-c5ccc(-c6nnc(-c7ccc(-c8ccc(C(C)(C)C)cc8)cc7)o6)cc5)cc4)o3)cc2)cc1 ; c1ccc(-c2ccc(-c3nnc(-c4ccc(Oc5ccc(-c6nnc(-c7ccc(-c8ccccc8)cc7)o6)cc5)cc4)o3)cc2)cc1]

A compound having a nitrogen-containing heterocyclic group represented by any of the following formulae is also preferred as the electron transporting material for the electron transporting layer.

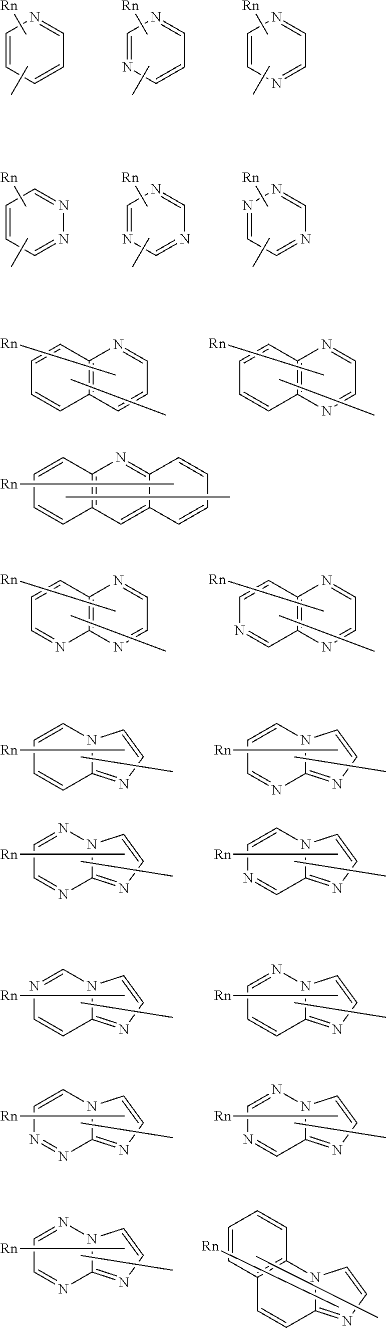

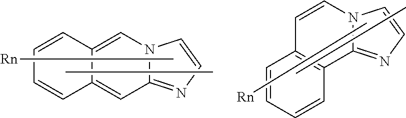

wherein:

R is a non-fused aryl group having 6 to 40 ring carbon atoms, a fused aromatic hydrocarbon group having 10 to 40 ring carbon atoms, a non-fused heteroaryl group having 3 to 40 ring atoms, a used heteroaryl group having 3 to 40 ring atoms, an alkyl group having 1 to 20 carbon atoms, or an alkoxy group having 1 to 20 carbon atoms;

n is an integer of 0 to 5; and

when n is an integer of 2 or more, groups R may be the same or different.

The electron transporting layer particularly preferably comprises at least one compound selected from the nitrogen-containing heterocyclic derivatives represented by formulae (60) to (62):

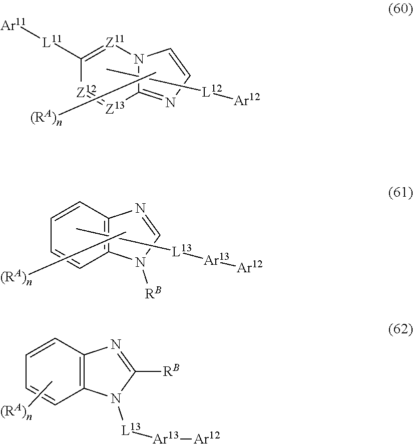

wherein:

Z¹¹, Z¹², and Z¹³ are each independently a nitrogen atom or a carbon atom;

R^{A} and R^{B} are each independently a substituted or unsubstituted aryl group having 6 to 50, preferably 6 to 30, more preferably 6 to 20, and still more preferably 6 to 12 ring carbon atoms, a substituted or unsubstituted heteroaryl group having 5 to 50, preferably 5 to 30, more preferably 5 to 20, and still more preferably 5 to 12 ring atoms, a substituted or unsubstituted alkyl group having 1 to 20, preferably 1 to 10, and more preferably 1 to 6 carbon atoms, a substituted or unsubstituted haloalkyl group having 1 to 20, preferably 1 to 10, and more preferably 1 to 6 carbon atoms, or a substituted or unsubstituted alkoxyl group having 1 to 20, preferably 1 to 10, and more preferably 1 to 6 carbon atoms;

n is an integer of 0 to 5, when n is an integer of 2 or more, R^{A}'s may be the same or different, and adjacent two R^{A}'s may be bonded to each other to form a substituted or unsubstituted hydrocarbon ring;

Ar¹¹ is a substituted or unsubstituted aryl group having 6 to 50, preferably 6 to 30, more preferably 6 to 20, and still more preferably 6 to 12 ring carbon atoms or a substituted or unsubstituted heteroaryl group having 5 to 50, preferably 5 to 30, more preferably 5 to 20, and still more preferably 5 to 12 ring atoms;

Ar¹² is a hydrogen atom, a substituted or unsubstituted alkyl group having 1 to 20, preferably 1 to 10, and more preferably 1 to 6 carbon atoms, a substituted or unsubstituted haloalkyl group having 1 to 20, preferably 1 to 10, and more preferably 1 to 6 carbon atoms, a substituted or unsubstituted alkoxyl group having 1 to 20, preferably 1 to 10, and more preferably 1 to 6 carbon atoms, a substituted or unsubstituted aryl group having 6 to 50, preferably 6 to 30, more preferably 6 to 20, and still more preferably 6 to 12 ring carbon atoms, or a substituted or unsubstituted heteroaryl group having 5 to 50, preferably 5 to 30, more preferably 5 to 20, and still more preferably 5 to 12 ring atoms;

provided that one of Ar¹¹ and Ar¹² is a substituted or unsubstituted fused aryl group having 10 to 50, preferably 10 to 30, more preferably 10 to 20, and still more preferably 10 to 14 ring carbon atoms or a substituted or unsubstituted fused heteroaryl group having 9 to 50, preferably 9 to 30, more preferably 9 to 20, and still more preferably 9 to 14 ring atoms;

Ar¹³ is a substituted or unsubstituted arylene group having 6 to 50, preferably 6 to 30, more preferably 6 to 20, and still more preferably 6 to 12 ring carbon atoms or a substituted or unsubstituted heteroarylene group having 5 to 50, preferably 5 to 30, more preferably 5 to 20, and still more preferably 5 to 12 ring atoms; and

L¹¹, L¹², and L¹³ each independently represent a single bond, a substituted or unsubstituted arylene group having 6 to 50, preferably 6 to 30, more preferably 6 to 20, and still more preferably 6 to 12 ring carbon atoms or a substituted or unsubstituted divalent fused aromatic heterocyclic group having 9 to 50, preferably 9 to 30, more preferably 9 to 20, and still more preferably 9 to 14 ring atoms.

Examples of the nitrogen-containing heterocyclic derivative represented by formulae (60) to (62) are shown below.

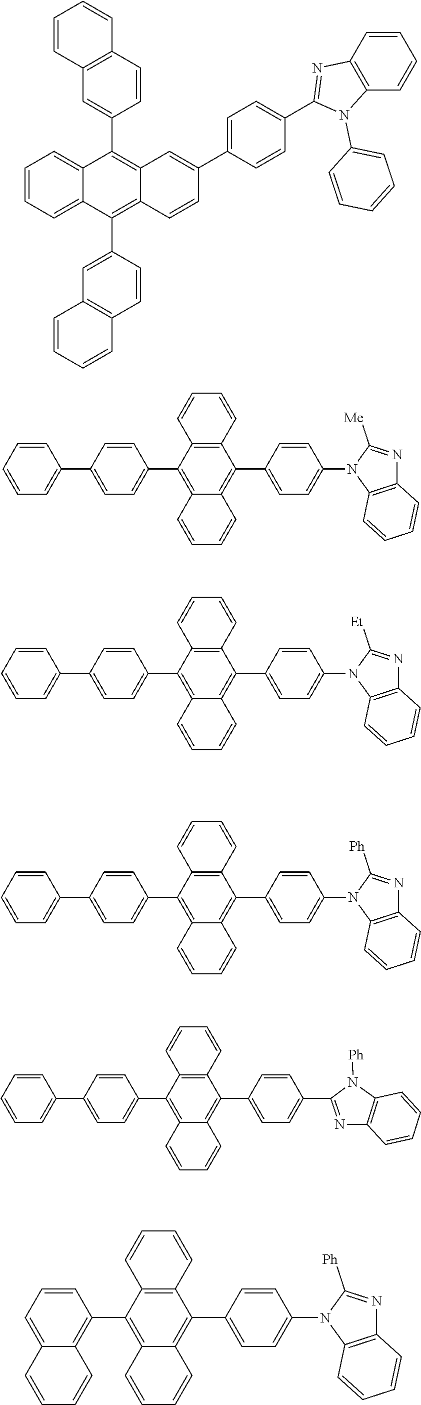 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; Cc1nc2ccccc2n1-c1ccc(-c2c3ccccc3c(-c3ccc(-c4ccccc4)cc3)c3ccccc23)cc1 ; CCc1nc2ccccc2n1-c1ccc(-c2c3ccccc3c(-c3ccc(-c4ccccc4)cc3)c3ccccc23)cc1 ; c1ccc(-c2ccc(-c3c4ccccc4c(-c4ccc(-n5c(-c6ccccc6)nc6ccccc65)cc4)c4ccccc34)cc2)cc1 ; c1ccc(-c2ccc(-c3c4ccccc4c(-c4ccc(-c5nc6ccccc6n5-c5ccccc5)cc4)c4ccccc34)cc2)cc1 ; c1ccc(-c2nc3ccccc3n2-c2ccc(-c3c4ccccc4c(-c4cccc5ccccc45)c4ccccc34)cc2)cc1]

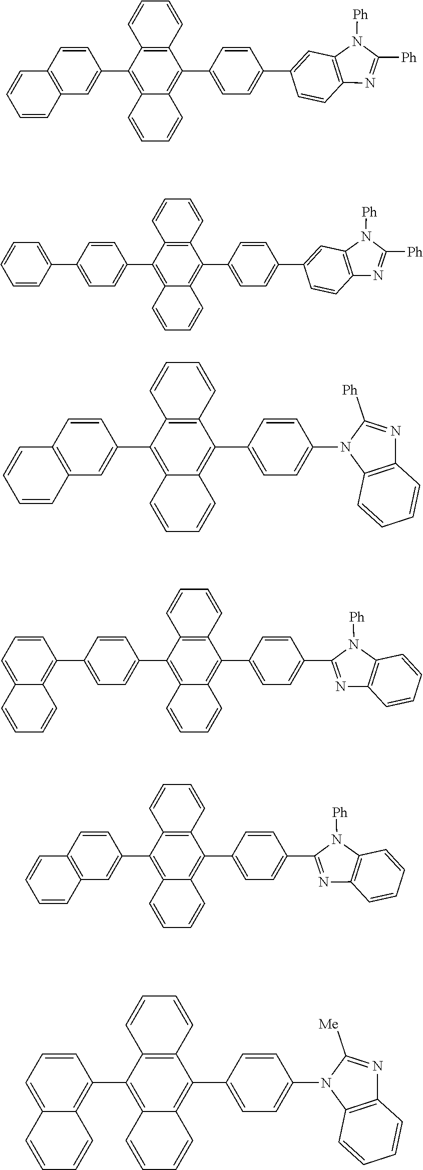

The electron transporting layer of the organic EL device of the invention may be made into two-layered structure of a first electron transporting layer (anode side) and a second electron transporting layer (cathode side).

The thickness of the electron transporting layer is preferably 1 to 100 nm, although not particularly limited thereto. If the electron transporting layer is of a two-layered structure of a first electron transporting layer (anode side) and a second electron transporting layer (cathode side), the thickness is preferably 5 to 60 nm and more preferably 10 to 40 nm for the first electron transporting layer, and preferably 1 to 20 nm and more preferably 1 to 10 nm for the second electron transporting layer.

The electron injecting layer is a layer for transporting electrons from the cathode to the organic layer unit efficiently.

The material for the electron injecting layer may be selected from the nitrogen-containing heterocyclic derivative. In addition, an inorganic compound, such as an insulating material and a semiconductor is preferably used. The electron injecting layer formed by the insulating material or the semiconductor effectively prevents the leak of electric current to enhance the electron injecting properties.

The insulating material is preferably at least one metal compound selected from the group consisting of an alkali metal chalcogenide, an alkaline earth metal chalcogenide, an alkali metal halide and an alkaline earth metal halide. The alkali metal chalcogenide, etc. mentioned above are preferred because the electron injecting properties of the electron injecting layer are further enhanced. Example of preferred alkali metal chalcogenide includes Li₂O, K₂O, Na₂S, Na₂Se and Na₂O, and example of preferred alkaline earth metal chalcogenide includes CaO, BaO, SrO, BeO, BaS and CaSe. Example of preferred alkali metal halide includes LiF, NaF, KF, LiCl, KCl and NaCl. Example of the alkaline earth metal halide includes a fluoride, such as CaF₂, BaF₂, SrF₂, MgF₂ and BeF₂, and a halide other than the fluoride.

Example of the semiconductor includes an oxide, a nitride or an oxynitride of at least one element selected from the group consisting of Ba, Ca, Sr, Yb, Al, Ga, In, Li, Na, Cd, Mg, Si, Ta, Sb and Zn. The semiconductor may be used alone or in combination of two or more. The inorganic compound in the electron injecting layer preferably forms a microcrystalline or amorphous insulating thin film. If the electron injecting layer is formed from such an insulating thin film, the pixel defects, such as dark spots, can be decreased because a more uniform thin film is formed.

The thickness of the electron injecting layer including the insulating material or the semiconductor is preferably about 0.1 to 15 nm. The electron injecting layer preferably contains the electron-donating dopant mentioned below.

The electron mobility in the electron injecting layer is preferably 10⁻⁶ cm²/Vs or more at an electric field strength of 0.04 to 0.5 MV/cm, because the electron injection from the cathode to the electron transporting layer is promoted to promote the electron injection to the adjacent blocking layer and the light emitting layer, thereby enabling the operation at a lower driving voltage.

### Electron-Donating Dopant

The organic EL device of the invention preferably comprises an electron-donating dopant at an interfacial region between the cathode and the emitting unit. With such a construction, the organic EL device has an improved luminance and an elongated lifetime. The electron-donating dopant is a metal having a work function of 3.8 eV or less and a compound including such a metal. Examples thereof include at least one selected from alkali metal, alkali metal complex, alkali metal compound, alkaline earth metal, alkaline earth metal complex, alkaline earth metal compound, rare earth metal, rare earth metal complex, and rare earth metal compound.

Examples of the alkali metal include Na (work function: 2.36 eV), K (work function: 2.28 eV), Rb (work function: 2.16 eV), and Cs (work function: 1.95 eV), with those having a work function of 2.9 eV or less being particularly preferred. Examples of the alkaline earth metal include Ca (work function: 2.9 eV), Sr (work function: 2.0 to 2.5 eV), and Ba (work function: 2.52 eV), with those having a work function of 2.9 eV or less being particularly preferred. Examples of the rare earth metal include Sc, Y, Ce, Tb, and Yb, with those having a work function of 2.9 eV or less being particularly preferred.

Examples of the alkali metal compound include alkali oxide, such as Li₂O, Cs₂O, K₂O, and alkali halide, such as LiF, NaF, CsF, and KF, with LiF, Li₂O, and NaF being preferred. Examples of the alkaline earth metal compound include BaO, SrO, CaO, and mixture thereof, such as BaₓSr₁₋ₓO (0<x<1) and BaₓCA¹₋ₓO (0<x<1), with BaO, SrO, and CaO being preferred. Examples of the rare earth metal compound include YbF₃, ScF₃, ScO₃, Y₂O₃, Ce₂O₃, GdF₃, and TbF₃, with YbF₃, ScF₃, and TbF₃ being preferred.

Examples of the alkali metal complex, alkaline earth metal complex, and rare earth metal are not particularly limited as long as containing at least one metal ion selected from an alkali metal ion, an alkaline earth metal ion, and a rare earth metal ion, respectively. The ligand is preferably, but not limited to, quinolinol, benzoquinolinol, acridinol, phenanthridinol, hydroxyphenyloxazole, hydroxyphenylthiazole, hydroxydiaryloxadiazole, hydroxydiarylthiadiazole, hydroxyphenylpyridine, hydroxyphenylbenzimidazole, hydroxybenzotriazole, hydroxyfulborane, bipyridyl, phenanthroline, phthalocyanine, porphyrin, cyclopentadiene, β-diketones, azomethines, and derivative thereof.

The electron-donating dopant material is preferably formed into a layer or island in the interfacial region, which is formed by co-depositing the electron-donating dopant material with an organic compound (light emitting material, electron injecting material) for forming the interfacial region by a resistance heating deposition method, thereby dispersing the electron-donating dopant material into the organic material. The disperse concentration expressed by the ratio of organic material: electron-donating dopant material is 100:1 to 1:100 by mole.

When the electron-donating dopant material is formed into a form of layer, a light emitting material or an electron injecting material is formed into an interfacial organic layer, and then, the electron-donating dopant material alone is deposited by a resistance heating deposition method into a layer having a thickness of preferably 0.1 to 15 nm. When the electron-donating dopant material is formed into a form of island, a light emitting material or an electron injecting material is made into an interfacial island, and then, the electron-donating dopant material alone is deposited by a resistance heating deposition method into a form of island having a thickness of preferably 0.05 to 1 nm.

The molar ratio of the main component and the electron-donating dopant in the organic EL device of the invention is preferably 5:1 to 1:5.

### N/P Doping

As described in JP 3695714B, the carrier injecting properties into the hole transporting layer and the electron transporting layer is controlled by the doping (n) with a donor material or the doping (p) with an acceptor material.

A typical example of the n-doping is an electron transporting material doped with a metal, such as Li and Cs, and a typical example of the p-doping is a hole transporting material doped with an acceptor material, such as F₄TCNQ.

### Space Layer

For example, in an organic EL device wherein a fluorescent emitting layer and a phosphorescent emitting layer are stacked, a space layer is disposed between the fluorescent emitting layer and the phosphorescent emitting layer to prevent the diffusion of excitons generated in the phosphorescent emitting layer to the fluorescent emitting layer or to control the carrier balance. The space layer may be disposed between two or more phosphorescent emitting layers.

Since the space layer is disposed between the light emitting layers, a material combining the electron transporting ability and the hole transporting ability is preferably used for forming the space layer. To prevent the diffusion of triplet energy in the adjacent phosphorescent emitting layer, the triplet energy of the material for the space layer is preferably 2.6 eV or more. The materials described with respect to the hole transporting layer are usable as the material for the space layer.

### Blocking Layer

A blocking layer, such as an electron blocking layer, a hole blocking layer, and a triplet blocking layer, is preferably disposed adjacent to the light emitting layer. The electron blocking layer is a layer for preventing the diffusion of electrons from the light emitting layer to the hole transporting layer and disposed between the light emitting layer and the hole transporting layer. The hole blocking layer is a layer for preventing the diffusion of holes from the light emitting layer to the electron transporting layer and disposed between the light emitting layer and the electron transporting layer. The triplet blocking layer prevents the diffusion of triplet excitons generated in the light emitting layer to adjacent layers and confines the triplet excitons in the light emitting layer, thereby preventing the energy of the triplet excitons from being deactivated on the molecules other than the emitting dopant, i.e., on the molecules in the electron transporting layer.

### Electronic Device

The organic EL device comprising the compound of the invention is of high performance and is usable in electronic device, for example, as display parts, such as organic EL panel module, display devices of television sets, mobile phones, personal computer, etc., and light emitting sources of lighting equipment and vehicle lighting equipment.

### EXAMPLES

The present invention will be described below in more details with reference to the examples. However, it should be noted that the scope of the invention is not limited thereto.

### Synthesis Example 1

### Synthesis of Compound BD-1

### (1) Synthesis of Intermediate 3

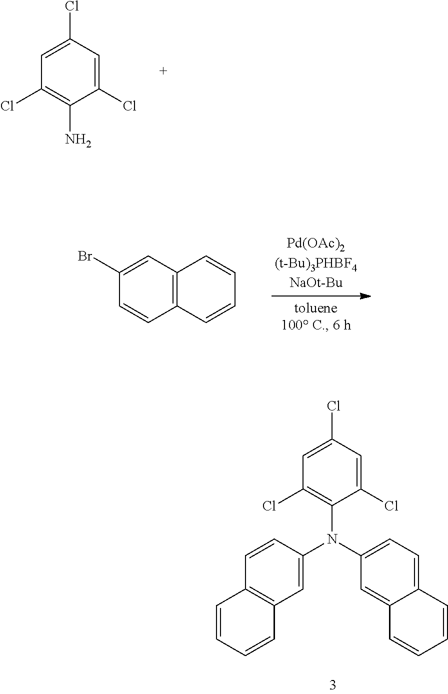 [SMILES: Nc1c(Cl)cc(Cl)cc1Cl ; Brc1ccc2ccccc2c1 ; Clc1cc(Cl)c(N(c2ccc3ccccc3c2)c2ccc3ccccc3c2)c(Cl)c1]

Under argon atmosphere, a solution of 2,4,6-trichloroaniline (1.0 g, 5.09 mmol), 2-bromonaphthalene (2.21 g, 10.7 mmol), palladium acetate (22 mg, 0.102 mmol), tri-t-butylphosphine tetrafluoroborate (59 mg, 0.204 mmol), and sodium t-butoxide (1.38 g, 15.3 mmol) in toluene (15 mL) was stirred at 100° C. for 6 h. After the reaction, water was added and the reaction solution was extracted with dichloromethane. The collected organic layers were concentrated. The obtained solid was purified by column chromatography to obtain a white solid (1.5 g), which was identified as the target Intermediate 3 by the result of mass spectrometric analysis (m/e=448 to the molecular weight of 448.77). (yield: 66%)

### (2) Synthesis of Intermediate 4

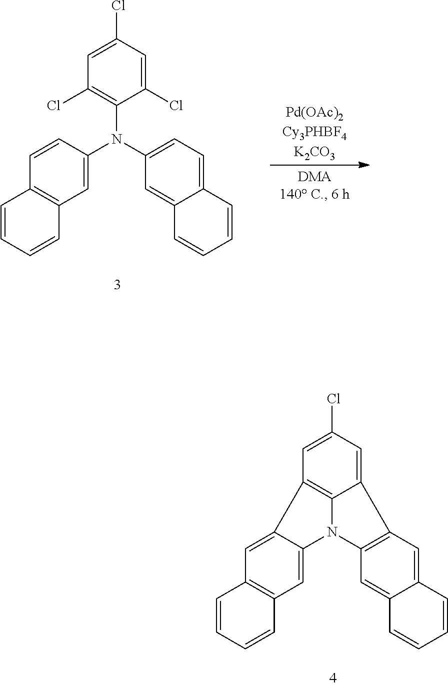 [SMILES: Clc1cc(Cl)c(N(c2ccc3ccccc3c2)c2ccc3ccccc3c2)c(Cl)c1 ; Clc1cc2c3cc4ccccc4cc3n3c4cc5ccccc5cc4c(c1)c23]

Under argon atmosphere, a solution of Intermediate 3 (100 mg, 0.223 mmol), palladium acetate (2.5 mg, 0.0111 mmol), tricyclohexylphosphine tetrafluoroborate (6.4 mg, 0.0222 mmol), and potassium carbonate (92 mg, 0.669 mmol) in dimethylacetamide (3 mL) was heated at 140° C. for 6 h. After the reaction, water was added and the reaction solution was extracted with dichloromethane. The collected organic layers were concentrated. The obtained solid was purified by flash column chromatography to obtain a yellow solid (26 mg), which was identified as the target Intermediate 4 by the result of mass spectrometric analysis (m/e=375 to the molecular weight of 375.85). (yield: 30%)

### (3) Synthesis of Compound BD-1

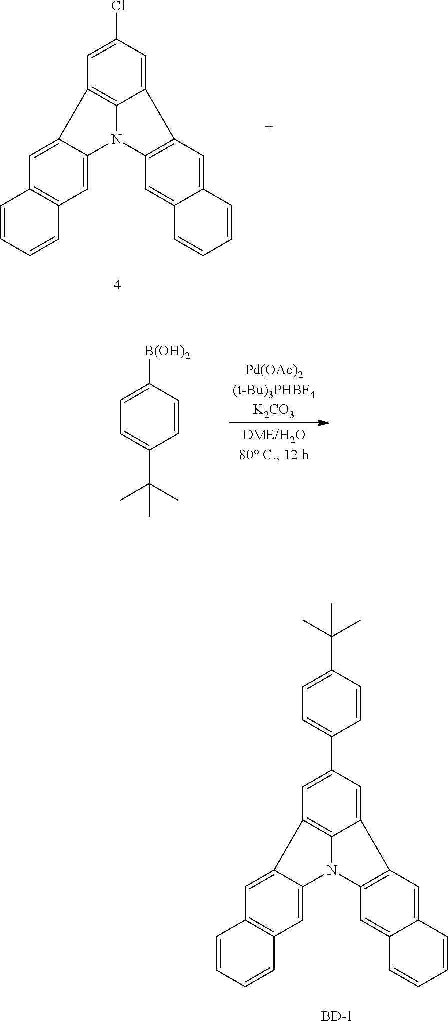 [SMILES: Clc1cc2c3cc4ccccc4cc3n3c4cc5ccccc5cc4c(c1)c23 ; CC(C)(C)c1ccc(B(O)O)cc1 ; CC(C)(C)c1ccc(-c2cc3c4cc5ccccc5cc4n4c5cc6ccccc6cc5c(c2)c34)cc1 ; "BD-1"]

Under argon atmosphere, a mixture of Intermediate 4 (20 mg, 0.0532 mmol), 4-tert-butylphenylboronic acid (9.3 mg, 0.0639 mmol), palladium acetate (1.2 mg, 0.00532 mmol), tri-t-butylphosphine tetrafluoroborate (3.1 mg, 0.0106 mmol), and potassium carbonate (14.7 mg, 0.106 mmol) in dimethoxyethane (2 mL) and water (0.5 mL) was stirred at 80° C. for 12 h. After the reaction, water was added and the reaction solution was extracted with dichloromethane. The collected organic layers were concentrated. The obtained solid was purified by column chromatography to obtain a yellow solid (16 mg), which was identified as the target Compound BD-1 by the result of mass spectrometric analysis (m/e=473 to the molecular weight of 473.61). (yield: 64%)

### Synthesis Example 2

### Synthesis of Compound BD-2

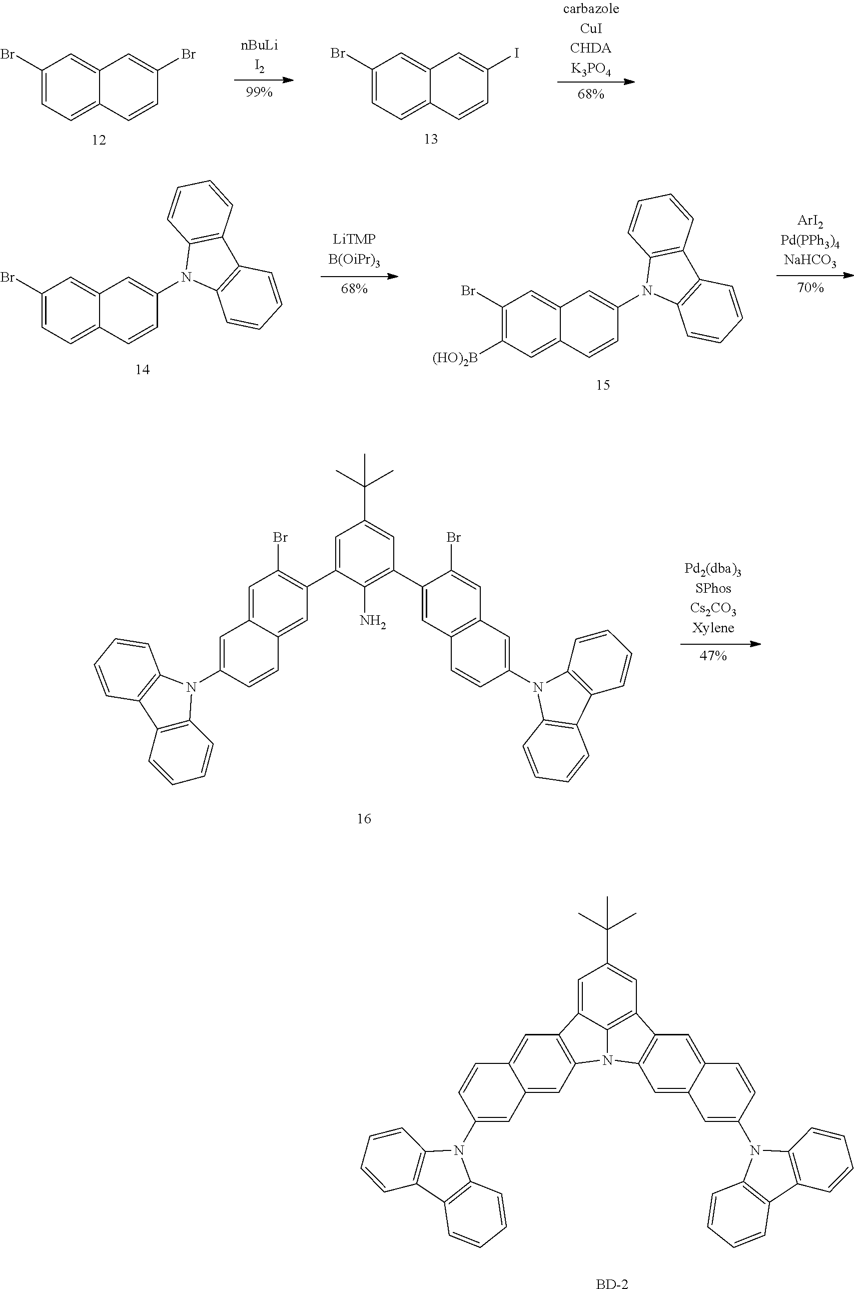

### (1) Synthesis of Intermediate 13

Under argon atmosphere, a solution of 2,7-dibromonaphthalene (5.0 g, 17 mmol) in a mixed solvent of anhydrous tetrahydrofuran (80 mL) and anhydrous toluene (40 mL) was cooled to −48° C. in a dry ice/acetone bath, to which a n-butyllithium/hexane solution (10.6 mL, 1.64 mol/L, 17 mmol) was added. The resultant solution was stirred at −45° C. for 20 min, and then stirred at −72° C. for 30 min. After adding a tetrahydrofuran solution of iodine (4.9 g, 19 mmol), the reaction mixture was stirred at −72° C. for one hour and then stirred at room temperature for 2.5 h. The reaction was deactivated by adding a 10% by mass aqueous solution of sodium sulfite (60 mL) and then extracted with toluene (150 mL). The organic layer was washed with a saturated brine (30 mL) and dried over magnesium sulfate. The solvent was evaporated off and the residue was dried under reduced pressure to obtain a pale yellow solid (5.66 g), which was identified as the target Intermediate 13 by the result of mass spectrometric analysis (m/e=339 to the molecular weight of 339). (yield: 99%)

### (2) Synthesis of Intermediate 14

Under argon atmosphere, into a suspension of 9H-carbazole (2.55 g, 15 mmol), 2-bromo-7-iodonaphthalene (5.7 g, 17 mmol), copper iodide (30 mg, 0.16 mmol), and tripotassium phosphate (7.5 g, 35 mmol) in anhydrous 1,4-dioxane (20 mL), trans-1,2-diaminocyclohexane (0.19 mL, 1.6 mmol) was added, and the resultant mixture was refluxed for 10 h. After the reaction, toluene (200 mL) was added and the inorganic substances were removed by filtration. The filtrate was concentrated and the obtained brawn solid (6.5 g) was purified by column chromatography to obtain a white acicular crystal (3.8 g), which was identified as the target Intermediate 14 by the result of mass spectrometric analysis (m/e=332 to the molecular weight of 332). (yield: 68%)

### (3) Synthesis of Intermediate 15

Under argon atmosphere, a solution of 2,2,6,6-tetramethylpiperidine (2.9 g, 20.6 mmol) in anhydrous tetrahydrofuran (30 mL) was cooled to −43° C. in a dry ice/acetone bath, to which a n-butyllithium/hexane solution (12.5 mL, 1.64 mol/L, 20.5 mmol) was added. The resultant solution was stirred at −36° C. for 20 min and then cooled to −70° C., to which triisopropoxyborane (7 mL, 30 mmol) was added dropwise and then a solution of Intermediate 14 (3.8 g, 10.2 mmol) in tetrahydrofuran (20 mL) was added. The resultant solution was stirred in a cooling bath for 10 h. After the reaction, a 5% by mass hydrochloric acid (100 mL) was added. The resultant solution was stirred at room temperature for 30 min and then extracted with ethyl acetate (150 mL). The organic layer was washed with a saturated brine (30 mL) and dried over magnesium sulfate. The solvent was evaporated off to obtain a yellow amorphous solid (4.9 g). The obtained solid was purified by column chromatography to obtain a yellow solid (2.9 g), which was identified as the target Intermediate 15 by the result of mass spectrometric analysis (m/e=415 to the molecular weight of 415). (yield: 68%)

### (4) Synthesis of Intermediate 16

Under argon atmosphere, into a suspension of 2,6-diiodo-4-tert-butylaniline (1.27 g, 3.2 mmol), Intermediate 15 (2.9 g, 7.0 mmol), tetrakis(triphenylphosphine)palladium (0.36 g, 0.31 mmol), and sodium hydrogen carbonate (2.1 g, 25 mmol) in 1,2-dimethoxyethane (40 mL), water (21 mL) was added and the resultant suspension was refluxed for 11 h. After the reaction, the reaction mixture was extracted with dichloromethane (200 mL). The organic layer was dried over magnesium sulfate and the solvent was evaporated off to obtain a yellow amorphous solid (3.5 g). The obtained solid was purified by column chromatography to obtain a white solid (2.0 g), which was identified as the target Intermediate 16 by the result of mass spectrometric analysis (m/e=887 to the molecular weight of 887). (yield: 70%)

### (5) Synthesis of Compound BD-2

Under argon atmosphere, a suspension in Intermediate 16 (1.0 g, 1.1 mmol), tris(dibenzylideneacetone)dipalladium(0) (41 mg, 45 μmol), SPhos (5 mg, 0.18 mmol), cesium carbonate (2.2 g, 6.7 mmol) in anhydrous xylene (100 mL) was refluxed for 10 h. After the reaction, the suspension was filtered and the residue was washed with water and methanol and dried under reduced pressure to obtain a pale green solid (0.427 g). The obtained solid was purified by column chromatography to obtain a yellow solid (0.37 g), which was identified as the target Compound BD-2 by the result of mass spectrometric analysis (m/e=727 to the molecular weight of 727). (yield: 47%)

### Synthesis Example 3

### Synthesis of Compound BD-3

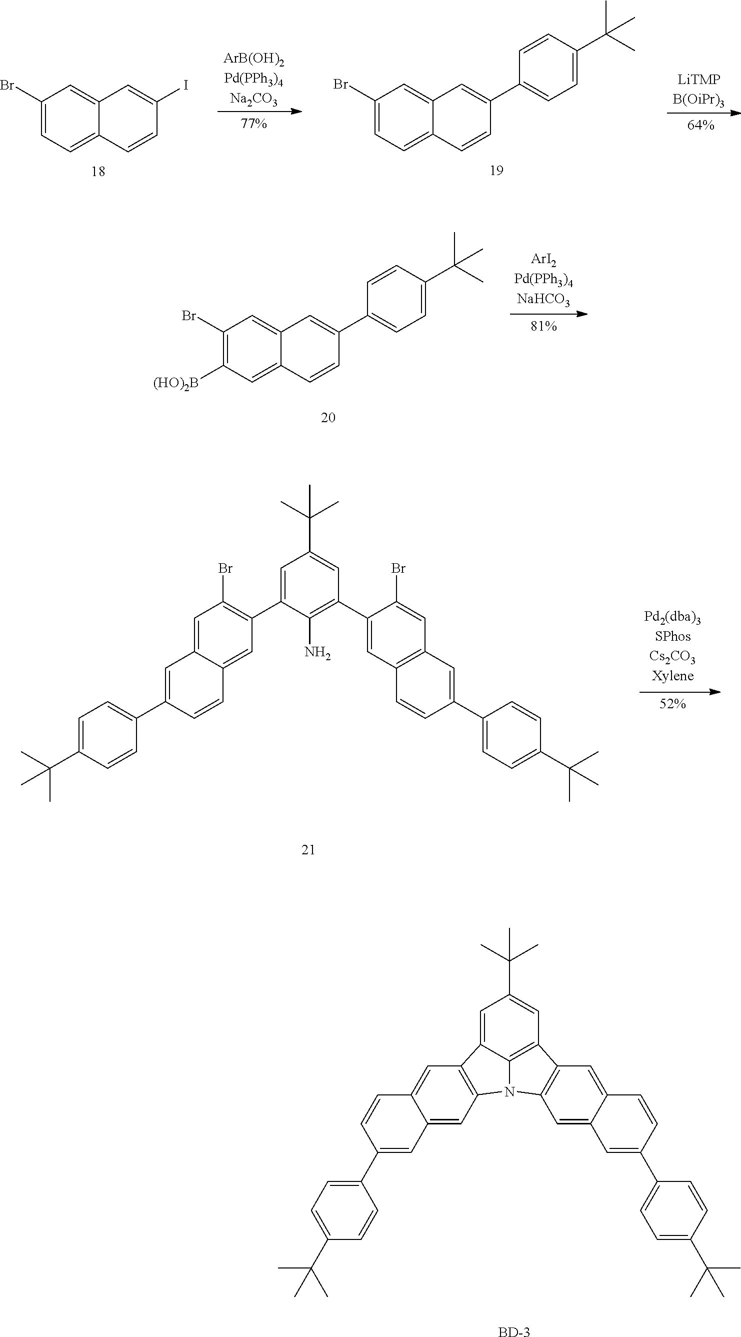

### (1) Synthesis of Intermediate 19

Under argon atmosphere, into a solution of 4-tert-butylphenylboronic acid (3.0 g, 17 mmol), 2-bromo-7-iodonaphthalene (5.66 g, 17 mmol), and tetrakis(triphenylphosphine)palladium (0.35 g, 0.30 mmol) 1,2-dimethoxyethane (45 mL), a 2 M aqueous solution of sodium carbonate (23 mL, 45 mmol) was added and the resultant solution was refluxed for 11 h. After the reaction, the reaction solution was extracted with toluene (150 mL). The organic layer was washed with a saturated brine (30 mL) and dried over magnesium sulfate. The solvent was evaporated off to obtain a brown solid (9.2 g). The obtained solid was purified by column chromatography to obtain a white solid (4.45 g), which was identified as the target Intermediate 19 by the result of mass spectrometric analysis (m/e=338 to the molecular weight of 338). (yield: 77%)

### (2) Synthesis of Intermediate 20

Under argon atmosphere, a solution of 2,2,6,6-tetramethylpiperidine (2.8 g, 20 mmol) in anhydrous tetrahydrofuran (30 mL) was cooled to −40° C. in a dry ice/acetone bath, to which a n-butyllithium/hexane solution (12 mL, 1.64 mol/L, 20 mmol) was added, and the resultant solution was stirred at −54° C. for 20 min. After the reaction, the solution was cooled to −65° C., to which triisopropoxyborane (6 mL, 26 mmol) was added dropwise and then a solution of Intermediate 19 (4.45 g, 13 mmol) in tetrahydrofuran (20 mL) was added. The resultant solution was stirred for 10 h in a cooling bath. After the reaction, a 5% by mass hydrochloric acid (70 mL) was added and the reaction solution was stirred at room temperature for 30 min and extracted with ethyl acetate (200 mL). The organic layer was washed with a saturated brine (30 mL) and dried over magnesium sulfate. The solvent was evaporated off to obtain a yellow amorphous solid (5.5 g). The obtained solid was purified by column chromatography to obtain a white solid (3.19 g), which was identified as the target Intermediate 20 by the result of mass spectrometric analysis (m/e=382 to the molecular weight of 382). (yield: 64%)

### (3) Synthesis of Intermediate 21

Under argon atmosphere, into a suspension of Intermediate 20 (3.19 g, 8.3 mmol), 2,6-diiodo-4-tert-butylaniline (1.5 g, 3.7 mmol), tetrakis(triphenylphosphine)palladium (0.43 g, 0.37 mmol), and sodium hydrogen carbonate (2.5 g, 30 mmol) in 1,2-dimethoxyethane (50 mL), water (25 mL) was added and the resultant suspension was stirred for 11 h. The reaction mixture was extracted with dichloromethane (200 mL). The organic layer was dried over magnesium sulfate and the solvent was evaporated off to obtain a yellow amorphous solid (4.14 g). The obtained solid was purified by column chromatography to obtain a white solid (2.47 g), which was identified as the target Intermediate 21 by the result of mass spectrometric analysis (m/e=821 to the molecular weight of 821). (yield: 81%)

### (4) Synthesis of Compound BD-3

Under argon atmosphere, a suspension of Intermediate 21 (2.47 g, 3.0 mmol), tris(dibenzylideneacetone)dipalladium(0) (0.11 g, 0.12 mmol), SPhos (0.20 g, 0.49 mmol), and cesium carbonate (5.9 g, 18 mmol) in anhydrous xylene (250 mL) was refluxed for 11 h. After the reaction, the reaction mixture was filtered and the residue was successively washed with water and methanol and dried under reduced pressure to obtain a pale yellow acicular crystal (1.88 g). The obtained crystal was purified by column chromatography to obtain a yellow solid (1.03 g), which was identified as the target Compound BD-3 by the result of mass spectrometric analysis (m/e=661 to the molecular weight of 661). (yield: 52%)

### Synthesis Example 4

### Synthesis of Compound BD-4

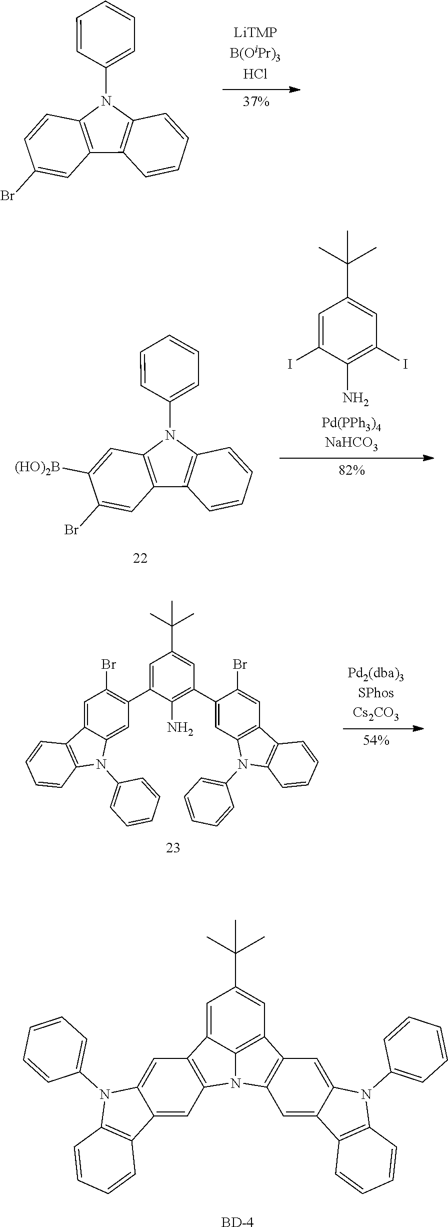 [SMILES: Brc1ccc2c(c1)c1ccccc1n2-c1ccccc1 ; OB(O)c1cc2c(cc1Br)c1ccccc1n2-c1ccccc1 ; CC(C)(C)c1cc(I)c(N)c(I)c1 ; CC(C)(C)c1cc(-c2cc3c(cc2Br)c2ccccc2n3-c2ccccc2)c(N)c(-c2cc3c(cc2Br)c2ccccc2n3-c2ccccc2)c1 ; CC(C)(C)c1cc2c3cc4c(cc3n3c5cc6c7ccccc7n(-c7ccccc7)c6cc5c(c1)c23)c1ccccc1n4-c1ccccc1 ; "BD-4"]

### (1) Synthesis of Intermediate 22

Under argon atmosphere, a solution of 2,2,6,6-tetramethylpiperidine (8.80 g, 62.4 mmol, 2 eq) in anhydrous tetrahydrofuran (THF) (90 mL) was cooled to −50° C. in a dry ice/acetone bath, to which a n-butyllithium/hexane solution (1.55 mol/L, 40.3 mL, 62.5 mmol, 1 eq) was added. The resultant solution was stirred at −50° C. for 30 min and cooled to −70° C. Triisopropoxyborane (20.0 mL, 86.7 mmol, 2.8 eq) was added dropwise and 5 min thereafter a 3-bromo-9-phenylcarbazole/THF solution (10.1 g, 31.4 mmol/45 mL) was added. The reaction mixture was stirred for 10 h in a cooling bath, to which a 10% HCl (130 mL) was added. The reaction mixture was stirred at room temperature for 30 min and extracted with ethyl acetate (200 mL). The organic layer was washed with a saturated brine (30 mL) and dried over magnesium sulfate. The solvent was evaporated off and the residue was dried under reduced pressure to obtain a yellow amorphous solid (10.6 g). The obtained solid was purified by column chromatography to obtain a pale yellow solid (4.20 g, yield: 37%), which was identified as the target Intermediate 22 by the result of mass spectron analysis (m/e=366 to the molecular weight of 366.02).

### (2) Synthesis of Intermediate 23

Under argon atmosphere, a suspension of Intermediate 22 (4.20 g, 11.5 mmol, 2.3 eq), 4-(tert-butyl)-2,6-diiodoaniline (2.00 g, 4.99 mmol), Pd(PPh₃)₄ (0.58 g, 0.50 mmol, 5% Pd), and sodium hydrogen carbonate (3.5 g, 3.6 eq) in 1,2-dimethoxyethane (70 mL) was refluxed for 11 h after adding water (35 mL). The reaction mixture was extracted with dichloromethane (250 mL) and the extract was dried over magnesium sulfate. The solvent was evaporated off and the residue was dried under reduced pressure to obtain a yellow amorphous solid (5.6 g). The obtained solid was purified by column chromatography to obtain a white solid (3.25 g, yield: 82%), which was identified as the target Intermediate 23 by the result of mass spectrometric analysis (m/e=789 to the molecular weight of 789.6).

### (3) Synthesis of Compound BD-4

Under argon atmosphere, a suspension of Intermediate 23 (3.25 g, 4.12 mmol), tris(dibenzylideneacetone)dipalladium(0) (0.15 g, 0.16 mol, 4% Pd), SPhos (0.27 g, 0.66 mmol), and cesium carbonate (8.1 g, 24.8 mmol) in anhydrous xylene (320 mL) was refluxed for 11 h. The reaction mixture was filtered and the solvent of the filtrate was evaporated off. The residue was dried under reduced pressure to obtain a brown solid (3.27 g). The obtained solid was purified by column chromatography to obtain a yellow solid (1.40 g). The obtained solid was recrystallized from toluene (40 mL) to obtain a yellow plate crystal (1.14 g, yield: 54%), which was identified as the target Compound BD-4 by the result of mass spectrometric analysis (m/e=627 to the molecular weight of 627.77).

### Synthesis Example 5

### Synthesis of Compound BD-5

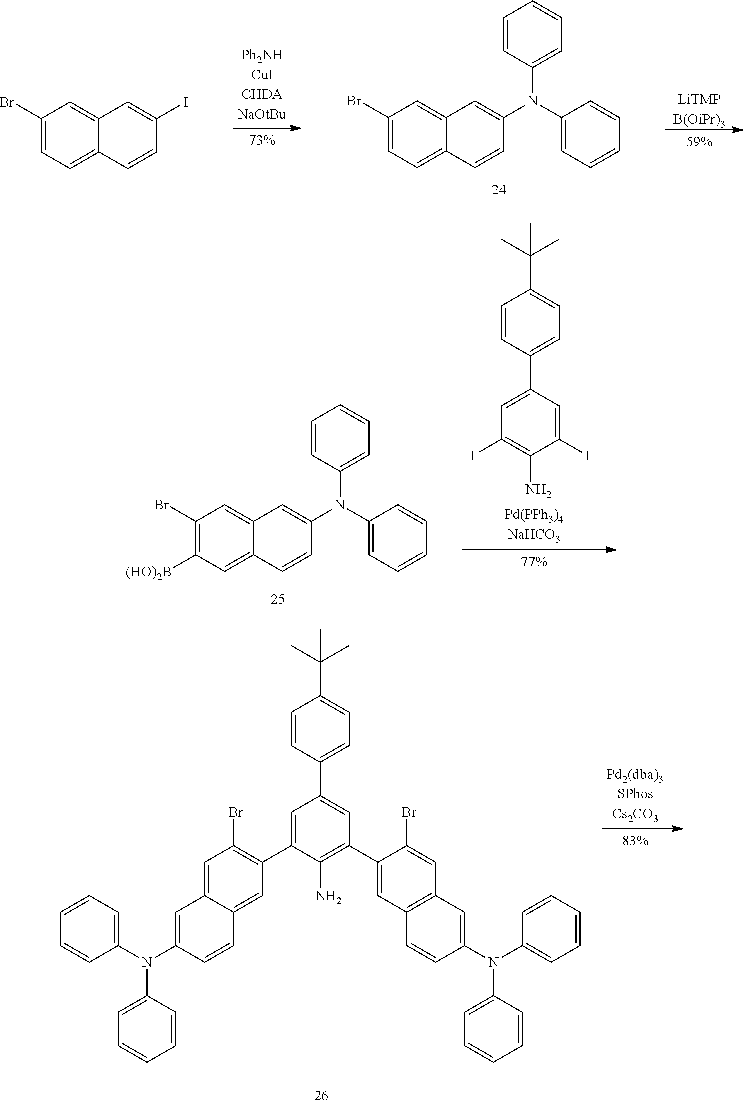 [SMILES: Brc1ccc2ccc(I)cc2c1 ; Brc1ccc2ccc(N(c3ccccc3)c3ccccc3)cc2c1 ; OB(O)c1cc2ccc(N(c3ccccc3)c3ccccc3)cc2cc1Br ; CC(C)(C)c1ccc(-c2cc(I)c(N)c(I)c2)cc1 ; CC(C)(C)c1ccc(-c2cc(-c3cc4ccc(N(c5ccccc5)c5ccccc5)cc4cc3Br)c(N)c(-c3cc4ccc(N(c5ccccc5)c5ccccc5)cc4cc3Br)c2)cc1]

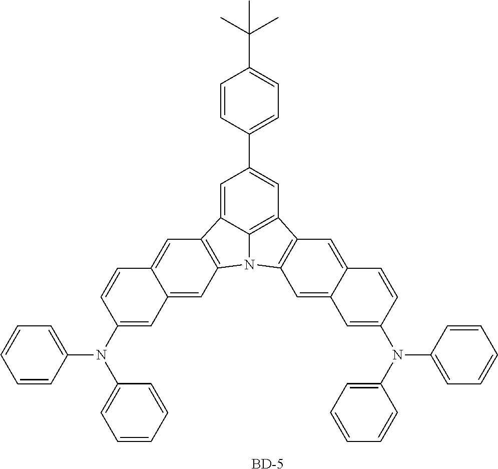 [SMILES: CC(C)(C)c1ccc(-c2cc3c4cc5ccc(N(c6ccccc6)c6ccccc6)cc5cc4n4c5cc6cc(N(c7ccccc7)c7ccccc7)ccc6cc5c(c2)c34)cc1 ; "BD-5"]

### (1) Synthesis of Intermediate 24

Under argon atmosphere, into a suspension of 2-bromo-7-iodonaphthalene (2.83 g, 16.7 mmol), diphenylamine (5.57 g, 16.7 mmol), cupper iodide (30mg, 0.16 mmol), and sodium t-butoxide (2.2 g, 23 mmol) in anhydrous 1,4-dioxane (20 mL), trans-1,2-diaminocyclohexane (0.19 mL, 1.6 mmol) was added. The resultant suspension was stirred at 110° C. for 10 h. The reaction mixture was filtered through a silica pad and the residue was washed with toluene (100 mL). The solvent of the filtrate was evaporated off and the residue was dried under reduced pressure to obtain a dark brown oil (6.7 g). The obtained oil was purified by column chromatography to obtain a white solid (4.56 g), which was identified as the target Intermediate 24 by the result of mass spectrometric analysis (m/e=373 to the molecular weight of 373). (yield: 68%)

### (2) Synthesis of Intermediate 25

Under argon atmosphere, a solution of 2,2,6,6-tetramethylpiperidine (3.4 g, 24 mmol) in anhydrous tetrahydrofuran (35 mL) was cooled to −30° C. in a dry ice/acetone bath, to which a n-butyllithium/hexane solution (14.7 mL, 1.64 mol/L, 24 mmol) was added. The resultant solution was stirred at −20° C. for 20 min and cooled to −75° C., to which triisopropoxyborane (8.3 mL, 36 mmol) was added dropwise and 5 min thereafter a solution of Intermediate 24 (4.5 g, 12 mmol) in tetrahydrofuran (20 mL) was added. The resultant solution was stirred for 10 h in a cooling bath. After the reaction, a 5% by mass hydrochloric acid (100 mL) was added and the solution was stirred at room temperature for 30 min and extracted with ethyl acetate (150 mL). The organic layer was washed with a saturated brine (30 mL) and dried over magnesium sulfate. The solution was evaporated off to obtain a reddish brown amorphous solid (5.8 g). The obtained solid was purified by column chromatography to obtain a pale yellow solid (2.94 g), which was identified as the target Intermediate 25 by the result of mass spectrometric analysis (m/e=417 to the molecular weight of 417). (yield: 59%)

### (3) Synthesis of Intermediate 26

Under argon atmosphere, into a suspension of Intermediate 25 (2.94 g, 7.0 mmol, 2.2 eq), 4-(4-tert-butylphenyl)-2,6-diiodoaniline (3.05 g, 6.40 mmol), Pd(PPh₃)₄ (0.74 g, 0.64 mmol, 5% Pd), and NaHCO₃ (4.3 g, 51 mmol, 3.6 eq) in 1,2-dimethoxyethane (80 mL), water (40 mL) was added and the resultant suspension was refluxed for 11 h. The reaction mixture was extracted with dichloromethane (200 mL) and the extract was dried over magnesium sulfate. The solvent was evaporated off and the residue was dried under reduced pressure to obtain a brown amorphous solid (7.78 g). The obtained solid was purified by column chromatography to obtain a yellow solid (4.80 g, yield: 77%), which was identified as the target Intermediate 26 by the result of mass spectrometric analysis (m/e=969 to the molecular weight of 969.8).

### (4) Synthesis of Compound BD-5

Under argon atmosphere, a suspension of Intermediate 26 (4.00 g, 4.12 mmol), tris(dibenzylideneacetone)dipalladium(0) (0.15 g, 0.164 mmol, 4% Pd), SPhos (0.27 g, 0.658 mmol), and cesium carbonate (8.1 g, 24.8 mmol) in anhydrous xylene (400 mL) was refluxed for 11 h. The reaction mixture was filtered. The solvent of the filtrate was evaporated off and the residue was dried under reduced pressure to obtain a dark yellow solid. The obtained solid was purified by column chromatography to obtain a yellow solid (2.43 g, yield: 73%), which was identified as the target Compound BD-5 by the result of mass spectrometric analysis (m/e=808 to the molecular weight of 808.04).

### Measurement of Half Width

Each of Compounds BD-1 to BD-6 (dopant material) used in the examples and the comparative examples was measured for the half width in the following manner.

The dopant material was dissolved in toluene in a concentration of 10⁻⁶ mol/L or more and 10⁻⁵ mol/L or less to prepare a test sample. The fluorescence spectrum (vertical coordinate: fluorescence intensity, horizontal coordinate: wavelength) was measured by irradiating the test sample in a quartz cell with an excitation light at room temperature (300 K) by using Fluorescent Spectrophotometer F-7000 manufactured by Hitachi High-Tech Science Corporation.

The half width (nm) of the dopant material was determined from the obtained fluorescence spectrum. The results are shown in Tables 1 to 3.

### Measurement of Affinity

The affinity (Af, electron affinity) is defined as the amount of energy released or absorbed when one electron is added to a molecule and expressed by a positive value if energy is released and a negative value if energy is absorbed.

The affinity (Af) of the first compound and the second compound was calculated from the following formula using the measured values of the ionization potential (Ip) and the singlet energy (Eg(S)):

Af (eV)=Ip−Eg(S).

### Ionization Potential (Ip)

Ionization potential Ip was determined by measuring the amount of electrons generated by charge separation of the test compound upon the irradiation of light. Atmospheric photoelectron spectrometer (AC-3 manufactured by Riken Keiki Co., Ltd.) was used for the measurement.

### Singlet Energy Eg(S)

A test compound was dissolved in toluene at a concentration of 10⁻⁵ mol/L to prepare a measuring sample. The absorption spectrum (vertical axis: absorbance, horizontal axis: wavelength) of the measuring sample in a quartz cell was taken at room temperature (300 K). A line tangent to the falling portion at a side of longer wavelength of the spectrum was drawn, and the wavelength λ_{edge} (nm) at the intersection of the tangent line and the horizontal axis was obtained. By using the obtained wavelength, the singlet energy was calculated from the following equation:

Eg (S) (eV)=1239.85/λ_{edge}.

The absorption spectrum was taken by using Spectrophotometer U-3310 of Hitachi High-Tech Science Corporation.

The measured affinities of the first compounds and the second compounds are shown in Tables 1 to 3.

### Example 1

A 25 mm×75 mm×1.1 mm glass substrate having ITO transparent electrode (anode) (product of Geomatec Company) was ultrasonically cleaned in isopropyl alcohol for 5 min and then UV/ozone cleaned for 30 min. The thickness of ITO transparent electrode was 130 nm.

The cleaned glass substrate was mounted to a substrate holder of a vacuum vapor deposition apparatus. First, Compound HI-1 was vapor-deposited on the surface having the transparent electrode so as to cover the transparent electrode to form a hole injecting layer with a thickness of 5 nm.

On the hole injecting layer, Compound HT-1 was vapor-deposited to form a first hole transporting layer with a thickness of 80 nm.

Then, on the first hole transporting layer, Compound HT-2 was vapor-deposited to form a second hole transporting layer with a thickness of 10 nm.

Successively after forming the second hole transporting layer, Compound BH1-2 (first compound), Compound BH3-1 (second compound), and Compound BD-1 (dopant material) were vapor co-deposited to form a light emitting layer with a thickness of 25 nm. The concentration in the light emitting layer was 80% by mass for Compound BH1-2, 18% by mass for BH3-1, and 2% by mass for Compound BD-1.

On the light emitting layer, Compound ET-1 was vapor-deposited to form a first electron transporting layer with a thickness of 10 nm.

Successively after forming the first electron transporting layer, Compound ET-2 was vapor-deposited to form a second electron transporting layer with a thickness of 15 nm.

Then, on the second electron transporting layer, lithium fluoride (LiF) was vapor-deposited to form an electron injecting electrode with a thickness of 1 nm.

Finally, metallic aluminum (Al) was vapor-deposited on the electron injecting electrode to form a metallic cathode with a thickness of 80 nm.

The layered structure of the organic EL device is shown below.

ITO (130)/HI-1 (5)/HT-1 (80)/HT-2 (10)/BH1-2:BH3-1:BD-1 (25, 80:18:2% by mass)/ET-1 (10)/ET-2 (15)/LiF (1)/Al (80)

The numeral in each parenthesis is the thickness (nm).

### Evaluation of Organic EL Device

The organic EL device thus produced was measured for the main peak wavelength λp and the lifetime LT90 in the following manners.

A spectral radiance spectrum was measured by applying direct voltage to the organic EL device so as to reach a current density of 10 mA/cm². The main peak wavelength λp (unit of measure: nm) was determined from the obtained spectral radiance spectrum. The spectral radiance spectrum was measured by using a spectroradiometer CS-1000 manufactured by Konica Minolta.

A direct current was allowed to continuously flow the organic EL device at an initial current density of 50 mA/cm² and the time taken until the luminance was reduced to 90% of the initial luminance was measured. The measured time was taken as the lifetime LT90.

The results are shown in Table 1.

### Examples 2 to 19 and Comparative Examples 1 to 10

Each organic EL device containing the first compound, the second compound, and the dopant material in the ratio by mass shown in Table 1 was produced and evaluated in the same manner as in Example 1. The results are shown in Table 1.

The materials used in Examples 1 to 19 and Comparative Examples 1 to 10 are shown below.

### Hole Injecting Layer and Hole Transporting Layer Materials

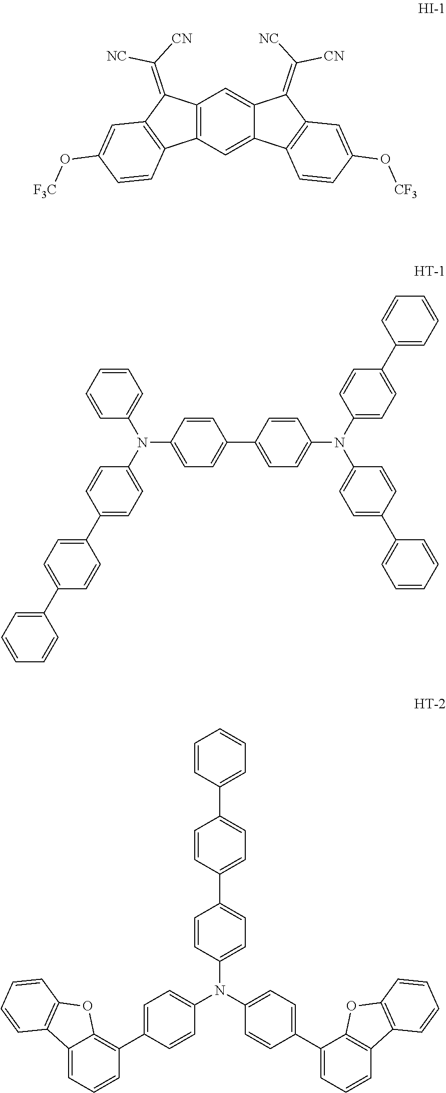 [SMILES: N#CC(C#N)=C1c2cc(OC(F)(F)F)ccc2-c2cc3c(cc21)C(=C(C#N)C#N)c1cc(OC(F)(F)F)ccc1-3 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1]

### Electron Transporting Layer Materials

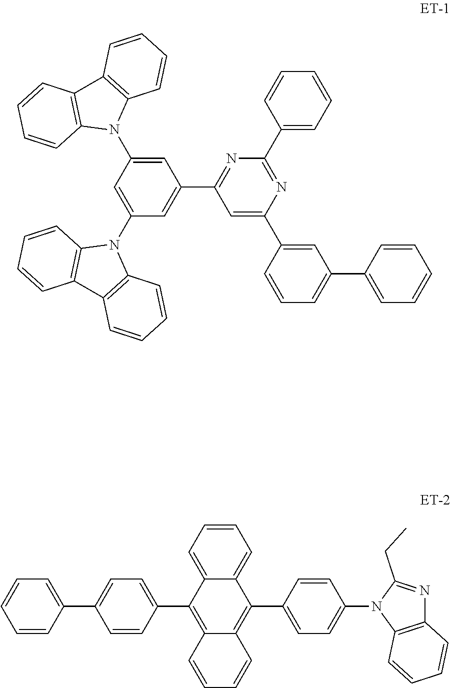 [SMILES: c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; CCc1nc2ccccc2n1-c1ccc(-c2c3ccccc3c(-c3ccc(-c4ccccc4)cc3)c3ccccc23)cc1]

### Dopant Materials

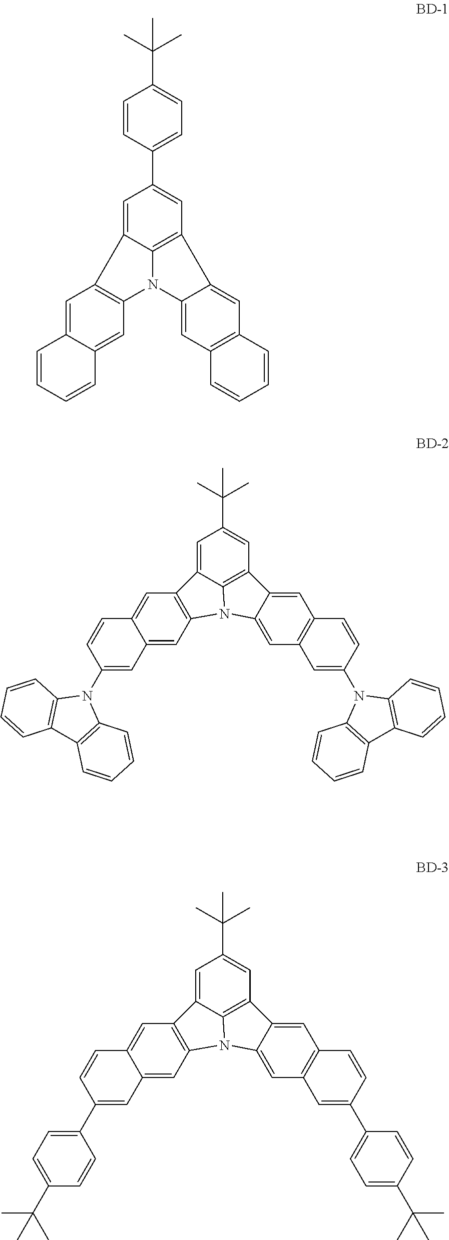 [SMILES: CC(C)(C)c1ccc(-c2cc3c4cc5ccccc5cc4n4c5cc6ccccc6cc5c(c2)c34)cc1 ; CC(C)(C)c1cc2c3cc4ccc(-n5c6ccccc6c6ccccc65)cc4cc3n3c4cc5cc(-n6c7ccccc7c7ccccc76)ccc5cc4c(c1)c23 ; CC(C)(C)c1ccc(-c2ccc3cc4c5cc(C(C)(C)C)cc6c7cc8ccc(-c9ccc(C(C)(C)C)cc9)cc8cc7n(c4cc3c2)c56)cc1]

### First Compounds

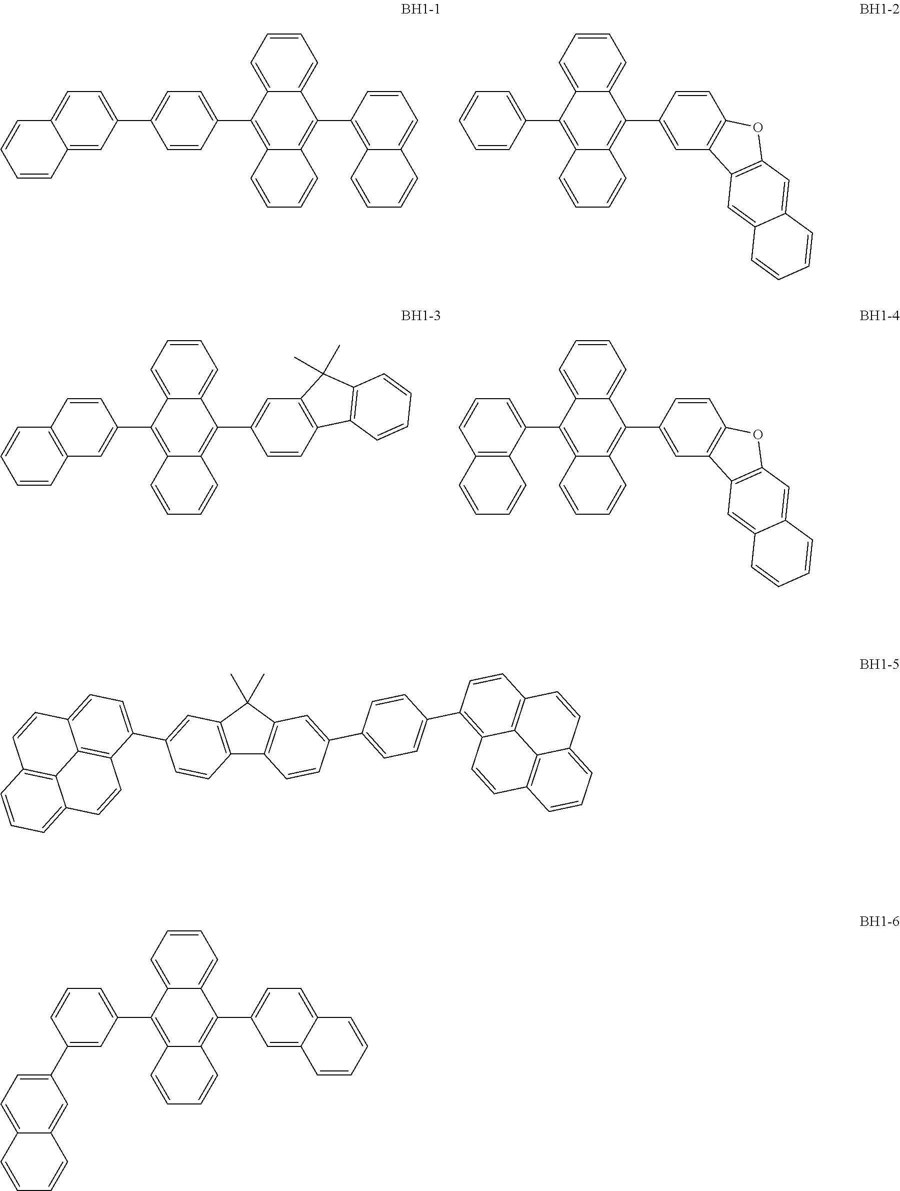

### Second Compounds

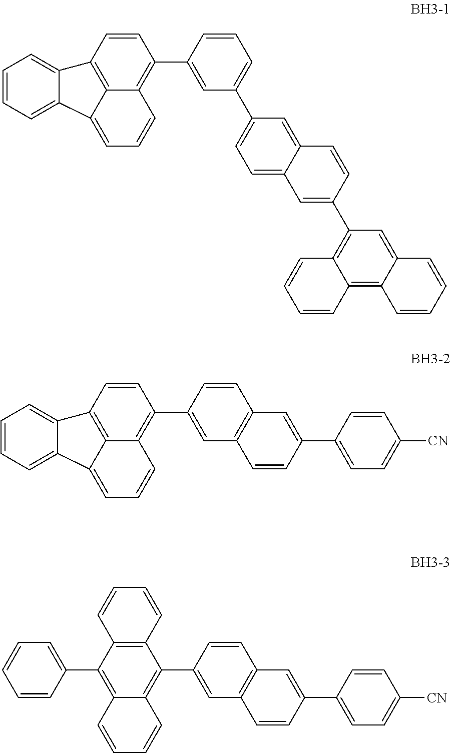

TABLE 1

| | Material Parameter | | | | | | | | | | |
|---|---|---|---|---|---|---|---|---|---|---|---|
| | | | | | | | Dopant Material | | | Device | |
| | First Compound | | | Second Compound | | | | | half | Performance | |
| | | % by | | | % by | | | % by | width | λp | LT90 |
| | material | mass | Af | material | mass | Af | material | mass | (nm) | (nm) | (h) |
| Example 1 | BH1-2 | 80 | 2.92 | BH3-1 | 18 | 3.12 | BD-1 | 2 | 18 | 450 | 219 |
| Example 2 | BH1-2 | 92 | 2.92 | BH3-1 | 6 | 3.12 | BD-1 | 2 | 18 | 449 | 216 |
| Comparative | BH1-2 | 98 | 2.92 | — | — | — | BD-1 | 2 | 18 | 449 | 190 |
| Example 1 | | | | | | | | | | | |
| Example 3 | BH1-3 | 80 | 2.87 | BH3-1 | 18 | 3.12 | BD-1 | 2 | 18 | 450 | 145 |
| Example 4 | BH1-3 | 92 | 2 87 | BH3-1 | 6 | 3.12 | BD-1 | 2 | 18 | 449 | 151 |
| Example 5 | BH1-3 | 80 | 2.87 | BH3-2 | 18 | 3.25 | BD-1 | 2 | 18 | 452 | 263 |
| Example 6 | BH1-3 | 92 | 2.87 | BH3-2 | 6 | 3.25 | BD-1 | 2 | 18 | 450 | 288 |
| Example 7 | BH1-3 | 92 | 2.87 | BH3-3 | 6 | 3.04 | BD-1 | 2 | 18 | 449 | 117 |
| Comparative | BH1-3 | 98 | 2.87 | — | — | — | BD-1 | 2 | 18 | 448 | 80 |
| Example 2 | | | | | | | | | | | |
| Example 8 | BH1-4 | 80 | 3.00 | BH3-1 | 18 | 3.12 | BD-1 | 2 | 18 | 450 | 179 |
| Example 9 | BH1-4 | 92 | 3.00 | BH3-1 | 6 | 3 12 | BD-1 | 2 | 18 | 450 | 185 |
| Example 10 | BH1-4 | 80 | 3.00 | BH3-2 | 18 | 3.25 | BD-1 | 2 | 18 | 452 | 356 |
| Example 11 | BH1-4 | 92 | 3 00 | BH3-2 | 6 | 3.25 | BD-1 | 2 | 18 | 451 | 210 |
| Comparative | BH1-4 | 98 | 3.00 | — | — | — | BD-1 | 2 | 18 | 449 | 117 |
| Example 3 | | | | | | | | | | | |
| Example 12 | BH1-5 | 92 | 2.80 | BH3-2 | 6 | 3.25 | BD-1 | 2 | 18 | 453 | 132 |
| Example 13 | BH1-5 | 80 | 2.80 | BH3-3 | 18 | 3.04 | BD-1 | 2 | 18 | 451 | 134 |
| Comparative | BH1-5 | 98 | 2.80 | — | — | — | BD-1 | 2 | 18 | 452 | 43 |
| Example 4 | | | | | | | | | | | |
| Example 14 | BH1-1 | 92 | 3.02 | BH3-1 | 6 | 3.12 | BD-2 | 2 | 15 | 442 | 147 |
| Comparative | BH1-1 | 98 | 3.02 | — | — | — | BD-2 | 2 | 15 | 442 | 93 |
| Example 5 | | | | | | | | | | | |
| Example 15 | BH1-2 | 92 | 2.92 | BH3-1 | 6 | 3.12 | BD-2 | 2 | 15 | 443 | 132 |
| Comparative | BH1-2 | 98 | 2.92 | — | — | — | BD-2 | 2 | 15 | 442 | 112 |
| Example 6 | | | | | | | | | | | |
| Example 16 | BH1-6 | 92 | 3.00 | BD3-1 | 6 | 3.12 | BD-2 | 2 | 15 | 442 | 122 |
| Comparative | BH1-6 | 98 | 3.00 | — | — | — | BD-2 | 2 | 15 | 442 | 63 |
| Example 7 | | | | | | | | | | | |
| Example 17 | BH1-1 | 92 | 3.02 | BH3-1 | 6 | 3.12 | BD-3 | 2 | 17 | 451 | 197 |
| Comparative | BH1-1 | 98 | 3.02 | — | — | — | BD-3 | 2 | 17 | 451 | 136 |
| Example 8 | | | | | | | | | | | |
| Example 18 | BH1-2 | 92 | 2 92 | HB3-1 | 6 | 3.12 | BD-3 | 2 | 17 | 452 | 214 |
| Comparative | BH1-2 | 98 | 2.92 | — | — | — | BD-3 | 2 | 17 | 451 | 166 |
| Example 9 | | | | | | | | | | | |
| Example 19 | BH1-6 | 92 | 3.00 | BH3-1 | 6 | 3.12 | BD-3 | 2 | 17 | 451 | 217 |
| Comparative | BH1-6 | 98 | 3.00 | — | — | — | BD-3 | 2 | 17 | 451 | 124 |
| Example 10 | | | | | | | | | | | |

As compared with the single-host organic EL devices of Comparative Examples 1 to 10 each containing the first compound and the dopant material, i.e., comparing the organic EL devices that are different from each other only in the presence or absence of the second compound, the co-host organic EL devices of Examples 1 to 19 each containing the second compound in addition to the first compound and the dopant material had longer lifetimes.

Like the single-host organic EL device, the co-host organic EL devices emitted light in a blue region.

### Examples 20 to 22 and Comparative Examples 11 to 13

Each organic EL device containing the first compound, the second compound, and the dopant material in the ratio by mass shown in Table 2 or 3 was produced and evaluated in the same manner as in Example 1. The results are shown in Tables 2 and 3. In Table 3, LT90 of the device of Example 22 is shown by a relative value taking LT90 of the device of Comparative Example 13 as 1.00.

The materials used in Examples 20 to 22 and Comparative Examples 11 to 13 are shown below, wherein the compounds described above are omitted.

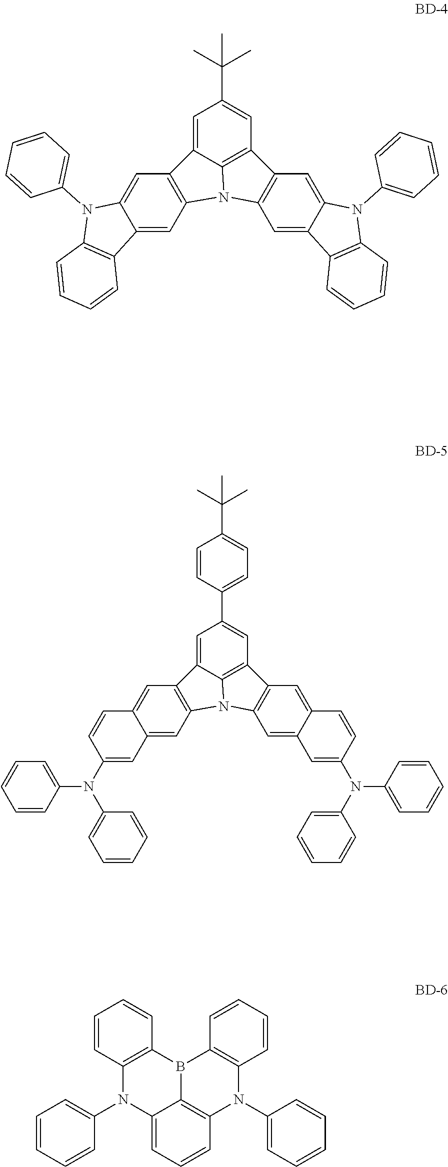 [SMILES: CC(C)(C)c1cc2c3cc4c(cc3n3c5cc6c7ccccc7n(-c7ccccc7)c6cc5c(c1)c23)c1ccccc1n4-c1ccccc1 ; CC(C)(C)c1ccc(-c2cc3c4cc5ccc(N(c6ccccc6)c6ccccc6)cc5cc4n4c5cc6cc(N(c7ccccc7)c7ccccc7)ccc6cc5c(c2)c34)cc1 ; c1ccc(N2c3ccccc3B3c4ccccc4N(c4ccccc4)c4cccc2c43)cc1]

TABLE 2

| | Material Parameter | | | | | | | | | | |
|---|---|---|---|---|---|---|---|---|---|---|---|
| | | | | | | | Dopant Material | | | Device | |
| | First Compound | | | Second Compound | | | | | half | Performance | |
| | | % by | | | % by | | | % by | width | λp | LT90 |
| | material | mass | Af | material | mass | Af | material | mass | (nm) | (nm) | (h) |
| Example 20 | BH1-1 | 92 | 3.02 | BH3-1 | 6 | 3.12 | BD-4 | 2 | 18 | 462 | 280 |
| Comparative | BH1-1 | 98 | 3.02 | — | — | — | BD-4 | 2 | 18 | 462 | 238 |
| Example 11 | | | | | | | | | | | |
| Example 21 | BH1-1 | 92 | 3.02 | BH3-1 | 6 | 3.12 | BD-5 | 2 | 17 | 454 | 185 |
| Comparative | BH1-1 | 98 | 3.02 | — | — | — | BD-5 | 2 | 17 | 454 | 176 |
| Example 12 | | | | | | | | | | | |

TABLE 3

| | Material Parameter | | | | | | | | | | |
|---|---|---|---|---|---|---|---|---|---|---|---|
| | | | | | | | Dopant Material | | | Device | |
| | First Compound | | | Second Compound | | | | | half | Performance | |
| | | % by | | | % by | | | % by | width | λp | LT90 |
| | material | mass | Af | material | mass | Af | material | mass | (nm) | (nm) | (relative value) |
| Example 22 | BH1-1 | 90 | 3.02 | BH3-1 | 6 | 3.12 | BD-6 | 4 | 22 | 459 | 1.11 |
| Comparative | BH1-1 | 96 | 3.02 | — | — | — | BD-6 | 4 | 22 | 460 | 1.00 |
| Example 13 | | | | | | | | | | | |

As compared with the single-host organic EL devices of Comparative Examples 11 to 13 each containing the first compound and the dopant material, i.e., comparing the organic EL devices that are different from each other only in the presence or absence of the second compound, the co-host organic EL devices of Examples 20 to 22 each containing the second compound in addition to the first compound and the dopant material had longer lifetimes.

Like the single-host organic EL device, the co-host organic EL devices emitted light in a blue region.

### REFERENCE SIGNS LIST

1: Organic EL device
2: Substrate
3: Anode
4: Cathode
5: Light emitting layer
6: Hole injecting layer/Hole transporting layer
7: Electron injecting layer/Electron transporting layer
10: Emission unit

1. An organic electroluminescence device comprising a cathode, an anode and an organic layer disposed between the cathode and the anode, wherein the organic layer comprises a fluorescent emitting layer and the fluorescent emitting layer comprises:
at least one first compound selected from the compounds represented by formulae (19), (21), (22), and (23);
a second compound selected from the compounds represented by formula (3a); and
a dopant material selected from the compounds represented by formulae (D1) and (D2); 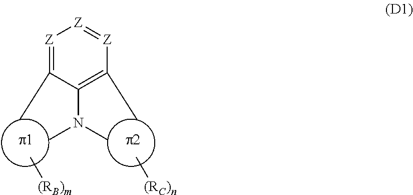
wherein:
Z is CR_{A} or N;
π1 is a substituted or unsubstituted aromatic hydrocarbon ring having 6 to 50 ring carbon atoms or a substituted or unsubstituted aromatic heterocyclic ring having 5 to 50 ring atoms;
π2 is a substituted or unsubstituted aromatic hydrocarbon ring having 6 to 50 ring carbon atoms or a substituted or unsubstituted aromatic heterocyclic ring having 5 to 50 ring atoms;
R_{A}, R_{B}, and R_{C} are each independently a hydrogen atom or a substituent, wherein the substituent is a halogen atom, a cyano group, a substituted or unsubstituted alkyl group having 1 to 20 carbon atoms, a substituted or unsubstituted alkenyl group having 1 to 20 carbon atoms, a substituted or unsubstituted alkynyl group having 1 to 20 carbon atoms, a substituted or unsubstituted cycloalkyl group having 3 to 20 ring carbon atoms, an amino group, a substituted or unsubstituted alkoxy group having 1 to 20 carbon atoms, a substituted or unsubstituted aryloxy group having 6 to 50 ring carbon atoms, a substituted or unsubstituted alkylthio group having 1 to 20 carbon atoms, a substituted or unsubstituted arylthio group having 6 to 50 ring carbon atoms, a group represented by —Si(R₁₀₁)(R₁₀₂)(R₁₀₃), a group represented by —N(R₁₀₄)(R₁₀₅), a substituted or unsubstituted aryl group having 6 to 50 ring carbon atoms, or a substituted or unsubstituted heteroaryl group having 5 to 50 ring atoms;
R₁₀₁ to R₁₀₅ are each independently a hydrogen atom, a substituted or unsubstituted alkyl group having 1 to 20 carbon atoms, a substituted or unsubstituted cycloalkyl group having 3 to 20 ring carbon atoms, a substituted or unsubstituted aryl group having 6 to 50 ring carbon atoms, or a substituted or unsubstituted heteroaryl group having 5 to 50 ring atoms;
n and m are each independently an integer of 1 to 4;
adjacent two R_{A}'s are bonded to each other to form a substituted or unsubstituted ring structure or not bonded to each other, thereby failing to form a ring structure;
adjacent two R_{B}'s are bonded to each other to form a substituted or unsubstituted ring structure or not bonded to each other, thereby failing to form a ring structure; and
adjacent two R_{C}'s are bonded to each other to form a substituted or unsubstituted ring structure or not bonded to each other, thereby failing to form a ring structure; 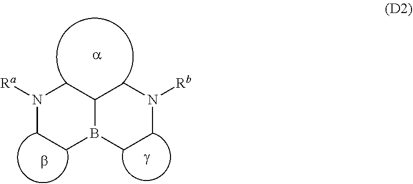
wherein:
a ring α, a ring β, and a ring γ are each independently a substituted or unsubstituted aromatic hydrocarbon ring having 6 to 50 ring carbon atoms or a substituted or unsubstituted aromatic heterocyclic ring having 5 to 50 ring atoms;
R^{a} and R^{b} are each independently a substituted or unsubstituted aryl group having 6 to 50 ring carbon atoms, a substituted or unsubstituted heteroaryl group having 5 to 50 ring atoms, or a substituted or unsubstituted alkyl group having 1 to 20 carbon atoms;
R^{a} may be bonded to one or both of the ring α and the ring β directly or via a linker; and
R^{b} may be bonded to one or both of the ring α and the ring γ directly or via a linker; 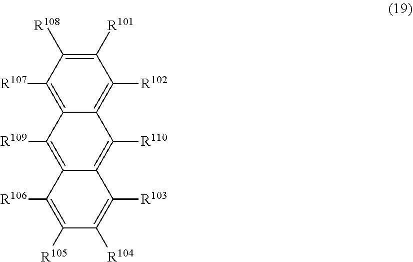
wherein:
R₁₀₁ to R₁₁₀ are each independently a hydrogen atom or a substituent, wherein the substituent is as described above with respect to the substituent of R_{A}, R_{B}, and R_{C};
provided that at least one of R¹⁰¹ to R¹¹⁰ is -L-Ar;
each L is independently a single bond or a linker, wherein the linker is a substituted or unsubstituted arylene group having 6 to 30 ring carbon atoms or a substituted or unsubstituted heteroarylene group having 5 to 30 ring atoms; and
each Ar is independently a substituted or unsubstituted single ring group having 5 to 50 ring atoms, a substituted or unsubstituted fused ring group having 8 to 50 ring atoms, or a monovalent group wherein two or more selected from the single ring and the fused ring are bonded to each other via a single bond; 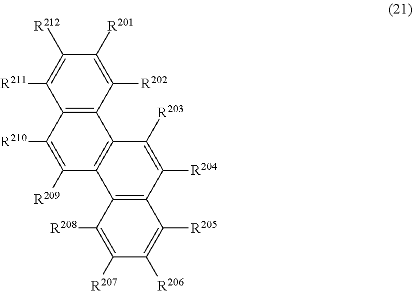
wherein:
R²⁰¹ to R²¹² are each independently a hydrogen atom or a substituent, wherein the substituent is as described above with respect to the substituent of R_{A}, R_{B}, and R_{C};
provided that at least one of R²⁰¹ to R²¹² is -L²-Ar²¹;
each L² is independently a single bond or a linker, wherein the linker is a substituted or unsubstituted arylene group having 6 to 30 ring carbon atoms or a substituted or unsubstituted heteroarylene group having 5 to 30 ring atoms; and
each Ar²¹ is independently a substituted or unsubstituted single ring group having 5 to 50 ring atoms, a substituted or unsubstituted fused ring group having 8 to 50 ring atoms, or a monovalent group wherein two or more selected from the single ring and the fused ring are bonded to each other via a single bond; 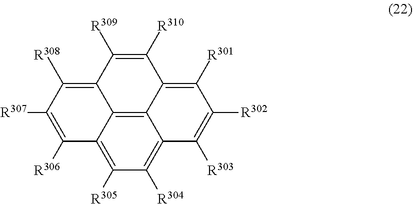
wherein:
R³⁰¹ to R³¹⁰ are each independently a hydrogen atom or a substituent, wherein the substituent is as described above with respect to the substituent of R_{A}, R_{B}, and R_{C};
provided that at least one of R³⁰¹ to R³¹⁰ is -L³-Ar³¹;
each L³ is independently a single bond or a linker, wherein the linker is a substituted or unsubstituted arylene group having 6 to 30 ring carbon atoms or a substituted or unsubstituted heteroarylene group having 5 to 30 ring atoms; and
each Ar³¹ is independently a substituted or unsubstituted single ring group having 5 to 50 ring atoms, a substituted or unsubstituted fused ring group having 8 to 50 ring atoms, or a monovalent group wherein two or more selected from the single ring and the fused ring are bonded to each other via a single bond; 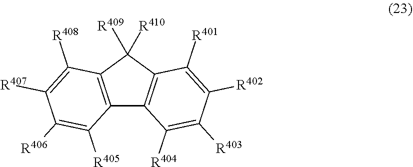
wherein:
R⁴⁰¹ to R⁴¹⁰ are each independently a hydrogen atom or a substituent, wherein the substituent is as described above with respect to the substituent of R_{A}, R_{B}, and R_{C};
provided that at least one of R⁴⁰¹ to R⁴¹⁰ is -L⁴-Ar⁴¹;
each L⁴ is each independently a single bond or a linker, wherein the linker is a substituted or unsubstituted arylene group having 6 to 30 ring carbon atoms or a substituted or unsubstituted heteroarylene group having 5 to 30 ring atoms;
each Ar⁴¹ is independently a substituted or unsubstituted single ring group having 5 to 50 ring atoms, a substituted or unsubstituted fused ring group having 8 to 50 ring atoms, or a monovalent group wherein two or more selected from the single ring and the fused ring are bonded to each other via a single bond; and
adjacent two selected from R⁴⁰¹ and R⁴⁰², R⁴⁰² and R⁴⁰³, R⁴⁰³ and R⁴⁰⁴, R⁴⁰⁵ and R⁴⁰⁶, R⁴⁰⁶ and R⁴⁰⁷, and R⁴⁰⁷ and R⁴⁰⁸ may be bonded to each other to form a substituted or unsubstituted ring structure; 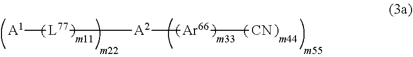
wherein:
L⁷⁷ is a substituted or unsubstituted arylene group having 6 to 50 ring carbon atoms or a substituted or unsubstituted heteroarylene group having 5 to 50 ring atoms;
Ar⁶⁶ is a di- to tetra-valent residue of an aromatic hydrocarbon ring having 6 to 50 ring carbon atoms or an aromatic heterocyclic ring having 5 to 50 ring atoms, each optionally having a substituent;
m11 is 0, 1, or 2, when m11 is 0, L⁷⁷ is a single bond, and when m11 is 2, two L⁷⁷'s may be the same or different;
m22 is 0 or 1, when m22 is 0, A¹-(L⁷⁷)ₘ₁₁- is not present and a hydrogen atom is bonded to A²;
m33 is 0, 1, 2, or 3, when m33 is 0, Ar⁶⁶ is a single bond, and when m33 is 2 or 3, two or three Ar⁶⁶'s may be the same or different;
m44 is 0, 1, 2, or 3, when m44 is 0, CN is not present and a hydrogen atom is bonded to A⁶⁶;
m55 is 1, 2, or 3, when m55 is 2 or 3, two or three —(Ar⁶⁶)ₘ₃₃—(CN)ₘ₅₅ may be the same or different;
A¹ is a monovalent group selected from formulae (A-1) to (A-12); and
A² is a di- to tetra-valent group selected from formulae (A-1) to (A-12): 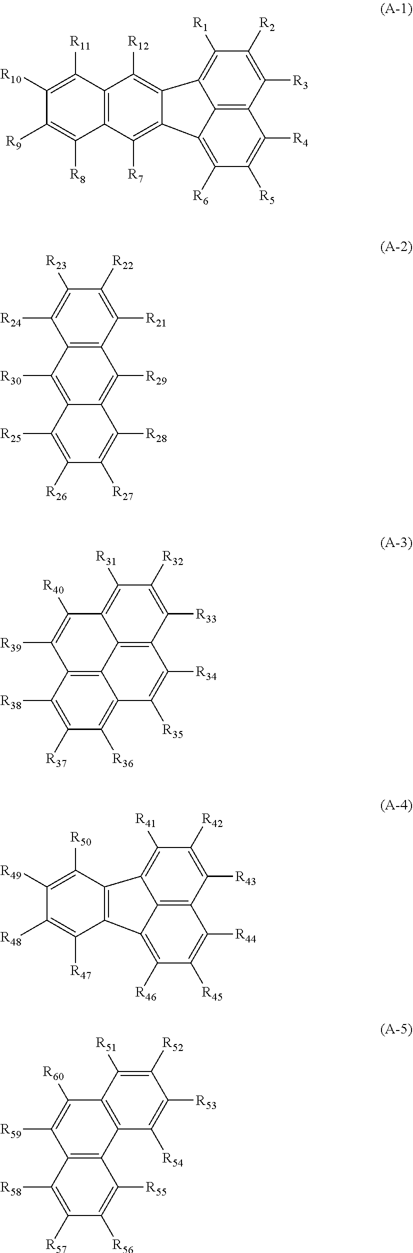 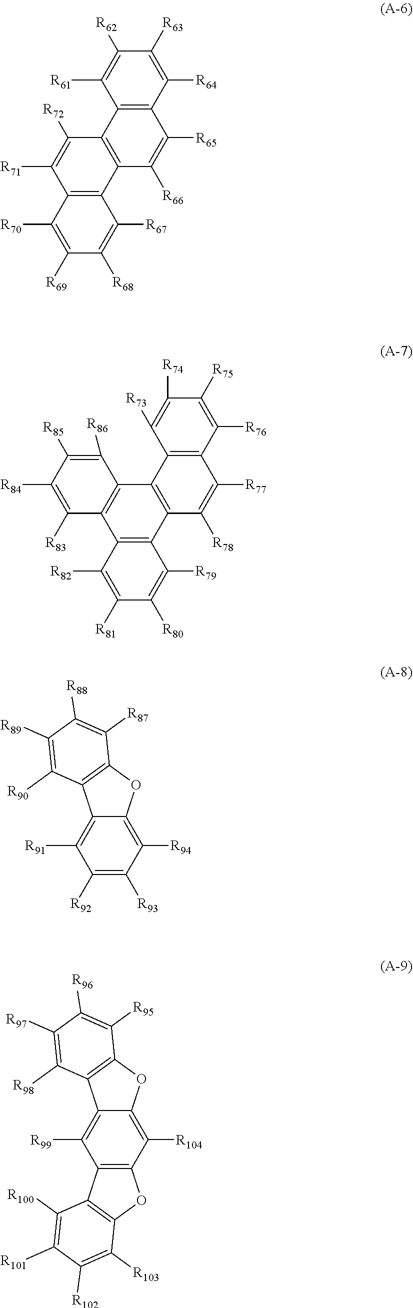 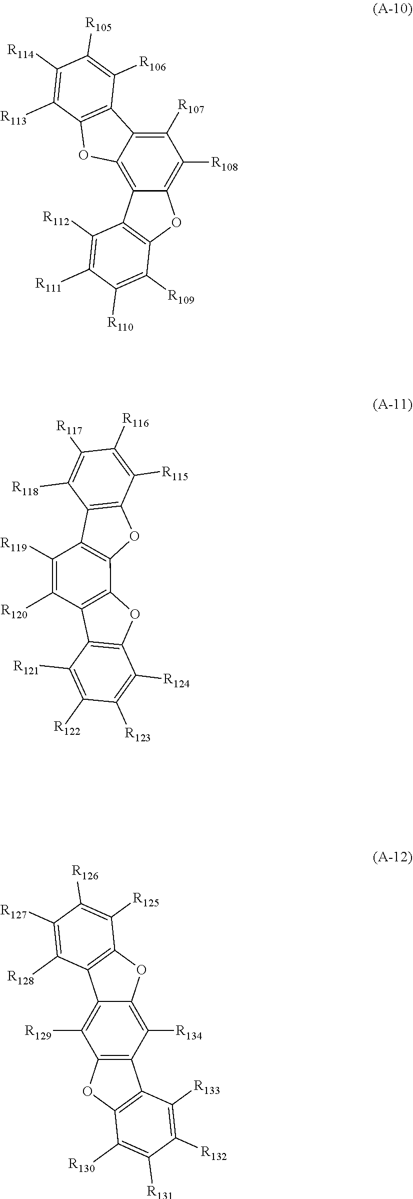
one selected from R₁ to R₁₂, one selected from R₂₁ to R₃₀, one selected from R₃₁ to R₄₀, one selected from R₄₁ to R₅₀, one selected from R₅₁ to R₆₀, one selected from R₆₁ to R₇₂, one selected from R₇₃ to R₈₆, one selected from R₈₇ to R₉₄, one selected from R₉₅ to R₁₀₄, one selected from R₁₀₅ to R₁₁₄, one selected from R₁₁₅ to R₁₂₄, and one selected from R₁₂₅ to R₁₃₃ are single bonds each bonded to L⁷⁷;
or, two to four selected from R₁ to R₁₂, two to four selected from R₂₁ to R₃₀, two to four selected from R₃₁ to R₄₀, two to four selected from R₄₁ to R₅₀, two to four selected from R₅₁ to R₆₀, two to four selected from R₆₁ to R₇₂, two to four selected from R₇₃ to R₈₆, two to four selected from R₈₇ to R₉₄, two to four selected from R₉₅ to R₁₀₄, two to four selected from R₁₀₅ to R₁₁₄, two to four selected from R₁₁₅ to R₁₂₄, and two to four selected from R₁₂₅ to R₁₃₃ are single bonds, wherein one of the single bonds is bonded to L⁷⁷ and the other single bonds are bonded to Ar⁶⁶;
R₁ to R₁₂, R₂₁ to R₃₀, R₃₁ to R₄₀, R₄₁ to R₅₀, R₅₁ to R₆₀, R₆₁ to R₇₂, R₇₃ to R₈₆, R₈₇ to R₉₄, R₉₅ to R₁₀₄, R₁₀₅ to R₁₁₄, R₁₁₅ to R₁₂₄, and R₁₂₅ to R₁₃₃ each not the single bond are each independently a hydrogen atom, a halogen atom, a cyano group, a substituted or unsubstituted alkyl group having 1 to 20 carbon atoms, a substituted or unsubstituted cycloalkyl group having 3 to 20 ring carbon atoms, a group represented by —Si(R₁₀₁)(R₁₀₂)(R₁₀₃), or a substituted or unsubstituted aryl group having 6 to 50 ring carbon atoms; and
adjacent two selected from R₁ to R₁₂, R₂₁ to R₃₀, R₃₁ to R₄₀, R₄₁ to R₅₀, R₅₁ to R₆₀, R₆₁ to R₇₂, R₇₃ to R₈₆, R₈₇ to R₉₄, R₉₅ to R₁₀₄, R₁₀₅ to R₁₁₄, R₁₁₅ to R₁₂₄, and R₁₂₅ to R₁₃₃ each not the single bond may be bonded to each other to form a substituted or unsubstituted ring structure.

2. The organic electroluminescence device according to claim 1, wherein the content of the second compound in the fluorescent emitting layer is less than that of the first compound in the fluorescent emitting layer.

3. The organic electroluminescence device according to claim 1, wherein the content of the second compound in the fluorescent emitting layer is 30% by mass or less based on a total amount of the first compound, the second compound, and the dopant material.

4. The organic electroluminescence device according to claim 1, wherein the content of the dopant material in the fluorescent emitting layer is 10% by mass or less based on a total amount of the first compound, the second compound, and the dopant material.

5. The organic electroluminescence device according to claim 1, wherein formula (19) is represented by formula (20): 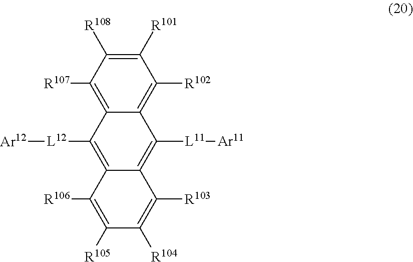
wherein:
R¹⁰¹ to R¹⁰⁸ are as defined above;
Ar¹¹ and Ar¹² are each independently as defined above with respect to Ar; and
L¹¹ and L¹² are each independently as defined above with respect to L.

6. The organic electroluminescence device according to claim 1, wherein the dopant material represented by formula (D1) includes a compound represented by formula (D1a): 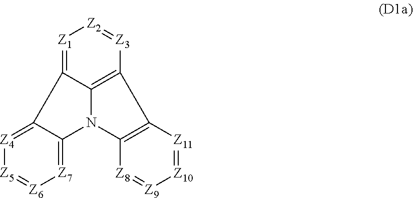
wherein:
Z₁ is CR₁ or N, Z₂ is CR₂ or N, Z₃ is CR₃ or N, Z₄ is CR₄ or N, Z₅ is CR₅ or N, Z₆ is CR₆ or N, Z₇ is CR₇ or N, Z₈ is CR₈ or N, Z₉ is CR₉ or N, Z₁₀ is CR₁₀ or N, and Z₁₁ is CR₁₁ or N;
R₁ to R₁₁ are each independently a hydrogen atom or a substituent, wherein the substituent is as described above with respect to the substituent of R_{A}, R_{B}, and R_{C};
adjacent two selected from R₁ to R₃ may be bonded to each other to form a substituted or unsubstituted ring structure or not bonded to each other, thereby failing to form a ring structure;
adjacent two selected from R₄ to R₇ may be bonded to each other to form a substituted or unsubstituted ring structure or not bonded to each other, thereby failing to form a ring structure; and
adjacent two selected from R₈ to R₁₁ may be bonded to each other to form a substituted or unsubstituted ring structure or not bonded to each other, thereby failing to form a ring structure.

7. The organic electroluminescence device according to claim 1, wherein the dopant material represented by formula (D1) includes a compound represented by formula (1): 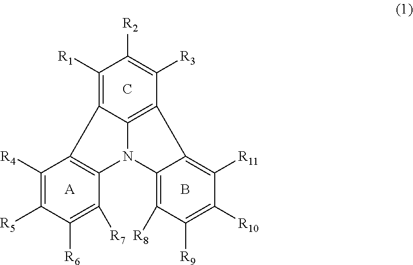
wherein:
Rₙ and Rₙ₊₁, wherein n is an integer selected from 1, 2, 4 to 6, and 8 to 10, may be bonded to each other to form, together with two ring carbon atoms to which Rₙ and Rₙ₊₁ are bonded, a substituted or unsubstituted ring structure having 3 or more ring atoms or Rₙ and Rₙ₊₁ may be not bonded to each other, thereby failing to form a ring structure;
the ring atom is selected from a carbon atom, an oxygen atom, a sulfur atom, and a nitrogen atom;
an optional substituent of the ring structure having 3 or more ring atoms is as defined above with respect to the substituent of R_{A}, R_{B}, and R_{C} and adjacent two optional substituents may be bonded to each other to form a substituted or unsubstituted ring structure; and
R₁ to R₁₁ not forming the substituted or unsubstituted ring structure having 3 or more ring atoms are as defined above.

8. The organic electroluminescence device according to claim 7, wherein the substituted or unsubstituted ring structure having 3 or more ring atoms is selected from formula (2) to (8): 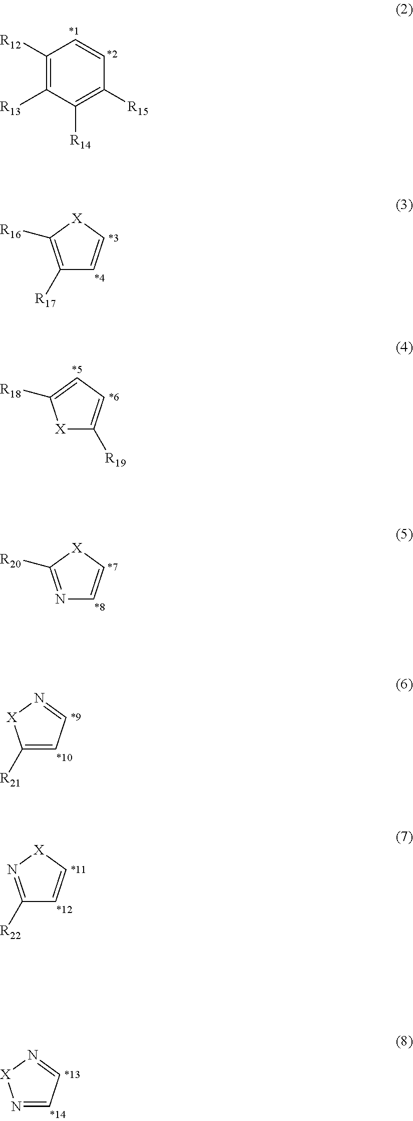
wherein:
*1 and *2, *3 and *4, *5 and *6, *7 and *8, *9 and *10, *11 and *12, and *13 and *14 are two ring carbon atoms to which Rₙ and Rₙ₊₁ are bonded, wherein Rₙ may be bonded to either of the two ring carbon atoms;
X is selected from C(R₂₃)(R₂₄), NR₂₅, O, and S;
R₁₂ to R₂₅ are each independently a hydrogen atom or a substituent, wherein the substituent is as described above with respect to the substituent of R_{A}, R_{B}, and R_{C}; and
adjacent two selected from R₁₂ to R₁₅, R₁₆ and R₁₇, and R₂₃ and R₂₄ may be bonded to each other to form a substituted or unsubstituted ring structure.

9. The organic electroluminescence device according to claim 7, wherein the substituted or unsubstituted ring structure having 3 or more ring atoms is selected from formulae (9) to (11): 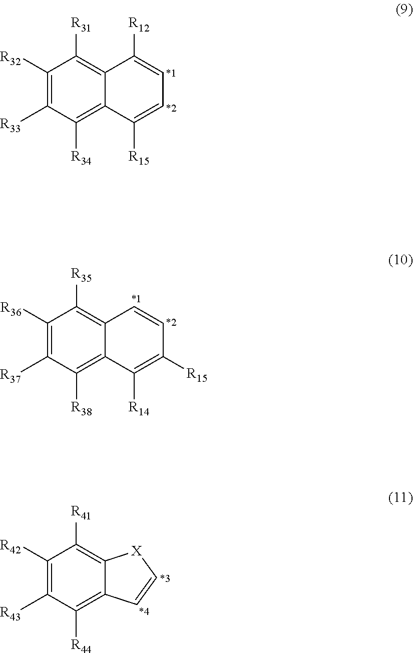
wherein:
*1 and *2, and *3 and *4 are as defined above;
R₁₂, R₁₄, R₁₅, and X are as defined above;
R₃₁ to R₃₈ and R₄₁ to R₄₄ are each independently a hydrogen atom or a substituent, wherein the substituent is as described above with respect to the substituent of R_{A}, R_{B}, and R_{C}; and
adjacent two selected from R₁₂, R₁₅, and R₃₁ to R₃₄, adjacent two selected from R₁₄, R₁₅, and R₃₅ to R₃₈, and adjacent two selected from R₄₁ to R₄₄ may be bonded to each other to form a substituted or unsubstituted ring structure.

10. The organic electroluminescence device according to claim 7, wherein at least one of R₂, R₄, R₅, R₁₀, and R₁₁ of formula (1) does not form a substituted or unsubstituted ring structure having 3 or more ring atoms.

11. The organic electroluminescence device according to claim 7, wherein an optional substituent of the ring structure having 3 or more ring atoms in formula (1) is independently a substituted or unsubstituted alkyl group having 1 to 20 carbon atoms, a group represented by —N(R₁₀₄)(R₁₀₅), wherein R₁₀₄ and R₁₀₅ are as defined above, a substituted or unsubstituted aryl group having 6 to 50 ring carbon atoms, a substituted or unsubstituted heteroaryl group having 5 to 50 ring atoms, or any one selected from the following groups: 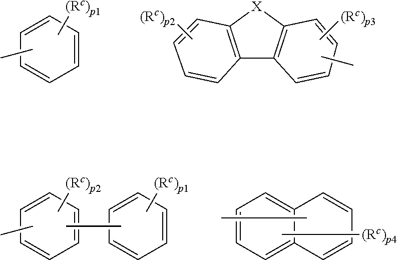
wherein:
each R^{c} is independently a hydrogen atom or a substituent, wherein the substituent is as described above with respect to the substituent of R_{A}, R_{B}, and R_{C};
X is as defined above; and
p1 is an integer of 0 to 5, p2 is an integer of 0 to 4, p3 is an integer of 0 to 3, and p4 is an integer of 0 to 7.

12. The organic electroluminescence device according to claim 7, wherein R₁ to R₁₁ of formula (1) not forming the substituted or unsubstituted ring structure having 3 or more ring atoms are each independently a hydrogen atom, a substituted or unsubstituted alkyl group having 1 to 20 carbon atoms, a group represented by —N(R₁₀₄)(R₁₀₅), wherein R₁₀₄ and R₁₀₅ are as defined above, a substituted or unsubstituted aryl group having 6 to 50 ring carbon atoms, a substituted or unsubstituted heteroaryl group having 5 to 50 ring atoms, or any one selected from the following groups: 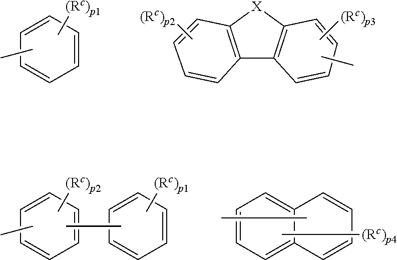
wherein:
each R^{c} is independently a hydrogen atom or a substituent, wherein the substituent is as described above with respect to the substituent of R_{A}, R_{B}, and R_{C};
X is as defined above; and
p1 is an integer of 0 to 5, p2 is an integer of 0 to 4, p3 is an integer of 0 to 3, and p4 is an integer of 0 to 7.

13. The organic electroluminescence device according to claim 8, wherein R₁₂ to R₂₂, R₃₁ to R₃₈, and R₄₁ to R₄₄ of formulae (2) to (11) are each independently a hydrogen atom, a substituted or unsubstituted alkyl group having 1 to 20 carbon atoms, a group represented by —N(R₁₀₄)(R₁₀₅), wherein R₁₀₄ and R₁₀₅ are as defined above, a substituted or unsubstituted aryl group having 6 to 50 ring carbon atoms, a substituted or unsubstituted heteroaryl group having 5 to 50 ring atoms, or any one selected from the following groups: 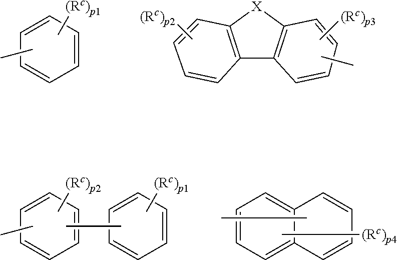
wherein:
each R^{c} is independently a hydrogen atom or a substituent, wherein the substituent is as described above with respect to the substituent of R_{A}, R_{B}, and R_{C};
X is as defined above; and
p1 is an integer of 0 to 5, p2 is an integer of 0 to 4, p3 is an integer of 0 to 3, and p4 is an integer of 0 to 7.

14. The organic electroluminescence device according to claim 7, wherein the dopant material represented by formula (1) includes a compound represented by any of formulae (1-1) to (1-3) and (1-5): 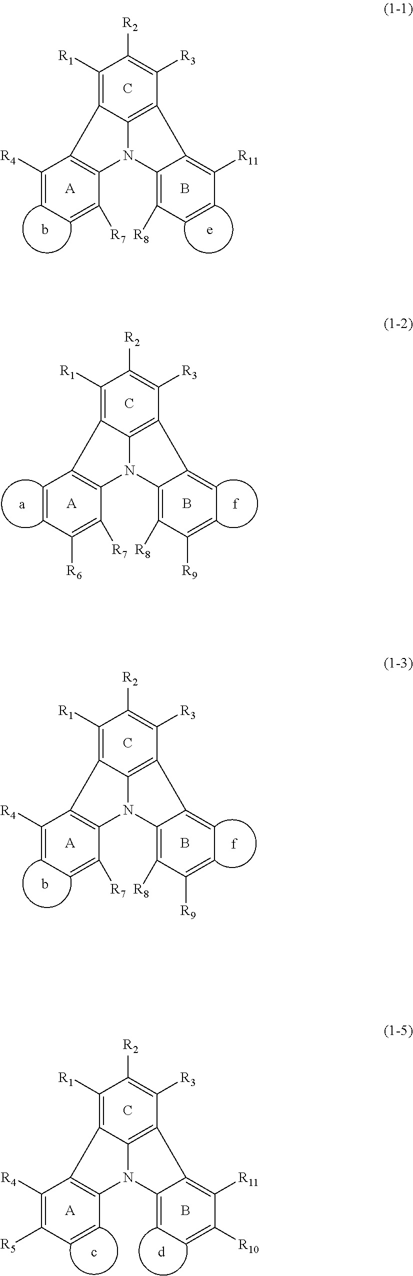
wherein:
R₁ to R₁₁ are as defined above; and
the rings a to f are each independently the substituted or unsubstituted ring structure having 3 or more ring atoms.

15. The organic electroluminescence device according to claim 7, wherein the dopant material represented by formula (1) includes a compound represented by any of formulae (2-2) and (2-5): 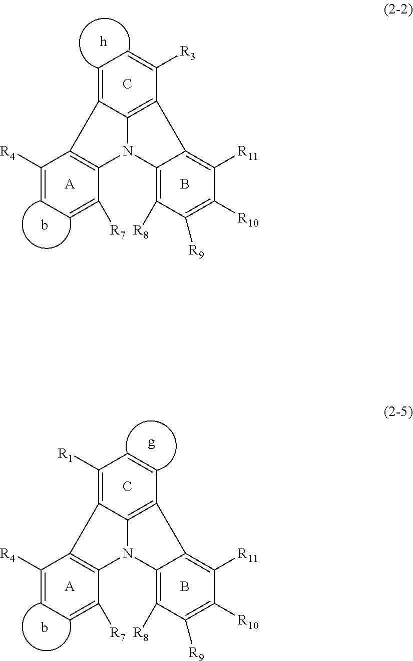
wherein:
R₁, R₃, R₄, and R₇ to R₁₁ are as defined above; and
the rings b and g to h are each independently the substituted or unsubstituted ring structure having 3 or more ring atoms.

16. The organic electroluminescence device according to claim 7, wherein the dopant material represented by formula (1) includes a compound represented by formula (3-1): 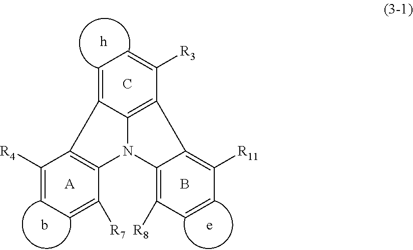
wherein:
R₃, R₄, R₇, R₈, and R₁₁ are as defined above; and
the rings b, e, and h are each independently the substituted or unsubstituted ring structure having 3 or more ring atoms.

17. The organic electroluminescence device according to claim 14, wherein an optional substituent of the rings a to f is independently a substituted or unsubstituted alkyl group having 1 to 20 carbon atoms, a group represented by —N(R₁₀₄)(R₁₀₅), wherein R₁₀₄ and R₁₀₅ are as defined above, a substituted or unsubstituted aryl group having 6 to 50 ring carbon atoms, a substituted or unsubstituted heteroaryl group having 5 to 50 ring atoms, or any one selected from the following groups: 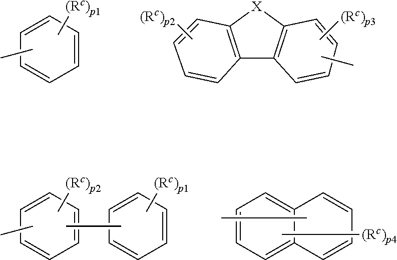
wherein:
each R^{c} is independently a hydrogen atom or a substituent, wherein the substituent is as described above with respect to the substituent of R_{A}, R_{B}, and R_{C};
X is as defined above; and
p1 is an integer of 0 to 5, p2 is an integer of 0 to 4, p3 is an integer of 0 to 3, and p4 is an integer of 0 to 7.

18. The organic electroluminescence device according to claim 7, wherein the dopant material represented by formula (1) includes a compound represented by any of formulae (4-1) to (4-4): 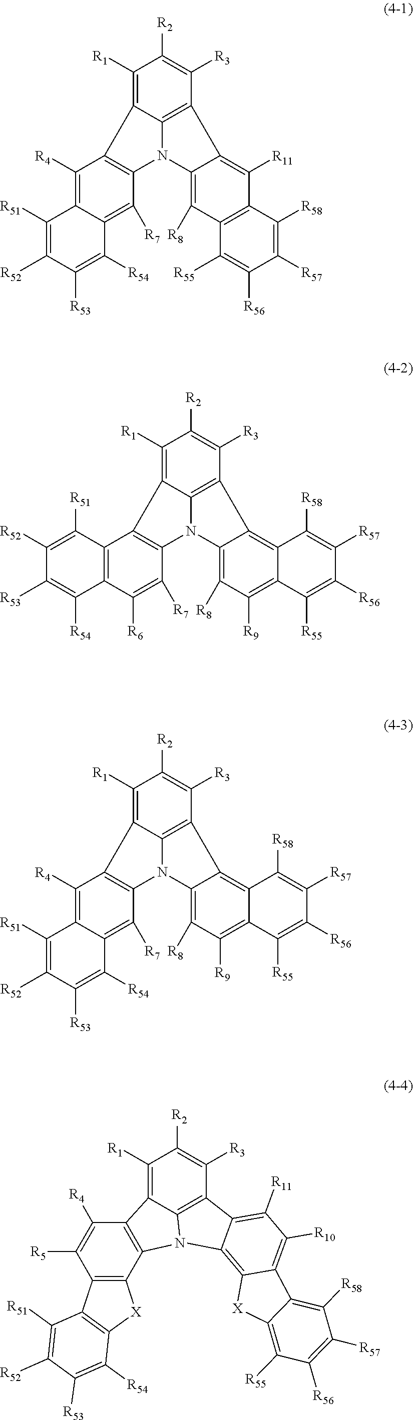
wherein:
X and R₁ to R₁₁ are as defined above;
R₅₁ to R₅₈ are each independently a hydrogen atom or a substituent, wherein the substituent is as described above with respect to the substituent of R_{A}, R_{B}, and R_{C}.

19. The organic electroluminescence device according to claim 7, wherein the dopant material represented by formula (1) includes a compound represented by formula (5-1): 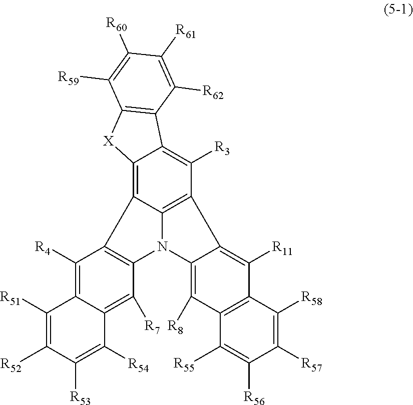
wherein:
X, R₃, R₄, R₇, R₈, and R₁₁ are as defined above; and
R₅₁ to R₆₂ are each independently a hydrogen atom or a substituent, wherein the substituent is as described above with respect to the substituent of R_{A}, R_{B}, and R_{C}.

20. The organic electroluminescence device according to claim 7, wherein Rₙ and Rₙ₊₁ of formula (1) are bonded to each other to form at least two substituted or unsubstituted ring structures each having 3 or more ring atoms.

21. The organic electroluminescence device according to claim 7, wherein
a pair of R₁ and R₂ and a pair of R₂ and R₃;
a pair of R₄ and R₅ and a pair of R₅ and R₆;
a pair of R₅ and R₆ and a pair of R₆ and R₇;
a pair of R₈ and R₉ and a pair of R₉ and R₁₀; and
a pair of R₉ and R₁₀ and a pair of R₁₀ and R₁₁
do not form the substituted or unsubstituted ring structure having 3 or more ring atoms at the same time.

22. The organic electroluminescence device according to claim 1, wherein the dopant material represented by formula (D2) includes a compound represented by formula (D2a): 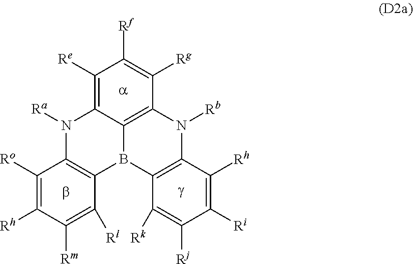
wherein:
R^{a} and R^{b} are as defined above;
R^{e} to R^{o} are each independently a hydrogen atom; a substituted or unsubstituted aryl group having 6 to 50 ring carbon atoms; a substituted or unsubstituted heteroaryl group having 5 to 50 ring atoms; a diarylamino group, a diheteroarylamino group or an arylheteroarylamino group each having a substituent selected from a substituted or unsubstituted aryl group having 6 to 50 ring carbon atoms and a substituted or unsubstituted heteroaryl group having 5 to 50 ring atoms; a substituted or unsubstituted alkyl group having 1 to 20 carbon atoms; a substituted or unsubstituted alkoxy group having 1 to 20 carbon atoms; or a substituted or unsubstituted aryloxy group having 6 to 50 ring carbon atoms; and
adjacent two selected from R^{e} to R^{g}, adjacent two selected from R^{h} to R^{k}, and adjacent two selected from R^{l} to R^{o} may be bonded to each other to form a substituted or unsubstituted aromatic hydrocarbon ring having 6 to 50 ring carbon atoms or a substituted or unsubstituted aromatic heterocyclic ring having 5 to 50 ring atoms.

23. The organic electroluminescence device according to claim 1, wherein the fluorescent emitting layer does not include a heavy metal complex.

24. The organic electroluminescence device according to claim 1, which emits blue light.

25. An electronic device comprising the organic electroluminescence device according to claim 1.
